SEQUENCE LISTING (8 sequences)

SEQ ID NO: 1 - protein, 414 aa, Artificial Sequence
  GST-hK-RasG12C (G12C mutation according to numbering in P01116-2)
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDPITSLYKKAGSDYDIPTTENLYFQGMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 2 - protein, 533 aa, Artificial Sequence
  His10-hSOS1
MGHHHHHHHHHHSSGHIEGRHMLETSLYKKAGSDYDIPTTENLYFQGEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 3 - protein, 536 aa, Artificial Sequence
  His10-hSOS1_12
MGHHHHHHHHHHSSGHIEGRHMLETSLYKKAGSDYDIPTTENLYFQGAMAEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 4 - protein, 490 aa, Artificial Sequence
  hSOS1_12 (tag-free)
GAMAEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 5 - protein, 527 aa, Artificial Sequence
  His10-hSOS2
MGHHHHHHHHHHSSGHIEGRHMLETSLYKKAGSDYDIPTTENLYFQGPLRLPSPEVYRFVVKDSEENIVFEDNLQSRSGIPIIKGGTVVKLIERLTYHMYADPNFVRTFLTTYRSFCKPQELLSLLIERFEIPEPEPTDADKLAIEKGEQPISADLKRFRKEYVQPVQLRILNVFRHWVEHHFYDFERDLELLERLESFISSVRGKAMKKWVESIAKIIRRKKQAQANGVSHNITFESPPPPIEWHISKPGQFETFDLMTLHPIEIARQLTLLESDLYRKVQPSELVGSVWTKEDKEINSPNLLKMIRHTTNLTLWFEKCIVEAENFEERVAVLSRIIEILQVFQDLNNFNGVLEIVSAVNSVSVYRLDHTFEALQERKRKILDEAVELSQDHFKKYLVKLKSINPPCVPFFGIYLTNILKTEEGNNDFLKKKGKDLINFSKRRKVAEITGEIQQYQNQPYCLRIEPDMRRFFENLNPMGSASEKEFTDYLFNKSLEIEPRNCKQPPRFPRKSTFSLKSPGIRPNTG

SEQ ID NO: 6 - protein, 536 aa, Artificial Sequence; identical to SEQ ID NO: 3
  hSOS1_12 with N-terminal His tag (His10-hSOS1_12) before cleavage by TEV protease

SEQ ID NO: 7 - protein, 490 aa, Artificial Sequence; identical to SEQ ID NO: 4
  hSOS1_12 after cleavage by TEV protease

SEQ ID NO: 8 - protein, 14 aa, Artificial Sequence
  Synthetic Construct
  VARIANT 1: Modified by Biotin-Aminocaproic acid (Biotin-Ahx)
  AMIDATION 14
AEEEEYFELVAKKK

(19) United States
(12) Patent Application Publication
(10) Pub. No.: US 2022/0274979 A1
(43) Pub. Date: Sep. 1, 2022

(54) 2-METHYL-AZA-QUINAZOLINES

(71) Applicants: Bayer Pharma Aktiengesellschaft, Berlin (DE); Bayer Healthcare China, Beijing (CN)

(72) Inventors: Lars WORTMANN, Biberbach an der Riß (DE); Brice SAUTIER, Roques (FR); Knut EIS, Newton, MA (US); Hans BRIEM, Berlin (DE); Niels BÖHNKE, Berlin (DE); Franz VON NUSSBAUM, Berlin (DE); Roman HILIG, Berlin (DE); Benjamin BADER, Berlin (DE); Jens SCHRÖDER, Berlin (DE); Kirstin PETERSEN, Berlin (DE); Philip LIENAU, Berlin (DE); Antje Margret WENGNER, Berlin (DE); Dieter MOOSMAYER, Berlin (DE); Qiuwen WANG, Beijing (CN); Hans SCHICK, Berlin (DE)

(73) Assignees: Bayer Pharma Aktiengesellschaft, Berlin (DE); Bayer Healthcare China, Beijing (CN)

(21) Appl. No.: 17/048,561

(22) PCT Filed: Apr. 15, 2019
(86) PCT No.: PCT/EP2019/059650

§ 371 Date: Oct. 16, 2020

(30) Foreign Application Priority Data
Apr. 18, 2018 (CN) PCT/CN2018/083496

(51) Int. Cl. C07D 471/04; C07D 487/04

(52) U.S. Cl. CPC C07D 471/04; C07D 487/04

(57) ABSTRACT

The present invention covers 2-methyl-aza-quinazoline compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of hyperproliferative disorders, as a sole agent or in combination with other active ingredients

### CROSS REFERENCE TO RELATED APPLICATIONS

This application is a national stage application under 35 U.S.C. § 371 of International Application No. PCT/EP2019/059650, filed internationally on Apr. 15, 2019, which claims benefit of PCT/CN2018/083496, filed Apr. 18, 2018.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 777052044800SEQLIST.TXT, date recorded: Oct. 14, 2020, 2020, size: 30 KB).

The present invention covers 2-methyl-aza-quinazoline compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of hyperproliferative disorders, as a sole agent or in combination with other active ingredients.

### BACKGROUND

The present invention covers 2-methyl-aza-quinazoline compounds of general formula (I) which inhibit the Ras-Sos interaction.

US 2011/0054173 A1 discloses certain 1- or 2-(4-(aryloxy)-phenyl)ethylamino-, oxy- or sulfanyl)pteridines and 1- or 2-(4-(heteroaryloxy)-phenyl)ethylamino-, oxy- or sulfanyl)pteridines and their use as agrochemicals and animal health products.

In the 2-position substituted quinazoline compounds are described e.g. in EP 0326328, EP 0326329, WO93/007124, WO2003/087098 and U.S. Pat. No. 5,236,925. These compounds are either not described as pharmaceutically active compounds or, if they are described as pharmacologically active compounds, they are described as compounds having affinity to the Epidermal Growth Factor Receptor (EGFR).

In the majority (45-100%) of patients receiving EGFR inhibitors skin toxicity is a class-specific side effect that is typically manifested as a papulopustular rash. The skin toxicity is related to the inhibition of EGFR in the skin, which is crucial for the normal development and physiology of the epidermis.

However, the state of the art does not describe:
the 2-methyl substituted quinazoline compounds of general formula (I) of the present invention as described and defined herein, i.e. compounds having a quinazoline core bearing a methyl group on the carbon atom 2 which effectively and selectively inhibit the Ras-Sos interaction without significantly targeting the EGFR receptor.

Ras proteins play an important role in human cancer. Mutations in Ras proteins can be found in 20-30% of all human tumors and are recognized as tumorigenic drivers especially in lung, colorectal and pancreatic cancers (Malumbres & Barbacid 2002 Nature Reviews Cancer, Pylayeva-Gupta et al. 2011 Nature Reviews Cancer). Three human Ras genes are known that encode four different Ras proteins of 21 kDa size: H-Ras, N-Ras, and two splice variants of K-Ras, namely K-Ras 4A and K-Ras-4B. All Ras isoforms are highly conserved within the GTP-binding domain and differ mainly in the hypervariable C-terminal region. The C-termini of the different Ras-isoforms are posttranslationally modified by lipidation (farnesylation, palmitoylation) to facilitate membrane anchorage. The localization of Ras-proteins at the cytoplasmic membrane provides vicinity to transmembrane growth receptors and has been shown to be essential for transmitting growth signals from extracellular growth factor binding to intracellular downstream pathways. A variety of upstream signals may activate Ras proteins depending on the cellular context, such as epidermal growth factor receptor (EGFR), platelet-derived growth factor receptor (PDGFR), nerve growth factor receptor (NGFR) and others. Activated Ras can signal through various downstream pathways, e.g. the Raf-MEK-ERK or the PI3 K-PD Kl-Akt pathways.

On the molecular level, Ras proteins function as molecular switches. By binding GTP and GDP they exist in an active (GTP-bound) and inactive (GDP-bound) state in the cell. Active GTP-loaded Ras recruits other proteins by binding of their cognate Ras-binding domains (RBDs) resulting in activation of the effector protein followed by downstream signalling events of diverse functions, e.g. cytoskeletal rearrangements or transcriptional activation. The activity status of Ras is tightly regulated by guanine nucleotide exchange factors (GEFs) and GTPase activating proteins (GAPs). GEFs function as activators of Ras by promoting the nucleotide exchange from GDP to GTP. GAPs deactivate Ras-GTP by catalyzing the hydrolysis of the bound GTP to GDP. In a cancer cell, point mutations, typically within the GTP-binding region at codon 12, eliminate the ability of RAS to efficiently hydrolyse bound GTP, even in the presence of a GAP. Therefore, cancer cells comprise increased levels of active mutated Ras-GTP, which is thought to be a key factor for driving cancer cell proliferation.

Three main families of RAS-specific GEFs have been identified so far (reviewed in Vigil 2010 Nature Reviews Cancer; Rojas et al 2011, Genes & Cancer 2(3) 298-305). There are two son of sevenless proteins (SOS1 and SOS2), 4 different isoforms of Ras guanine nucleotide releasing proteins (Ras-GRP1-4) and two Ras guanine nucleotide releasing factors (Ras-GRF1 and 2). The SOS proteins are ubiquitously expressed and are recruited to sites of activated growth factors. Ras-GRFs are expressed mainly in the nervous system, where they are involved in Calcium-dependent activation of Ras. In contrast, Ras GRP proteins are expressed in hematopoietic cells and act in concert with non-receptor tyrosine kinases. In the context of cancer, mainly SOS proteins have been found to be involved.

Targeting Ras for cancer therapy has been a dream since the 1990s (Downward 2002 Nature Reviews Cancer, Krens et al. 2010 Drug Discovery Today). Due to the compact nature, the high affinity towards GDP and GTP in combination with high intracellular GTP concentrations, the Ras protein itself has always been considered to be undruggable, i.e. the chance to identify small chemical molecules that would bind to and inhibit active Ras was rated extremely low. Alternative approaches have been undertaken to reduce Ras signaling, e.g. by addressing more promising drug targets such as enzymes involved in the posttranslational modification of Ras proteins, especially farnesyltransferase and geranylgeranyltransferase (Berndt 2011 Nature Reviews Cancer). Inhibitors of farnesyltransferase (FTIs) were identified and developed with promising antitumor effects in preclinical models. Unexpectedly, in clinical trials these inhibitors have been of limited efficacy. Targeting upstream and downstream kinases involved in Ras signaling pathways has been more successful. Several drugs are and have been in clinical trials that inhibit different kinases, e.g. EGFR, Raf, MEK, Akt, PI3K (Takashima & Faller 2013 Expert Opin. Ther. Targets). Marketed cancer drugs are available that inhibit Raf, EGFR or MEK.

Nevertheless, there is still a large unmet need for the treatment of Ras-dependent tumors that are resistant against current therapies. Many research groups have been active to identify small molecules that target Ras directly (Ras small molecules have been reviewed in: Cox et al. 2014 Nature Reviews Drug Discovery; Stephen et al. 2014 Cancer Cell; Hattum & Waldmann 2014 Chemistry & Biology, Spiegel et al. 2014 Nature Chemical Biology). One group of inhibitors comprises small molecules that inhibit the interaction of Ras with its effectors Raf or PI3K. Another group of compounds acts as covalent inhibitors of a specific cysteine mutant form of K-Ras (glycine to cysteine point mutation G12C). The specific targeting of the Ras-G12C mutant might have the benefit of reduced side effects, as the wildtype Ras proteins should not be affected. Furthermore, several reports show small molecules and peptides that interrupt the GEF assisted activation of Ras. There seem to be several different binding sites possible that result in this mode of action. Inhibitors may bind to Ras or to the GEF in an allosteric or orthosteric fashion. All these approaches of direct Ras-targeting are in preclinical research stage and the affinity of published small molecule inhibitors is still in the micromolar range. Stabilized peptides have been shown to be active in the nanomolar range. (Leshchiner et al. 2015 PNAS). Their usefulness as drugs in a clinical setting has to be awaited.

The Epidermal Growth Factor Receptor (EGFR) is a tyrosine kinase (TK) receptor that is activated upon binding to the Epidermal Growth Factor and other growth factor ligands, triggering several downstream pathways, including RAS/MAPK, PI3K/Akt and STAT that regulate different cellular processes, including DNA synthesis and proliferation (Russo A, Oncotarget.4254, 2015). The family of HER (ErbB) receptor tyrosine kinases consists of four members, ie, epidermal growth factor receptors [EGFR (HER1 or ErbB1), HER2 (ErbB2, neu), HERS (ErbB3), and HER4 (ErbB4)]. Overexpression, mutation, or aberrant activity of these receptors has been implicated in various types of cancer (Feldinger K, Breast Cancer (Dove Med Press), 2015, 7, 147).

First-Generation Inhibitors

Erlotinib and Gefitinib are small molecule inhibitors of the EGFR/HER-1 (human epidermal growth factor receptor) tyrosine kinase. Erlotinib and Gefitinib were developed as reversible and highly specific small-molecule tyrosine kinase inhibitors that competitively block the binding of adenosine triphosphate to its binding site in the tyrosine kinase domain of EGFR, thereby inhibiting autophosphorylation and blocking downstream signaling (Cataldo V D, N Engl J Med, 2011, 364, 947).

Second-Generation Inhibitors

Afatinib is an oral tyrosine kinase inhibitor (TKI) approved for the first-line treatment of patients with NSCLC whose tumors are driven by activating mutations of genes coding for epidermal growth factor receptor (EGFR). Afatinib is also an inhibitor of a specific EGFR mutation (T790M) that causes resistance to first-generation EGFR-targeted TKIs in about half of patients receiving those drugs. (Engle J A, Am J Health Syst Pharm 2014, 71 (22), 1933).

Neratinib, a pan-HER inhibitor, irreversible tyrosine kinase inhibitor binds and inhibits the tyrosine kinase activity of epidermal growth factor receptors, EGFR (or HER1), HER2 and HER4, which leads to reduced phosphorylation and activation of downstream signaling pathways. Neratinib has been shown to be effective against HER2-overexpressing or mutant tumors in vitro and in vivo. Neratinib is currently being investigated in various clinical trials in breast cancers and other solid tumors, including those with HER2 mutation (Feldinger K, Breast Cancer (Dove Med Press), 2015, 7, 147).

Dacomitinib is an irreversible inhibitor of EGFR, HER2, and HER4. In preclinical cell lines and xenograft studies, dacomitinib demonstrated activities against both activating EGFR mutations and EGFR T790M (Liao B C, Curr Opin Oncol. 2015, 27(2), 94).

Third-Generation Inhibitors

The third-generation EGFR-TKIs were designed to inhibit EGFR T790M while sparing wild-type EGFR.

AZD9291 (AstraZeneca, Macclesfield, UK), a mono-anilino-pyrimidine compound, is an irreversible mutant selective EGFR-TKI. This drug is structurally different from the first and second-generation EGFR-TKIs. In preclinical studies, it potently inhibited phosphorylation of EGFR in cell lines with activating EGFR mutations (EGFR dell9 and EGFR L858R) and EGFR T790M. AZD9291 also caused profound and sustained tumor regression in tumor xenograft and transgenic mouse models harboring activating EGFR mutations and EGFR T790M. AZD9291 was less potent in inhibiting phosphorylation of wild-type EGFR cell lines (Liao B C, Curr Opin Oncol. 2015, 27(2), 94).

Rociletinib (CO-1686) (Clovis Oncology, Boulder, Colo), a 2,4-disubstituted pyrimidine molecule, is an irreversible mutant selective EGFR-TKI. In preclinical studies, CO-1686 led to tumor regression in cell-lines, xenograft models, and transgenic mouse models harboring activating EGFR mutations and EGFR T790M (Walter A O, Cancer Discov, 2013, 3(12), 1404).

HM61713 (Hanmi Pharmaceutical Company Ltd, Seoul, South Korea) is an orally administered, selective inhibitor for activating EGFR mutations and EGFR T790M. It has low activity against wild-type EGFR (Steuer C E, Cancer. 2015, 121(8), E1).

It has now been found, and this constitutes the basis of the present invention, that the compounds of the present invention have surprising and advantageous properties.

In particular, the compounds of the present invention have surprisingly been found to effectively and selectively inhibit the Ras-Sos interaction without significantly targeting the EGFR receptor and may therefore be used for the treatment or prophylaxis of hyper-proliferative disorders, in particular cancer.

### DESCRIPTION OF THE INVENTION

In accordance with a first aspect, the present invention covers compounds of general formula (I):

1.

2.

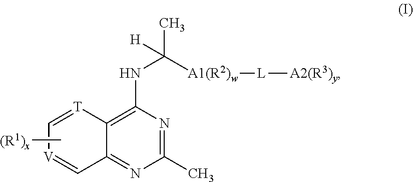

3. in which

4. R¹ stands for
a substituent independently selected from: a hydrogen atom, a halogen atom, a hydroxy, cyano, nitro, C₁-C₆-alkylsulfanyl or an amino group NR^{a}R^{b},
   wherein R^{a} and R^{b} are selected independently from a hydrogen atom or a C₁-C₆-alkyl,
a substituent selected from: a C₁-C₆-alkyl, C₁-C₆-alkoxy-, C₂-C₆-alkenyl, C₂-C₆-alkynyl, C₃-C₈-cycloalkyl, C₄-C₈-cycloalkenyl, 4- to 7-membered heterocycloalkyl, 5- to 10-membered heterocycloalkenyl, heterospirocycloalkyl, fused heterocycloalkyl, bridged heterocycloalkyl, phenyl, heteroaryl, C₁-C₆-haloalkyl,
—C(═O)OH, —C(═O)OR^{c}, and wherein R^{e} stands for C₁-C₆-alkyl, C₃-C₆-alkenyl, C₃-C₆-alkynyl, C₃-C₈-cycloalkyl or C₄-C₈-cycloalkenyl,
—N═S(═O)(R^{d})R^{e}, and wherein R^{d} and R^{e} are selected independently from hydrogen, C₁-C₆-alkyl, C₂-C₆-alkenyl, C₂-C₆-alkynyl, C₃-C₈-cycloalkyl or C₄-C₈-cycloalkenyl,
—NH—C(O)—C₁-C₆-alkyl, —NH—C(O)—N R^{a}R^{b}, wherein R^{a} and R^{b} are selected independently from a hydrogen atom or a C₁-C₆-alkyl, —NH—(CH₂)ₖ—NH—C(O)—C₁-C₆-alkyl, wherein k is 1 or 2, —NH—(CH₂)₁—R^{f}, wherein
   1 is 0, 1 or 2 and R^{f} stands for a 4- to 7-membered heterocycloalkyl, heteroaryl, C₁-C₆-alkylsulfonyl,
   whereby in all foregoing definitions the C₁-C₆-alkyl-, C₁-C₆-alkoxy-, the 4- to 7-membered heterocycloalkyl and the heteroaryl can be optionally substituted, one, two or three times, identically or differently, with:
      a halogen atom, or a group selected from hydroxy, oxo (═O), a cyano, nitro, C₁-C₆-alkyl, C₂-C₆-alkenyl, C₂-C₆-alkynyl, C₃-C₈-cycloalkyl, 4- to 7-membered heterocycloalkyl, C₁-C₆-alkoxy, C₁-C₆-haloalkyl, C₁-C₆-haloalkoxy-, C₁-C₆-alkylsulfonyl, phenyl, benzyl-, heteroaryl, -(CH₂)-heteroaryl-, C₃-C₈-cycloalkoxy-, phenyloxy-, heteroaryloxy-, —NH—C(O)—C₁-C₆-alkyl or an amino group NR^{a}R^{b}, wherein R^{a} and R^{b} are selected independently from a hydrogen atom or a C₁-C₆-alkyl, or
a substituent

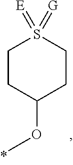

wherein E and G each stands for an electron pair, or one of E and G stands for an electron pair and the other for an oxygen atom or a group ═NH or ═N—C₁-C₄-alkyl, or one of E and G stands for an oxygen atom and the other one for a group ═NH or ═N—C₁-C₄-alkyl, or E and G each stands for an oxygen atom or each stands for a group ═NH or ═N—C₁-C₄-alkyl, or
a substituent —O—(CH₂)_{z}-phenyl, —O—(CH₂)_{z}—C₄-C₇-heterocycloalkyl, —O—(CH₂)_{z}-heteroaryl, whereby
   z is 0, 1 or 2, and the phenyl, heterocycloalkyl and heteroaryl can optionally be substituted with a group selected from hydroxy, heterocycloalkyl or heterocycloalkenyl, which both can be substituted with a methyl- and/or oxo-group,
and wherein xis 1, 2 or 3,
A1 stands for
   a C₄ to C₁₂ carbocyclic, heterocyclic, optionally bicyclic, optionally aromatic or optionally heteroaromatic ring system, wherein in a bicyclic aromatic or heteroaromatic ring system one or two double bonds can be hydrogenated,
R² stands for
   a hydrogen atom, a hydroxy group, oxo (═O), a halogen atom, a cyano group, a substituent selected from: a C₁-C₆-alkyl, C₁-C₆-alkoxy-, C₂-C₆-alkenyl, C₂-C₆-alkynyl, C₃-C₈-cycloalkyl, C₄-C₈-cycloalkenyl, 4- to 7-membered heterocycloalkyl, —O—CH₂-4- to 7-membered heterocycloalkyl, 5- to 10-membered heterocycloalkenyl, heterospirocycloalkyl, fused heterocycloalkyl, bridged heterocycloalkyl, phenyl, heteroaryl, C₁-C₆-haloalkyl, C₁-C₆-alkylsulfonyl,
   —NR^{a}R^{b}, wherein R^{a} and R^{b} are selected independently from a hydrogen atom or a C₁-C₆-alkyl,
   —C(O)—NR^{a}R^{b}, wherein R^{a} and R^{b} are selected independently from a hydrogen atom or a C₁-C₆-alkyl, —C(O)—O—R^{g}, wherein R^{g} is a hydrogen atom or a C₁-C₆-alkyl, —O—R^{h}, wherein R^{h} is a C₁-C₆-alkyl or CH₂—N R^{a} R^{b}, wherein R^{a} and R^{b} are selected independently from a hydrogen atom or a C₁-C₆-alkyl,
and w is 1 or 2,
and wherein
A2(R³)_{y} stands either for a hydrogen atom or
A2 has the same meanings as the substituent A1 and
R³ stands for
   a hydrogen atom, a halogen atom, a hydroxy, oxo, cyano, nitro group, a substituent selected from a C₁-C₆-alkyl, C₂-C₆-alkenyl, C₂-C₆-alkynyl, C₃-C₈-cycloalkyl, C₄-C₈-cycloalkenyl, C₇-C₈-cycloalkynyl, 4- to 7-membered heterocycloalkyl, 5- to 10-membered heterocycloalkenyl, phenyl, heteroaryl, C₁-C₆-haloalkyl,
      which substituent is optionally substituted, one, two or three times, identically or differently, with a substituent selected from:
         a halogen atom, or a group selected from hydroxy, oxo (═O), cyano, C₁-C₆-alkyl, C₁-C₆-alkoxy, C₁-C₆-haloalkyl, phenyl, —C(O)NRⁱR^{j}, wherein
         Rⁱ and R^{j} are selected independently from a hydrogen atom or a C₁-C₆-alkyl, heteroaryl,
      or with amino NR^{k}R^{l}, wherein R^{k} and R^{l} are selected independently from
         a hydrogen atom, a substituent selected from a C₁-C₆-alkyl, C₂-C₆-alkenyl, C₂-C₆-alkynyl, C₃-C₈-cycloalkyl, C₁-C₆-alkylsulfonyl, phenyl, heteroaryl, 4- to 7-membered heterocycloalkyl, which are optionally substituted
         one, two or three times, identically or differently, with a substituent selected from C₁-C₆-haloalkyl, hydroxyl, oxo (═O), phenyl, cyano , C₁-C₆-alkoxy, heteroaryl, wherein
         the heteroaryl can optionally be substituted with a methyl group, or —CH₂—C(O)—R^{m}, wherein
         3 R^{m} is a bicyclic heteroaryl, which can be partially hydrogenated, a C₁-C₆-alkoxy or a group NRⁿR^{o}, in which
         Rⁿ and R^{o} are selected independently from hydrogen, C₁-C₆-alkyl, phenyl, wherein the C₁-C₆-alkyl can optionally be substituted with a C₁-C₆-alkoxy or a phenyl, or
         —NRⁿR^{o} stands for a 4- to 7-membered azacycloalkyl, bound via the nitrogen atom to the rest of the molecule and which optionally contains one more heteroatom selected from nitrogen and oxygen;
         —C(═O)R^{p}, wherein R^{p} is selected from
         the group of a C₁-C₆-alkoxy, a C₁-C₆-alkyl, which is optionally substituted, one, two or three times, identically or differently, with a substituent selected from hydroxyl or C₁-C₆-alkoxy, a mono- or bicyclic heteroaryl, a 4- to 7-membered heterocycloalkyl or R^{p} is a group CH₂—NR^{q}R^{r}; wherein R^{q} and R^{r} are selected independently from hydrogen, phenyl or a C₁-C₆-alkyl, which may optionally be substituted up to threefold with fluorine,
      —NR^{s}R^{t} is
         a 4- to 7-membered azacycloalkyl, bound via the nitrogen atom to the rest of the molecule, or a 6- to 10-membered azaspirocycloalkyl, which both may contain up to 2 further heteroatoms selected from nitrogen and oxygen and which both are optionally substituted one, two or three times, identically or differently, with a substituent selected from: hydroxy, oxo (═O), C₁-C₆-alkyl, C₁-C₆-hydroxyalkyl, —C(═O)OR^{u}, wherein R^{u} is a C₁-C₆-alkyl, halogen, —N(C₁-C₆-alkyl)₂, —CH₂—N(C₁-C₆-alkyl)₂, —C(O)NR^{a}R^{b}, wherein R^{a} and R^{b} are selected independently from a hydrogen atom or a C₁-C₆-alkyl,
   —C(═O)R^{v}, —C(═O)NH₂, —C(═O)N(H)R^{v}, —C(═O)N(R^{v})R^{w}, —C(═O)OR^{v}, wherein
      R^{v} and R^{w} represent, independently from each other, a group selected from hydrogen, C₁-C₄-alkyl, C₁-C₄-haloalkyl, phenyl, or a group (CH₂)₂—NR^{x}R^{y}, wherein R^{x} and R^{y} independently from each other stand for hydrogen, a C₁-C₄-alkyl or a group —CH₂)₂N(CH₃)₂;
   —NH₂, —NHR^{z}, —N(R^{z})R^{za}, —N(H)C(═O)R^{z}, —N(H)C(═O)OR^{z}, —N(H)S(═O)₂R^{z}, 4- to 7-membered heterocycloalkyl, heteroaryl, heterospirocycloalkyl, fused heterocycloalkyl, bridged heterocycloalkyl, wherein
      R^{z} and R^{za} represent, independently from each other, a group selected from C₁-C₄-alkyl, C₁-C₄-haloalkyl and phenyl,
   C₁-C₆-alkoxy-, C₁-C₆-haloalkoxy-, —O—(CH₂), —C₃-C₈-cycloalkyl, —O—(CH₂),-phenyl, —O—(CH₂)ₛ-heterocycloalkyl, —O—(CH₂)ₛ-heteroaryl, s is 0, 1, 2 or 3,
   —S(═O)₂R_{z}, —S(═O)₂NH₂, —S(═O)₂NHRz, —S(═O)₂N(R^{z})R^{za}, wherein R^{z} and R^{za} represent, independently from each other, a group selected from C₁-C₄-alkyl, C₁-C₄-haloalkyl and phenyl,
wherein y is 1, 2 or 3, and
L stands either for a bond or for O—(CH₂)ₖ, wherein k is 0, 1, 2 or 3, or a group CH═CH—(CH₂)., wherein n is 0, 1 or 2,
and either both T and V stand for nitrogen or T stands for carbon and V for nitrogen or T for nitrogen and V for carbon,
or a tautomer, an N-oxide, a hydrate, a solvate, or a salt thereof, or a mixture of same.

### BRIEF DESCRIPTION OF THE FIGURES

FIG. 1 shows the sequence of hSOS1_12 with N-terminal His tag (His10-hSOS1_12) before cleavage by TEV protease (SEQ ID NO: 6).

FIG. 2 shows the sequence of hSOS1_12 after cleavage by TEV protease (SEQ ID NO: 7).

FIG. 3 shows the structure of Example 81 in complex with human hSOS1_12. Carbon atom C22 unambiguously features R configuration.

### DEFINITIONS

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

Should a composite substituent be composed of more than one parts, e.g. (C₁-C₄-alkoxy)-(C₁-C₄-alkyl)-, it is possible for the position of a given part to be at any suitable position of said composite substituent, i.e. the C₁-C₄-alkoxy part can be attached to any carbon atom of the C₁-C₄-alkyl part of said (C₁-C₄-alkoxy)-(C₁-C₄-alkyl)- group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tent-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₆-hydroxyalkyl” means a linear or branched, saturated, monovalent hydrocarbon group in which the term “C₁-C₆-alkyl” is defined supra, and in which 1, 2 or 3 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2, 3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₆-alkylsulfanyl” means a linear or branched, saturated, monovalent group of formula (C₁-C₆-alkyl)-S-, in which the term “C₁-C₆-alkyl” is as defined supra, e.g. a methylsulfanyl, ethylsulfanyl, propylsulfanyl, isopropylsulfanyl, butylsulfanyl, sec-butylsulfanyl, isobutylsulfanyl, tent-butylsulfanyl, pentylsulfanyl, isopentylsulfanyl, hexylsulfanyl group.

The term “C₁-C₆-alkylsulfonyl” means a linear or branched, saturated, monovalent group of formula (C₁-C₆-alkyl)-SO₂—, in which the term “C₁-C₆-alkyl” is as defined supra, e.g. a methylsulfonyl, ethylsulfonyl, propylsulfonyl, isopropylsulfonyl, butylsulfonyl, sec-butylsulfonyl, isobutylsulfonyl, tent-butylsulfonyl, pentylsulfonyl, isopentylsulfonyl, hexylsulfonyl group.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, see-butoxy, isobutoxy, tert-butoxy, pentyloxy, isopentyloxy or n-hexyloxy group, or an isomer thereof.

The term “C₂-C₆-alkenyl” means a linear or branched, monovalent hydrocarbon group, which contains one or two double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then it is possible for said double bonds to be isolated from, or conjugated with, each other. Said alkenyl group is, for example, an ethenyl (or “vinyl”), prop-2-en-1-yl (or “allyl”), prop-I-en-1-yl , but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-i sopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl , 1-propylprop-1-enyl, 2-isopropylprop-1-enyl , 1-isopropylprop-1-enyl, 3 ,3-dimethylprop-1-enyl, 1-(1,1-dimethylethyl)ethenyl, buta-1,3-dienyl, penta-1,4-dienyl or hexa-1,5-dienyl group. Particularly, said group is vinyl or allyl.

The term “C₂-C₆-alkynyl” means a linear or branched, monovalent hydrocarbon group which contains one triple bond, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl , 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1, 1-dimethylbut-3-ynyl, 1,1-dimethylbut-2-ynyl or 3,3-dimethylbut-1-ynyl group. Particularly, said alkynyl group is ethynyl, prop-1-ynyl or prop-2-ynyl.

The term “C₃-C₈-cycloalkyl” means a saturated, monovalent, mono- or bicyclic hydrocarbon ring which contains 3, 4, 5, 6, 7 or 8 carbon atoms (“C₃-C₈-cycloalkyl”). Said C₃-C₈-cycloalkyl group is for example, a monocyclic hydrocarbon ring, e.g. a cyclopropyl, cyclobutyl, cyclopentyl, cyclohexyl, cycloheptyl or cyclooctyl group, or a bicyclic hydrocarbon ring, e.g. a bicyclol4.2.0loctyl or octahydropentalenyl.

The term “C₄-C₈-cycloalkenyl” means a monovalent, mono- or bicyclic hydrocarbon ring which contains 4, 5, 6, 7 or 8 carbon atoms and one double bond. Particularly, said ring contains 4, 5 or 6 carbon atoms (“C₄-C₆-cycloalkenyl”). Said C₄-C₈-cycloalkenyl group is for example, a monocyclic hydrocarbon ring, e.g. a cyclobutenyl, cyclopentenyl, cyclohexenyl, cycloheptenyl or cyclooctenyl group, or a bicyclic hydrocarbon ring, e.g. a bicyclo[2.2.1]hept-2-enyl or bicyclo [2.2.2]oct-2-enyl.

The term “C₃-C₈-cycloalkoxy” means a saturated, monovalent, mono- or bicyclic group of formula (C₃-C₈-cycloalkyl)-O—, which contains 3, 4, 5, 6, 7 or 8 carbon atoms, in which the term “C₃-C₈-cycloalkyl” is defined supra, e.g. a cyclopropyloxy, cyclobutyloxy, cyclopentyloxy, cyclohexyloxy, cycloheptyloxy or cyclooctyloxy group.

The term “spirocycloalkyl” means a saturated, monovalent bicyclic hydrocarbon group in which the two rings share one common ring carbon atom, and wherein said bicyclic hydrocarbon group contains 5, 6, 7, 8, 9, 10 or 11 carbon atoms, it being possible for said spirocycloalkyl group to be attached to the rest of the molecule via any one of the carbon atoms except the spiro carbon atom. Said spirocycloalkyl group is, for example, spiro[2.2]pentyl, spiro[2.3]hexyl, spiro[2.4]heptyl, spiro[2.5]octyl, spiro[2.6]nonyl, spiro[3.3]heptyl, spiro[3.4]octyl, spiro[3.5]nonyl, spiro[3.6]decyl, spiro[4.4]nonyl, spiro [4.5]decyl, spiro[4.6]undecyl or spiro[5.5]undecyl.

The terms “4- to 7-membered heterocycloalkyl” means a monocyclic, saturated heterocycle with 4, 5, 6 or 7 ring atoms in total, which contains one or two identical or different ring heteroatoms from the series N, O and S, it being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

Particularly, “4- to 6-membered heterocycloalkyl” means a 4- to 6-membered heterocycloalkyl as defined supra containing one ring nitrogen atom and optionally one further ring heteroatom from the series:N, O, S. More particularly, “5- or 6-membered heterocycloalkyl” means a monocyclic, saturated heterocycle with 5 or 6 ring atoms in total, containing one ring nitrogen atom and optionally one further ring heteroatom from the series: N, O.

The term “4- to 7-memebered azacycloalkyl” means a monocyclic saturated heterocycly with 4, 5, 6 or 7 ring atoms in total which is attached to the rest of the molecule via the nitrogen atom and which optionally contains one more heteroatom selected from nitrogen and oxygen.

Said 4- to 7-membered azacycloalkyl group, without being limited thereto, can be a 4-membered ring, such as azetidin-1-yl, for example; or a 5-membered ring, such as pyrrolidin-1-yl, imidazolidin-1-yl, pyrazolidin-1-yl, 1,2-oxazolidin-2-yl or 1,3-oxazolidin-3-yl, for example; or a 6-membered ring, such as piperidin-1-yl, morpholin-4-yl, piperazin-1-yl or 1,2-oxazinan-2-yl, for example, or a 7-membered ring, such as azepan-1-yl, 1,4-diazepan-1-yl or 1,4-oxazepan-4-yl, for example.

The term “5- to 10-membered heterocycloalkenyl” means a monocyclic, unsaturated, non-aromatic heterocycle with 5, 6, 7, 8, 9 or 10 ring atoms in total, which contains one or two double bonds and one or two identical or different ring heteroatoms from the series:N, O, S; it being possible for said heterocycloalkenyl group to be attached to the rest of the molecule via any one of the carbon atoms or, if present, a nitrogen atom.

Said heterocycloalkenyl group is, for example, 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl or 4H-[1,4]thiazinyl.

The term “heterospirocycloalkyl” means a bicyclic, saturated heterocycle with 6, 7, 8, 9, 10 or 11 ring atoms in total, in which the two rings share one common ring carbon atom, which “heterospirocycloalkyl” contains one, two or three identical or different ring heteroatoms from the series:N, O, S; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[3.3]heptyl, oxaazaspiro [3 .3]heptyl, thi aazaspiro [3 .3]heptyl , oxaspiro [3 .3]heptyl, oxazaspiro [5 .3]nonyl, oxazaspiro[4.3]octyl, azaspiro[4,5]decyl, oxazaspiro [5.5]undecyl, diazaspiro[3.3]heptyl, thiazaspiro[3.3]heptyl, thiazaspiro[4.3]octyl, azaspiro[5.5]undecyl, or one of the further homologous scaffolds such as spiro [3.4]-, spiro [4.4]-, spiro [2.4]spiro [2.5]-, spiro [2.6]-, spiro [3.5]-, spiro [3. 6]spiro [4.5]- and spiro [4.6]-.

The term “6- to 10-membered azaspirocycloalkyl” means a bicyclic, saturated heterocycle with 6, 7, 8, 9 or 10 ring atoms in total, in which the two rings share one common ring carbon atom and which is bound to the rest of the molecule via the nitrogen atom and which azaspirocycloalkyl may contain up to 2 further heteroatoms selected from nitrogen and oxygen.

Said azaspirocycloalkyl is for example, azaspiro[2.3]hexyl, azaspiro[3.3]heptyl, oxaazaspiro [3 .3]heptyl, oxazaspiro [5 .3]nonyl, oxazaspiro [4.3]octyl, azaspiro [4,5]decyl, oxazaspiro[5.5]undecyl, diazaspiro[3.3]heptyl, triazaspiro[3.4]octyl or one of the further homologous scaffolds such as spiro [3.4]-, spiro [4.4]-, spiro [2.4]-, spiro [2.5]-, spiro [2.6]-, spiro[3.5]-, spiro[3.6]- and spiro[4.5]-, whereby these azaspirocycloalkyl groups are always bound via the nitrogen atom to the rest of the molecule.

Of these groups preference is given to 2-oxa-6-azaspiro[3.3]hept-6-yl and 2,5,7-triazaspiro[3.4]octan-2-yl.

The term “fused heterocycloalkyl” means a bicyclic, saturated heterocycle with 6, 7, 8, 9 or 10 ring atoms in total, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom.

Said fused heterocycloalkyl group is, for example, azabicyclo[3.3.0]octyl, azabicyclo[4.3.0]nonyl, diazabicyclo [4.3 .0]nonyl, oxazabicyc lo [4.3 .0]nonyl, thiazabicyclo[4.3.0]nonyl or azabicyclo[4.4.0]decyl.

The term “bridged heterocycloalkyl” means a bicyclic, saturated heterocycle with 7, 8, 9 or 10 ring atoms in total, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, except the spiro carbon atom, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl, diazabicyclo[2.2.1]heptyl, azabicyclo-[2.2.2]octyl, diazabicyclo[2.2.2]octyl, oxazabicyclo[2.2.2]octyl, thiazabicyclo[2.2.2]octyl, azabicyclo[3.2.1]octyl, diazabicyclo [3 .2.1]octyl, oxazabicyclo[3.2.1]octyl, thiazabicyclo[3.2.1]octyl, azabicyclo[3.3.1]nonyl, diazabicyc lo [3 .3.1]nonyl, oxazabicyc lo [3 .3.1]nonyl, thiazabicyclo [3 .3.1]nonyl , azabicyclo [4.2.1]nonyl, diazabicyclo [4.2.1]nonyl, oxazabicyclo[4.2.1]nonyl, thiazabicyclo[4.2.1]nonyl, azabicyclo[3.3.2]decyl, diazabicyclo [3 .3 .2]decyl, oxazabic yclo [3 .3 .2]decyl, thiazabicyclo[3.3.2]decyl or azabicyclo[4.2.2]decyl.

The term “heteroaryl” means a monovalent, monocyclic, bicyclic or tricyclic aromatic ring having 5, 6, 8, 9, 10, 11, 12, 13 or 14 ring atoms (a “5- to 14-membered heteroaryl” group), particularly 5, 6, 9 or 10 ring atoms, which contains at least one ring heteroatom and optionally one, two or three further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; a 8-membered heteroaryl group, such as for example 6,7-dihydro-5H-pyrrolol1,2-alimidazolyl or a 9-membered heteroaryl group, such as, for example, benzofuranyl, benzothienyl, benzoxazolyl, benzisoxazolyl, benzimidazolyl, benzothiazolyl, benzothiadiazolyl, benzotriazolyl, indazolyl, indolyl, isoindolyl, indolizinyl, thienopyridinyl, 1H-pyrrolo[2,3-b]pyridinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

A C4 to C12 carbocyclic, heterocyclic, optionally bicyclic, optionally aromatic or optionally heteroaromatic ring system, wherein in a bicyclic, aromatic or heteroaromatic ring system one or two double bonds can be hydrogenated is selected from the group of the substituents phenyl, naphthyl, 1,2,3,4-tetrahydronaphthyl, 1,3-benzodioxolyl, quinolinyl, isoquinolinyl, 2,3-dihydro-1,4-benzodioxinyl, imidazol1,2-alpyridinyl, furanyl, thienyl, pyridinyl, 2H-1,4-benzoxazinyl-3(4H)-one, 2,1,3-benzothiadiazolyl, 1-benzofuranyl, 1-benzothienyl, 1H-indazolyl, 1H-indolyl, 1H-benzimidazolyl, 1,3-benzothiazolyl, thieno[2,3-b]pyridinyl, thieno[2,3-c]pyridinyl, thieno[3,2-c]pyridinyl, pyrimidinyl, 1H-pyrazolyl, 6,7-dihydro-5H-pyrrolo[1,2-a]imidazolyl, 1,2-oxazolyl, 1H-imidazolyl, 1,3,4-oxadiazolyl, 1H-tetrazolyl, 1H-pyrrolyl, 1H-pyrrolo[2,3-b]pyridinyl or 3,4-dihydro-2H-1,4-benzoxazinyl

Particularly, the heteroaryl group is a quinolinyl, isoquinolinyl, imidazo[1,2-a]pyridinyl, furanyl, thienyl, pyridinyl, 2,1,3-benzothiadiazolyl, 1-benzofuranyl, 1-benzothiophenyl, 1H-indazolyl, 1H-indolyl, 1H-benzimidazolyl, 1,3-benzothiazolyl, thieno[2,3-b]pyridinyl, thieno[2,3-c]pyridinyl, thieno[3,2-c]pyridinyl, pyrimidinyl, 1H-pyrazolyl, 6,7-dihydro-5H-pyrrolo[1,2-a]imidazolyl, 1,2-oxazolyl, 1H-imidazolyl, 1,3,4-oxadiazolyl, 1H-tetrazolyl, 1H-pyrrolyl, 1H-pyrrolo[2,3-b]pyridinyl or 3,4-dihydro-2H-1,4-benzoxazinyl group.

In composite substituents such asC₁-C₆-haloalkyl, C₁-C₄-haloalkyl, C₁-C₆-haloalkoxy, —(CH₂)-heteroaryl, heteroaryloxy, —O—(CH₂)ₓ-heteroaryl, —O—(CH₂)_{z}-heteroaryl, O-(CH₂)-4- to 7-membered heterocycloalkyl, bicyclic heteroaryl, C₁-C₆-hydroxyalkyl, —O—(CH₂)ₓ-C₃-C₈-cycloalkyl, O—(CH₂)ₓ-phenyl, —O—(CH₂)ₓ-heterocyclyl and C₃-C₈-cycloalkyloxy the definition of the residue to which the further substituent is attached is the same as given for the residues which do not bear a further substituent, e.g. inC₁-C₆-haloalkyl the C₁-C₆-alkyl has the same meanings as given for the C₁-C₆-alkyl earlier.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₈-cycloalkyl”, means a cycloalkyl group having a finite number of carbon atoms of 3 to 8, i.e. 3, 4, 5, 6, 7 or 8 carbon atoms.

When a range of values is given, said range encompasses each value and sub-range within said range.

For example:

“C₁-C₆” encompasses C₁, C₂, C₃, C₄, C₅, C₆, C₁-C₆, C₁-C₂, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;

“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;

“C₃-C₁₀” encompasses C₃, C₄, C₅, C₆, C₇, C₈, C₉, C₁₀, C₃-C₁₀, C₃-C₉, C₃-C₇, C₃-C₆, C₃-C₅, C₃-C₄, C₄-C₁₀, C₄-C_{9,} C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;

“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;

“C₃-C₆” e ncompasses C₃, C₄, C₅, C₆, C₃-C₆, C₃-C₅, C₃-C₄, C₄-C₆, C₄-C₅, and C₅-C₆;

“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;

“C₄-C₇” encompasses C₄, C₅, C₆, C₇, C₄-C₇, C₄-C₆, C₄-C₅, C₅-C₇, C₅-C₆ and C₆-C₇;

“C₄-C₆” encompasses C₄, C₅, C₆, C₄-C₆, C₄-C₅ and C₅-C₆;

“C₅-C₁₀” encompasses C₅, C₆, C₇, C₈, C₉, C₁₀, C₅-C₁₀, C₅-C_{9,} C₅-C₈, C₅-C₇, C₅-C₆, C₆- C₁₀, C₆-C₉, C₆-C₈, C₆-C₇, C₇-C₁₀, C₇-C₉, C₇-C₈, C₈-C₁₀, C₈-C₉ and C₉-C₁₀;

“C₆-C₁₀” encompasses C₆, C₇, C₈, C₉, C₁₀, C₆-C₁₀, C₆-C_{9,} C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)-sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4, 6-trimethylphenyl)sulfonyl]oxy, [(4-tert-butylphenyl)sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹³C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, 123I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

In another embodiment the present invention concerns a deuterium-containing compound of general formula (I), in which one, two or three of the hydrogen atom(s) in either one or both of the methyl groups shown in general formula (I) is/are replaced with a deuterium atom. Also the hydrogen atom on the carbon atom between the nitrogen atom and the group A1 can be replaced with a deuterium atom either as the single replacement of a hydrogen by a deuterium or in addition to the beforementioned replacements in either one or both of the methyl groups shown in general formula (I).

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention contain at least one or optionally even more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred isomers are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel O D and Chiracel O J, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)— or (S)— isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely:

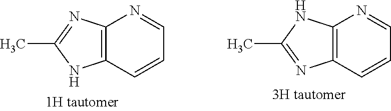

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanes ulfonic, itaconic, trifluoromethanesulfonic, dodecylsulfuric, ethanesulfonic, benzenes ulfonic , para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanol amine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

As used herein, the term “in vivo hydrolysable ester” means an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl ; 1,3-dioxolen-2-onylmethyl esters, e.g. 5-methyl-1,3-dioxolen-2-onylmethyl ; and C₁-C₆-alkoxycarbonyloxyethyl esters, e.g. 1-methoxycarbonyloxyethyl, it being possible for said esters to be formed at any carboxy group in the compounds of the present invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

In accordance with another embodiment of the first aspect, the present invention covers compounds of general formula (2)

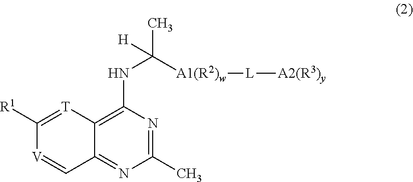

in which:
R¹ stands for a substituent selected from:
   a halogen atom,
   a C₁-C₆-alkylsulfanyl group,
   —R^{a}R^{b}, wherein R^{a} and R^{b} are independently selected from a hydrogen atom or C₁-C₆-alkyl,
   C₁-C₆-alkoxy,
   4- to 7-membered heterocycloalkyl,
   5- to 10 membered heterocycloalkenyl;
   —NH—(CH₂)ₖ—NH—C(O)—C₁-C₆-alkyl, wherein k is 1 or 2;
   —NH—(CH₂)ₗ—R^{f}, wherein 1 is 0, 1 or 2 and R^{f} stands for a 4- to 7-membered heterocycloalkyl, heteroaryl or C₁-C₆-alkylsulfonyl;
      whereby in all foregoing definitions the C₁-C₆-alkyl-, C₁-C₆-alkoxy-, the 4- to 7-membered heterocycloalkyl and the heteroaryl can be optionally substituted, one or two or three times, identically or differently, with a hydroxy, oxo (═O), C₁-C₆-alkyl, C₃-C₈-cycloalkyl, 4- to 7-membered heterocycloalkyl, C₁-C₆-alkoxy, C₁-C₆-alkylsulfonyl, phenyl, benzyl, heteroaryl, —CH₂-heteroaryl, C₃-C₈-cycloalkoxy, phenyloxy, heteroaryloxy, —NH—C(O)—C₁-C₆-alkyl or —NR^{a}R^{b}, wherein R^{a} and R^{b} are independently selected from a hydrogen atom or C₁-C₆-alkyl;
   —O—(CH₂)z-phenyl, wherein z is 0, 1 or 2, and the phenyl, can optionally be substituted with a group selected hydroxyheterocycloalkyl heterocaclyoalkenyl, which both can be substituted with a methylgroup;

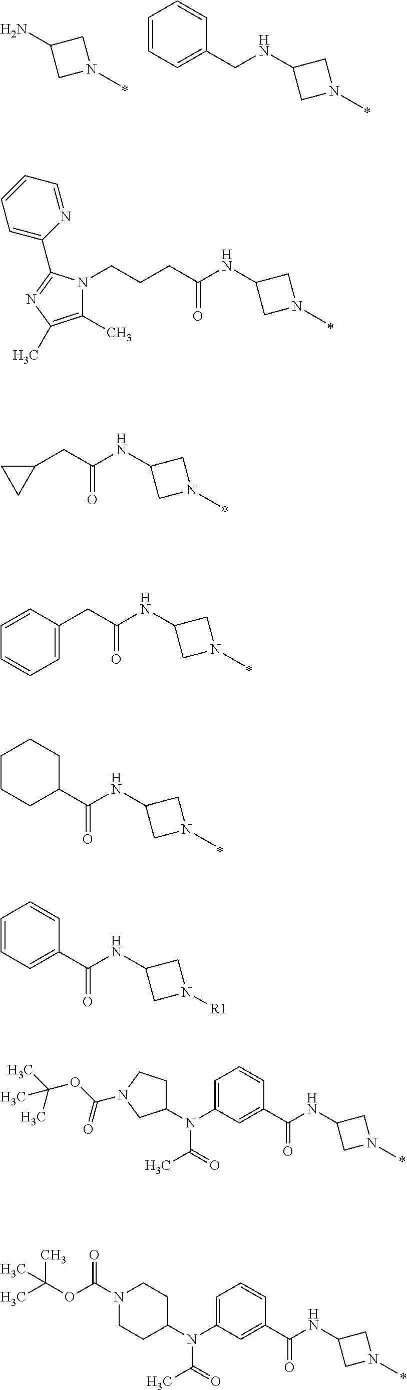

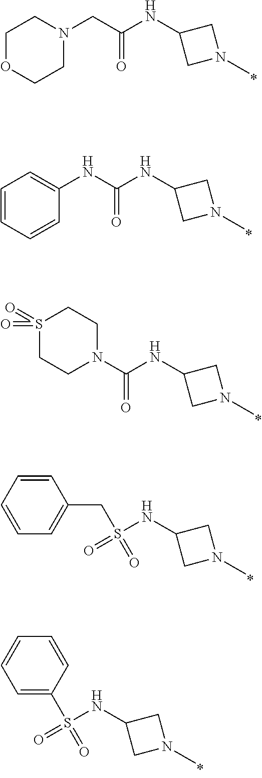 [SMILES: CN1CC(NC(=O)CN2CCOCC2)C1 ; CN1CC(NC(=O)Nc2ccccc2)C1 ; CN1CC(NC(=O)N2CCS(=O)(=O)CC2)C1 ; CN1CC(NS(=O)(=O)Cc2ccccc2)C1 ; CN1CC(NS(=O)(=O)c2ccccc2)C1]

A1 stands for
   an optionally bicyclic C₅-C₉-aromatic or an optionally bicyclic C₅-C₉-heteroaromatic ring system
R² stands for a substituent selected from:
   a hydrogen atom,
   a halogen atom,
   C₁-C₆-alkyl,
   C₃-C₈-cycloalkyl,
   C₁-C₆-alkylsulfonyl,
and wherein w is 0, 1 or 2,
and wherein A2(R³)_{y} stands either for a hydrogen atom or
A2 is phenyl and
R³ stands for a substituent selected from:
   C₁-C₆-alkyl,
      which is substituted, with a substituent selected from:
         a hydroxy group,
         NR^{k}R^{l}, wherein R^{k} and R^{l} are independently selected from
         a hydrogen atom,
         C₁-C₆-alkyl,
         wherein y is 1 and
         L stands either for a bond
      and either both T and V stand for nitrogen or T stands for carbon and V for nitrogen or T for nitrogen and V for carbon,
   or a tautomer, an N-oxide, a hydrate, a solvate, or a salt thereof, or a mixture of same.

In accordance with a second embodiment of the first aspect, the present invention covers compounds of general formula (I), supra, in which:
R¹ stands for
   a substituent independently selected from: a hydrogen atom, a halogen atom, a hydroxy, nitro, C₁-C₆-alkylsulfanyl or an amino group —NR^{a}R^{b},
      wherein R^{a} and R^{b} are selected independently from a hydrogen atom or a C₁-C₆-alkyl,
   a substituent selected from: a C₁-C₆-alkyl, C₁-C₆-alkoxy-, C₃-C₈-cycloalkyl, 4- to 7-membered heterocycloalkyl, heteroaryl,
   —C(═O)OH, —C(═O)OR^{c}, and wherein R^{c} stands for C₁-C₆-alkyl or C₃-C₈-cycloalkyl,
   —N═S(═O)(R^{d})R^{e}, and wherein R^{d} and R^{e} are selected independently from C₁-C₆-alkyl,
   —NH—C(O)—C₁-C₆-alkyl, —NH—C(O)—N R^{a}R^{b}, wherein R^{a} and R^{b} are selected independently from a hydrogen atom or a C₁-C₆-alkyl, —NH—(CH₂)ₖ—NH—C(O)—C₁-C₆-alkyl, wherein k is 2, —NH—(CH₂)₁—R^{f}, wherein
      1 is 0 or 2 and R^{f} stands for a 4- to 7-membered heterocycloalkyl or C₁-C₆-alkylsulfonyl,
      whereby in all foregoing definitions the C₁-C₆-alkyl-, C₁-C₆-alkoxy-, the 4- to 7-membered heterocycloalkyl and the heteroaryl can be optionally substituted, one or two or three times, identically or differently, with:
         a group selected from hydroxy, oxo (═O), C₁-C₆-alkyl, C₃-C₈-cycloalkyl, 4- to 7-membered heterocycloalkyl, C₁-C₆-alkoxy, C₁-C₆-alkylsulfonyl, benzyl, —(CH₂)-heteroaryl- or an amino group —NR^{a}R^{b}, wherein R^{a} and R^{b} are selected independently from C₁-C₆-alkyl, or
   a substituent —O—(CH₂)_{z}-phenyl, whereby z is 0, 1 or 2,
and wherein xis 1, 2 or 3,
A1 stands for
   a C₄ to C₁₂ carbocyclic, heterocyclic, optionally bicyclic, optionally aromatic or optionally heteroaromatic ring system, wherein in a bicyclic aromatic or heteroaromatic ring system one or two double bonds can be hydrogenated,
R² stands for
   a hydrogen atom, a hydroxy group, oxo (═O), a halogen atom, a cyano group, a substituent selected from: a C₁-C₆-alkyl, C₁-C₆-alkoxy-, C₂-C₆-alkenyl, C₃-C₈-cycloalkyl, 4- to 7-membered heterocycloalkyl, —O—CH₂-4- to 7-membered heterocycloalkyl, C₁-C₆-alkylsulfonyl,
   —C(O)—NR^{a}R^{b}, wherein R^{a} and R^{b} are both hydrogen atoms, —C(O)—O—R^{g}, wherein R^{g} is a C₁-C₆-alkyl, or —CH₂—N R^{a}R^{b}, wherein R^{a} and R^{b} are both hydrogen atoms,
and w is 1 or 2,
and wherein
A2(R³)_{y} stands either for a hydrogen atom or
A2 has the same meanings as the substituent A1 and
R³ stands for
   a hydrogen atom, a halogen atom, a hydroxy, oxo, cyano, nitro group,
   a substituent selected from a C₁-C₆-alkyl, C₂-C₆-alkenyl, C₂-C₆-alkynyl, C₃-C₈-cycloalkyl, C₄-C₈-cycloalkenyl, C₇-C₈-cycloalkynyl, 4- to 7-membered heterocycloalkyl, 5- to 10-membered heterocycloalkenyl, phenyl, heteroaryl, C₁-C₆-haloalkyl,
      which substituent is optionally substituted, one, two or three times, identically or differently, with a substituent selected from:
         a halogen atom, or a group selected from hydroxy, oxo (═O), cyano, C₁-C₆-alkyl, C₁-C₆-alkoxy, C₁-C₆-haloalkyl, phenyl, —C(O)NRⁱR^{j}, wherein
         Rⁱ and R^{j} are selected independently from a hydrogen atom or a C₁-C₆-alkyl, heteroaryl,
      or with amino —NR^{k}R^{l}, wherein R^{k} and R^{l} are selected independently from a hydrogen atom, a substituent selected from a C₁-C₆-alkyl, C₂-C₆-alkenyl, C₂-C₆-alkynyl, C₃-C₈-cycloalkyl, C₁-C₆-alkylsulfonyl, phenyl, heteroaryl, 4- to 7-membered heterocycloalkyl, which are optionally substituted
         one, two or three times, identically or differently, with a substituent selected from C₁-C₆-haloalkyl, hydroxyl, oxo (═O), phenyl, cyano , C₁-C₆-alkoxy, heteroaryl, wherein
         the heteroaryl can optionally be substituted with a methyl group, or
      —CH₂—C(O)—R^{m}, wherein
         R^{m} is a bicyclic heteroaryl, which can be partially hydrogenated, a C₁-C₆-alkoxy or a group —NRⁿR^{o}, in which
         Rⁿ and R^{o} are selected independently from hydrogen, C₁-C₆-alkyl, phenyl, wherein the C₁-C₆-alkyl can optionally be substituted with a C₁-C₆-alkoxy or a phenyl, or
         —NRⁿR^{o} stands for a 4- to 7-membered azacycloalkyl, bound via the nitrogen atom to the rest of the molecule and which optionally contains one more heteroatom selected from nitrogen and oxygen;
      —C(═O)R^{p}, wherein R^{p} is selected from
         the group of a C₁-C₆-alkoxy, a C₁-C₆-alkyl, which is optionally substituted, one, two or three times, identically or differently, with a substituent selected from hydroxyl or C₁-C₆-alkoxy,
         a mono- or bicyclic heteroaryl, a 4- to 7-membered heterocycloalkyl or R^{p} is a group —CH₂—NR^{q}R^{r}; wherein R^{q} and R^{r} are selected independently from hydrogen, phenyl or a C₁-C₆-alkyl, which may optionally be substituted up to threefold with fluorine,
      —NR^{s}R^{t} is
         a 4- to 7-membered azacycloalkyl, bound via the nitrogen atom to the rest of the molecule, or a 6- to 10-membered azaspirocycloalkyl, which both may contain up to 2 further heteroatoms selected from nitrogen and oxygen and which both are optionally substituted one, two or three times, identically or differently, with a substituent selected from: hydroxy, oxo (═O), C₁-C₆-alkyl, C₁-C₆-hydroxyalkyl, —C(═O)OR^{u}, wherein Rⁿ is a C₁-C₆-alkyl, halogen, —N(C₁-C₆-alkyl)₂, —CH₂—N(C₁-C₆-alkyl)₂, —C(O)NR^{a}R^{b}, wherein R^{a} and R^{b} are selected independently from a hydrogen atom or a C₁-C₆-alkyl,
   —C(═O)R^{v}, —C(═O)NH₂, —C(═O)N(H)R^{v}, —C(═O)N(R^{v})R^{w}, —C(═O)OR^{v}, wherein
      R^{v} and R^{w} represent, independently from each other, a group selected from hydrogen, C₁-C₄-alkyl, C₁-C₄-haloalkyl, phenyl, or a group (CH₂)₂—NR^{x}R^{y}, wherein R^{x} and R^{y} independently from each other stand for hydrogen, a C₁-C₄-alkyl or a group (CH₂)₂N(CH₃)₂;
   —NH₂,—NHR^{z}, —N(R^{z})R^{za}, —N(H)C(═O)R^{z}, —N(H)C(═O)OR^{z}, —N(H)S(═O)₂R^{z}, 4- to 7-membered heterocycloalkyl, heteroaryl, heterospirocycloalkyl, fused heterocycloalkyl, bridged heterocycloalkyl, wherein
      R^{z} and R^{za} represent, independently from each other, a group selected from C₁-C₄-alkyl, C₁-C₄-haloalkyl and phenyl,
   C₁-C₆-alkoxy-, C₁-C₆-haloalkoxy-, —O—(CH₂),-C₃-C₈-cycloalkyl, —O—(CH₂),-phenyl, —O—(CH₂)ₛ-heterocycloalkyl, —O—(CH₂)ₛ-heteroaryl, s is 0, 1, 2 or 3,
   —S(═O)₂R^{z}, —S(═O)₂NH₂, —S(═O)₂NHRz, —S(═O)₂N(R^{z})R^{za}, wherein R^{z} and R^{za} represent, independently from each other, a group selected from C₁-C₄-alkyl, C₁-C₄-haloalkyl and phenyl,
wherein y is 1, 2 or 3, and
L stands either for a bond or for O—(CH₂)ₖ, wherein k is 0, 1, 2 or 3, or a group CH═CH—(CH₂)ₙ, wherein n is 0, 1 or 2,
or a tautomer, an N-oxide, a hydrate, a solvate, or a salt thereof, or a mixture of same.

In accordance with a third embodiment of the first aspect, the present invention covers compounds of general formula (I), supra, in which:

R¹ is selected from the list of the following substituents H, *—OCH₃, *—OC₂H₅,

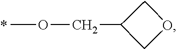

*-CH₂OH, *-C(O)OH, *-C(O)OCH₃, —Br, *—O—CH(CH₃)_{2·}, *-O—(CH₂)₂CH(CH₃)₂, *-O—(CH₂)₃CH₃, *-O—(CH₂)₂O—CH₃,

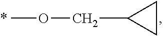

*—O—CH₂-Phenyl, *-N═S(O)(CH₃)₂, *-CH₃,

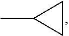

*-NH(CH₃), *-N(CH₃)₂, *-NH₂,

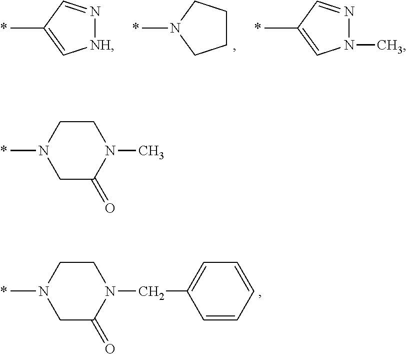

*-C(CH₃)₂—OH,

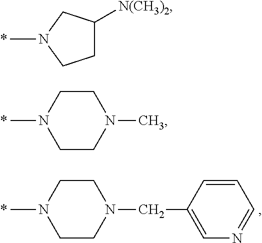

*-NH—(CH₂)₂—NH—C(O)—CH_{3·}, *-NH—(CH₂)₂-morpholino, *-NH—C(O)—CH₃, *-NH—C(O)—NH—CH₃,

*-NH—C(O)—N(CH₃)₂, *-NO₂, *-NH—S(O)₂—CH₃, *-N=S(O)(CH₃)₂, *-OH, —O—(CH₂)₂—S(O)₂—CH₃, *-fluorine,

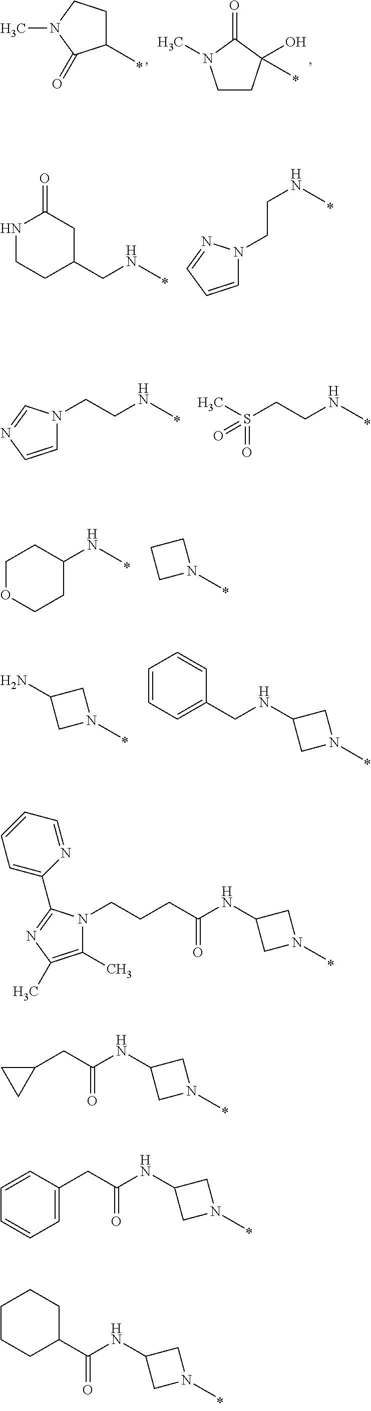

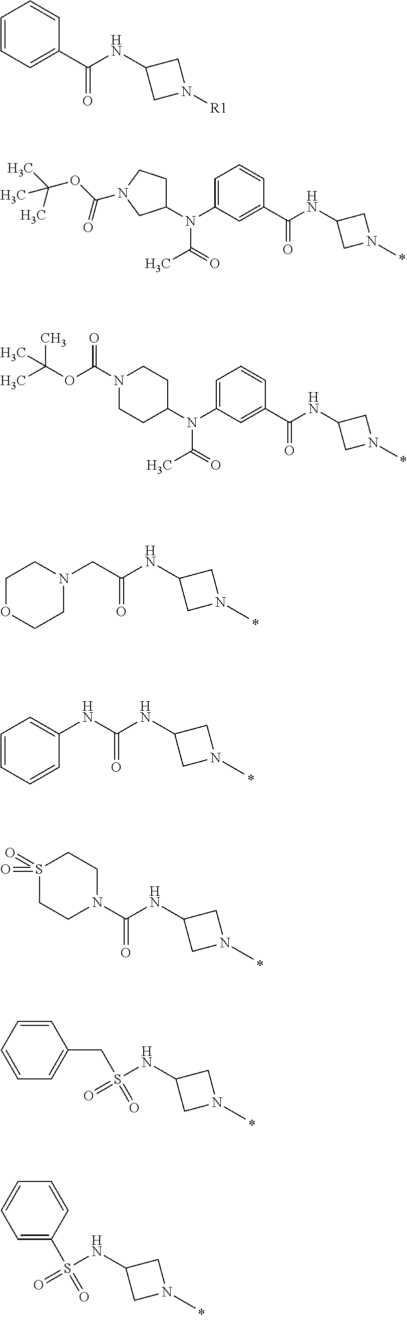

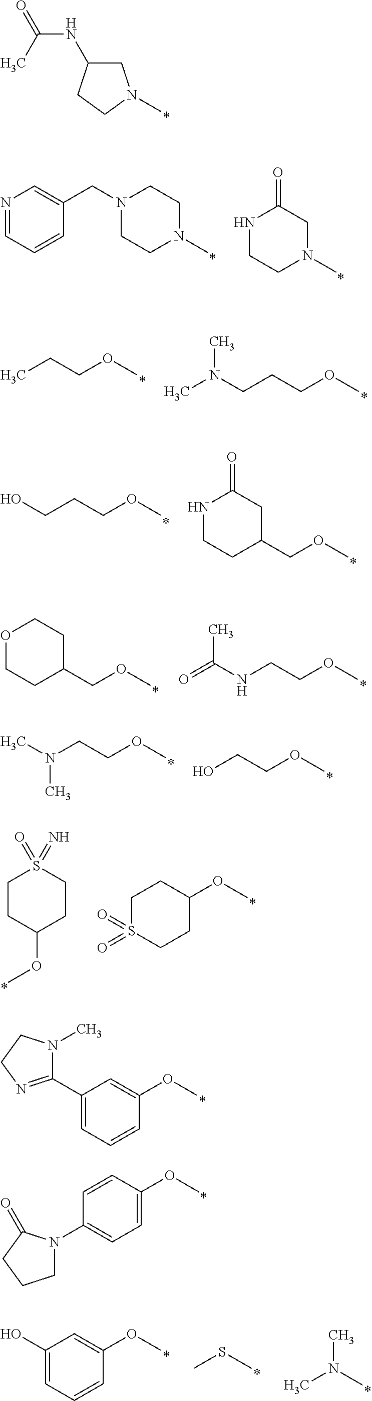

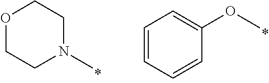

and
z is 1 or 2 and
x is 1 or 2 and wherein
A1 is selected from the group

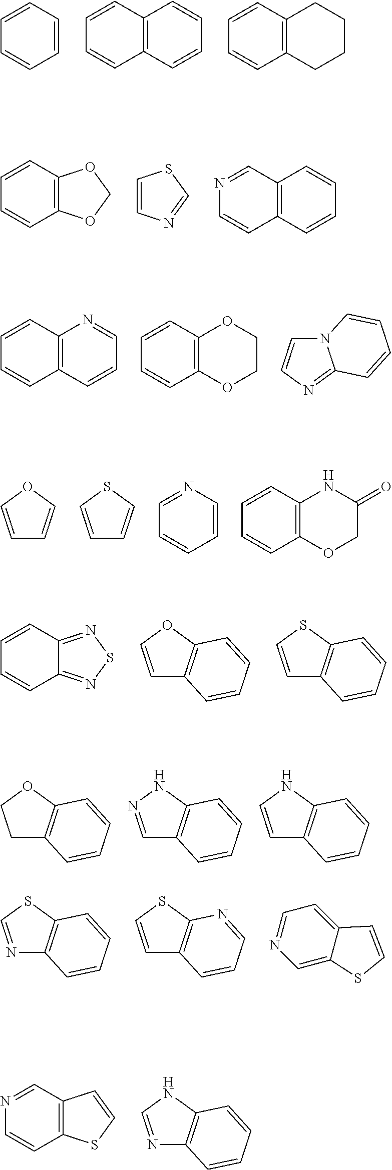

and
R² is selected from the group of hydrogen, hydroxy, oxo (═O), cyano, cyclopropyl, 1,1-dimethylcyclopropyl,
—C(═CH₂)CH₃, —C(CH₃)═CHCH₃, —CH═CH—(CH₂)₂CH₃, CH═CHCH₃, —CH═CH-cyclopropyl), —C(O)NH₂, C(O)OCH₃, —S(O)₂CH₃, —OCH₃, —CH₂NH₂, a halogen atom (F, Cl; Br),and
w is 1 or 2 and
A2 is selected from the group

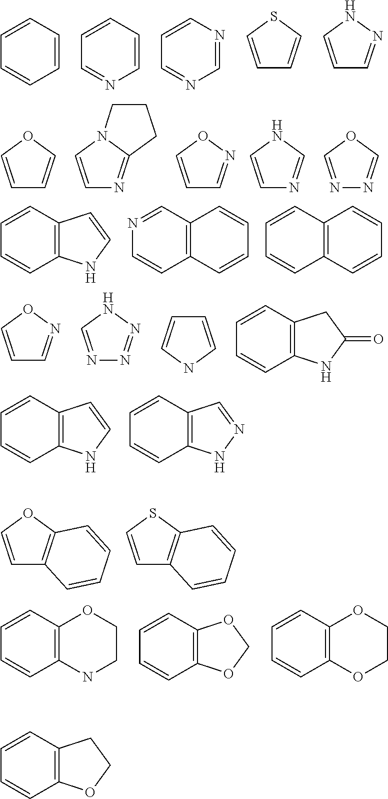

and
R³ is selected from the group of the following substituents
*-C(O)NH—(CH₂)₂CH₃
*-C(O)—N(CH₃)₂
*-C(O)—NH₂
*-C(O)—NH—(CH₂)₂N(CH₃)₂
*-CH₂—C(O)—NH₂
hydrogen
*-F, *-Cl, *-Br
*-CH₃, *-C₂H₅, *-CH═CH₂;
*-CH₂—CN; *-CH(CH₃)—NH₂; *-CH═CH—CN;
—C(O)—OH; *-C(O)—OCH₃, *-C(O)—CH₃, *-C(CH₃)₂-C(O)-OCH₃, *-C(CH₃)₂—CN;
Oxo(═O); hydroxy;

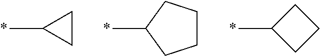

*-NH₂
*-NH—C(O)CH₃
*-NH—SO₂—CH₃
*-NH—C(O)—O—C(CH₃)₃

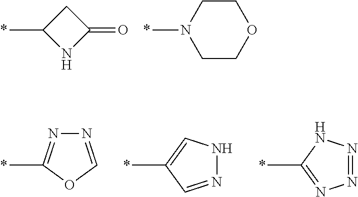

*-SO₂—CH₃
*-SO₂—N(CH₃)₂
*-SO₂—NH₂
*-O—CH₂—CH₃, *O—(CH₂)₂—CH₃, *-O—CF₃;

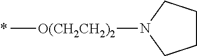

*-OCH₂-Cyclopropyl; *-OCH₃;
*-O(CH₂)₃—CH₃, *-OCH₂-Phenyl; *-O-Phenyl;
*-(CH₂)-OH
*-(CH₂)₂—OH
*-(CH₂)—O—CH₃
*-(CH₂)—O—CH₂—CH₃
*-CH(OH)—CH₂-Phenyl
*-CH(OH)—CH₂—CH₃
*-CH(OH)—(CH₂)₂—CH₃*-CH(OH)—(CH₂)₃—CH₃
*-CH(OH)—CH—(CH₃)₂
*-CH(OH)-Phenyl
*-CH(OH)—CN
*-CH(OH)—CH₂O H
*-CH(OH)—CF₃
*-CH(OH)—(CH₂)₂-Phenyl
*-CH(OH)—C≡CH
*-CH(NH₂)—CH₂—COOH
*-CH₂—NH—SO₂—CH₃
*-CH₂—NH—(CH₂)₃—CH₃
*-CH₂—NH—CH₃
*-CH₂—N(CH₃)₂
*-CH₂—NH—C₂H₅*-CH(CH₃)—NH₂
*-CH₂—NH₂
*-(CH₂)₂-NH₂
*-CH₂—NH—CH₂-Phenyl
*-CH₂—N(C₂H₅)₂
*-CH₂—NH—Cyclopropyl
*-CH₂—NH—Cyclobutyl
*-CH₂—NH—Cyclopentyl
*-CH₂—NH-Pyridyl
*-CH₂—NH-Phenyl
*-CH₂—NH—(CH₂)₂—OH
*-CH₂—N(CH₃)(CH₂)₂OH*-CH₂—NH—CH₂—CN
*-CH₂—N(CH₃)—CH₂—CN
*-CH₂—N(CH₃)—CH₂—CF₃
*-CH₂—N(CH₃)—CH₂—CF₂H
*-CH₂—NH—CH₂—CF₂H
*-CH₂—NH—CH₂—CF₃
*-CH₂—NH—(CH₂)₂—OCH₃

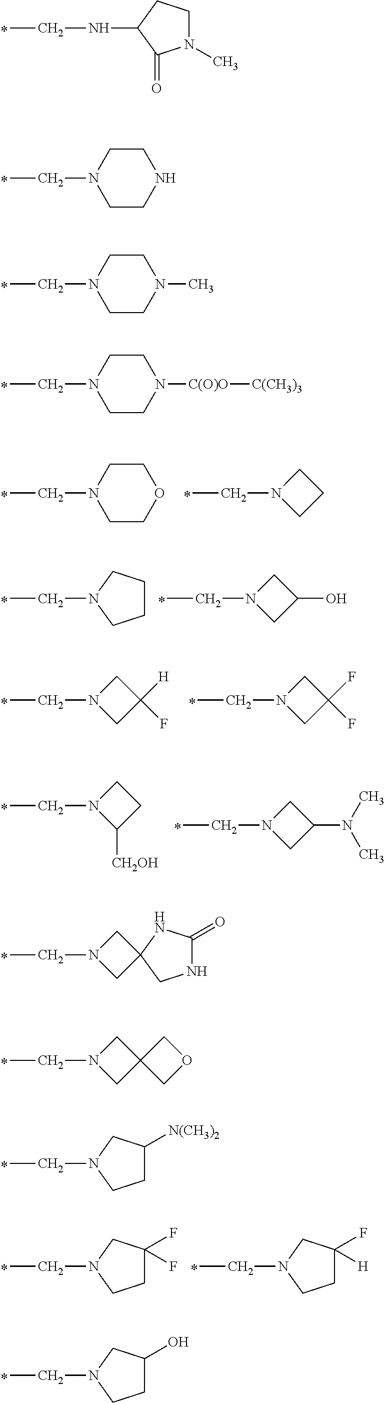

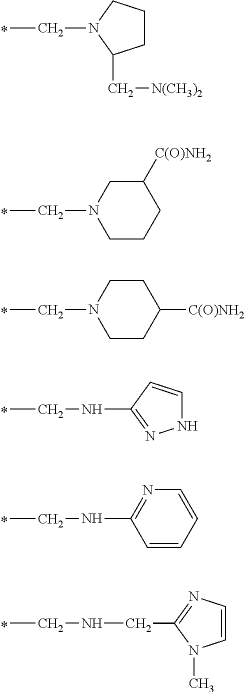

*-CH₂—NH—C(O)—O—C(CH₃)₃
*-(CH₂)₂—NH—C(O)—O—C(CH₃)₃
*-CH₂—NH—C(O)—CH₂—OH
*-CH₂—NH—C(O)—CH₂—OCH₃
*-CH—(CH₃)—NH—C(O)—O—C(CH₃)₃
*-CH₂—NH—C(O)—CH₃

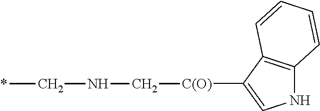

*-CH₂—NH—CH₂—C(O)—NH₂
*-CH₂—NH—CH₂—C(O)—N(CH₃)₂
*-CH₂—NH—CH₂—C(O)—OCH₃
*-CH₂—NH—CH₂—C(O)—NHCH₃
*-CH₂—NH—CH₂—C(O)—NH—(CH₂)₂—O—CH₃
*-CH₂—NH—CH₂—C(O)—NH-CH₂-Phenyl

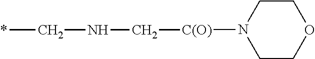

*-CH₂—NH—CH₂—C(O)—NH-Phenyl

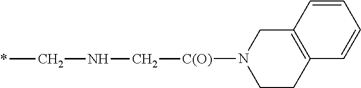

*-CH₂—NH—C(O)—CH₂—NH-Phenyl

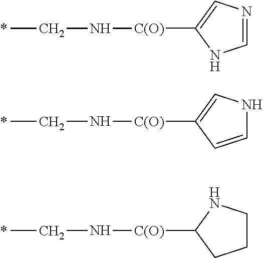

*-CH₂—NH—C(O)—CH₂—NH—CH₂-CF₃

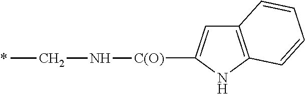

and
y is 1 or 2 and
k is 1 or 2 and
n is 0 or 1
and stereoisomers, tautomers, N-oxides, hydrates, solvates, and salts thereof, and mixtures of same.

In accordance with a further embodiment of the first aspect, the present invention covers the following compounds of general formula (I), supra, namely:
6,7-dimethoxy-2-methyl-N-[(1R)-1-(naphthalen-1-yl)ethyl]quinazolin-4-amine
N-[(1R)-1-(3-chlorophenyl)ethyl]-6,7-dimethoxy-2-methylquinazolin-4-amine
methyl 4-{1-[(6,7-dimethoxy-2-methylquinazolin-4-yl)amino]ethyl}-1-benzothiophene-2-carboxylate
N-[1-(1-benzofuran-7-yl)ethyl]-6,7-dimethoxy-2-methylquinazolin-4-amine
N-[1-(7-fluoro-1H-indazol-4-yl)ethyl]-6,7-dimethoxy-2-methylquinazolin-4-amine
N-[1-(6-fluoro-1H-indazol-4-yl)ethyl]-6,7-dimethoxy-2-methylquinazolin-4-amine
6,7-dimethoxy-2-methyl-N-[1-(5-methyl-1H-indazol-4-yl)ethyl]quinazolin-4-amine
6,7-dimethoxy-2-methyl-N-[1-(2-methyl-2H-indazol-7-yl)ethyl]quinazolin-4-amine
6,7-dimethoxy-2-methyl-N-[1-(2-methyl-2H-indazol-4-yl)ethyl]quinazolin-4-amine
6,7-dimethoxy-2-methyl-N-[1-(1-methyl-1H-indazol-7-yl)ethyl]quinazolin-4-amine
6,7-dimethoxy-2-methyl-N-[1-(5,6,7,8-tetrahydronaphthalen-1-yl)ethyl]quinazolin-4-amine
N-[(1R)-1-(4-fluorophenyl)ethyl]-6,7-dimethoxy-2-methylquinazolin-4-amine
6,7-dimethoxy-2-methyl-N-[1-(3-methyl-1H-indazol-4-yl)ethyl]quinazolin-4-amine
N-[1-(1,3-benzothiazol-4-yl)ethyl]-6,7-dimethoxy-2-methylquinazolin-4-amine
N-[1-(1-benzothiophen-7-yl)ethyl]-6,7-dimethoxy-2-methylquinazolin-4-amine
6,7-dimethoxy-2-methyl-N-[1-(6-methyl-1H-indazol-4-yl)ethyl]quinazolin-4-amine
6,7-dimethoxy-2-methyl-N-[1-(1-methyl-1H-indazol-4-y1)ethyl]quinazolin-4-amine
N-[1-(5-fluoro-1H-indazol-4-yeethyl]-6,7-dimethoxy-2-methylquinazolin-4-amine
N-[1-(2,3-dihydro-1,4-benzodioxin-6-yl)ethyl]-6,7-dimethoxy-2-methylquinazolin-4-amine
N-[1-(1-benzofuran-2-yl)ethyl]-6,7-dimethoxy-2-methylquinazolin-4-amine
N-[1-(2,3-dimethoxyphenyl)ethyl]-6,7-dimethoxy-2-methylquinazolin-4-amine
N-[1-(2,3-dihydro-1-benzofuran-4-yl)ethyl]-6,7-dimethoxy-2-methylquinazolin-4-amine
N-[1-(1,3-benzodioxo1-5-yl)ethyl]-6,7-dimethoxy-2-methylquinazolin-4-amine
N-[1-(2,3-dihydro-1-benzofuran-5-yl)ethyl]-6,7-dimethoxy-2-methylquinazolin-4-amine
6,7-dimethoxy-2-methyl-N-[1-(5,6,7,8-tetrahydronaphthalen-2-yl)ethyl]quinazolin-4-amine
6,7-dimethoxy-2-methyl-N-[1-(2-methylimidazo[1,2-a]pyridin-3-yl)ethyl]quinazolin-4-amine
N-[1-(1-benzothiophen-3-yl)ethyl]-6,7-dimethoxy-2-methylquinazolin-4-amine
2-{1-[(6,7-dimethoxy-2-methylquinazolin-4-yl)amino]ethyl}-1-benzofuran-7-ol
6-bromo-2-methyl-N-[(1R)-1-phenylethyl]quinazolin-4-amine
6-{[dimethyl(oxido)-lambda6-sulfanylidene]amino}-2-methyl-N-[(1R)-1-phenylethyl]quinazolin-4-amine
6-bromo-N-[(1R)-1-(4-fluorophenyl)ethyl]-2-methylquinazolin-4-amine
6-{[dimethyl(oxido)-lambda6-sulfanylidene]amino}-N-[(1R)-1-(4-fluorophenyl)ethyl]-2-methylquinazolin-4-amine
6,7-dimethoxy-N-[1-(7-methoxy-1-benzofuran-2-yl)ethyl]-2-methylquinazolin-4-amine
6-{1-[(6,7-dimethoxy-2-methylquinazolin-4-yl)amino]ethyl}-2H-1,4-benzoxazin-3(4H)-one
6,7-dimethoxy-N-[1-(6-methoxy-2-naphthyl)ethyl]-2-methylquinazolin-4-amine
N-[(1 R)-1-(5′-amino-2′-methylbiphenyl-3-yl)ethyl]-6,7-dimethoxy-2-methylquinazolin-4-amine
6,7-dimethoxy-2-methyl-N-{(1R)-1-[3-(pyrimidin-5-yl)phenyl]ethyl}quinazolin-4-amine
N-{(1R)-1-[3′-(cyclopropylmethoxy)biphenyl-3-yl]ethyl}-6,7-dimethoxy-2-methylquinazolin-4-amine
N-{(1R)-1-[3-(isoquinolin-5-yl)phenyl]ethyl}-6,7-dimethoxy-2-methylquinazolin-4-amine
N-[(1R)-1-(2′-chloro-6′-fluoro-3′-methylbiphenyl-3-yl)ethyl]-6,7-dimethoxy-2-methylquinazolin-4-amine
6,7-dimethoxy-2-methyl-N-{(1R)-1-[3-(5-methylpyridin-3-yl)phenyl]ethyl}quinazolin-4-amine
6,7-dimethoxy-2-methyl-N-{1-[5-(pyrimidin-5-yl)thiophen-2-yl]ethyl}quinazolin-4-amine
6,7-dimethoxy-2-methyl-N-{1-[5-[4-(morpholin-4-yl)phenyl]thiophen-2-yl]ethyl}quinazolin-4-amine
6,7-dimethoxy-2-methyl-N-[1-{5-[3-(morpholin-4-yl)phenyl]thiophen-2-yl}ethyl]quinazolin-4-amine
N-{1-[5-(isoquinolin-5-yl)thiophen-2-yl]ethyl}-6,7-dimethoxy-2-methylquinazolin-4-amine
6,7-dimethoxy-2-methyl-N-{1-[5-(5-methylpyridin-3-yl)thiophen-2-yl]ethyl}quinazolin-4-amine
6,7-dimethoxy-2-methyl-N-{1-[5-(2-propoxyphenyl)thiophen-2-yl]ethyl}quinazolin-4-amine
2-(5-{1-[(6,7-dimethoxy-2-methylquinazolin-4-yl)amino]ethyl}thiophen-2-yl)-N,N-dimethylbenzamide
6,7-dimethoxy-2-methyl-N-{1-[5-(1-methyl-1H-indol-5-yl)thiophen-2-yl]ethyl}quinazolin-4-amine
6,7-dimethoxy-N-[1-{5-[2-(methoxymethyl)phenyl]thiophen-2-yl}ethyl]-2-methylquinazolin-4-amine
3-(5-{1-[(6,7-dimethoxy-2-methylquinazolin-4-yl)amino]ethyl}thiophen-2-yl)-N,N-dimethylbenzamide
(5′-{1-[(6,7-dimethoxy-2-methylquinazolin-4-yl)amino]ethyl}-2,2′-bithiophen-5-yl)methanol
6,7-dimethoxy-2-methyl-N-{1-[5-(3-methylpyridin-4-yl)thiophen-2-yl]ethyl}quinazolin-4-amine
N-{1-[5-(1H-indol-6-yl)thiophen-2-yl]ethyl}-6,7-dimethoxy-2-methylquinazolin-4-amine
6,7-dimethoxy-2-methyl-N-{(1R)-1-[3′-(5-methyl-1,3,4-oxadiazol-2-yl)biphenyl-3-yl]ethyl}quinazolin-4-amine
6,7-dimethoxy-2-methyl-N-[(1R)-1-{3-[5-methylsulfonyl)pyridin-3-yl]phenyl}ethyl]quinazolin-4-amine
5-(3-{(1R)-1-[(6,7-dimethoxy-2-methylquinazolin-4-yl)amino]ethyl}phenyl)-1,3-dihydro-2H-indol-2-one
N-{(1R)-1-[3-(2,2-dimethylcyclopropyl)phenyl]ethyl}-6,7-dimethoxy-2-methylquinazolin-4-amine
6,7-dimethoxy-2-methyl-N-{(1R)-1-[4′-(5-methyl-1,3,4-oxadiazol-2-y1)biphenyl-3-yl]ethyl}quinazolin-4-amine
6,7-dimethoxy-2-methyl-N-{(1R)-1-[3-(1H-pyrrolo[2,3-b]pyridin-5-yl)phenyl]ethyl}quinazolin-4-amine
3′-{(1R)-1-[(6,7-dimethoxy-2-methylquinazolin-4-yl)amino]ethyl}biphenyl-3-sulfonamide
N-{(1R)-1-[3-(2-aminopyrimidin-5-yl)phenyl]ethyl}-6,7-dimethoxy-2-methylquinazolin-4-amine
N-[(1R)-1-{3-[(E)-2-cyclopropylethenyl]phenyl}ethyl]-6,7-dimethoxy-2-methylquinazolin-4-amine
N-{(1R)-1-[2′-(ethoxymethyl)biphenyl-3-yl]ethyl}-6,7-dimethoxy-2-methylquinazolin-4-amine
N-[(1R)-1-(3′-fluoro-5′-methoxybiphenyl-3-yl)ethyl]-6,7-dimethoxy-2-methylquinazolin-4-amine
6,7-dimethoxy-N-{(1R)-1-[3-(5-methoxy-1-benzofuran-2-yl)phenyl]ethyl}-2-methylquinazolin-4-amine
N-[(1R)-1-(2′-butoxy-6′-fluorobiphenyl-3-yl)ethyl]-6,7-dimethoxy-2-methylquinazolin-4-amine
5-(3-{(1R)-1-[(6,7-dimethoxy-2-methylquinazolin-4-yl)amino]ethyl}phenyl)pyridin-2-ol
2-(3′-{(1R)-1-[(6,7-dimethoxy-2-methylquinazolin-4-yl)amino]ethyl}biphenyl-4-yl)-2-methylpropanenitrile
6,7-dimethoxy-2-methyl-N-[1-(5-phenylthiophen-2-yl)ethyl]quinazolin-4-amine
N-[(3′-{(1R)-1-[(6,7-dimethoxy-2-methylquinazolin-4-yl)amino]ethyl}biphenyl-3-yl)methyl]methanesulfonamide
N-[(3′-{(1R)-1-[(6,7-dimethoxy-2-methylquinazolin-4-yl)amino]ethyl}biphenyl-4-yl)methyl]methanesulfonamide
3′-{(1R)-1-[(6,7-dimethoxy-2-methylquinazolin-4-yl)amino]ethyl}-N-propylbiphenyl-4-carboxamide
3′-{(1R)-1-[(6,7-dimethoxy-2-methylquinazolin-4-yl)amino]ethyl}-N-[2-(dimethylamino)ethyl]biphenyl-4-carboxamide
6,7-dimethoxy-2-methyl-N-{(1R)-1-[3-(1H-pyrazo1-3-yl)phenyl]ethyl}quinazolin-4-amine
N-[(1R)-1-{3-[(2E)-but-2-en-2-yl]phenyl}ethyl]-6,7-dimethoxy-2-methylquinazolin-4-amine
N-[(1R)-1-(5′-chloro-2′-propoxybiphenyl-3-yl)ethyl]-6,7-dimethoxy-2-methylquinazolin-4-amine
6,7-dimethoxy-2-methyl-N-[(1R)-1-{3-[(1E)-3-phenylprop-1-en-1-yl]phenyl}ethyl]quinazolin-4-amine
6,7-dimethoxy-2-methyl-N-{(1R)-1-[4′-(morpholin-4-yl)biphenyl-3-yl]ethyl}quinazolin-4-amine
6,7-dimethoxy-2-methyl-N-{(1R)-1-[3′-(morpholin-4-yl)biphenyl-3-yl]ethyl}quinazolin-4-amine
N-{(1R)-1-[2′-(benzyloxy)biphenyl-3-yl]ethyl}-6,7-dimethoxy-2-methylquinazolin-4-amine
6,7-dimethoxy-2-methyl-N-{(1R)-1-[4′-(methylsulfonyl)biphenyl-3-yl]ethyl}quinazolin-4-amine
6,7-dimethoxy-2-methyl-N-{(1R)-1-[2′-(trifluoromethoxy)biphenyl-3-yl]ethyl}quinazolin-4-amine
6,7-dimethoxy-2-methyl-N-{(1R)-1-[3′-(trifluoromethoxy)biphenyl-3-yl]ethyl}quinazolin-4-amine
N-{(1R)-1-[3-(1H-indol-5-yl)phenyl]ethyl}-6,7-dimethoxy-2-methylquinazolin-4-amine
N-{(1R)-1-[3-(furan-3-yl)phenyl]ethyl}-6,7-dimethoxy-2-methylquinazolin-4-amine
N-{(1R)-1-[3-(1-benzothiophen-3-yl)phenyl]ethyl}-6,7-dimethoxy-2-methylquinazolin-4-amine
6,7-dimethoxy-2-methyl-N-{(1R)-1-[3-(1-methyl-1H-indol-2-yl)phenyl]ethyl}quinazolin-4-amine
6,7-dimethoxy-2-methyl-N-[(1R)-1-{3-[(1E)-pent-1-en-1-yl]phenyl}ethyl]quinazolin-4-amine
N-[(1R)-1-{3-[(E)-2-cyclohexylethenyl]phenyl}ethyl]-6,7-dimethoxy-2-methylquinazolin-4-amine
6,7-dimethoxy-2-methyl-N-[(1R)-1-(2′-phenoxybiphenyl-3-yl)ethyl]quinazolin-4-amine
tert-butyl (3′-{(1R)-1-[(6,7-dimethoxy-2-methylquinazolin-4-yl)amino]ethyl}biphenyl-4-yl)carbamate
(2E)-3-(3′-{(1R)-1-[(6,7-dimethoxy-2-methylquinazolin-4-yl)amino]ethyl}biphenyl-3-yl)prop-2-enenitrile
N-[(1R)-1-(2′,4′-dimethylbiphenyl-3-yl)ethyl]-6,7-dimethoxy-2-methylquinazolin-4-amine
1-[5-(3-{(1R)-1-[(6,7-dimethoxy-2-methylquinazolin-4-yl)amino]ethyl}phenyl)thiophen-2-yl]ethanone
N-{(1R)-1-[3-(1,3-benzodioxol-5-yl)phenyl]ethyl}-6,7-dimethoxy-2-methylquinazolin-4-amine
N-{(1R)-1-[4′-(benzyloxy)biphenyl-3-yl]ethyl}-6,7-dimethoxy-2-methylquinazolin-4-amine
N-{1-[5-(2,3-dihydro-1,4-benzodioxin-6-yl)thiophen-2-yl]ethyl}-6,7-dimethoxy-2-methylquinazolin-4-amine
6,7-dimethoxy-N-[(1R)-1-(3′-methoxybiphenyl-3-yl)ethyl-]-2-methylquinazolin-4-amine
6,7-dimethoxy-2-methyl-N-{(1R)-1-[3′-(trifluoromethyl)biphenyl-3-yl]ethyl}quinazolin-4-amine
3′-{(1R)-1-[(6,7-dimethoxy-2-methylquinazolin-4-yl)amino]ethyl}-N,N-dimethylbiphenyl-2-sulfonamide
6,7-dimethoxy-2-methyl-N-[(1R)-1-(2′-propoxybiphenyl-3-yl)ethyl]quinazolin-4-amine
3′-{(1R)-1-[(6,7-dimethoxy-2-methylquinazolin-4-yl)amino]ethyl}-N,N-dimethylbiphenyl-2-carboxamide
6,7-dimethoxy-N-{(1R)-1-[2′-(methoxymethyl)biphenyl-3-yl]ethyl}-2-methylquinazolin-4-amine
6,7-dimethoxy-2-methyl-N-{(1R)-1-[3-(1-methyl-1H-indol-5-yl)phenyl]ethyl}quinazolin-4-amine
3′-{(1R)-1-[(6,7-dimethoxy-2-methylquinazolin-4-yl)amino]ethyl}-N,N-dimethylbiphenyl-3-carboxamide
[5-(3-{(1R)-1-[(6,7-dimethoxy-2-methylquinazolin-4-yl)amino]ethyl}phenyl)thiophen-2-yl]methanol
6,7-dimethoxy-2-methyl-N-{(1R)-1-[3-(3-methylpyridin-4-yl)phenyl]ethyl}quinazolin-4-amine
N-{(1R)-1-[3-(1H-indol-6-yl)phenyl]ethyl}-6,7-dimethoxy-2-methylquinazolin-4-amine
N-{(1R)-1-[3-(1H-indol-4-yl)phenyl]lethyl}-6,7-dimethoxy-2-methylquinazolin-4-amine
6,7-dimethoxy-N-{(1R)-1-[3-(2-methoxypyrimidin-5-yl)phenyl]ethyl}-2-methylquinazolin-4-amine
6,7-dimethoxy-2-methyl-N-{(1R)-1-[3′-(methylsulfonyl)biphenyl-3-yl]ethyl}quinazolin-4-amine
N-{(1R)-1-[3-(2,3-dihydro-1-benzofuran-5-yl)phenyl]ethyl}-6,7-dimethoxy-2-methylquinazolin-4-amine
6,7-dimethoxy-2-methyl-N-[(1R)-1-{3-[(E)-2-phenyletheny]phenyl}ethyl]quinazolin-4-amine
3′-{(1R)-1-[(6,7-dimethoxy-2-methylquinazolin-4-yl)amino]ethyl}-N,N-dimethylbiphenyl-4-carboxamide
6,7-dimethoxy-2-methyl-N-[(1R)-1-{3-[(1E)-prop-1-en-1-yl]phenyl}ethyl]quinazolin-4-amine
N-{(1R)-1-[3-(cyclopent-1-en-1-yl)phenyl]ethyl}-6,7-dimethoxy-2-methylquinazolin-4-amine
N-(3′-{(1R)-1-[(6,7-dimethoxy-2-methylquinazolin-4-yl)amino]ethyl}biphenyl-3-yl)methanesulfonamide
N-(3′-{(1R)-1-[(6,7-dimethoxy-2-methylquinazolin-4-yl)amino]ethyl}biphenyl-2-yl)acetamide
6,7-dimethoxy-2-methyl-N-{(1R)-1-[2′-(methylsulfonyl)biphenyl-3-yl]ethyl}quinazolin-4-amine
N-(3′-{(1R)-1-[(6,7-dimethoxy-2-methylquinazolin-4-yl)amino]ethyl}biphenyl-2-yl)methanesulfonamide
N-{1-[5-(3,5-dichlorophenyl)thiophen-2-yl]ethyl}-6,7-dimethoxy-2-methylquinazolin-4-amine
N-{(1R)-1-[3′-(benzyloxy)biphenyl-3-yl]ethyl}-6,7-dimethoxy-2-methylquinazolin-4-amine
N-[(1R)-1-(3′,5′-dichlorobiphenyl-3-yl)ethyl]-6,7-dimethoxy-2-methylquinazolin-4-amine
N-{(1R)-1-[3-(2,3-dihydro-1,4-benzodioxin-6-yl)phenyl]ethyl}-6,7-dimethoxy-2-methylquinazolin-4-amine
6,7-dimethoxy-2-methyl-N-[1-{5-[3-(5-methyl-1,3,4-oxadiazol-2-yl)phenyl]thiophen-2-yl}ethyl]quinazolin-4-amine
6,7-dimethoxy-2-methyl-N-[1-{5-[5-(methylsulfonyl)pyridin-3-yl]thiophen-2-yl}ethyl]quinazolin-4-amine
6,7-dimethoxy-2-methyl-N-[1-{5-[4-(5-methyl-1,3,4-oxadiazol-2-yl)phenyl]thiophen-2-yl}ethyl]quinazolin-4-amine
6,7-dimethoxy-2-methyl-N-{1-[5-(1H-pyrrolo[2,3-b]pyridin-5-yl)thiophen-2-yl]ethyl}quinazolin-4-amine
3-(5-{1-[(6,7-dimethoxy-2-methylquinazolin-4-yl)amino]ethyl}thiophen-2-yl)benzenesulfonamide
N-{1-[5-(2-aminopyrimidin-5-yl)thiophen-2-yl]ethyl}-6,7-dimethoxy-2-methylquinazolin-4-amine
N-[1-{5-[(E)-2-cyclopropylethenyl]thiophen-2-yl}ethyl]-6,7-dimethoxy-2-methylquinazolin-4-amine
N-[1-{5-[2-(ethoxymethyl)phenyl]thiophen-2-yl}ethyl]-6,7-dimethoxy-2-methylquinazolin-4-amine
N-{1-[5-(3-fluoro-5-methoxyphenyl)thiophen-2-yl]ethyl}-6,7-dimethoxy-2-methylquinazolin-4-amine
N-[1-{5-[3-(benzyloxy)phenyl]thiophen-2-yl}ethyl]-6,7-dimethoxy-2-methylquinazolin-4-amine
N-{1-[5-(2-butoxy-6-fluorophenyethiophen-2-yl]ethyl}-6,7-dimethoxy-2-methylquinazolin-4-amine
2-[4-(5-{1-[(6,7-dimethoxy-2-methylquinazolin-4-yl)amino]ethyl}thiophen-2-yl)phenyl]-2-methylpropanenitrile
N-[4-(5-{1-[(6,7-dimethoxy-2-methylquinazolin-4-yl)amino]ethyl}thiophen-2-yl)benzyl]acetamide
N-[3-(5-{1-[(6,7-dimethoxy-2-methylquinazolin-4-yl)amino]ethyl}thiophen-2-yl)benzyl]methanesulfonamide
N-[4-(5-{1-[(6,7-dimethoxy-2-methylquinazolin-4-yl)amino]ethyl}thiophen-2-yl)benzyl]methanesulfonamide
4-(5-{1-[(6,7-dimethoxy-2-methylquinazolin-4-yl)amino]ethyl}thiophen-2-yl)-N-propylbenzamide
4-(5-{1-[(6,7-dimethoxy-2-methylquinazolin-4-yl)amino]ethyl}thiophen-2-yl)-N-[2-(dimethylamino)ethyl]benzamide
N-[1-{5-[(2E)-but-2-en-2-yl]thiophen-2-yl}ethyl]-6,7-dimethoxy-2-methylquinazolin-4-amine
N-{1-[5-(5-chloro-2-propoxyphenyl)thiophen-2-yl]ethyl}-6,7-dimethoxy-2-methylquinazolin-4-amine
6,7-dimethoxy-2-methyl-N-[1-{5-[(1E)-3-phenylprop-1-en-1-yl]thiophen-2-yl}ethyl]quinazolin-4-amine
N-{1-[5-(5-amino-2-methylphenyl)thiophen-2-yl]ethyl}-6,7-dimethoxy-2-methylquinazolin-4-amine
N-{1-[5-(3,5-dimethyl-1,2-oxazol-4-yl)thiophen-2-yl]ethyl}-6,7-dimethoxy-2-methylquinazolin-4-amine
6,7-dimethoxy-2-methyl-N-[1-{5-[2-(methylsulfonyl)phenyl]thiophen-2-yl}ethyl]quinazolin-4-amine
6,7-dimethoxy-2-methyl-N-[1-{5-[4-(methylsulfonyl)phenyl]thiophen-2-yl}ethyl]quinazolin-4-amine
6,7-dimethoxy-2-methyl-N-[1-{5-[2-(trifluoromethoxy)phenyl]thiophen-2-yl}ethyl]quinazolin-4-amine
6,7-dimethoxy-2-methyl-N-[1-{5-[3-(trifluoromethoxy)phenyl]thiophen-2-yl}ethyl]quinazolin-4-amine
N-{1-[5-(1H-indol-5-yl)thiophen-2-yl]ethyl}-6,7-dimethoxy-2-methylquinazolin-4-amine
N-{1-[5-(furan-3-yl)thiophen-2-yl]ethyl}-6,7-dimethoxy-2-methylquinazolin-4-amine
N-{1-[5-(1-benzothiophen-3-yl)thiophen-2-yl]ethyl}-6,7-dimethoxy-2-methylquinazolin-4-amine
6,7-dimethoxy-2-methyl-N-{1-[5-(1-methyl-1H-indol-2-yl)thiophen-2-yl]ethyl}quinazolin-4-amine
6,7-dimethoxy-2-methyl-N-[1-{5-[(1E)-pent-1-en-1-yl]thiophen-2-yl}ethyl]quinazolin-4-amine
N-[1-{5-[(E)-2-cyclohexylethenyl]thiophen-2-yl}ethyl]-6,7-dimethoxy-2-methylquinazolin-4-amine
6,7-dimethoxy-2-methyl-N-{1-[5-(2-phenoxyphenyl)thiophen-2-yl]ethyl}quinazolin-4-amine
tert-butyl [4-(5-{1-[(6,7-dimethoxy-2-methylquinazolin-4-yl)amino]ethyl}thiophen-2-yl)phenyl]carbamate
(2E)-3-[3-(5-{1-[(6,7-dimethoxy-2-methylquinazolin-4-yl)amino]ethyl}thiophen-2-yl)phenyl]prop-2-enenitrile
N-{1-[5-(2,4-dimethylphenyl)thiophen-2-yl]ethyl}-6,7-dimethoxy-2-methylquinazolin-4-amine
1-(5′-{1-[(6,7-dimethoxy-2-methylquinazolin-4-yl)amino]ethyl}-2,2′-bithiophen-5-yl)ethanone
N-{1-[5-(1,3-benzodioxo1-5-yl)thiophen-2-yl]ethyl}-6,7-dimethoxy-2-methylquinazolin-4-amine
6,7-dimethoxy-2-methyl-N-{1-[5-(4-methyl-3,4-dihydro-2H-1,4-benzoxazin-7-yl)thiophen-2-yl]ethyl}quinazolin-4-amine
N-[3-(5-{1-[(6,7-dimethoxy-2-methylquinazolin-4-yl)amino]ethyl}thiophen-2-yl)phenyl]methanesulfonamide
N-[2-(5-{1-[(6,7-dimethoxy-2-methylquinazolin-4-yl)amino]ethyl}thiophen-2-yl)phenyl]acetamide
6,7-dimethoxy-N-{1-[5-(3-methoxyphenyethiophen-2-yl]ethyl}-2-methylquinazolin-4-amine
6,7-dimethoxy-2-methyl-N-[1-{5-[3-(trifluoromethyl)phenyl]thiophen-2-yl}ethyl]quinazolin-4-amine
2-(5-{1-[(6,7-dimethoxy-2-methylquinazolin-4-yl)amino]ethyl}thiophen-2-yl)-N,N-dimethylbenzenesulfonamide
N-[1-{5-[3-(cyclopropylmethoxy)phenyl]thiophen-2-yl}ethyl]-6,7-dimethoxy-2-methylquinazolin-4-amine
N-{1-[5-(1H-indol-4-yl)thiophen-2-yl]ethyl}-6,7-dimethoxy-2-methylquinazolin-4-amine
6,7-dimethoxy-N-{1-[5-(2-methoxypyrimidin-5-yl)thiophen-2-yl]ethyl}-2-methylquinazolin-4-amine
6,7-dimethoxy-2-methyl-N-[1-{5-[3-(methylsulfonyl)phenyl]thiophen-2-yl]ethyl}quinazolin-4-amine
N-{1-[5-(2,3-dihydro-1-benzofuran-5-yl)thiophen-2-yl]ethyl}-6,7-dimethoxy-2-methylquinazolin-4-amine
6,7-dimethoxy-2-methyl-N-[1-{5-[(E)-2-phenylethenyl]thiophen-2-yl}ethyl]quinazolin-4-amine
4-(5-{1-[(6,7-dimethoxy-2-methylquinazolin-4-yl)amino]ethyl}thiophen-2-yl)-N,N-dimethylbenzamide
6,7-dimethoxy-2-methyl-N-[1-{5-[(1E)-prop-1-en-1-yl]thiophen-2-yl}ethyl]quinazolin-4-amine
methyl 2-(5-{1-[(6,7-dimethoxy-2-methylquinazolin-4-yl)amino]ethyl}thiophen-2-yl)benzoate
1-[2-(5-{1-[(6,7-dimethoxy-2-methylquinazolin-4-yl)amino]ethyl}thiophen-2-yl)benzyl]piperidine-4-carboxamide
2-[2-(5-{1-[(6,7-dimethoxy-2-methylquinazolin-4-yl)amino]ethyl}thiophen-2-yl)phenyl]ethanol
2-[3-(5-{1-[(6,7-dimethoxy-2-methylquinazolin-4-yl)amino]ethyl}thiophen-2-yl)phenyl]ethanol
6,7-dimethoxy-2-methyl-N-[1-{5-[2-(2-oxa-6-azaspiro[3.3]hept-6-ylmethyl)phenyl]thiophen-2-yl}ethyl]quinazolin-4-amine
N-[1-(5-bromo-4-methylthiophen-2-yl)ethyl]-6,7-dimethoxy-2-methylquinazolin-4-amine
6,7-dimethoxy-2-methyl-N-[1-{5-[2-(pyrrolidin-1-ylmethyl)phenyl]thiophen-2-yl}ethyl]quinazolin-4-amine
N-[1-{5-[2-({2-[(dimethylamino)methyl]pyrrolidin-1-yl}methyl)phenyl]thiophen-2-yl}ethyl]-6,7-dimethoxy-2-methylquinazolin-4-amine
2-methyl-N-[(1R)-1-(naphthalen-1-yl)ethyl]quinazolin-4-amine
N-[(1R)-1-(4-bromophenyl)ethyl]-6,7-dimethoxy-2-methylquinazolin-4-amine
6,7-dimethoxy-2-methyl-N-{(1R)-1-[4-(methylsulfonyl)phenyl]ethyl}quinazolin-4-amine
4-{(1R)-1-[(6,7-dimethoxy-2-methylquinazolin-4-yl)amino]ethyl}benzonitrile
6,7-dimethoxy-2-methyl-N-[(1R)-1-(3-methylphenyl)ethyl]quinazolin-4-amine
N-[(1R)-1-(3-bromophenyl)ethyl]-6,7-dimethoxy-2-methylquinazolin-4-amine
4-{(1R)-1-[(6,7-dimethoxy-2-methylquinazolin-4-yl)amino]ethyl}benzamide
N-[(1R)-1-(biphenyl-3-yl)ethyl]-6,7-dimethoxy-2-methylquinazolin-4-amine
3-{(1R)-1-[(6,7-dimethoxy-2-methylquinazolin-4-yl)amino]ethyl}benzonitrile
6,7-dimethoxy-2-methyl-N-[(1R)-1-(4-methylphenyl)ethyl]quinazolin-4-amine
N-[(1R)-1-(biphenyl-4-yl)ethyl]-6,7-dimethoxy-2-methylquinazolin-4-amine
N-[(1R)-1-(4-cyclopropylphenyl)ethyl]-6,7-dimethoxy-2-methylquinazolin-4-amine
6,7-dimethoxy-2-methyl-N-{(1R)-1-[3-(methylsulfonyl)phenyl]ethyl}quinazolin-4-amine
3-{(1R)-1-[(6,7-dimethoxy-2-methylquinazolin-4-yl)amino]ethyl}benzamide
6,7-dimethoxy-2-methyl-N-{(1R)-1-[3-(1H-pyrazol-4-yl)phenyl]ethyl}quinazolin-4-amine
6,7-dimethoxy-2-methyl-N-{(1R)-1-[4-(1-methyl-1,2,3,6-tetrahydropyridin-4-yl)phenyl]ethyl}quinazolin-4-amine
6,7-dimethoxy-2-methyl-N-{(1R)-1-[3-(1-methyl-1,2,3,6-tetrahydropyridin-4-yl)phenyl]ethyl}quinazolin-4-amine
6,7-dimethoxy-2-methyl-N-{(1R)-1-[4-(prop-1-en-2-yl)phenyl]ethyl}quinazolin-4-amine
N-[(1R)-1-(3-cyclopropylphenyl)ethyl]-6,7-dimethoxy-2-methylquinazolin-4-amine
N-[1-(1-benzothiophen-4-yl)ethyl]-6,7-dimethoxy-2-methylquinazolin-4-amine
6,7-dimethoxy-2-methyl-N-[(1R)-1-phenylethyl]quinazolin-4-amine
6,7-dimethoxy-2-methyl-N-[1-(thiophen-2-yl)ethyl]quinazolin-4-amine
N-[1-(5-bromofuran-2-yl)ethyl]-6,7-dimethoxy-2-methylquinazolin-4-amine
N-[1-(5-bromothiophen-2-yl)ethyl]-6,7-dimethoxy-2-methylquinazolin-4-amine
1-[2-(5-{1-[(6,7-dimethoxy-2-methylquinazolin-4-yl)amino]ethyl}thiophen-2-yl)benzyl]pyrrolidin-3-ol
N-{1-[5-(2-{[(3S)-3-fluoropyrrolidin-1-yl]methyl}phenyl)thiophen-2-yl]ethyl}-6,7-dimethoxy-2-methylquinazolin-4-amine
6,7-dimethoxy-2-methyl-N-[1-(quinolin-5-yl)ethyl]quinazolin-4-amine
6,7-dimethoxy-2-methyl-N-[1-(5-phenylfuran-2-yl)ethyl]quinazolin-4-amine
N-[1-(5-bromo-2,3-dihydro-1-benzofuran-7-yl)ethyl]-6,7-dimethoxy-2-methylquinazolin-4-amine
6,7-dimethoxy-2-methyl-N-[1-(3-phenoxyphenyl)ethyl]quinazolin-4-amine
6,7-dimethoxy-2-methyl-N-{1-[3-(2H-tetrazol-5-yl)phenyl]ethyl}quinazolin-4-amine
6,7-dimethoxy-2-methyl-N-[1-(quinolin-8-yl)ethyl]quinazolin-4-amine
2-[4-(5-{1-[(6,7-dimethoxy-2-methylquinazolin-4-yl)amino]ethyl}thiophen-2-yl)-1H-pyrazol-1-yl]ethanol
N-{1-[5-(6,7-dihydro-5H-pyrrolo [1,2-a]imidazol-3-yl)thiophen-2-yl]ethyl}-6,7-dimethoxy-2-methylquinazolin-4-amine
6,7-dimethoxy-2-methyl-N-[1-(5-{1-[2-(pyrrolidin-1-yl)ethyl]-1H-pyrazol-4-yl}thiophen-2-yl)ethyl]quinazolin-4-amine
N-{1-[5-(1-cyclopentyl-1H-pyrazol-4-yl)thiophen-2-yl]ethyl}-6,7-dimethoxy-2-methylquinazolin-4-amine
6,7-dimethoxy-2-methyl-N-{1-[5-(1H-pyrazol-3-yl)thiophen-2-yl]ethyl}quinazolin-4-amine
N-[1-(5-{2-[(3,3-difluoropyrrolidin-1-yl)methyl]phenyl}thiophen-2-yl)ethyl]-6,7-dimethoxy-2-methylquinazolin-4-amine
6,7-dimethoxy-2-methyl-N-[1-(5-phenylfuran-2-yeethyl]quinazolin-4-amine
N-[1-(5-bromo-2,3-dihydro-1-benzofuran-7-yl)ethyl]-6,7-dimethoxy-2-methylquinazolin-4-amine
2-[1-(5-{1-[(6,7-dimethoxy-2-methylquinazolin-4-yl)amino]ethyl}furan-2-yl)-1H-pyrazol-3-yl]ethanol
5-{1-[(6,7-dimethoxy-2-methylquinazolin-4-yl)amino]ethyl}pyridin-2(1H)-one
6,7-dimethoxy-2-methyl-N-[1-(3-phenoxyphenyl)ethyl]quinazolin-4-amine
N-[1-(2,1,3-benzothiadiazol-5-yl)ethyl]-6,7-dimethoxy-2-methylquinazolin-4-amine
6,7-dimethoxy-2-methyl-N-[(1R)-1-(quinolin-8-yl)ethyl]quinazolin-4-amine
N-{1-[5-(cyclopent-1-en-1-yl)thiophen-2-yl]ethyl}-6,7-dimethoxy-2-methylquinazolin-4-amine
N-{1-[5-(2-ethoxyphenyl)thiophen-2-yl]ethyl }-6,7-dimethoxy-2-methylquinazolin-4-amine
N-{1-[5-(4-fluoronaphthalen-1-yl)thiophen-2-yl]ethyl}-6,7-dimethoxy-2-methylquinazolin-4-amine
N-[1-{5-[2-(aminomethyl)-4-fluorophenyl]thiophen-2-yl}ethyl]-6,7-dimethoxy-2-methylquinazolin-4-amine
N-[1-{5-(3, 6-dihydro-2H-pyran-4-yl)thiophen-2-yl]ethyl }-6,7-dimethoxy-2-methylquinazolin-4-amine
tert-butyl {[5-(5-{1-[(6,7-dimethoxy-2-methylquinazolin-4-yl)amino]ethyl}thiophen-2-yl)furan-2-yl]methyl}carbamate
methyl 3-(5-{1-[(6,7-dimethoxy-2-methylquinazolin-4-yl)amino]ethyl}thiophen-2-yl)-1-methyl-1H-pyrazole-5-carboxylate
N-{1-[5-(2-{[3-(dimethylamino)pyrrolidin-1-yl]methyl}phenyl)thiophen-2-yl]ethyl}-6,7-dimethoxy-2-methylquinazolin-4-amine
N-[1-(5-bromothiophen-3-yl)ethyl]-6,7-dimethoxy-2-methylquinazolin-4-amine
2-(5-{1-[(6,7-dimethoxy-2-methylquinazolin-4-yl)amino]ethyl}thiophen-2-yl)benzamide
3-(5-{1-[(6,7-dimethoxy-2-methylquinazolin-4-yl)amino]ethyl}thiophen-2-yl)benzamide
4-(5-{1-[(6,7-dimethoxy-2-methylquinazolin-4-yl)amino]ethyl}thiophen-2-yl)benzamide
N-{1-[5-(2-aminophenyl)thiophen-2-yl]ethyl}-6,7-dimethoxy-2-methylquinazolin-4-amine
[2-(5-{1-[(6,7-dimethoxy-2-methylquinazolin-4-yl)amino]ethyl}thiophen-2-yl)phenyl]methanol
2-(5-{1-[(6,7-dimethoxy-2-methylquinazolin-4-yl)amino]ethyl}thiophen-2-yl)benzonitrile
N-{1-[5-(1H-indazol-7-yl)thiophen-2-yl]ethyl}-6,7-dimethoxy-2-methylquinazolin-4-amine
N-{1-[5-(1H-indazol-4-yl)thiophen-2-yl]ethyl}-6,7-dimethoxy-2-methylquinazolin-4-amine
N-{1-[5-(2-ethenylphenyl)thiophen-2-yl]ethyl}-6,7-dimethoxy-2-methylquinazolin-4-amine
2-[4-(5-{1-[(6,7-dimethoxy-2-methylquinazolin-4-yl)amino]ethyl}thiophen-2-yl)-1H-pyrazol-1-yl]acetamide
6,7-dimethoxy-2-methyl-N-[1-(5-{2-[(methylamino)methyl]phenyl}thiophen-2-yl)ethyl]quinazolin-4-amine
2-[4-(4-{1-[(6,7-dimethoxy-2-methylquinazolin-4-yl)amino]ethyl}thiophen-2-yl)-1H-pyrazol-1-yl]ethanol
N-[1-{5-[2-(aminomethyl)phenyl]thiophen-2-yl}ethyl]-6,7-dimethoxy-2-methylquinazolin-4-amine
N-{1-[5-(6,7-dihydro-5H-pyrrolo [1,2-a]imidazol-3-yl)thiophen-3-yl]ethyl}-6,7-dimethoxy-2-methylquinazolin-4-amine
6,7-dimethoxy-2-methyl-N-[1-(5-{1-[2-(pyrrolidin-1-yl)ethyl]-1H-pyrazol-4-yl}thiophen-3-yl)ethyl]quinazolin-4-amine
N-[1-{5-[2-(aminomethyl)-4-fluorophenyl]thiophen-3-yl}ethyl]-6,7-dimethoxy-2-methylquinazolin-4-amine
2-[4-(3-{(1R)-1-[(6,7-dimethoxy-2-methylquinazolin-4-yl)amino]ethyl}phenyl)-1H-pyrazol-1-yl]ethanol
6,7-dimethoxy-2-methyl-N-[(1R)-1-(3-{1-[2-(pyrrolidin-1-yl)ethyl]-1H-pyrazol-4-yl}phenyl)ethyl]quinazolin-4-amine
N-{(1R)-1-[2′-(aminomethyl)biphenyl-3-yl]ethyl}-6,7-dimethoxy-2-methylquinazolin-4-amine
N-[1-{5-[2-(aminomethyl)phenyl]thiophen-3-yl}ethyl]-6,7-dimethoxy-2-methylquinazolin-4-amine
N-{(1R)-1-[3-(aminomethyl)phenyl]ethyl}-6,7-dimethoxy-2-methylquinazolin-4-amine
N-{(1R)-1-[3-(6,7-dihydro-5H-pyrrolo[1,2-a]imidazol-3-yl)phenyl]ethyl}-6,7-dimethoxy-2-methylquinazolin-4-amine
N-[1-(5-{2-[(dimethylamino)methyl]phenyl}thiophen-2-yl)ethyl]-6,7-dimethoxy-2-methylquinazolin-4-amine
6,7-dimethoxy-2-methyl-N-[1-(5-{2-[(methylamino)methyl]phenyl}thiophen-3-yl)ethyl]quinazolin-4-amine
N-[1-(4-bromothiophen-2-yl)ethyl]-6,7-dimethoxy-2-methylquinazolin-4-amine
N-[1-{5-[3-(aminomethyl)phenyl]thiophen-2-yl}ethyl]-6,7-dimethoxy-2-methylquinazolin-4-amine
N-[1-{5-[4-(aminomethyl)phenyl]thiophen-2-yl}ethyl]-6,7-dimethoxy-2-methylquinazolin-4-amine
6,7-dimethoxy-2-methyl-N-[1-(4-{1-[2-(pyrrolidin-1-yl)ethyl]-1H-pyrazol-3-yl}thiophen-2-yl)ethyl]quinazolin-4-amine
6,7-dimethoxy-2-methyl-N-[(1R)-1-{2′-[(methylamino)methyl]biphenyl-3-yl}ethyl]quinazolin-4-amine
N-[1-{4-[2-(aminomethyl)-4-fluorophenyl]thiophen-2-yl}ethyl]-6,7-dimethoxy-2-methylquinazolin-4-amine
N-[1-{4-[2-(aminomethyl)phenyl]thiophen-2-yl}ethyl]-6,7-dimethoxy-2-methylquinazolin-4-amine
6,7-dimethoxy-2-methyl-N-[1-(4-{2-[(methylamino)methyl]phenyl}thiophen-2-yl)ethyl]quinazolin-4-amine
N-{1-[4-(6,7-dihydro-5H-pyrrolo [1,2-a]imidazol-3-yl)thiophen-2-yl]ethyl}-6,7-dimethoxy-2-methylquinazolin-4-amine
2-[3-(5-{1-[(6,7-dimethoxy-2-methylquinazolin-4-l)amino]ethyl}thiophen-3-yl)-1H-pyrazol-1-yl]ethanol
N-{(1R)-1-[2′-(aminomethyl)-4′-fluorobiphenyl-3-yl]ethyl}-6,7-dimethoxy-2-methylquinazolin-4-amine
N-[1-{5-[5-(aminomethyl)furan-2-yl]thiophen-2-yl}ethyl]-6,7-dimethoxy-2-methylquinazolin-4-amine
N-{1-[5′-(aminomethyl)-2,2′-bithiophen-5-yl-]ethyl}-6,7-dimethoxy-2-methylquinazolin-4-amine
2-{[2-(5-{1-[(6,7-dimethoxy-2-methylquinazolin-4-yl)amino]ethyl}thiophen-2-yl)benzyl]amino}-1-(1H-indol-3-yl)ethanone
3-amino-4-(5-{1-[(6,7-dimethoxy-2-methylquinazolin-4-yl)amino]ethyl}thiophen-2-yl)-1-benzothiophene-2-carboxamide
2-[2-(5-{1-[(6,7-dimethoxy-2-methylquinazolin-4-yl)amino]ethyl}thiophen-2-yl)benzyl]glycinamide
2-[2-(5-{1-[(6,7-dimethoxy-2-methylquinazolin-4-yl)amino]ethyl}thiophen-2-yl)benzyl]-N,N-dimethylglycinamide
methyl N-[2-(5-{1-[(6,7-dimethoxy-2-methylquinazolin-4-yl)amino]ethyl}thiophen-2-yl)benzyl]glycinate
2-[2-(5-{1-[(6,7-dimethoxy-2-methylquinazolin-4-yl)amino]ethyl}thiophen-2-yl)benzyl]-N-methylglycinamide
2-[2-(5-{1-[(6,7-dimethoxy-2-methylquinazolin-4-yl)amino]ethyl}thiophen-2-yl)benzyl]-N-(2-methoxyethyl)glycinamide
N-benzyl-2-[2-(5-{1-[(6,7-dimethoxy-2-methylquinazolin-4-yl)amino]ethyl}thiophen-2-yl)benzyl]glycinamide
2-{[2-(5-{1-[(6,7-dimethoxy-2-methylquinazolin-4-yl)amino]ethyl}thiophen-2-yl)benzyl]amino}-1-(morpholin-4-yl)ethanone
3-(5-{1-[(6,7-dimethoxy-2-methylquinazolin-4-yl)amino]ethyl}thiophen-2-yl)-1,5-dimethyl-1H-pyrrole-2-carbonitrile
5-{1-[(6,7-dimethoxy-2-methylquinazolin-4-yl)amino-]ethyl}-2,3′-bithiophene-4′-carbonitrile
N-[1-(5-{2-[(diethylamino)methyl]phenyl}thiophen-2-yl)ethyl]-6,7-dimethoxy-2-methylquinazolin-4-amine
2-[2-(5-{1-[(6,7-dimethoxy-2-methylquinazolin-4-yl)amino]ethyl}thiophen-2-yl)benzyl]-N-phenylglycinamide
1-[2-(5-{1-[(6,7-dimethoxy-2-methylquinazolin-4-yl)amino]ethyl}thiophen-2-yl)benzyl]piperidine-3-carboxamide
N-{1-[5-(2-{[(2,2-difluoroethyl)(methyl)amino]methyl}phenyl)thiophen-2-yl]ethyl}-6,7-dimethoxy-2-methylquinazolin-4-amine
N-[1-{5-[2-(aminomethyl)-5-chlorophenyl]thiophen-2-yl}ethyl]-6,7-dimethoxy-2-methylquinazolin-4-amine
N-[2-(5-{1-[(6,7-dimethoxy-2-methylquinazolin-4-yl)amino]ethyl}thiophen-2-yl)-5-fluorobenzyl]-1H-imidazole-2-carboxamide
N-[2-(5-{1-[(6,7-dimethoxy-2-methylquinazolin-4-yl)amino]ethyl}thiophen-2-yl)-5-fluorobenzyl]-1H-imidazole-5-carboxamide
N-[245-{1-[(6,7-dimethoxy-2-methylquinazolin-4-yl)amino]ethyl}thiophen-2-yl)-5-fluorobenzyl]-N&lt;sup&gt;2&lt;/sup&gt;-(2,2,2-trifluoroethyl)glycinamide
N-[2-(5-{1-[(6,7-dimethoxy-2-methylquinazolin-4-yl)amino]ethyl}thiophen-2-yl)-5-fluorobenzyl]-1H-indole-2-carboxamide
2-{[2-(5-{1-[(6,7-dimethoxy-2-methylquinazolin-4-yl)amino]ethyl}thiophen-2-yl)benzyl]amino}ethanol
2-{[2-(5-{1-[(6,7-dimethoxy-2-methylquinazolin-4-yl)amino]ethyl}thiophen-2-yl)benzyl](methyl)amino}ethanol
N-[2-(5-{1-[(6,7-dimethoxy-2-methylquinazolin-4-yl)amino]ethyl}thiophen-2-yl)-5-fluorobenzyl]-N2-phenylglycinamide
6,7-dimethoxy-2-methyl-N-{1-[5-(2-{[(2,2,2-trifluoroethyl)amino]methyl}phenyl)thiophen-2-yl]ethyl}quinazolin-4-amine
6,7-dimethoxy-2-methyl-N-[1-(5-{2-[(pyridin-2-ylaminonnethyl]phenyl}thiophen-2-yl)ethyl]quinazolin-4-amine
6,7-dimethoxy-2-methyl-N-[1-(5-{2-[(1H-pyrazol-3-ylamino)nethyl]phenyl}thiophen-2-yl)ethyl]quinazolin-4-amine
1-(3,4-dihydroisoquinolin-2(1H)-yl)-2-{[2-(5-{1-[(6,7-dimethoxy-2-methylquinazolin-4-yl)amino]ethyl}thiophen-2-yl)benzyl]amino}ethanone
N-[1-{5-[4-fluoro-2-({[(1-methyl-1H-imidazol-2-yl)methyl]amino}methyl)phenyl]thiophen-2-yl}ethyl]-6,7-dimethoxy-2-methylquinazolin-4-amine
6,7-dimethoxy-2-methyl-N-[1-{5-[2-(piperazin-1-ylmethyl)phenyl]thiophen-2-yl}ethyl]quinazolin-4-amine hydrochloride
tert-butyl 4-[2-(5-{1-[(6,7-dimethoxy-2-methylquinazolin-4-yl)amino]ethyl}thiophen-2-yl)benzyl]piperazine-1-carboxylate
N-[2-(5-{1-[(6,7-dimethoxy-2-methylquinazolin-4-yl)amino]ethyl}thiophen-2-yl)-5-fluorobenzyl]acetamide
6,7-dimethoxy-2-methyl-N-[1-(5-{2-[(4-methylpiperazin-1-yl)methyl]phenyl}thiophen-2-yl)ethyl]quinazolin-4-amine
(3S)-{[2-(5-{1-[(6,7-dimethoxy-2-methylquinazolin-4-yl)amino]ethyl}thiophen-2-yl)benzyl]amino}-1-methylpyrrolidin-2-one
N-[2-(5-{1-[(6,7-dimethoxy-2-methylquinazolin-4-yl)amino]ethyl}thiophen-2-yl)-5-fluorobenzyl]-1H-pyrazole-3-carboxamide
6,7-dimethoxy-2-methyl-N-[1-{5-[2-(morpholin-4-ylmethyl)phenyl]thiophen-2-yl}ethyl]quinazolin-4-amine
1-[2-(5-{1-[(6,7-dimethoxy-2-methylquinazolin-4-yl)amino]ethyl}thiophen-2-yl)benzyl]azetidin-3-ol
2-[2-(5-{1-[(6,7-dimethoxy-2-methylquinazolin-4-yl)amino]ethyl}thiophen-2-yl)benzyl]-2,5,7-triazaspiro [3.4]octan-6-one
N-[2-(5-{1-[(6,7-dimethoxy-2-methylquinazolin-4-yl)amino]ethyl}thiophen-2-yl)-5-fluorobenzyl]-L-prolinamide
N-{1-[5-(2-{[(2,2-difluoroethyl)amino]methyl}phenyl)thiophen-2-yl]ethyl}-6,7-dimethoxy-2-methylquinazolin-4-amine
N-[2-(5-{1-[(6,7-dimethoxy-2-methylquinazolin-4-yl)amino]ethyl}thiophen-2-yl)-5-fluorobenzyl]-prolinamide
N-[1-{5-[2-(azetidin-1-ylmethyl)phenyl]thiophen-2-yl}ethyl]-6,7-dimethoxy-2-methylquinazolin-4-amine
{1-[(2S)-(5-{1-[(6,7-dimethoxy-2-methylquinazolin-4-yl)amino]ethyl}thiophen-2-yl)benzyl]azetidin-2-yl}methanol
N-{1-[5-(2-{[3-(dimethylamino)azetidin-1-yl]methy}phenyl)thiophen-2-yl]ethyl}-6,7-dimethoxy-2-methylquinazolin-4-amine
N-[1-(5-{2-[(3,3-difluoroazetidin-1-yl)methyl]phenyl}thiophen-2-yl)ethyl]-6,7-dimethoxy-2-methylquinazolin-4-amine
6,7-dimethoxy-2-methyl-N-{1-[5-(2-{[methyl(2,2,2-trifluoroethyl)amino]methyl}phenyl)thiophen-2-yl]ethyl}quinazolin-4-amine
N-[1-(5-{2-[(3-fluoroazetidin-1-yl)nethyl]phenyl}thiophen-2-yl)ethyl]-6,7-dimethoxy-2-methylquinazolin-4-amine
N-[1-(5-{4-chloro-2-[(dimethylamino)methyl]phenyl}thiophen-2-yl)ethyl]-6,7-dimethoxy-2-methylquinazolin-4-amine
1-[2-(5-{1-[(6,7-dimethoxy-2-methylquinazolin-4-yl)amino]ethyl}thiophen-2-yl)phenyl]ethanone
6,7-dimethoxy-2-methyl-N-[1-(5-{2-[2-(pyrrolidin-1-yl)ethoxy]phenyl}thiophen-2-yl)ethyl]quinazolin-4-amine
6,7-dimethoxy-2-methyl-N-[1-(4-{2-[(methylamino)methyl]phenyl}thiophen-2-yl)ethyl]quinazolin-4-amine, enantiomer 1
6,7-dimethoxy-2-methyl-N-[1-(4-{2-[(methylamino)methyl]phenyl}thiophen-2-yl)ethyl]quinazolin-4-amine, enantiomer 2
N-{1-[5-(6,7-dihydro-5H-pyrrolo[1,2-a]imidazol-3-yl)thiophen-2-yl]ethyl}-6,7-dimethoxy-2-methylquinazolin-4-amine, enantiomer 1
N-{1-[5-(6,7-dihydro-5H-pyrrolo [1,2-a]imidazol-3-yl)thiophen-2-yl]ethyl}-6,7-dimethoxy-2-methylquinazolin-4-amine, enantiomer 2
6,7-dimethoxy-2-methyl-N-[1-(2-methyl-1-benzothiophen-4-yl)ethyl]quinazolin-4-amine
6,7-dimethoxy-2-methyl-N-[1-(thieno[2,3-b]pyridin-4-yl)ethyl]quinazolin-4-amine
6,7-dimethoxy-2-methyl-N-[1-(thieno [2,3-c]pyridin-4-yl)ethyl]quinazolin-4-amine
6,7-dimethoxy-2-methyl-N-[1-(thieno[3,2-c]pyridin-4-yl)ethyl]quinazolin-4-amine
N-{(1R)-1-[3-(3,5-dimethyl-1H-pyrazol-4-yl)phenyl]ethyl}-6,7-dimethoxy-2-methylquinazolin-4-amine
6,7-dimethoxy-2-methyl-N-{(1R)-1-[3-(5-methyl-1H-pyrazol-4-yl)phenyl]ethyl}quinazolin-4-amine
N-{(1R)-1-[3-(3,5-dimethyl-1,2-oxazol-4-yl)phenyl]ethyl}-6,7-dimethoxy-2-methylquinazolin-4-amine
6,7-dimethoxy-2-methyl-N-{(1R)-1-[3-(1H-pyrazo1-5-yl)phenyl]ethyl}quinazolin-4-amine
6,7-dimethoxy-2-methyl-N-{(1R)-1-[3-(1-methyl-1H-pyrazol-4-yl)phenyl]ethyl}quinazolin-4-amine
6,7-dimethoxy-2-methyl-N-{(1R)-1-[3-(1-methyl-1H-pyrazol-5-yl)phenyl]ethyl}quinazolin-4-amine
N-{(1R)-1-[3-(1H-imidazol-1-yl)phenyl]ethyl}-6,7-dimethoxy-2-methylquinazolin-4-amine
6,7-dimethoxy-2-methyl-N-{(1R)-1-[3-(1H-pyrazol-1-yl)phenyl]ethyl}quinazolin-4-amine
N-{(1R)-1-[3-(1H-imidazol-4-yl)phenyl]ethyl}-6,7-dimethoxy-2-methylquinazolin-4-amine
6-(benzyloxy)-N-[(1R)-1-(3-bromophenyl)ethyl]-7-methoxy-2-methylquinazolin-4-amine
6-(benzyloxy)-7-methoxy-2-methyl-N-{(1R)-1-[3-(1H-pyrazo1-4-yl)phenyl]ethyl}quinazolin-4-amine
7-methoxy-2-methyl-4-({(1R)-1-[3-(1H-pyrazol-4-yl)phenyl]ethyl}amino)quinazolin-6-ol
6-(cyclopropylmethoxy)-7-methoxy-2-methyl-N-{(1R)-1-[3-(1H-pyrazol-4-yl)phenyl]ethyl}quinazolin-4-amine
6,7-dimethoxy-2-methyl-N-[1-(thiophen-2-yl)ethyl]quinazolin-4-amine
7-methoxy-6-(2-methoxyethoxy)-2-methyl-N-{(1R)-1-[3-(1H-pyrazol-4-yl)phenyl]ethyl}quinazolin-4-amine
(1R)-1-[2-(5-{1-1(6,7-dimethoxy-2-methylquinazolin-4-yl)amino]ethyl}thiophen-2-yl)phenyllpropan-1-ol
6-butoxy-7-methoxy-2-methyl-N-{(1R)-1-[3-(1H-pyrazol-4-yl)phenyl]ethyl}quinazolin-4-amine
7-methoxy-2-methyl-6-(3-methylbutoxy)-N-{(1R)-1-[3-(1H-pyrazol-4-yl)phenyl]ethyl}quinazolin-4-amine
tert-butyl {2-[2-(5-{1-[(6,7-dimethoxy-2-methylquinazolin-4-yl)amino]ethyl}thiophen-2-yl)phenyl]ethyl}carbamate
7-methoxy-2-methyl-6-(propan-2-yloxy)-N-{(1R)-1-[3-(1H-pyrazol-4-yl)phenyl]ethyl}quinazolin-4-amine
7-methoxy-2-methyl-6-(oxetan-3-ylmethoxy)-N-{(1R)-1-[3-(1H-pyrazol-4-yl)phenyl]ethyl}quinazolin-4-amine
6-ethoxy-7-methoxy-2-methyl-N-{(1R)-1-[3-(1H-pyrazol-4-yl)phenyl]ethyl}quinazolin-4-amine
6-ethoxy-N-{(1R)-1-[3-(1-ethyl-1H-pyrazol-4-yl)phenyl]ethyl}-7-methoxy-2-methylquinazolin-4-amine
N-[1-{5-[2-(2-aminoethyl)phenyl]thiophen-2-yl}ethyl]-6,7-dimethoxy-2-methylquinazolin-4-amine
tert-butyl {1-[2-(5-{1-[(6,7-dimethoxy-2-methylquinazolin-4-yl)amino]ethyl}thiophen-2-yl)phenyl]ethyl}carbamate
N-[1-(5-{2-[1-aminoethyl]phenyl}thiophen-2-yl)ethyl]-6,7-dimethoxy-2-methylquinazolin-4-amine
N-[1-(5-{2-[1-aminoethyl]phenyl}thiophen-2-yl)ethyl]-6,7-dimethoxy-2-methylquinazolin-4-amine
6,7-dimethoxy-2-methyl-N-[1-{5-[2-(1H-pyrazol-4-yl)phenyl]thiophen-2-yl}ethyl]quinazolin-4-amine
6,7-dimethoxy-2-methyl-N-[1-(5-{2-[(phenylamino)methyl]phenyl}thiophen-2-yl)ethyl]quinazolin-4-amine
6-bromo-N-[(1R)-1-(3-chlorophenyl)ethyl]-2-methylquinazolin-4-amine
N-[1-(5-{2-[(cyclopentylamino)methyl]phenyl}thiophen-2-yl)ethyl]-6,7-dimethoxy-2-methylquinazolin-4-amine
N-[1-(5-{2-[(benzylamino)methyl]phenyl}thiophen-2-yl)ethyl]-6,7-dimethoxy-2-methylquinazolin-4-amine
N-[1-(5-{2-[(butylamino)methyl]phenyl}thiophen-2-yl)ethyl]-6,7-dimethoxy-2-methylquinazolin-4-amine
N-[1-(5-{2-[(ethylamino)methyl]phenyl}thiophen-2-yl)ethyl]-6,7-dimethoxy-2-methylquinazolin-4-amine
6,7-dimethoxy-2-methyl-N-[1-{5-[2-(1H-tetrazol-5-yl)phenyl]thiophen-2-yl}ethyl]quinazolin-4-amine
6,7-dimethoxy-N-{1-[5-(2-{[(2-methoxyethyl)amino]methyl}phenyl)thiophen-2-yl]ethyl}-2-methylquinazolin-4-amine
N-[1-(5-{2-[(cyclopropylamino)methyl]phenyl}thiophen-2-yl)ethyl]-6,7-dimethoxy-2-methylquinazolin-4-amine
methyl 4-{[(1R)-1-(3-chlorophenyl)ethyl]amino}-2-methylquinazoline-6-carboxylate
4-{[(1R)-1-(3-chlorophenyl)ethyl]amino}-2-methylquinazoline-6-carboxylic acid
(4-{[(1R)-1-(3-chlorophenyl)ethyl]amino}-2-methylquinazolin-6-yl)methanol
[2-(5-{1-[(6,7-dimethoxy-2-methylquinazolin-4-yl)amino]ethyl}thiophen-2-yl)phenyl](phenyl)methanol
1-[2-(5-{1-[(6,7-dimethoxy-2-methylquinazolin-4-yl)amino]ethyl}thiophen-2-yl)phenyl]-3-phenylpropan-1-ol
1-[2-(5-{1-[(6,7-dimethoxy-2-methylquinazolin-4-yl)amino]ethyl}thiophen-2-yl)phenyl]-2-phenylethanol
1-[2-(5-{1-[(6,7-dimethoxy-2-methylquinazolin-4-yl)amino]ethyl}thiophen-2-yl)phenyl]pentan-1-ol
1-[2-(5-{1-[(6,7-dimethoxy-2-methylquinazolin-4-yl)amino]ethyl}thiophen-2-yl)phenyl]prop-2-yn-1-ol
1-[2-(5-{1-[(6,7-dimethoxy-2-methylquinazolin-4-yl)amino]ethyl}thiophen-2-yl)phenyl]-2-methylpropan-1-ol
1-[2-(5-{1-[(6,7-dimethoxy-2-methylquinazolin-4-yl)amino]ethyl}thiophen-2-yl)phenyl]-2,2,2-trifluoroethanol
N-{1-[5-(6,7-dihydro-5H-pyrrolo[1,2-a]imidazol-3-yl)-4-methylthiophen-2-yl]ethyl}-6,7-dimethoxy-2-methylquinazolin-4-amine
N-[2-(5-{1-[(6,7-dimethoxy-2-methylquinazolin-4-yl)amino]ethyl}thiophen-2-yl)-5-fluorobenzyl]-2-hydroxyacetamide
N-[2-(5-{1-[(6,7-dimethoxy-2-methylquinazolin-4-yl)amino]ethyl}thiophen-2-yl)-5-fluorobenzyl]-2-methoxyacetamide
N-(1-(5-(4-bromo-2-((dimethylamino)methyl)phenyl)thiophen-2-yl)ethyl)-6,7-dimethoxy-2-methylquinazolin-4-amine
N-(1-(5-(2-((Dimethylamino) methyl)-4-(trifluoromethyl) phenyl) thiophen-2-yl) ethyl)-6, 7-dimethoxy-2-methylquinazolin-4-amine
6,7-dimethoxy-2-methyl-N-(1-{5-[2-methyl-4-(trifluoromethyl)phenyl]-2-thienyl}ethyl)quinazolin-4-amine
tert-butyl [4-chloro-2-(5-{1-[(6,7-dimethoxy-2-methylquinazolin-4-yl)amino]ethyl}thiophen-2-yl)benzyl]carbamate
4-(3-{(1R)-1-[(6,7-dimethoxy-2-methylquinazolin-4-yl)amino]ethyl}phenyl)pyridin-2-ol
N-{1-[3-(benzyloxy)phenyl]lethyl}-6,7-dimethoxy-2-methylquinazolin-4-amine
N-[1-(5-{2-[(dimethylamino)methyl]phenyl}thiophen-2-yl)ethyl]-6,7-dimethoxy-2-methylquinazolin-4-amine (enantiomer 1)
N-[1-(5-{2-[(dimethylamino)methyl]phenyl}thiophen-2-yl)ethyl]-6,7-dimethoxy-2-methylquinazolin-4-amine (enantiomer 2)
2-(4-{[(1R)-1-(3-chlorophenyl)ethyl]amino}-2-methylquinazolin-6-yl)propan-2-ol
2-(3′-{(1R)-1-[(6,7-dimethoxy-2-methylquinazolin-4-yl)amino]ethyl}biphenyl-2-yl)acetamide
2-[2-(5-{1-[(6,7-dimethoxy-2-methylquinazolin-4-yl)amino]ethyl}thiophen-2-yl)phenyl]acetamide
5-(5-{1-[(6,7-dimethoxy-2-methylquinazolin-4-yl)amino]ethyl}thiophen-2-yl)pyridin-2-ol
N-[(1R)-1-(3-chlorophenyl)ethyl]-6-methoxy-2, 8-dimethylquinazolin-4-amine
N-[1-{5-[2-(aminomethyl)-3-chlorophenyl]thiophen-2-yl}ethyl]-6,7-dimethoxy-2-methylquinazolin-4-amine
N-[1-{5-[2-(aminomethyl)-4-chlorophenyl]thiophen-2-yl}ethyl]-6,7-dimethoxy-2-methylquinazolin-4-amine
N-(1-{5-[2-(aminomethyl)-4-fluorophenyl]-4-methyl-2-thienyl}ethyl)-6,7-dimethoxy-2-methylquinazolin-4-amine
N-[1-(5-{2-[(dimethylamino)methyl]phenyl}-4-methyl-2-thienyl)ethyl]-6,7-dimethoxy-2-methylquinazolin-4-amine
2-{[2-(5-{1-[(6,7-dimethoxy-2-methylquinazolin-4-yl)amino]ethyl}-3-methyl-2-thienyl)benzyl](methyl)amino}ethanol
6,7-dimethoxy-2-methyl-N-[1-(4-methyl-5-{2-[(methylamino)methyl]-phenyl}thiophen-2-yl)ethyl]quinazolin-4-amine
1-[245-{1-[(6,7-dimethoxy-2-methylquinazolin-4-yl)amino]ethyl}thiophen-2-yl)phenyl]ethane-1,2-diol
N-[(1R)-1-(3-chlorophenyl)ethyl]-2, 6-dimethylquinazolin-4-amine
N-[(1R)-1-(3-chlorophenyl)ethyl]-2-methyl-6-(1H-pyrazol-4-yl)quinazolin-4-amine
N-[(1R)-1-(3-chlorophenyl)ethyl]-2-methyl-6-(1-methyl-1H-pyrazol-4-yl)quinazolin-4-amine
N-[(1R)-1-(3-chlorophenyl)ethyl]-6-cyclopropyl-2-methylquinazolin-4-amine
tert-butyl [3-chloro-2-(5-{1-[(6,7-dimethoxy-2-methylquinazolin-4-yl)amino]ethyl}thiophen-2-yl)benzyl]carbamate
N-[1-{5-[2-(aminomethyl)-6-chlorophenyl]thiophen-2-yl}ethyl]-6,7-dimethoxy-2-methylquinazolin-4-amine
4-(5-{1-[(6,7-dimethoxy-2-methylquinazolin-4-yl)amino]ethyl}-2-thienyl)pyridin-2-ol
4-[2-(5-{1-[(6,7-dimethoxy-2-methylquinazolin-4-yl)amino]ethyl}thiophen-2-yl)phenyl]azetidin-2-one
N-[(1R)-1-(3-chlorophenyl)ethyl]-6-methoxy-2,7-dimethylquinazolin-4-amine
4-(5-{1-[(6,7-dimethoxy-2-methylquinazolin-4-yl)amino]ethyl}thiophen-2-yl)-3-[(dimethylamino)methyl]benzonitrile
N-[1(5-bromothiophen-2-yl)ethyl]-6-[3-(dimethylamino)pyrrolidin-1-yl]-2-methylquinazolin-4-amine
N-[1-(5-{2-[(dimethylamino)methyl]phenyl}thiophen-2-yl)ethyl]-2-methyl-6-(pyrrolidin-1-yl)quinazolin-4-amine
N-[1-(5-bromothiophen-2-yl)ethyl]-2-methyl-6-(4-methylpiperazin-1-yl)quinazolin-4-amine
N-[1-(5-{2-[(dimethylamino)methyl]phenyl}thiophen-2-yl)ethyl]-2-methyl-6-(4-methylpiperazin-1-yl)quinazolin-4-amine
N-[1-(5-{2-[(dimethylamino)methyl]phenyl}thiophen-2-yl)ethyl]-6-[3-(dimethylamino)pyrrolidin-1-yl]-2-methylquinazolin-4-amine
N-[(1R)-1-(5-bromothiophen-2-yl)ethyl]-2-methyl-6-(pyrrolidin-1-yl)quinazolin-4-amine
N-(4-{[1-(5-{2-[(dimethylamino)methyl]phenyl}thiophen-2-yl)ethyl]amino}-2-methylquinazolin-6-yl)acetamide
N-[1-(5-{2-[(dimethylamino)methyl]phenyl}thiophen-2-yl)ethyl]-2-methyl-6-nitroquinazolin-4-amine
6,7-dimethoxy-N-{1-[5-(4-methoxy-2-methylphenyl)thiophen-2-yl]ethyl}-2-methylquinazolin-4-amine
N4-[1-(5-{2-[(dimethylamino)methyl]phenyl}-2-thienyl)ethyl]-2-methylquinazoline-4,6-diamine
N-[1-(5-bromothiophen-2-yl)ethyl]-2-methyl-6-[4-(pyridin-3-ylmethyl)piperazin-1-yl]quinazolin-4-amine
N4-[1-(5-bromo-2-thienyl)ethyl]-2-methyl-N6-[2-(morpholin-4-yl)ethyl]quinazoline-4,6-diamine
N-[1-(5-{2-[(dimethylamino)methyl]phenyl}thiophen-2-yl)ethyl]-2-methyl-6-[4-(pyridin-3-ylmethyl)piperazin-1-yl]quinazolin-4-amine
N-{2-[(4-{[1-(5-{2-[(dimethylamino)methyl]phenyl}thiophen-2-yl)ethyl]amino}-2-methylquinazolin-6-yl)amino]ethyl}acetamide
N-[1-(5-bromo-3-chlorothiophen-2-yl)ethyl]-6,7-dimethoxy-2-methylquinazolin-4-amine
tert-butyl [2-(4-chloro-5-{1-[(6,7-dimethoxy-2-methylquinazolin-4-yl)amino]ethyl}thiophen-2-yl)benzyl]carbamate
N-[1-{5-[2-(aminomethyl)phenyl]-4-chlorothiophen-2-yl}ethyl]-6,7-dimethoxy-2-methylquinazolin-4-amine
N-[1-(4-chloro-5-{2-[(dimethylamino)methyl]phenyl}thiophen-2-yl)ethyl]-6,7-dimethoxy-2-methylquinazolin-4-amine
N-[1-(5-bromo-4-chlorothiophen-2-yl)ethyl]-6,7-dimethoxy-2-methylquinazolin-4-amine
N4-[1-(5-{2-[(dimethylamino)methyl]phenyl}-2-thienyl)ethyl]-2-methyl-N6-[2-(morpholin-4-yl)ethyl]quinazoline-4,6-diamine
4-(4-{[1(5-bromothiophen-2-yl)ethyl]amino}-2-methylquinazolin-6-yl)-1-methylpiperazin-2-one
4-(4-{[1-(5-{2-[(dimethylamino)methyl]phenyl}thiophen-2-yl)ethyl]amino}-2-methylquinazolin-6-yl)-1-methylpiperazin-2-one
methyl 2-[2-(5-{1-[(6,7-dimethoxy-2-methylquinazolin-4-yl)amino]ethyl}thiophen-2-yl)phenyl]-2-methylpropanoate
N-[1-{5-[2-(aminomethyl)phenyl]-3-chlorothiophen-2-yl}ethyl]-6,7-dimethoxy-2-methylquinazolin-4-amine
N-(4-{[1-(5-{2-[(dimethylamino)methyl]phenyl}thiophen-2-yl)ethyl]amino}-2-methylquinazolin-6-yl)methanesulfonamide
N-[1-(5-{2-[(dimethylamino)methyl]-4-methoxyphenyl}thiophen-2-yl)ethyl]-6,7-dimethoxy-2-methylquinazolin-4-amine
3-(4-{[1-(5-{2-[(dimethylamino)methyl]phenyl}thiophen-2-yl)ethyl]amino}-2-methylquinazolin-6-yl)-1,1-dimethylurea
1-benzyl-4-(4-{[1(5-bromothiophen-2-yl)ethyl]amino}-2-methylquinazolin-6-yl)piperazin-2-one
N-[1-(5-{2-[(dimethylamino)methyl]-4-methylphenyl}thiophen-2-yl)ethyl]-6,7-dimethoxy-2-methylquinazolin-4-amine
N-[1-(5-{4-cyclopropyl-2-[(dimethylamino)methyl]phenyl}thiophen-2-yl)ethyl]-6,7-dimethoxy-2-methylquinazolin-4-amine
6,7-dimethoxy-2-methyl-N-[1-(2-methyl-1,3-thiazol-4-yl)ethyl]quinazolin-4-amine
6,7-dimethoxy-2-methyl-N-[1-(4-methyl-1,3-thiazol-2-yl)ethyl]quinazolin-4-amine
3-(5-{1-[(6,7-dimethoxy-2-methylquinazolin-4-yl)amino]ethyl}thiophen-2-yl)-1-methyl-1H-pyrazole-5-carboxylic acid
tert-butyl [(5′-{1-[(6,7-dimethoxy-2-methylquinazolin-4-yl)amino-]ethyl}-2,2′-bithiophen-5-yl)methyl]carbamate
7-methoxy-2-methyl-6-[2-(methylsulfonyl)ethoxy]-N-{(1R)-1-[3-(1H-pyrazol-4-yl)phenyl]ethyl}quinazolin-4-amine
tert-butyl [5-chloro-2-(5-{1-[(6,7-dimethoxy-2-methylquinazolin-4-yl)amino]ethyl}thiophen-2-yl)benzyl]carbamate
tert-butyl [2-chloro-6-(5-{1-[(6,7-dimethoxy-2-methylquinazolin-4-yl)amino]ethyl}thiophen-2-yl)benzyl]carbamate
7-bromo-N-[(1R)-1-(3-chlorophenyl)ethyl]-2-methylquinazolin-4-amine
N-[1-(5-bromothiophen-2-yl)ethyl]-2-methyl-6-nitroquinazolin-4-amine
methyl 4-{[(1R)-1-(3-chlorophenyl)ethyl]amino}-2-methylquinazoline-7-carboxylate
3-amino-3-[2-(5-{1-[(6,7-dimethoxy-2-methylquinazolin-4-yl)amino]ethyl}thiophen-3-yl)phenyl]propanoic acid
N-[1-{5-[2-(2-aminopropan-2-yl)phenyl]thiophen-2-yl}ethyl]-6,7-dimethoxy-2-methylquinazolin-4-amine
{[2-(5-{1-[(6,7-dimethoxy-2-methylquinazolin-4-yl)amino]ethyl}thiophen-2-yl)benzyl](methyl)amino}acetonitrile
1-(4-{[1-(5-{2-[(dimethylamino)methyl]phenyl}thiophen-2-yl)ethyl]amino}-2-methylquinazolin-6-yl)-3-methylurea and
1-benzyl-4-(4-{[1-(5-{2-[(dimethylamino)methyl]phenyl}thiophen-2-yl)ethyl]amino}-2-methylquinazolin-6-yl)piperazin-2-one

In another embodiment of the first aspect, the present invention covers compounds of formula (I), supra, in which the carbon atom between the nitrogen atom and the substituent A1 is in (R)-configuration.

In yet another embodiment of the first aspect, the present invention covers compounds of formula (I), supra, wherein Rl is selected from the list of the following substituents

H, *-OCH₃,—OC₂H₅,

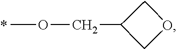

*-CH₂O H, —C(O)OH, *-C(O)OCH₃, —Br,

*-O-CH(CH₃)_{2.}, *-O—(CH₂)₂CH(CH₃)₂,*-O—(CH₂)₃CH₃,*-O—(CH₂)₂O—CH₃,

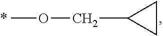

*-O—CH₂-Phenyl, *-N═S (O)(CH₃)₂, *-CH₃,

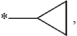 [SMILES: *C1CC1]

*-NH(CH₃), *-N(CH₃)₂, *-NH₂,

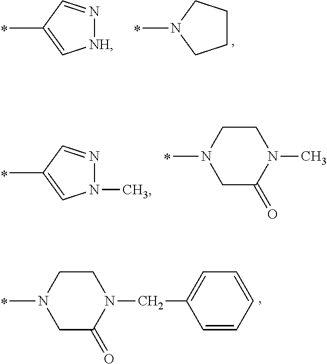

*-C(CH₃)₂—OH,

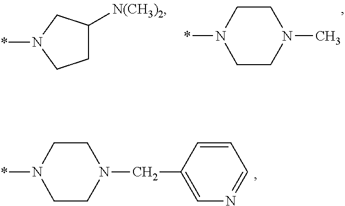

*-NH-(CH₂)₂₋NH—C(O)—CH₃,. *-NH—(CH₂)₂-morpholino, *-NH—C(O)—CH₃, *-NH—C(O)—NH—CH₃,

*-NH—C(O)—N(CH₃)₂, *-NO₂, *-NH—S(O)₂—CH₃, *-N═S(O)(CH₃)₂, *-OH, *-O—(CH₂)₂—S(O)₂—CH₃,

*-fluorine,

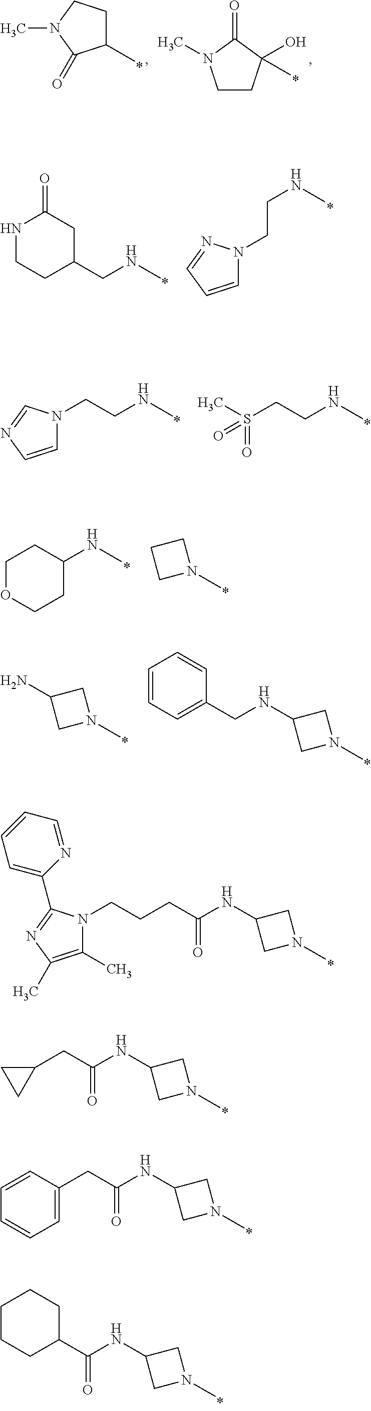

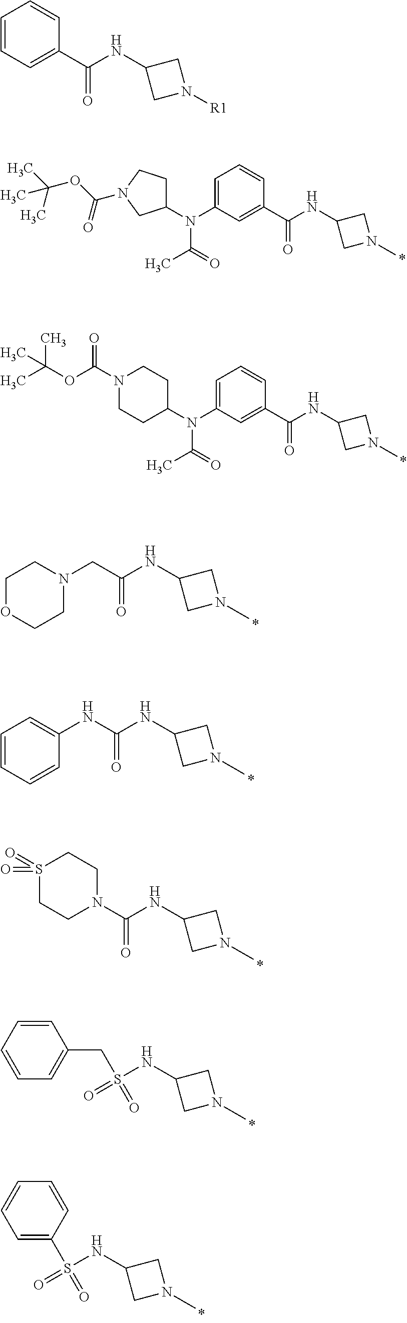

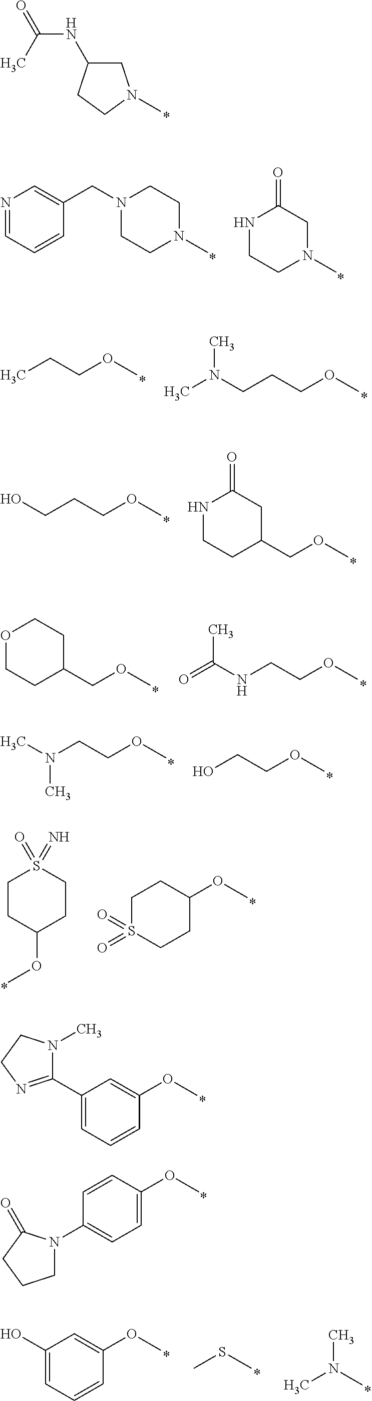

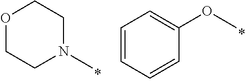

In a further embodiment of the first aspect, the present invention covers compounds of formula (I), supra, wherein R² is selected from the group of hydrogen, hydroxy, oxo (═O), cyano, cyclopropyl, 1,1-dimethylcyclopropyl, -C(═CH₂)CH₃, —C(CH₃)═CHCH₃, —CH═CH—(CH₂)₂CH₃, CH═CHCH₃, —CH═CH-cyclopropyl), —C(O)NH₂, C(O)OCH₃, —S(O)₂CH₃, —OCH₃, —CH₂NH₂, a halogen atom (F, Cl; Br),

In an even further embodiment of the first aspect, the present invention covers compounds of formula (I), supra, wherein A1 is selected from the group

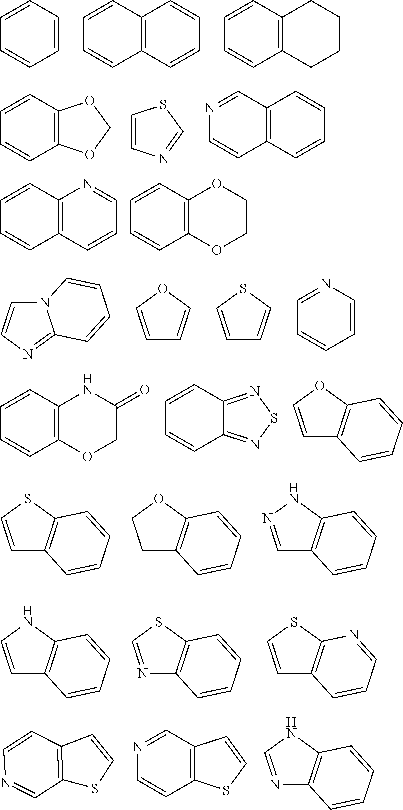

or a stereoisomer, a tautomer, an N-oxide, a hydrate, a solvate, or a salt thereof, or a mixture of same.

In a further another embodiment of the first aspect, the present invention covers compounds of formula (I), supra, wherein A1 is a phenyl ring or a thienyl ring.

In a particular embodiment of the first aspect, the present invention covers compounds of formula (I), supra, wherein A2 is selected from the group

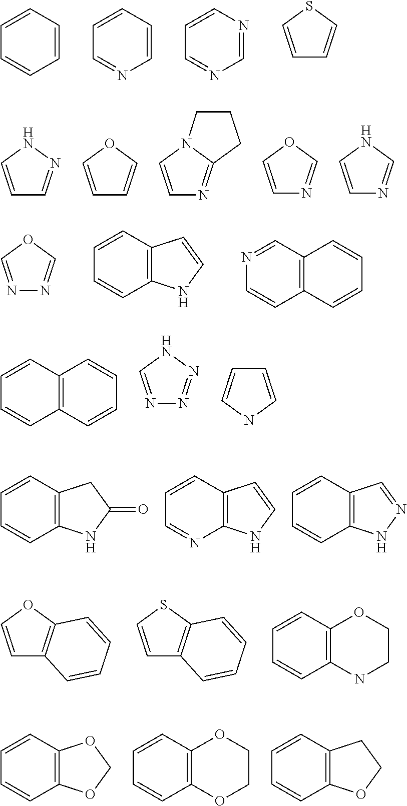

or a stereoisomer, a tautomer, an N-oxide, a hydrate, a solvate, or a salt thereof, or a mixture of same.

In a further particular embodiment of the first aspect, the present invention covers compounds of formula (I), supra, wherein A2 is a phenyl ring.

In another further particular embodiment of the first aspect, the present invention covers compounds of formula (I), supra, wherein R³ is selected from the group of the following substituents

*-C(O)NH—(CH₂)₂CH₃

*-C(O)—N(CH₃)₂

*-C(O)—NH₂

*-C(O)—NH—(CH₂)₂N(CH₃)₂

*-CH₂—C(O)—NH₂

hydrogen

*-F, *-Cl, *-Br

*-C≡N; *-CF₃, *-CH₃, *-C₂H₅, *-CH═CH₂;

*-CH₂—CN; *-CH(CH₃)—NH₂; *-CH═CH—CN;

*-C(O)—OH; *-C(O)—OCH₃, *-C(O)—CH₃, *-C(CH₃)₂-C(O)—OCH₃, *-C(CH₃)₂—CN; Oxo(═O); hydroxy;

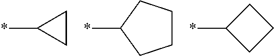

*-NH₂

*-NH—C(O)CH₃

*-NH—SO₂—CH₃

*-NH—C(O)—O—C(CH₃)₃

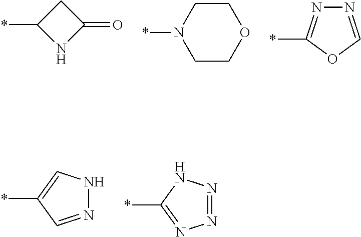

*-SO₂—CH₃

*-SO₂—N(CH₃)₂

*-SO₂—NH₂

*-O—CH₂—CH₃; *-O—(CH₂)₂—CH₃; *-O—CF₃;

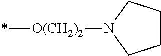

*-OCH₂-Cyclopropyl; *-OCH₃;

*-O(CH₂)₃—CH₃, *-OCH₂-Phenyl; *-O-Phenyl;

*-(CH₂)—OH

*-(CH₂)₂—OH

*-(CH₂)—O—CH₃

*-(CH₂)—O—CH₂—CH₃

*-CH(OH)—CH₂-Phenyl

*-CH(OH)—CH₂-CH₃

*-CH(OH)-(CH₂)₂—CH₃*-CH(OH)—(CH₂)₃—CH₃

*-CH(OH)—CH—(CH₃)₂

*-CH(OH)-Phenyl

*-CH(OH)—CN

*-CH(OH)—CH₂OH

*-CH(OH)—CF₃

*-CH(OH)—(CH₂)₂-Phenyl

*-CH(OH)—-C≡CH

*-CH(NH₂)—CH₂-COOH

*-CH₂—NH—SO₂—CH₃

*-CH₂—NH—(CH₂)₃—CH₃

*-CH₂—NH—CH₃

*-CH₂—N(CH₃)₂

*-CH₂—NH—C₂H₅*—CH(CH₃)—NH₂

*-CH₂—NH₂

*-(CH₂)₂—NH₂

*-CH₂—NH—CH₂-Phenyl

*-CH₂—N(C₂H₅)₂

*-CH₂—NH—Cyclopropyl

*-CH₂—NH—Cyclobutyl

*-CH₂—NH—Cyclopentyl

*-CH₂—NH-Pyridyl

*-CH₂—NH-Phenyl

*-CH₂—NH—(CH₂)₂—OH

*-CH₂—N(CH₃)(CH₂)₂OH*-CH₂—NH—CH₂—CN

*-CH₂—N(CH₃)—CH₂—CN

*-CH₂—N(CH₃)—CH₂—CF₃

*-CH₂—N(CH₃)—CH₂-CF₂H

*-CH₂—NH—CH₂—CF₂H

*-CH₂—NH—CH₂—CF₃

*-CH₂—NH—(CH₂)₂—OCH₃

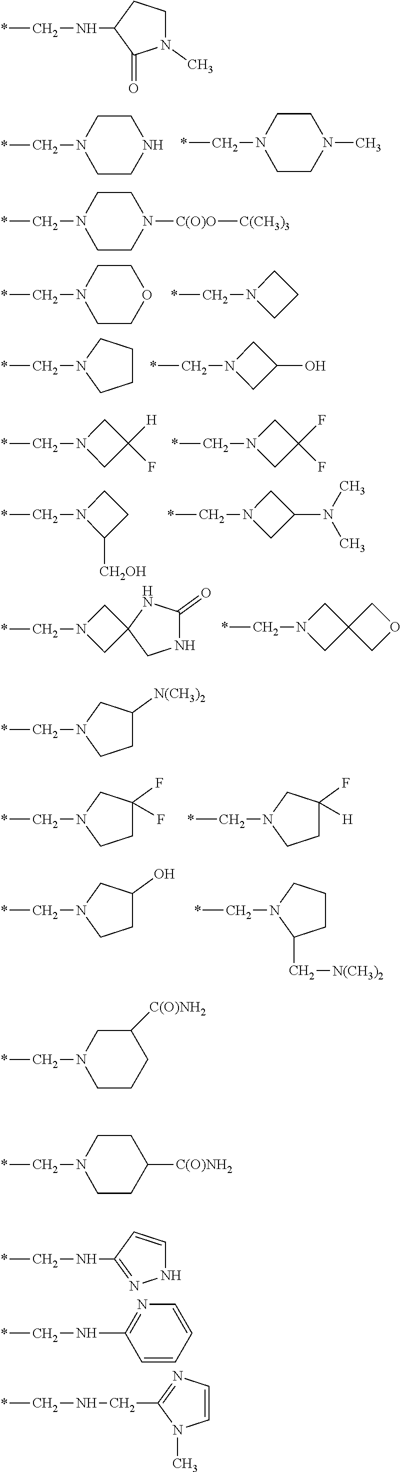

*-CH₂—NH—C(O)—O—C(CH₃)₃

*-(CH₂)₂—NH—C(O)—O—C(CH₃)₃

*-CH₂—NH—C(O)—CH₂—OH

*-CH₂—NH—C(O)—CH₂—OCH₃

*-CH—(CH₃)—NH—C(O)—O—C(CH₃)₃

*-CH₂—NH—C(O)—CH₃

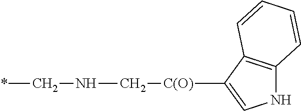

*-CH₂—NH—CH₂—C(O)—NH₂

*-CH₂—NH—CH₂—C(O)—N(CH₃)₂

*-CH₂—NH—CH₂—C(O)—OCH₃

*-CH₂—NH—CH₂—C(O)—NHCH₃

*-CH₂—NH—CH₂—C(O)—NH—(CH₂)₂—O—CH₃

*-CH₂—NH—CH₂—C(O)—NH—CH₂-Phenyl

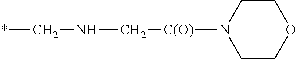

*-CH₂—NH—CH₂—C(O)—NH-Phenyl

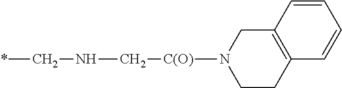

*-CH₂—NH—C(O)—CH₂—NH-Phenyl

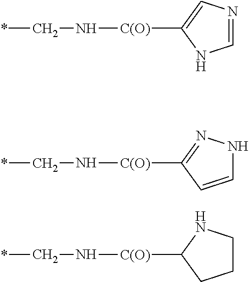

*-CH₂—NH—C(O)—CH₂—NH—CH₂—CF₃

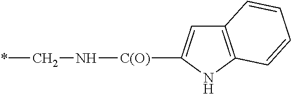

In yet another further particular embodiment of the first aspect, the present invention covers compounds of formula (I), supra, wherein R³ is a C₁ - or C₂-alkyl substituted with an amino group —NR^{k}R^{l}, wherein R^{k} and RI can have all the meanings as defined supra within the definition of R³ or wherein R³ is a C₁- or C₂-alkyl substituted with a hydroxyl or a C₁-C₆-alkoxy

or a stereoisomer, a tautomer, an N-oxide, a hydrate, a solvate, or a salt thereof, or a mixture of same.

In even further particular embodiments of the first aspect, the present invention covers compounds of formula (I), supra, wherein x is 1 or 2 and/or y is 1 or 2 and/or z is 1 or 2 or a stereoisomer, a tautomer, an N-oxide, a hydrate, a solvate, or a salt thereof, or a mixture of same.

In a particular further embodiment of the first aspect, the present invention covers combinations of two or more of the above mentioned embodiments under the heading “further embodiments of the first aspect of the present invention”.

The present invention covers any sub-combination within any embodiment or aspect of the present invention of compounds of general formula (I), supra.

The present invention covers any sub-combination within any embodiment or aspect of the present invention of intermediate compounds of general formula (II).The present invention covers the compounds of general formula (I) which are disclosed in the Example Section of this text, infra.

Synthesis of Compounds (Overview)

The compounds of the present invention can be prepared as described in the following section. The schemes and the procedures described below illustrate general synthetic routes to the compounds of general formula (I) of the invention and are not intended to be limiting. It is clear to the person skilled in the art that the order of transformations as exemplified in the schemes can be modified in various ways. The order of transformations exemplified in the schemes is therefore not intended to be limiting. In addition, interconversion of any of the substituents can be achieved before and/or after the exemplified transformations. These modifications can be such as the introduction of protecting groups, cleavage of protecting groups, exchange, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example P.G.M. Wuts and T. W. Greene in “Protective Groups in Organic Synthesis”, 4′″ edition, Wiley 2006). Specific examples are described in the subsequent paragraphs. Further, it is possible that two or more successive steps may be performed without work-up being performed between said steps, e.g. a “one-pot” reaction, as is well-known to the person skilled in the art.

The syntheses of the compounds of the present invention are preferably carried out according to the general synthetic sequence, shown in schemes 1-7.

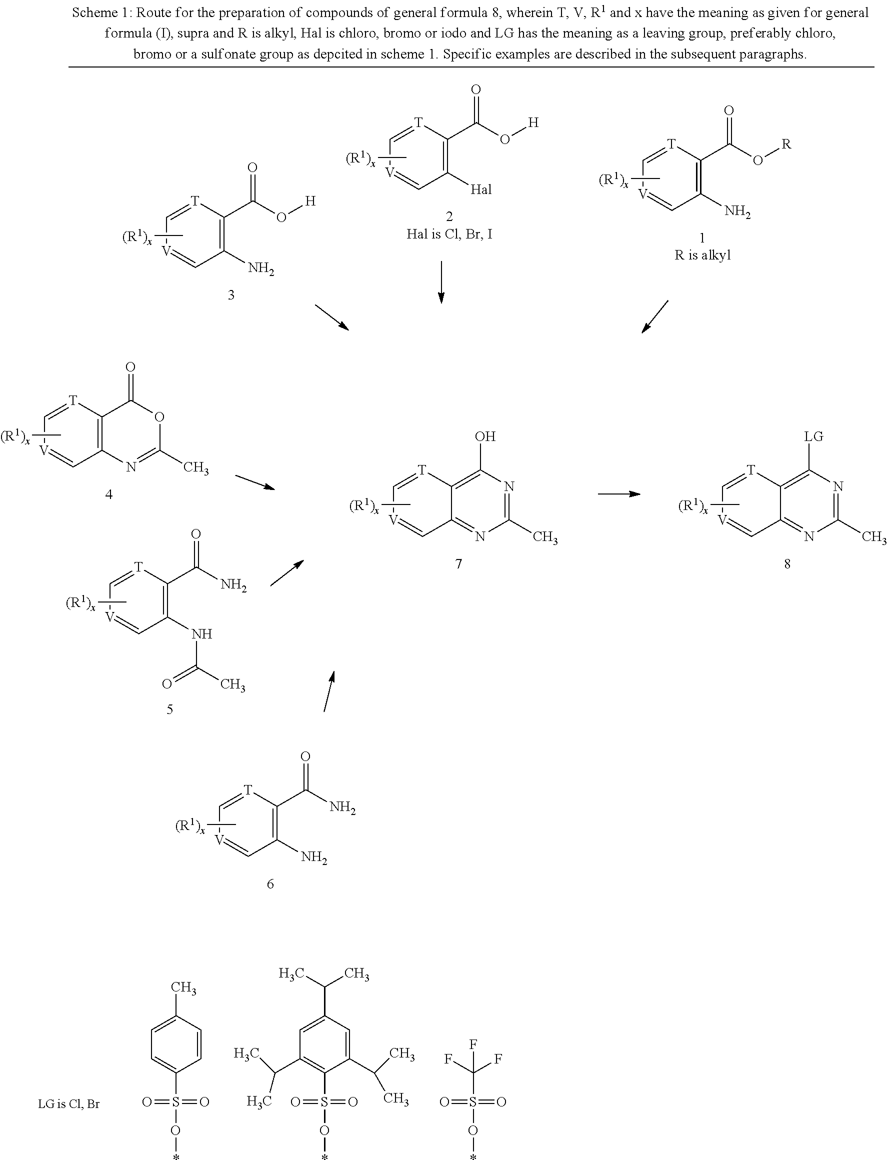

Step 1 → 7 (Scheme 1)

Azaquinazoline Formation

In the first step (scheme 1) amino acid ester derivative 1 (which is commercially available or described in the literature) can be converted to the corresponding azaquinazoline 7 in analogy to literature procedures. Typically acetonitrile and hydrochloric acid in organic solvent such as for example 1,4-dioxane at elevated temperatures is used. For example see ACS Medicinal Chemistry Letters, 2013, vol. 4, # 9 p. 846-851; Journal of Medicinal Chemistry, 2009, vol. 52, # 8 p. 2341-2351 or WO2015/54572 and references therein.

Step 2 → 7 (Scheme 1)

Azaquinazoline Formation

Alternatively halogen substituted benzoic acid derivative of general formula 2 (which is commercially available or described in the literature) can be converted to the corresponding azaquinazoline 7 in analogy to literature procedures. Typically derivative 2 is reacted with acetamidine, copper metal, a base such as for example potassium carbonate in an organic solvent such as for example DMF at elevated temperature. For example see WO2005/51410, US2008/107623 and references therein.

Step 3 → 7 (Scheme 1)

Azaquinalzoline Formation

Alternatively amino substituted benzoic acid derivative of general formula 3 (which is commercially available or described in the literature) can be converted to the corresponding azaquinazoline 7 in analogy to literature procedures. Typically derivative 3 is reacted with acetyl chloride or acetic anhydride, an ammonia source such as for example ammonia or ammonium acetate, a base such as for example triethylamine or pyridine with or without DMAP in an organic solvent such as for example DMF, toluene, 1,4-dioxane/water at elevated temperature. For example see Bioorganic and Medicinal Chemistry Letters, 2011, vol. 21, # 4 p. 1270-1274; Bioorganic and Medicinal Chemistry Letters, 2010, vol. 20, # 7 p. 2330-2334; WO2008/117079 or WO2006/74187 and references therein.

Step 4 → 7 (Scheme 1)

Azaquinazoline Formation

Alternatively benzoxazinone derivative of general formula 4 (which is commercially available or can be prepared in analogy to literature procedures) can be converted to the corresponding azaquinazoline 7 in analogy to literature procedures. Typically derivative 4 is reacted with ammonium acetate in a solvent at elevated temperature. For example see Bioorganic and Medicinal Chemistry Letters, 2011, vol. 21, # 4 p. 1270-1274 or U.S. Pat. No. 6,350,750 and references therein.

Step 5 → 7 (Scheme 1)

Azaquinazoline Formation

Alternatively benzoic acid amide derivative of general formula 5 (which is commercially available or described in the literature) can be converted to the corresponding azaquinazoline 7 in analogy to literature procedures. Typically derivative 5 is reacted with a base such as for example sodium hydroxide in a solvent such as for example water at elevated temperature. For example see Bioorganic and Medicinal Chemistry Letters, 2008, vol. 18, # 16 p. 4573-4577 and references therein.

Step 6 → 7 (Scheme 1)

Azaquinazoline Formation

Alternatively amino benzoic acid amide derivative of general formula 6 (which is commercially available or described in the literature) can be converted to the corresponding azaquinazoline 7 in analogy to literature procedures. Typically derivative 6 is reacted with acetic acid at elevated temperature. For example see Bioorganic and Medicinal Chemistry Letters, 2008, vol. 18, # 3 p. 1037-1041 and references therein.

Step 7 → 8 (Scheme 1)

Conversion of Hydroxyl Group into Leaving Group

In the next step (scheme 1) hydroxy azaquinazoline derivative 7 can be converted to the corresponding azaquinazoline 8 in analogy to literature procedures.

For W=chloro typically trichlorophosphate or thionylchloride, with or without N,N-dimethylaniline or N,N-diisopropylethylamine with or without an organic solvent such as for example toluene at elevated temperatures is used. For examples see Bioorganic and Medicinal Chemistry Letters, 2011, 1270; Journal of Medicinal Chemistry, 2009, 2341; ACS Medicinal Chemistry Letters, 2013, 846; Bioorganic and Medicinal Chemistry Letters, 2010, 2330; U.S. Pat. No. 6,350,750 or WO2015/54572 and references therein.

For W=bromo typically phosphorus oxytribromide, with or without N,N-dimethylaniline or N,N- diisopropylethylamine with or without an organic solvent such as for example toluene at elevated temperatures is used. For examples see US2012/53174; WO2012/30912 or WO2012/66122 and references therein.

For W=2,4,6-triisopropylsulfonate typically 2,4,6-triisopropylbenzenesulfonyl chloride, a base such as for example triethylamine and/or DMAP in an organic solvent such as for example dichloromethane is used. For examples see WO2010/99379 US2012/53176 and references therein.

For W=tosylate typically 4-methylbenzene-1-sulfonyl chloride, a base such as for example triethylamine or potassium carbonate and/or DMAP in an organic solvent such as for example dichloromethane or acetonitrile is used. For examples see Organic Letters, 2011, 4374 or Bioorganic and Medicinal Chemistry Letters, 2013, 2663 and references therein.

For W=trifluoromethanesulfonate typically N,N-bis(trifluoromethylsulfonyl)aniline or trifluoromethanesulfonic anhydride, a base such as for example triethylamine or 1,8-diazabicyclol5.4.01undec-7-ene and/or DMAP in an organic solvent such as for example dichloromethane is used. For examples see Journal of the American Chemical Society, 2015, 13433 or WO2014/100501and references therein.

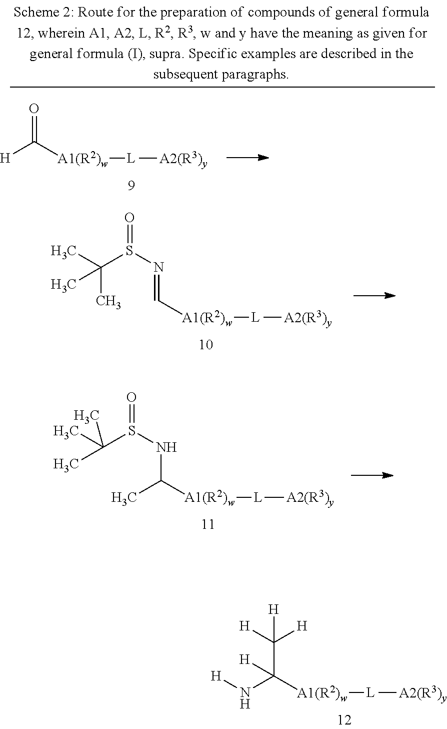

Step 9 → 10 (Scheme 2)

Sulfinimine Formation

In the first step (scheme 2) aldehyde derivative 9 (which is commercially available or described in the literature) can be converted to the corresponding sulfinimine 10 in analogy to the numerous literature procedures. For example the reaction can be performed at ambient temperature using Titanium(IV)ethoxide in an organic solvent as for example THF. For a review about sulfinimine chemistry see for example Chem. Rev. 2010, 110, 3600-3740; Chem. Soc. Rev. 2009, 38, 1162-1186; Tetrahedron 2004, 60, 8003or WO2013030138 and the references therein.

Step 10 → 11 (Scheme 2)

Sulfinamide Formation

In the next step (scheme 2) sulfinimine 10 can be converted to the corresponding sulfinamide 11 in analogy to the numerous literature procedures. For example the reaction can be performed using methylmagnesium bromide in an organic solvent as for example THF. For a review about sulfinimine and sulfinamide chemistry see for example Chem. Rev. 2010, 110, 3600-3740; Chem. Soc. Rev. 2009, 38, 1162-1186; Tetrahedron 2004, 60, 8003 or WO2013030138 and the references therein.

Step 11 → 12 (Scheme 2)

Formation of amine

In the next step (scheme 2) sulfinamide 11 can be converted to the corresponding amine 12 in analogy to the numerous literature procedures. For example the reaction can be performed using acetylchloride in a protic organic solvent as for example methanol. For a review about sulfinimine and sulfonamide chemistry see for example Chem. Rev. 2010, 110, 3600-3740; Chem. Soc. Rev. 2009, 38, 1162-1186; Tetrahedron 2004, 60, 8003 or WO2013030138 and the references therein.

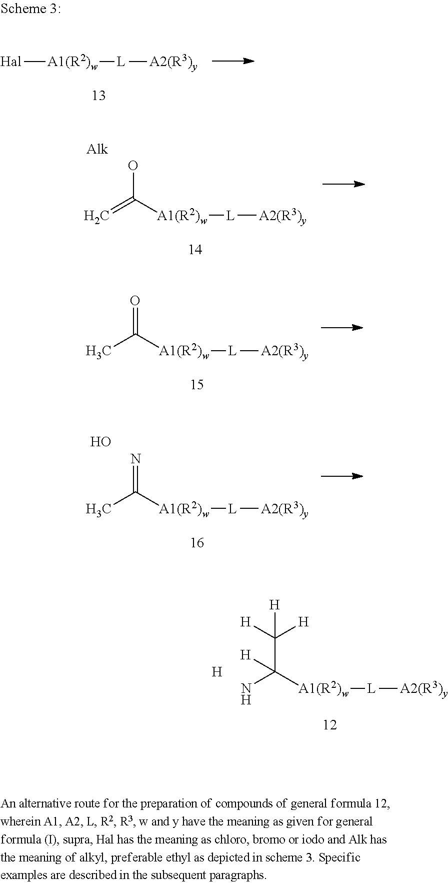

Step 13 → 14 (Scheme 3)

Stille Coupling

In the first step (scheme 3) halide derivative 13 (which is commercially available or described in the literature) can be converted to the corresponding enolester derivative 14 in analogy to literature procedures. Typically the reaction is performed with tributyl(1-ethoxyethenyl)stannane, a palladium catalyst such as for example bis-triphenylphosphine-palladium(II) chloride or dichloro(1,1′-bis(diphenylphosphanyl)ferrocene)palladium(II) dichloromethane adduct, with or without a base such as for example triethylamine in an organic solvent such as for example DMF, 1,4-dioxane or toluene at elevated temperature.

For W=bromo see for example the literature references WO2010/116282, WO2004/214, WO2013/185093 or Journal of the American Chemical Society, 2002, 6343 and references therein.

For W=chloro see for example the literature references Angewandte Chemie—International Edition, 1999, 2411-2413; Journal of the American Chemical Society, 2004, 16433, Organic Letters, 2004, 1421; Organic letters, 2001, 4295 and references therein.

For W=iodo see for example the literature references Bioorganic and Medicinal Chemistry Letters, 2003, 637, WO2011/100401, WO2007/38613 or US2005/143401 and references therein.

Step 14 → 15 (Scheme 3)

Formation of methylketone

In the next step (scheme 3) enolester derivative 14 can be converted to the corresponding methyl ketone 15 in analogy to literature procedures. Typically the reaction is performed with an acid such as for example aqueous hydrochloric acid in an organic solvent such as for example THF, 1,4-dioxane or acetone. See for example the literature references Journal of Organic Chemistry, 1992, 1486, WO2013/185103 or U.S. Pat. No. 7,361,789 (2008) and references therein.

Step 15 → 16 (Scheme 3)

Formation of oxime

In the next step (scheme 3) methyl ketone derivative 15 can be converted to the corresponding oxime 16 in analogy to literature procedures. Typically the reaction is performed with hydroxylamine hydrochloride with or without the addition of a base such as for example sodium acetate, pyridine, or KOH aq. in an organic solvent such as for example ethanol, DMSO, THF, dimethylether or methanol. See for example the literature references U.S. Pat. No. 5,332,757 (1994); US2004/157849 or International Journal of Pharmaceutics, 2016, 205 and references therein.

Step 16 → 12 (Scheme 3)

Reduction of oxime

In the next step (scheme 3) oxime derivative 16 can be reduced to the corresponding amine 12 in analogy to literature procedures. Typical reaction conditions include for example hydrogen, acetic acid, palladium on activated carbon in ethanol (see literature reference WO2006/82392 and references therein); ammonia, hydrogen, Raney nickel in methanol (see literature reference US2011/263626 (2011) and references therein); hydrogen, acetic acid, palladium on activated carbon in ethanol (see literature references WO2006/82392 and references therein) or acetic acid, zinc in methanol (see literature reference WO2013/26914 and references therein).

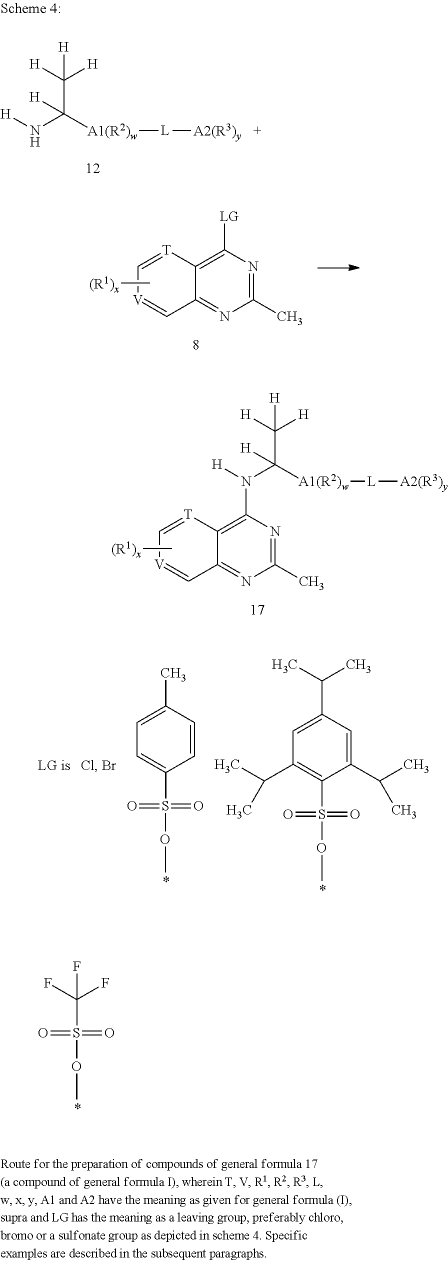

Step 12+8 → 17 (Scheme 4)

Amine Coupling

In the first step (scheme 4) amine derivative 12 and azaquinazoline derivative 8 are converted to amine 17 in analogy to literature procedures. Typically the reaction is performed in an organic solvent such as for example THF, DMF, acetonitrile dichloromethane or isopropyl alcohol with or without a base such as for example triethylamine, N-ethyl-N,N-diisopropylamine, potassium carbonate or potassium tert-butylate.

For LG=chloro see for example the literature references WO2008/86462; WO2008/86462 or European Journal of Medicinal Chemistry, 2015, 462 and references therein.

For LG=bromo see for example the literature references US2009/247519 or Journal of Organic Chemistry, 2009, 8460 and references therein.

For LG=tosylate see for example the literature references Synthetic Communications, 2012, 1715; Synthesis 2015, 2055 or Bioorganic and Medicinal Chemistry Letters, 2013, 2663 and references therein.

For LG=triflate see for example the literature references Bioorganic and Medicinal Chemistry Letters, 2013, 3325 and references therein.

For LG=2,4,6-triisopropylbenzenesulfonate see for example the literature reference WO2010/99379 and references therein.

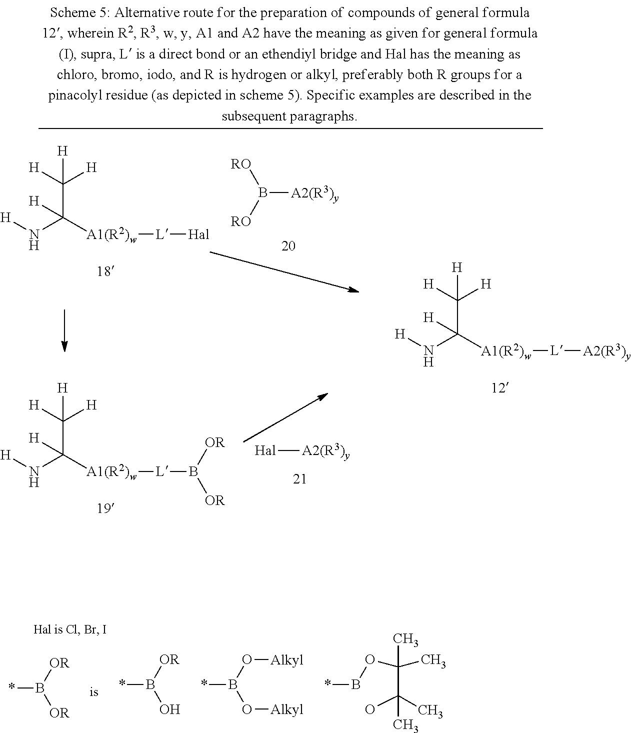

Step 18+20 →0 12 (Scheme 5)

C—C Cross Coupling Reaction

Halogen comounds of general formula 18′ (Scheme 5) can be reacted with a boronic acid derivative 20 to give a compound of formula 12′. The boronic acid derivative may be a boronic acid (R═H) or an alkyl ester of the boronic acid, e.g. its isopropyl ester (R═CH(CH₃)₂), preferably an ester derived from pinacol. The coupling reaction is catalyzed by palladium catalysts, e.g. by Pd(0) catalysts like tetrakis(triphenylphosphine)palladium(0) [Pd(PPh3)4], tris(dibenzylideneacetone)di-palladium(0) 15 [Pd&(dba)3], or by Pd(11) catalysts like dichlorobis(triphenylphosphine)-palladium(11) [Pd(PPh3)3Cl], palladium(11) acetate and triphenylphosphine or by [1,1′-bis(diphenylphosphino)ferrocene]palladium dichloride. The reaction is preferably carried out in a mixture of a solvent like 1,2-dimethoxyethane, dioxane, DMF, DME, THF, or isopropanol with water and in the presence of a base like potassium carbonate, sodium bicarbonate or potassium phosphate. For a review see D. G. Hall, Boronic Acids, 2005 WILEY-VCH Verlag GmbH & Co. KGaA, Weinheim, ISBN 3-527-30991-8 and references cited therein). The reaction is performed at temperatures ranging from room temperature to the boiling point of the solvent. Further on, the reaction can be performed at temperatures above the boiling point under pressure. The reaction is preferably completed after 1 to 36 hours.

Step 18 → 19 (Scheme 5)

Formation of Boronates/Boronic Acids

Halogogen derivative 18′ are converted to boronic acid derivative 22 in analogy to literature procedures (scheme 5).

For Hal =bromo or iodo and *-B(OR)₂=boronic acid pinacol ester the reaction is typically performed with bis(pinacolato)diboron, a palladium catalyst as for example palladium diacetate or dichloro(1,1′-bis(diphenylphosphanyl)ferrocene)palladium(II) dichloromethane adduct a base as for example potassium acetate or triethylamine in an organic solvent as for example DMF, DMSO, acetonitrile. See for example the literature references WO2010/150192, WO2012/158795 or Journal of Medicinal Chemistry, 2006, 5671 and references therein.

For Hal=chloro and *-B(OR)₂=boronic acid pinacole ester see for example the literature references Organic Letters, 2002, 543 or Journal of Organic Chemistry, 2012, 3543 and references therein.

For Hal=bromo or iodo and *-B(OR)₂=boronic acid or boronic acid methyl ester the reaction is typically performed with butyllithium or magnesium/iodine, boronic acid trimethylester in an organic solvent as for example THF, hexane. See for example the literature references Organic and Biomolecular Chemistry, 2012, 6693, Journal of the American Chemical Society, 2009, 17500 or Organic Letters, 2011, 4479 and references therein.

Step 19+21 → 12 (Scheme 5)

C—C Cross Coupling Reaction

Halogen comounds of general formula 21 (Scheme 5) can be reacted with a boronic acid derivative 19 to give a compound of formula 12′. The boronic acid derivative may be a boronic acid (R═H) or an alkyl ester of the boronic acid, e.g. its isopropyl ester (R═CH(CH₃)₂), preferably an ester derived from pinacol. The coupling reaction is catalyzed by palladium catalysts, e.g. by Pd(0) catalysts like tetrakis(triphenylphosphine)palladium(0) [Pd(PPh3)4], tris(dibenzylideneacetone)di-palladium(0) 15 [Pd&(dba)3], or by Pd(11) catalysts like dichlorobis(triphenylphosphine)-palladium(11) [Pd(PPh3)3Cl], palladium(11) acetate and triphenylphosphine or by [1,1′-bis(diphenylphosphino)ferrocene]palladium dichloride. The reaction is preferably carried out in a mixture of a solvent like 1,2-dimethoxyethane, dioxane, DMF, DME, THF, or isopropanol with water and in the presence of a base like potassium carbonate, sodium bicarbonate or potassium phosphate. For a review see D. G. Hall, Boronic Acids, 2005 WILEY-VCH Verlag GmbH & Co. KGaA, Weinheim, ISBN 3-527-30991-8 and references cited therein). The reaction is performed at temperatures ranging from room temperature to the boiling point of the solvent. Further on, the reaction can be performed at temperatures above the boiling point under pressure. The reaction is preferably completed after 1 to 36 hours.

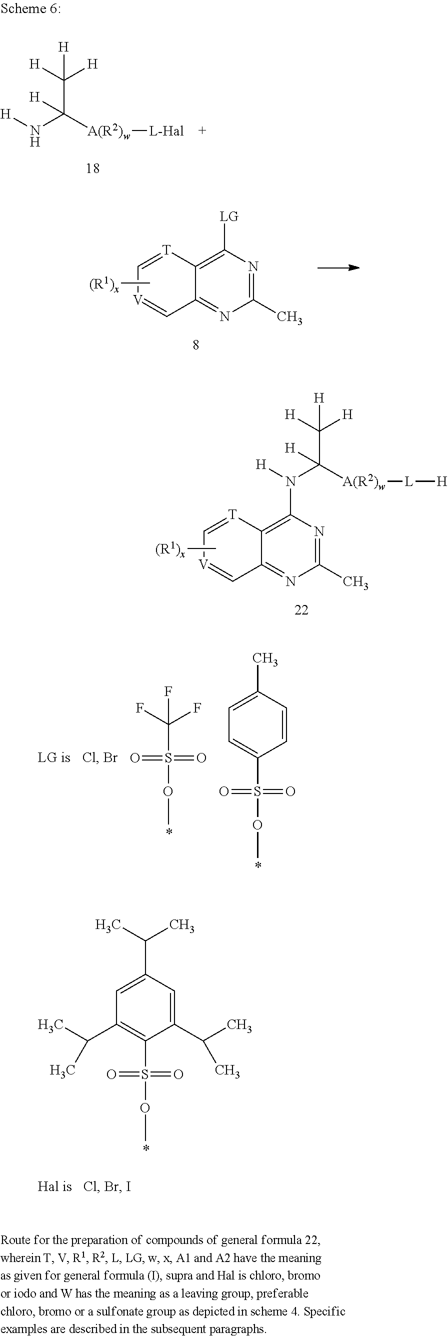

Step 18+8 → 22 (Scheme 6)

In the first step (scheme 6) amine derivative 18 and azaquinazoline derivative 8 are converted to amine 22 in analogy to literature procedures. Typically the reaction is performed in an organic solvent such as for example THF, DMF, acetonitrile dichloromethane or isopropyl alcohol with or without a base such as for example triethylamine, N-ethyl-N,N-diisopropylamine, potassium carbonate or potassium tert-butylate.

For LG=chloro see for example the literature references WO2008/86462; WO2008/86462 or European Journal of Medicinal Chemistry, 2015, 462 and references therein.

For LG=bromo see for example the literature references US2009/247519 or Journal of Organic Chemistry, 2009, 8460 and references therein.

For LG=tosylate see for example the literature references Synthetic Communications, 2012, 1715; Synthesis 2015, 2055 or Bioorganic and Medicinal Chemistry Letters, 2013, 2663 and references therein.

For LG=triflate see for example the literature references Bioorganic and Medicinal Chemistry Letters, 2013, 3325 and references therein.

For LG=2,4,6-triisopropylbenzenesulfonate see for example the literature reference WO2010/99379 and references therein.

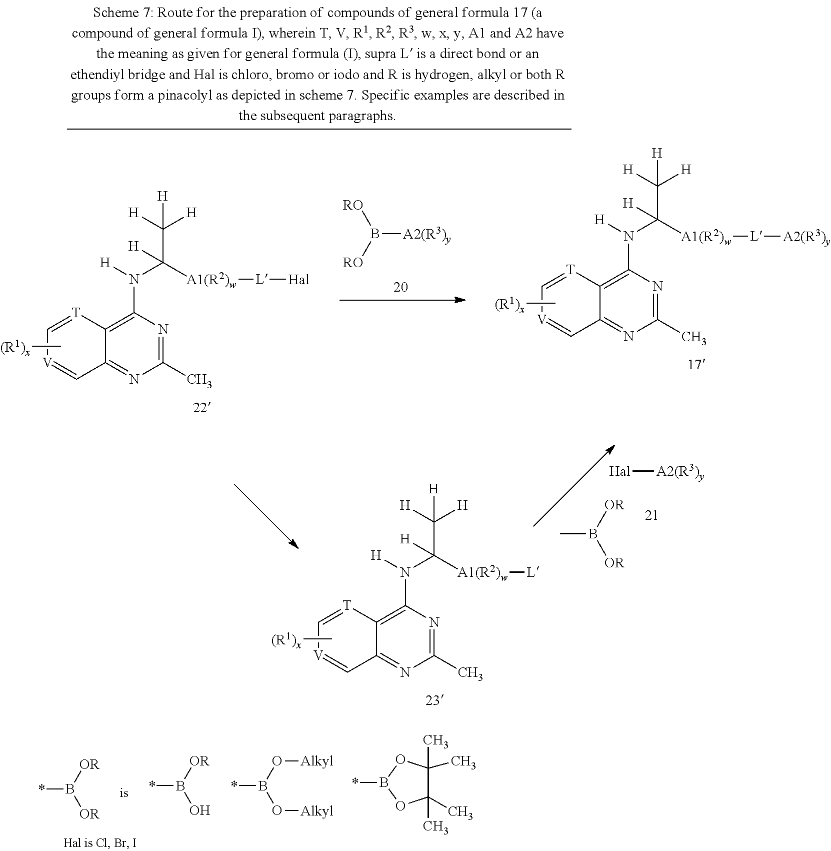

Step 22+20 → 17 (Scheme 7)

C—C Cross Coupling Reaction

Halogen comounds of general formula 22 (Scheme 7) can be reacted with a boronic acid derivative 20 to give a compound of formula 17. The boronic acid derivative may be a boronic acid (R═H) or an alkyl ester of the boronic acid, e.g. its isopropyl ester (R═CH(CH₃)₂), preferably an ester derived from pinacol. The coupling reaction is catalyzed by palladium catalysts, e.g. by Pd(0) catalysts like tetrakis(triphenylphosphine)palladium(0) [Pd(PPh3)4], tris(dibenzylideneacetone)di-palladium(0) [Pd2 (dba)3], or by Pd(11) catalysts like dichlorobis(triphenylphosphine)-palladium(11) [Pd(PPh3)3Cl], palladium(11) acetate and triphenylphosphine or by [1,1′-bis(diphenylphosphino)ferrocene]palladium dichloride. The reaction is preferably carried out in a mixture of a solvent like 1,2-dimethoxyethane, dioxane, DMF, DME, THF, or isopropanol with water and in the presence of a base like potassium carbonate, sodium bicarbonate or potassium phosphate. For a review see D. G. Hall, Boronic Acids, 2005 WILEY-VCH Verlag GmbH & Co. KGaA, Weinheim, ISBN 3-527-30991-8 and references cited therein). The reaction is performed at temperatures ranging from room temperature to the boiling point of the solvent. Further on, the reaction can be performed at temperatures above the boiling point under pressure. The reaction is preferably completed after 1 to 36 hours.

Step 22 → 23 (Scheme 7)

Formation of Boronates/Boronic Acids

Halogogen derivative 22 are converted to boronic acid derivative 23 in analogy to literature procedures (scheme 7).

For Hal =bromo or iodo and *-B(OR)₂=boronic acid pinacole ester the reaction is typically performed with bis(pinacolato)diboron, a palladium catalyst as for example palladium diacetate or dichloro(1,1′-bis(diphenylphosphanyl)ferrocene)palladium(II) dichloromethane adduct a base as for example potassium acetate or triethylamine in an organic solvent as for example DMF, DMSO, acetonitrile. See for example the literature references WO2010/150192, WO2012/158795 or Journal of Medicinal Chemistry, 2006, 5671 and references therein.

For Hal=bromo or iodo and *-B(OR)₂=boronic acid or boronic acid methyl ester the reaction is typically performed with butyllithium or magnesium/iodine, boronic acid trimethylester in an organic solvent as for example THF, hexane. See for example the literature references Organic and Biomolecular Chemistry, 2012, 6693, Journal of the American Chemical Society, 2009, 17500 or Organic Letters, 2011, 4479 and references therein.

Step 23+21 → 17 (Scheme 7)

C—C Cross Coupling Reaction

Halogen comounds of general formula 21 (Scheme 7) can be reacted with a boronic acid derivative 23 to give a compound of formula 17. The boronic acid derivative may be a boronic acid (R═H) or an alkyl ester of the boronic acid, e.g. its isopropyl ester (R═CH(CH₃)2), preferably an ester derived from pinacol. The coupling reaction is catalyzed by palladium catalysts, e.g. by Pd(0) catalysts like tetrakis(triphenylphosphine)palladium(0) [Pd(PPh3)4], tris(dibenzylideneacetone)di-palladium(0) 15 [Pd&(dba)3], or by Pd(ll) catalysts like dichlorobis(triphenylphosphine)-palladium(ll) [Pd(PPh3)3Cl], palladium(11) acetate and triphenylphosphine or by [1,1′-bis(diphenylphosphino)ferrocene]palladium dichloride. The reaction is preferably carried out in a mixture of a solvent like 1,2-dimethoxyethane, dioxane, DMF, DME, THF, or isopropanol with water and in the presence of a base like potassium carbonate, sodium bicarbonate or potassium phosphate. For a review see D. G. Hall, Boronic Acids, 2005 WILEY-VCH Verlag GmbH & Co. KGaA, Weinheim, ISBN 3-527-30991-8 and references cited therein). The reaction is performed at temperatures ranging from room temperature to the boiling point of the solvent. Further on, the reaction can be performed at temperatures above the boiling point under pressure. The reaction is preferably completed after 1 to 36 hours.

In accordance with a further aspect, the present invention covers intermediate compounds which are useful in the preparation of compounds of the present invention of general formula (I), particularly in the methods described herein. In particular, the present invention covers compounds of general formula II,

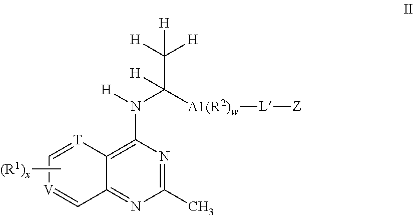

in which T, V, R¹, R², A1, R², L′, w and x have the same meanings as defined for the compound of general (I) supra, and Z is
Hal (iodo, bromo or chloro) or

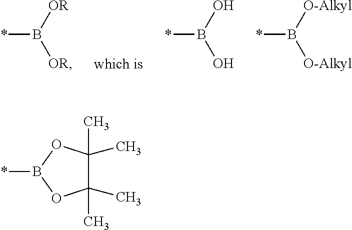

The present invention covers the intermediate compounds which are disclosed in the Example Section of this text, infra.

The present invention covers any sub-combination within any embodiment or aspect of the present invention of intermediate compounds of general formula (II), supra.

In accordance with another aspect, the present invention covers methods of preparing compounds of the present invention, said methods comprising the step as described below and/or the Experimental Section.

In particular, the present invention covers a method to prepare compounds of general formula I supra,

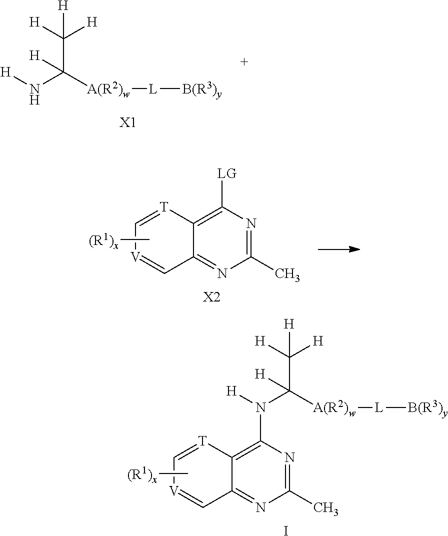

characterized in that compounds of general formula X1 and X2, in which T, V, R¹, R², R³, L, w, x, y, A1 and A2 have the same meaning as defined for compounds of general formula (I) and LG is a leaving group as chloro, bromo, iodo, fluoro, triflate, tosylate, mesitylate or nonaflate, are reacted in an organic solvent at a temperature between −20° C. and the boiling point of a solvent, preferably between ambient temperature and the boiling point of the solvent, with or without a base to obtain compounds of general formula I.

The preparation of compounds of general formula I can be performed in a protic or aprotic solvent, preferably in dioxan, tetrahydrofuran, N,N-dimethylformamide, dimethylsulfoxid, methanol, ethanol or 2-propanol.

Preferred bases which can be used for the preparation of compounds of the general formula I are N,N-diisopropylethylamin or triethylamin.

Said compound of general formula I can then optionally be converted into solvates, salts and/or solvates of such salts using the corresponding (i) solvents and/or (ii) bases or acids.

The present invention covers methods of preparing compounds of the present invention of general formula (I), said methods comprising the steps as described in the Experimental Section herein.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

For the synthesis of deuterated intermediates and test compounds of the general formula I the same general procedures as described before can be applied by using the corresponding deuterated reagents. For example, compound 3 can be converted to compound 7D by using deuterated acetyl chloride.

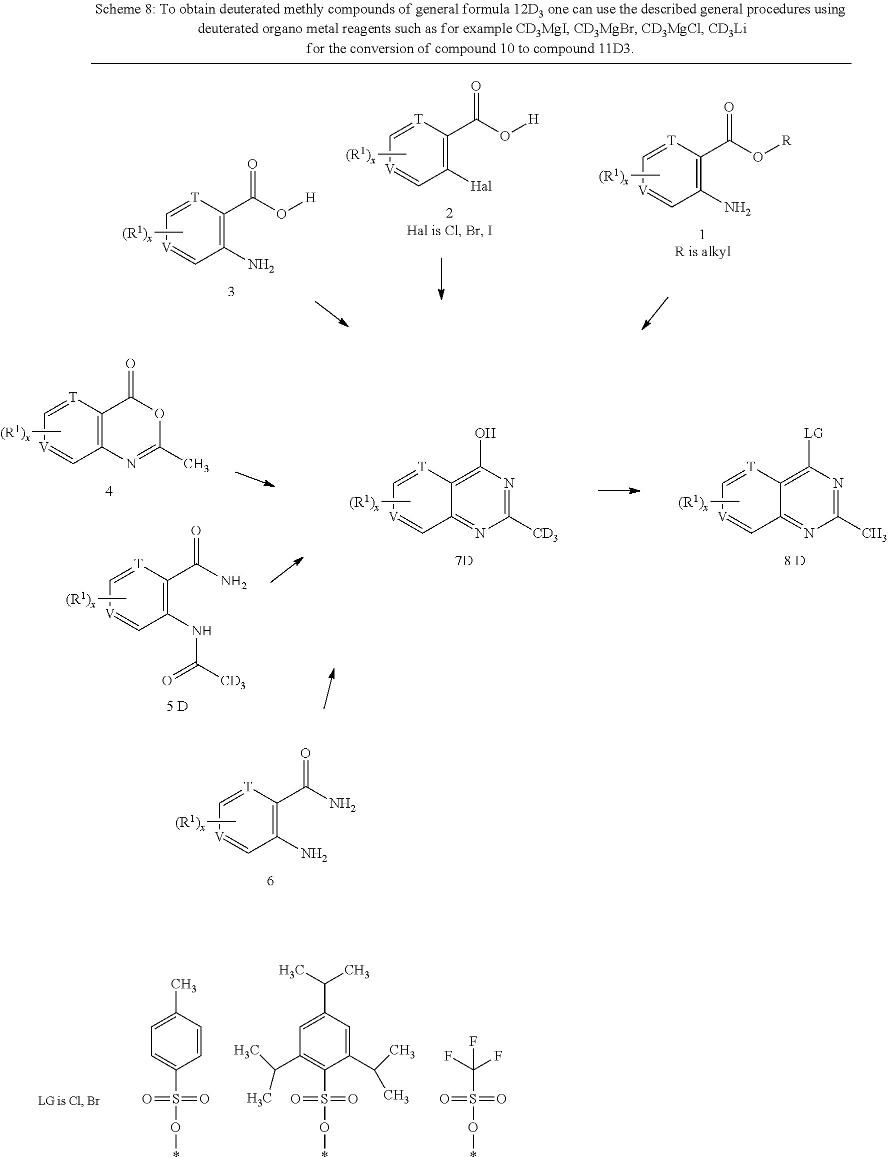

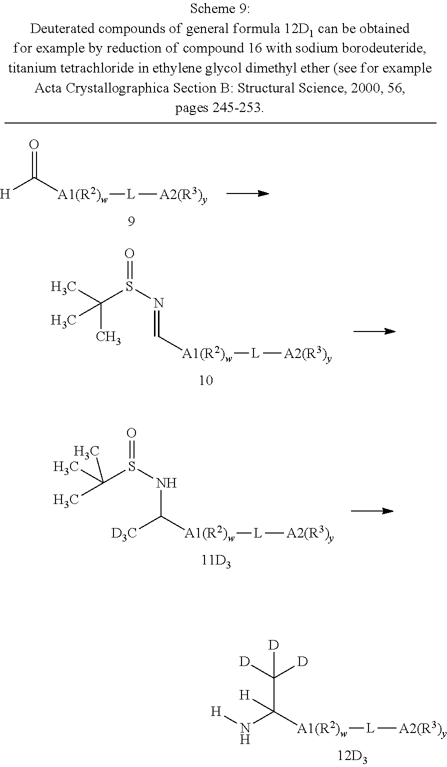

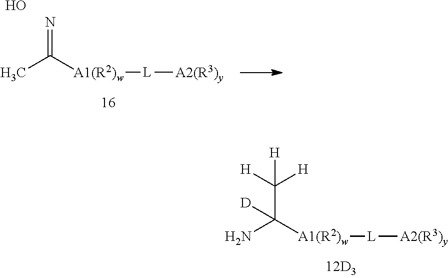

One of the most fundamental characteristics of cancer cells is their ability to sustain chronic proliferation whereas in normal tissues the entry into and progression through the cell division cycle is tightly controlled to ensure a homeostasis of cell number and maintenance of normal tissue function. Loss of proliferation control is emphasized as one of the six hallmarks of cancer [Hanahan D and Weinberg 15 RA, Cell 100, 57, 2000; Hanahan D and Weinberg R A, Cell 144, 646, 2011].

Compounds of general formula (I) of the present invention demonstrate a valuable pharmacological spectrum of action which could not have been predicted. Compounds of the present invention have surprisingly been found to effectively inhibit the Ras-Sos interaction and it is possible therefore that said compounds be used for the treatment or prophylaxis of diseases, preferably hyperproliferative disorders in humans and animals.

Compounds of the present invention can be utilized to inhibit, block, reduce, decrease, etc., cell proliferation and/or cell division, and/or produce apoptosis. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The present invention also provides methods of treating angiogenic disorders including diseases associated with excessive and/or abnormal angiogenesis.

Inappropriate and ectopic expression of angiogenesis can be deleterious to an organism. A number of pathological conditions are associated with the growth of extraneous blood vessels. These include, for example, diabetic retinopathy, ischemic retinal-vein occlusion, and retinopathy of prematurity [Aiello et al., New Engl. J. Med., 1994, 331, 1480 ; Peer et al., Lab. Invest., 1995, 72, 638], age-related macular degeneration (AMD) [Lopez et al., Invest. Opththalmol. Vis. Sci., 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of general formula (I) of the present invention can be utilized to treat and/or prevent any of the aforementioned angiogenesis disorders, for example by inhibiting and/or reducing blood vessel formation; by inhibiting, blocking, reducing, decreasing, etc. endothelial cell proliferation, or other types involved in angiogenesis, as well as causing cell death or apoptosis of such cell types.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of general formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of general formula (I) of the present invention is administered to a cell immediately after radiation or other induction of DNA damage in the cell has begun.

In another aspect, the cell is in vitro. In another embodiment, the cell is in vivo.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal, intralumbal or intratumoral) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tweed®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross- linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of a hyper-proliferative disorder, in particular cancer.

Particularly, the present invention covers a pharmaceutical combination, which comprises:
one or more first active ingredients, in particular compounds of general formula (I) as defined supra, and
one or more further active ingredients, in particular those used for treatment of hyper-proliferative disorder, in particular cancer.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known anti-tumor agents (cancer therapeutics).

Examples of anti-tumor agents (cancer therapeutics) include:

131I-chTNT, abarelix, abiraterone, aclarubicin, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib , crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin +estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, Iasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone +pentazocine, naltrexone, nartograstim, necitumumab, nedaplatin, nelarabine, neridronic acid, netupitant/palonosetron, nivolumabpentetreotide, nilotinib, nilutamide, nimorazole, nimotuzumab, nimustine, nintedanib, nitracrine, nivolumab, obinutuzumab, octreotide, ofatumumab, olaparib, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone +sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib , regorafenib, risedronic acid, rhenium-186 etidronate, rituximab, rolapitant, romidepsin, romiplostim, romurtide, roniciclib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur +gimeracil +oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine +tipiracil, trilostane, triptorelin, trametinib, trofosfamide, thrombopoietin, tryptophan, ubenimex, valatinib , valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

The following table lists the abbreviations used in this paragraph, and in the examples section.

| | |
|---|---|
| BuLi | Butyllithium |
| DCE | Dichloroethane |
| DCM | Dichloromethane |
| DMF | Dimethylformamide |
| DMSO | Dimethyl sulfoxide |
| EA | Ethyl acetate |
| FA | Formic acid |
| HPLC, LC | high performance liquid chromatography |
| h | hour |
| LiHMDS | Lithium bis(trimethylsilyl)amide |
| KHMDS | Potassium bis(trimethylsilyl)amide |
| KOtBu | Potassium tert-butoxide |
| min | minute |
| LDA | Lithiumdiisopropylamid |
| MS | mass spectroscopy |
| NMR | nuclear magnetic resonance |
| NaHMDS | Sodium bis(trimethylsilyl)amide |
| PE | Petrol ether |
| Rac | Racemate |
| R_{f} | Retardiation factor |
| Rₜ | Retention time |
| RT | Room temperature |
| TFA | Trifluoroacetic acid |
| THF | Tetrahydrofuran |
| TLC | thin-layer chromatography |

Chemical names were generated using ACD/Name Batch Version 12.01 or Autonom 2000. All reagents, for which the synthesis is not described in the experimental part, are either commercially available or synthesized as described in literature references.

Analytical Methods

LC-MS Method 1:
Column: Ascentis Express C18 2.7 μm, 30×2.1 mm
Fragment. potential: 50 V
Mass range: 80-800 m/z
Solvent: A=H₂O +0.1%vol HCOOH B=methanol +0.1%vol HCOOH
Gradient: 0-1 min. 5% B, 1-4 min. 5-100% B 4-5 min. 100% B, 5-6 min. 100-5% B, 6-6.5 min. 5% B
Flow: 0.8 mL/min
Temperature: 30° C.
Injection: 1.0 μL
Detection: MM-ES +APCI +DAD (254 nm)
System time delay: 0.2 min.

LC-MS Method 2:MS instrument type: Micromass Quatro Micro; HPLC instrument type: Agilent 1100 Series; UV DAD; column: Chromolith Flash RP-18E 25-2 mm; mobile phase A: 0.0375% TFA in water, mobile phase B: 0.01875% TFA in acetonitrile; gradient: 0.0 min. 100% A → 1.0 min. 95% A → 3.0 min. 95% A → 3.5 min. 5% A → 3.51 min. 5% A → 4.0 min. 95% A; flow rate: 0.8 mL/min; column temp: 50° C.; UV detection: 220 nm & 254 nm.

LC-MS Method 3:
System: Waters Acquity UPLC-MS: Binary Solvent Manager, Sample
Manager/Organizer, PDA, ELSD
Column: Acquity UPLC BEH C18 1.7 μm, 50×2.1 mm
Solvent: A=H₂O +H₂O +0.1%vol. HCOOC (99%) B=acetonitrile
Gradient: 0-1.6 min. 1-99% B, 1.6-2 min. 99% B
Flow: 0.8 mL/min
Temperature: 60° C.
Injection: 2.0 μL
Detection: DAD scan range 210-400 nm +ELSD

LC-MS Method 4:
System: Shimadzu LC-MS: UFLC 20-AD and LCMS 2020 MS detector
Column: Shim-pack XR-ODS 2.2 μm, 3.0×50 mm
Solvent: A=H₂O +0.05%vol. HCOOC (99%) B=acetonitrile+0.05%vol. HCOOC (99%)

LC-MS Method 5:
Ssytems: Waters Acquity UPLC-MS: Binary Solvent Manager, Sample Manager/Organizer, PDA, ELSD
Column: Acquity UPLC BEH C18 1.7 μm, 50×2.1 mm
Solvent: A=H₂O +0.2%vol. NH₃ (32%) B=acetonitrile
Gradient: 0-1.6 min. 1-99% B, 1.6-2 min. 99% B
Flow: 0.8 mL/min
Temperature: 60° C.
Injection: 2.0 μL
Detection: DAD scan range 210-400 nm +ELSD

LC-MS Method 6:
System: Instrument HPLC: Waters UPLC Acquity; Instrument MS: Waters ZQ
Column: Acquity UPLC BEH C18 1.7μm, 50×2.1 mm
Solvent: A=H₂O +0.1%vol. HCOOC (99%) B=acetonitrile
Gradient: 0-1.6 min. 1-99% B, 1.6-1.8 min. 99% B, 1.81-2 min. 1% B
Flow: 0.8 mL/min
Temperature: 60° C.
Detection: PDA scan range 210-400 nm

LC-MS Method 7:
System: Agilent 1290 UHPLC-MS Tof
Column: BEH C 18 (Waters) 1.7 μm, 50×2.1 mm
Solvent: A=H₂O +0.05%vol. HCOOC (99%) B=acetonitrile +0.05%vol. HCOOC (99%)
Gradient: 0-1.7 min. 2-90% B, 1.7-2 min. 90% B, 2-2.5 min. 90-2% B
Flow: 1.2 mL/min
Temperature: 60° C.
Detection: DAD scan range 210-400 nm

LC-MS Method 8:
System: Waters Acquity UPLC-MS: Binary Solvent Manager, Sample Manager/Organizer, PDA, ELSD
Column: Acquity UPLC BEH C18 1.7 μm, 50×2.1 mm
Solvent: A=H₂O +0.1%vol. HCOOC (99%) B=acetonitrile
Gradient: 0-1.6 min. 1-99% B, 1.6-2 min. 99% B
Flow: 0.8 mL/min
Temperature: 60° C.
Injection: 2.0 μL
Detection: DAD scan range 210-400 nm +ELSD

LC-MS Method 9:
System: Waters Acquity UPLC-MS SingleQuad
Column: Kinetex C 18 (Phenomenex) 2.6 μm, 50×2.1 mm
Solvent: A=H₂O +0.05%vol. HCOOC (99%) B=acetonitrile +0.05%vol. HCOOC (99%)
Gradient: 0-0.2 min. 2% B, 0.2-1.7 min. 2-90% B, 1.7-1.9 min. 90% B, 1.9-2 min. 90-2% B, 2-2.5 min. 2% B
Flow: 1.3 mL/min
Temperature: 60° C.
Detection: DAD scan range 210-400 nm

LC-MS method 10:
System: Waters Acquity UPLC-MS SingleQuad
Column: Acquity UPLC BEH C18 1.7 μm, 50×2.1 mm
Solvent: A=H₂O +0.2%vol. NH₃ (32%) B=acetonitrile
Gradient: 0-1.6 min. 1-99% B, 1.6-2 min. 99% B
Flow: 0.8 mL/min
Temperature: 60° C.
Detection: DAD scan range 210-400 nm

Preparative HPLC

a) Autopurifier: Acidic Conditions
System: Waters Autopurification system: Pump 2545, Sample Manager 2767, CFO, DAD 2996, ELSD 2424, SQD
Column: XBrigde C18 5.0 μm 100×30 mm
Solvent: A=H₂O +0.1%vol. HCOOH (99%) B=acetonitrile
Gradient: 0-0.5 min. 5% B 25 mL/min, 0.51-5.5 min. 10-100% B 70 mL/min, 5.51-6.5 min. 100% B 70 mL/min
Temperature: RT
Solution: max. 250 mg/max. 2.5 mL DMSO or DMF
Injection: 1×2.5 mL
Detection: DAD scan range 210-400 nm, MS ESI+, ESI−, scan range 160-1000 m/z

b) Autopurifier: basic conditions
System: Waters Autopurification system: Pump 2545, Sample Manager 2767, CFO, DAD 2996, ELSD 2424, SQD
Column: XBrigde C18 5.0 μm 100×30 mm
Solvent: A=H₂O +0.2%vol. NH₃ (32%) B=acetonitrile
Gradient: 0-0.5 min. 5% B 25 mL/min, 0.51-5.5 min. 10-100% B 70 mL/min, 5.51-6.5 min. 100% B 70 mL/min
Temperature: RT
Solution: max. 250 mg/max. 2.5 mL DMSO or DMF
Injection: 1×2.5 mL
Detection: DAD scan range 210-400 nm, MS ESI+, ESI−, scan range 160-1000 m/z

Method X1:

Instrument: Labomatic HD5000, Labocord-5000; Gilson GX-241, Labcol Vario 4000; Column: Chiralpak IE 5 μm 250×20 mm; Eluent A: MTBE +0.1%vol. Diethylamine (99%); Eluent B: Ethanol; Isocratic: 90%A +10%B; Flow 30.0 mL/min; UV 254 nm.

Method X2:

Instrument: Labomatic HD5000, Labocord-5000; Gilson GX-241, Labcol Vario 4000; Column: Chiralpak IA 5 μm 250×30 mm; Eluent A: MTBE +0.1%vol. Diethylamine (99%); Eluent B: Ethanol; Isocratic: 85%A +15%B; Flow 40.0 mL/min; UV 254 nm.

Method X3:

Instrument: Labomatic HD5000, Labocord-5000; Gilson GX-241, Labcol Vario 4000, Column: Chiralpak IA 5.0 μm 250×30 mm; Eluent: 100% Acetonitrile; Flow 50.0 mL/min; UV 280 nm.

Method X4:

Instrument: Waters Autopurification system; Column: Waters XBrigde C18 5.0 μm 100×30 mm; Eluent A: H₂O +0.2%vol. NH₃ (32%), Eluent B: Acetonitrile; Gradient: 0.00-0.50 min. 8% B (25->70 mL/min), 0.51-5.50 min. 8-15% B (70 mL/min), DAD scan: 210-400 nm.

Method X5:

Instrument: Labomatic HD5000, Labocord-5000; Gilson GX-241, Labcol Vario 4000, Column: Chiralpak IF 5.0 μm 250×30 mm; Eluent A: Hexane +0.1%vol. Diethylamine (99%); Eluent B: Ethanol; Isocratic: 90%A +10%B; Flow 50.0 mL/min; UV 280 nm.

Method X6:

Instrument: Waters Autopurification system; Column: Waters XBrigde C18 5.0 μm 100×30 mm; Eluent A: H₂O +0.2%vol. NH₃ (32%), Eluent B: Acetonitrile; Gradient: 0.00-0.50 min. 30% B (25->70 mL/min), 0.51-5.50 min. 30-45% B (70 mL/min), DAD scan: 210-400 nm.

Method X7:

Instrument: Labomatic HD5000, Labocord-5000; Gilson GX-241, Labcol Vario 4000, Column: Chiralpak ID 5.0 μm 250×30 mm; Eluent A: Hexane +0.1%vol Diethylamin (99%); Eluent B: 2-Propanol; Isocratic: 85%A +15%B; Flow 50.0 mL/min; UV 254 nm.

### EXAMPLE 1

### 6-ethoxy-N-[(1R)-1-(4-fluorophenyl)ethyl]-2-methylpyrido [3,4-d]pyrimidin-4-amine

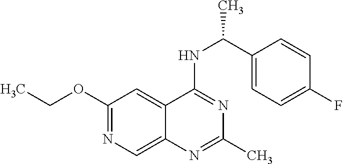 [SMILES: CCOc1cc2c(N[C@H](C)c3ccc(F)cc3)nc(C)nc2cn1]

### Step a

### 6-ethoxy-2-methylpyrido[3,4-d]pyrimidin-4-ol

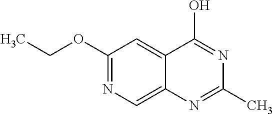

A round-bottom flask was charged with ethanol (110 ml) and cooled with an ice bath. To the ethanol was carefully added sodium (3.73 g, 163 mmol) and stirred for 5 min. 6-fluoro-2-methylpyrido[3,4-d]pyrimidin-4-ol (5.85 g, 32.7 mmol, described in example 35, step a) was added and the mixture was stirred at 110° C. for 16 h. The course of the reaction was monitored by LC/MS, nearly complete conversion was detected. The solution was cooled to room temperature and concentrated in vacuo. Under cooling in an ice-bath the residue was diluted with 500 ml of water, then acidified with 2M hydrochloric acid (200 mL) to pH=1 and extracted with dichloromethane (2×200 ml) and a mixture of dichlormethane/isopropanol (4:1, 5×200 ml). The combined organic layers were dried over sodium sulfate and then concentrated in vacuo. The title compound (4.83 g, 77%) was obtained in form of a beige/brown-coloured solid. 1H-NMR (400 MHz, DMSO): d [ppm]=8.62 (s, 1H), 7.17 (s, 1H), 4.34 (q, 2H), 1.34 (t, 3H).

### Step b

### 6-ethoxy-2-methylpyrido[3,4-d]pyrimidin-4-yl 2,4,6-triisopropylbenzenesulfonate

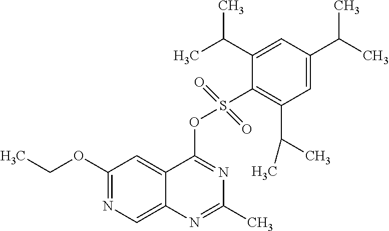 [SMILES: CCOc1cc2c(OS(=O)(=O)c3c(C(C)C)cc(C(C)C)cc3C(C)C)nc(C)nc2cn1]

A microwave vial was charged with 6-ethoxy-2-methylpyrido[3,4-d]pyrimidin-4-ol (150 mg, 0.73 mmol, described in example 1, step a), 2,4,6-triisopropyl-benzensulfonylchlorid (244 mg, 0.80 mmol, commercially available), triethylamine (0.32 ml, 2.27 mmol) and 4-dimethylaminopyridine (9 mg, 0.073 mmol). The mixture was suspended in dry N,N-dimethylformamide (1.5 ml) and stirred at room temperature for 3 h. The course of the reaction was monitored by LC/MS. Conversion was observed. The mixture was used without further purification in the next step.

### Step c

### 6-ethoxy-N-[(1R)-1-(4-fluorophenypethyl]-2-methylpyrido[3,4-d]pyrimidin-4-amine

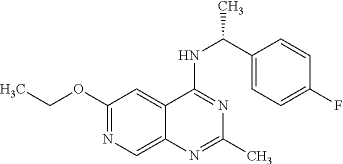 [SMILES: CCOc1cc2c(N[C@H](C)c3ccc(F)cc3)nc(C)nc2cn1]

(R)-(+)-1-(4-Fluorophenyl)ethylamine (122 mg, 0.88 mmol, commercially available) was added to a mixture of 6-ethoxy-2-methylpyrido[3,4-d]pyrimidin-4-yl 2,4,6-triisopropylbenzene-sulfonate (0.73 mmol, described in example 1, step b) and stirred at room temperature for 18 h. The course of the reaction was monitored by LC/MS. The mixture was diluted with dichloromethane (50 ml) and washed with water (30 ml). The organic layer was dried over sodium sulfate, filtered, and concentrated in vacuo. The crude product was purified by flash chromatography [silica gel 60 (40 g, 30 μm); dichloromethane]. The title compound (112 mg, 47%) was isolated in form of an orange solid. ¹H-NMR (400 MHz, CDCl₃): δ [ppm]=1.39 (t, 3H), 1.66 (d, 3H), 2.60 (s, 3H), 4.40 (q, 2H), 5.60 (m, 1H), 5.93 (s br, 1H), 6.89 (s, 1H), 7.03 (m, 2H), 7.41 (m, 2H), 8.85 (s, 1H). LC-MS (method 1): Rₜ=3.11 min.; MS (ESI/APCIpos) m/z=327.2 [M+H]⁺.

### EXAMPLE 2

### N-[(1R)-1-(4-fluorophenyl)ethyl]-6-methoxy-2-methylpyrido[3,4-d]pyrimidin-4-amine

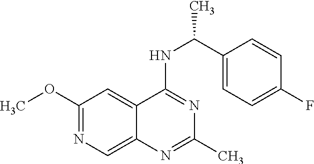 [SMILES: COc1cc2c(N[C@H](C)c3ccc(F)cc3)nc(C)nc2cn1]

### Step a

### 6-methoxy-2-methylpyrido[3,4-d]pyrimidin-4-ol

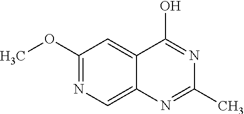

2,5 g (14.867 mmol) of 5-amino-2-methoxy-4-pyridinecarboxylic acid, 2.81 g (29.735 mmol, commercially available) of acetamidine hydrochloride and 2.44 g (29.735 mmol) of anhydrous sodium acetate were suspended in 40 ml of 2-methoxyethanol and stirred under reflux for 6 hours. After cooling to ambient temperature water (50 ml) was added to the reaction. The precipitate was filtered, washed with cold water (3×10 ml) and dried in vucuum. The title compound was obtained in 2.31 g as a grey solid. 1H-NMR (400 MHz, DMSO): d [ppm]=12.27 (br s, 1H), 8.60 (d, 1H), 7.19 (d, 1H), 3.91 (s, 3H), 2.32 (s, 3H).

### Step b

### 6-methoxy-2-methylpyrido[3,4-d]pyrimidin-4-yl 2,4,6-triisopropylbenzenesulfonate

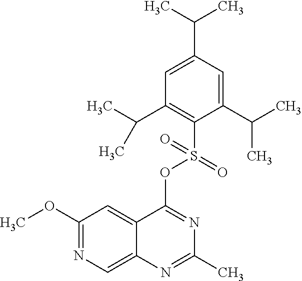 [SMILES: COc1cc2c(OS(=O)(=O)c3c(C(C)C)cc(C(C)C)cc3C(C)C)nc(C)nc2cn1]

6-methoxy-2-methylpyrido[3,4-d]pyrimidin-4-ol (200 mg, 1.046 mmol, described in example 2, step a), triethylamine (0.452 ml, 3.243 mmol), 2,4,6-triisopropyl-benzenesulfonyl chloride (349 mg, 1.151 mmol) and 4-dimethylaminopyridine (13 mg, 0.105 mmol) were combined in N,N-dimethylformamide (2 ml) and stirred 2 h at room temperature. The progress of the reaction was monitored by LC/MS. Complete conversion. The mixture was used without further treatment in the next step.

### Step c

### N-[(1R)-1-(4-fluorophenyl)ethyl]-6-methoxy-2-methylpyrido[3,4-d]pyrimidin-4-amine

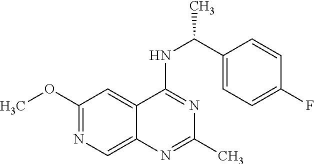 [SMILES: COc1cc2c(N[C@H](C)c3ccc(F)cc3)nc(C)nc2cn1]

(R)-(+)-1-(4-Fluorphenyl)ethylamine 145 mg, 1.043 mmol, commercially available) was added to the reaction mixture of 2,4,6-trisopropylbenzenesulfonic acid 6-methoxy-2-methylpyrido-[3,4d]pyrimidin-4-yl ester (246 mg, 0.522 mmol, described in example 2, step b) in dimethylformamide (1 ml) and stirred at room temperature overnight. The progress of the reaction was monitored by LC/MS. The reaction mixture was concentrated under pressure and the residue was partitioned between dichlormethane (20 ml) and water (10 ml). The organic phase was dried over sodium sulfate, filtered, and concentrated under reduced pressure. The crude product was purified by flash chromatography [silica gel 60 (25 g, 30 μm); chloroform/methanol 98:2]. 145 mg (89% d. Th.) of the title compound were isolated in form of a white solid.

1H-NMR (400 MHz, CDCl₃): δ[ppm]=1.64-1.66 (m, 3H), 2.57 (s, 3H), 3.98 (s, 3H), 5.56-5.63 (m, 1H), 5.85-5.86 (m, 1H), 6.86 (s, 1H), 6.97-7.03 (m, 2H), 7.36-7.41 (m, 2H), 8.84 (s, 1H). LC-MS (method 1): m/z: [M+H]⁺=313.2, Rₜ=3.46 min.

### EXAMPLE 3

### N-[1-(1-benzothiophen-4-yl)ethyl]-6-ethoxy-2-methylpyrido[3,4-d]pyrimidin-4-amine

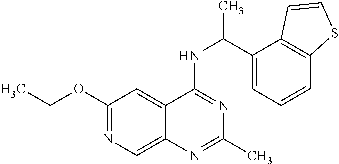 [SMILES: CCOc1cc2c(NC(C)c3cccc4sccc34)nc(C)nc2cn1]

### Step a

### 4-(1-ethoxyvinyl)-1-benzothiophene

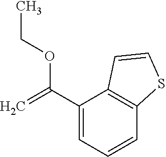

To a solution of 4-Bromobenzothiophene (1 g, 4.69 mmol, commercially available) in DMF (9.2 ml) were added 1-Ethoxyvinyltri-n-butyltin (2.2 g, 6.1 mmol) and Pd(PPh3)4 (1.085 g, 0.939 mmol). The reaction was stirred at 110° C. overnight. After cooling to ambient temperature the reaction was poured into brine and extracted with EtOAc (3×20 ml). The combined organic layers were washed with water (2×) and brine (1×), dried over sodium sulfate and the solvent was removed under reduced pressure. The crude product (2.5 g) was used without further purification in the next step.

### Step b

### 1-(1-benzothiophen-4-yl)ethanone

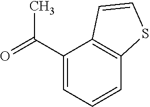

To a solution of crude 4-(1-ethoxyvinyl)-1-benzothiophene (959 mg, 4.69 mmol, described in example 3, step a), in THF (18 ml) was added HCl (2N, 7.04 ml) and the reaction was stirred at room temperature over the weekend (UPLC). The reaction was quenched with saturated aqueous NaHCO3 solution, extracted with ethyl acetate (3×). The combined organic layers were evaporated and the residue was purified via column chromatography (50 g silica, eluent: gradient hexanes/ethyl acetate (0-20%). The title compound (1.07 g) still contained impurities from the tin reagent used in step a and was used without further purification in the next step. 1H-NMR (400 MHz, DMSO-d6): d [ppm]=8.32 (dt, 1H), 8.22 (dd, 1H), 8.12 (dd, 1H), 8.00 (d, 1H), 7.53 (t, 1H), 2.70 (s, 3H).

### Step c

### 1-(1-benzothiophen-4-yl)-N-hydroxyethanimine

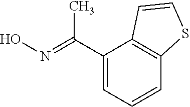

A solution of 1-(1-benzothiophen-4-yl)ethanone (827 mg, 4.69 mmol), hydroxylamine hydrochloride (1.63 g, 23.5 mmol) and sodium acetate (3.85 g, 46.9 mmol) in ethanol (19 ml) was stirred at 40° C. overnight. The reaction mixture was filtered and the solvent of the filtrate was removed under reduced pressure. The residue was taken up in ethyl acetate and HCl (1N in water, 15 ml) was added followed by brine. The layers were separated, the organic layer was dried over sodium sulfate and the solvent was removed under reduced pressure. The crude title compound was used in the next step without further purification.

### Step d

### 1-(1-benzothiophen-4-yl)ethanamine

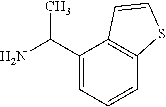

To a mixture of 1-(1-benzothiophen-4-yl)-N-hydroxyethanimine (898 mg, 4.69 mmol), zinc (15.34 g, 235 mmol) in methanol (67 ml) was added ammonium chloride (15 g, 282 mmol) and the reaction was stirred at 60° C. overnight. The reaction was filtered over celite and the solvent removed under reduced pressure. The residue was taken up in water, basified with ammonia and extracted with ethyl acetate (3×). The solvent was removed under reduced pressure and the title compound (832 mg) was used without further purification in the next step.1H-NMR (400 MHz, CDCl₃): d [ppm]=7.79 (dt, 1H), 7.55 (dd, 1H), 7.50-7.43 (m, 2H), 7.38-7.28 (m, 1H), 4.69 (q, 1H), 2.02 (s, 2H), 1.53 (d, 3H).

### Step e

### N-[1-(1-benzothiophen-4-yl)ethyl]-6-ethoxy-2-methylpyrido[3,4-d]pyrimidin-4-amine

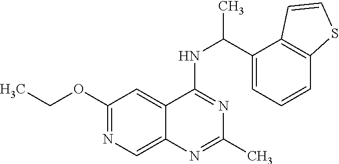 [SMILES: CCOc1cc2c(NC(C)c3cccc4sccc34)nc(C)nc2cn1]

1-(1-benzothiophen-4-yl)ethanamine (255 mg, 1.44 mmol, described in example 3, step d) was added to the mixture of 6-ethoxy-2-methylpyrido[3,4-d]pyrimidin-4-yl 2,4,6-triisopropyl¬benzene¬isulfonate (1.20 mmol, described in example 1, step b) and stirred at room temperature for 18 h. The course of the reaction was monitored by LC/MS. Conversion was observed. The mixture was diluted with dichloromethane (70 ml) and washed with water (5×20 ml). The organic layer was dried over sodium sulfate, filtered, and concentrated in vacuo. The crude product was purified by flash chromatography [silica gel 60 (40 g, 30 μm) dichloromethane/methanol (97:3)]. The title compound (158 mg, 36%) was isolated in form of an bright yellow-coloured solid. 1H-NMR (400 MHz, CDCl₃): d [ppm]=8.84 (s, 1H), 7.84 (d, 1H), 7.57 (dd, 1H), 7.47 (d, 1H), 7.44 (d, 1H), 7.37-7.32 (m, 1H), 6.77 (s, 1H), 6.17 (quin, 1H), 4.38 (q, 2H), 2.62 (s, 3H), 1.80 (d, 3H), 1.36 (t, 3H).

### EXAMPLE 4

### N-[1-(1-benzothiophen-4-yl)ethyl]-6-methoxy-2-methylpyrido[3,4-d]pyrimidin-4-amine

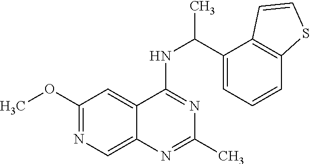 [SMILES: COc1cc2c(NC(C)c3cccc4sccc34)nc(C)nc2cn1]

1-(1-benzothiophen-4-yl)ethanamine (222 mg, 1.26 mmol, described in example 3, step d) was added to the mixture of 6-methoxy-2-methylpyrido[3,4-d]pyrimidin-4-yl 2,4,6-triisopropyl¬benzene-sulfonate (1.05 mmol, described in example 2, step b) and stirred at room temperature for 18 h. The course of the reaction was monitored by LC/MS. Conversion was observed. The mixture was diluted with dichloromethane (70 ml) and washed with water (5×20 ml). The organic layer was dried over sodium sulfate, filtered, and concentrated in vacuo. The crude product was purified by flash chromatography [silica gel 60 (40 g, 30 μm) dichloromethane/methanol (97:3)]. The title compound (120 mg, 33%) was isolated in form of an bright yellow-coloured solid. 1H-NMR (400 MHz, CDCl₃): d [ppm]=8.88 (s, 1H), 7.83 (d, 1H), 7.60 (d, 1H), 7.55-7.41 (m, 2H), 7.40-7.28 (m, 1H), 7.00 (br d, 1H), 6.20 (quin, 1H), 3.88 (br s, 3H), 2.64 (s, 3H), 1.84 (d, 3H).

### EXAMPLE 5

### N-[(1R)-1-(3-bromophenyl)ethyl]-6-ethoxy-2-methylpyrido[3,4-d]pyrimidin-4-amine

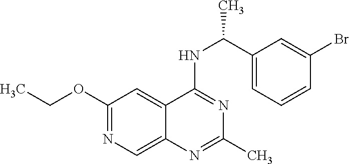 [SMILES: CCOc1cc2c(N[C@H](C)c3cccc(Br)c3)nc(C)nc2cn1]

(R)-3-Bromo-alpha-methylbenzylamine (273 mg, 1.365 mmol, commercially available) was added to the mixture of 6-ethoxy-2-methylpyrido[3,4-d]pyrimidin-4-yl 2,4,6-triisopropyl¬benzene¬sulfonate (0.975 mmol, described in example 1, step b) and stirred at room temperature for 18 h. The course of the reaction was monitored by LC/MS. Conversion was observed. The mixture was diluted with dichloromethane (70 ml) and washed with wather (30 ml). The organic layer was dried over sodium sulfate, filtered, and concentrated in vacuo. The crude product was purified by flash chromatography [silica gel 60 (40 g, 30 μm) dichloromethane/methanol (97:3)]. The title compound (223 mg, 59%) was isolated in form of an beige-coloured solid. 1H-NMR (400 MHz, CDCl₃): d [ppm]=8.85 (d, 1H), 7.57 (t, 1H), 7.44-7.30 (m, 2H), 7.28-7.14 (m, 2H), 6.89 (s, 1H), 5.58 (quin, 1H), 4.41 (q, 2H), 2.59 (s, 3H), 1.66 (d, 3H), 1.39 (t, 3H).

### EXAMPLE 6

### N-[(1R)-1-(3-chlorophenypethyl]-6-ethoxy-2-methylpyrido[3,4-d]pyrimidin-4-amine

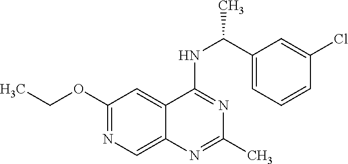 [SMILES: CCOc1cc2c(N[C@H](C)c3cccc(Cl)c3)nc(C)nc2cn1]

(R)-3-Chloro-alpha-methylbenzylamine (92 mg, 0.59 mmol, commercially available) was added to the mixture of 6-ethoxy-2-methylpyrido[3,4-d]pyrimidin-4-yl 2,4,6-triisopropyl¬benzene¬sulfonate (0.49 mmol, described in example 1, step b) and stirred at room temperature for 18 h. The course of the reaction was monitored by LC/MS. Conversion was observed. The mixture was diluted with dichloromethane (70 ml) and washed with water (30 ml). The organic layer was dried over sodium sulfate, filtered, and concentrated in vacuo. The crude product was purified by flash chromatography [silica gel 60 (40 g, 30 μm) dichloromethane/methanol (97:3)]. The title compound (60 mg, 36%) was isolated in form of an orange-colored solid. 1H-NMR (400 MHz, CDCl₃): d [ppm]=8.85 (s, 1H), 7.40 (s, 1H), 7.33-7.17 (m, 4H), 6.85 (s, 1H), 5.82 (br s, 1H), 5.58 (quin, 1H), 4.42 (q, 2H), 2.58 (s, 3H), 1.66 (d, 3H), 1.40 (t, 3H).

### EXAMPLE 7

### 6-ethoxy-2-methyl-N-{(1R)-1-[3-(methylsulfonyl)phenyl]ethyl}pyrido[3,4-d]pyrimidin-4-amine

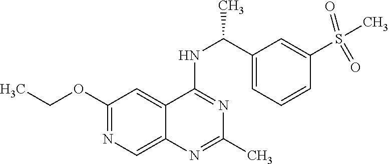 [SMILES: CCOc1cc2c(N[C@H](C)c3cccc(S(C)(=O)=O)c3)nc(C)nc2cn1]

A microwave vial was charged with N-[(1R)-1-(3-bromophenyl)ethyl]-6-ethoxy-2-methylpyrido[3,4-d]pyrimidin-4-amine (124 mg, 0.32 mmol, described in example 5), sodium methanesulphinate (65 mg, 0.64 mmol, commercially available), copper(II)trifluoormethanesulfonate (23 mg, 0.064 mmol), and racemic trans-1,2-diaminocyclohexane (15 mg, 0.128 mmol). The vial was sealed with a Teflon cap and the reaction mixture was dissolved in dry dimethylsulfoxide (1 ml). The vial was degassed (3×), refilled with argon and then stirred at 130° C. for 18 h. The course of the reaction was monitored by LC/MS. Conversion was observed. The mixture was cooled to room temperature, diluted with dichloromethane (50 ml), washed with water, (3×25 ml), which was then extracted with dichloromethane (3×15 ml). The combined organic layers were dried over sodium sulfate, filtered, and concentrated in vacuo. The crude product was purifed by flash chromatography [silica gel 60 (25 g, 30 μm); dichloromethane/methanol (97:3 to 90:10)]. The title compound (57 mg, 46%) was isolated in form of a white-colored solid. ¹H-NMR (400 MHz, CDCl₃): δ [ppm]=8.83 (s, 1H), 8.05 (t, 1H), 7.86-7.81 (m, 1H), 7.74 (d, 1H), 7.54 (t, 1H), 7.26 (s, 1H), 6.95 (s, 1H), 6.18 (br s, 1H), 5.66 (quin, 1H), 4.39 (q, 2H), 3.05 (s, 3H), 2.55 (s, 3H), 1.70 (d, 3H), 1.37 (t, 3H).

### EXAMPLE 8

### N-[(1R)-1-(3-chloro-4-fluorophenyl)ethyl]-6-methoxy-2-methylpyrido[3,4-(1]pyrimidin-4-amine

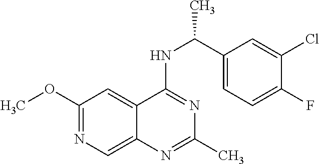 [SMILES: COc1cc2c(N[C@H](C)c3ccc(F)c(Cl)c3)nc(C)nc2cn1]

3′-Chlor-4′-fluorphenyl)ethylamine (181 mg, 1.046 mmol, commercially available) was added to the reaction mixture of 2,4,6-trisopropylbenzenesulfonic acid 6-methoxy-2-methylpyrido-[3,4d]pyrimidin-4-yl ester (246 mg, 0.539 mmol, described in example 2, step b) in dimethylformamide (1 ml) and stirred at room temperature overnight. The progress of the reaction was monitored by LC/MS. The reaction mixture was concentrated under reduced pressure and the residue was partitioned between dichlormethane (20 ml) and water (10 ml). The organic phase was dried over sodium sulfate, filtered, and concentrated under reduced pressure. The crude product was purified by flash chromatography [silica gel 60 (25 g, 30 ium); chloroform/methanol 98 : 2]. 47 mg (24%) of the title compound was isolated in form of a white solid. ¹H-NMR (400 MHz, CDCl₃): δ [ppm]=1.65-1.67 (m, 3H), 2.58 (s, 3H), 3.99 (s, 3H), 5.55-5.58 (m, 1H), 5.88-5.96 (m, 1H), 6.89 (s, 1H), 7.07-7.11 (m, 1H), 7.28-7.32 (m, 1H), 7.45-7.48 (m, 1H), 8.86 (s, 1H). LC-MS (method 1): m/z: [M+H]⁺=347.2, Rₜ=3.45 min

### EXAMPLE 9

### 6-fluoro-N-[(1R)-1-(4-fluorophenypethyl]-2-methylpyrido[3,4-d]pyrimidin-4-amine

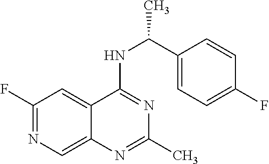 [SMILES: Cc1nc(N[C@H](C)c2ccc(F)cc2)c2cc(F)ncc2n1]

(R)-(+)-1-(4-fluorophenyl)ethylamine (1.40 g, 10.0 mmol, commercially available) was added to the mixture of 6-fluoro-2-methylpyrido[3,4-d]pyrimidin-4-yl2,4,6-tri(propan-2-yl)benzene-sulfonate (8.37 mmol, described in example 35, step b) and stirred at room temperature for 18 h. The course of the reaction was monitored by LC/MS. Conversion was observed. The mixture was concentrated in vacuo. the crude product was diluted with ethyl acetate (300 ml) and washed with wather (5×100 ml) and brine (100 ml). The organic layer was dried over sodium sulfate, filtered, and concentrated in vacuo. The crude product was purified by flash chromatography [silica gel 60 (400 g) dichloromethane/methanol (97:3)]. The title compound (1.61 g, 64%) was isolated in form of an white-coloured solid. 1H-NMR (400 MHz, <cdcl3>): d [ppm]=8.84 (s, 1H), 7.48-7.36 (m, 2H), 7.13 (d, 1H), 7.07-6.98 (m, 2H), 5.90 (br s, 1H), 5.62 (t, 1H), 2.62 (s, 3H), 1.68 (d, 3H).

### EXAMPLE 10

### N-[(1R)-1-(4-fluorophenypethyl]-6-(2-methoxyethoxy)-2-methylpyrido[3,4-d]pyrimidin-4-amine

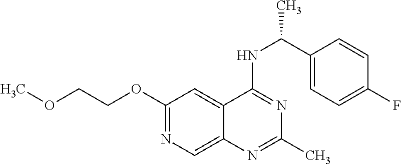 [SMILES: COCCOc1cc2c(N[C@H](C)c3ccc(F)cc3)nc(C)nc2cn1]

A microwave vial was charged with sodium hydride (60% dispersion in mineral oil, 32 mg, 0.79 mmol) under a flow of argon. 2-Methoxyethanol (61 mg, 0.80 mmol, commercially available) in N-methyl pyrrolidinone (4 ml) was added and the mixture was stirred at room temperature for 5 min. 6-fluoro-N-[(1R)-1-(4-fluorophenyl)ethyl]-2-methylpyrido[3,4-d]pyrimidin-4-amine (150 mg, 0.50 mmol, described in example 9) was added and the mixture was heated to 180° C. for 800 s using microwave irradiation. The course of the reaction was monitored by LC/MS. Complete conversion was observed. The mixture was poured (50 ml) into water. The aqueous layer was extracted with ethyl acetate (5×20 ml). The combined organic layers were washed with water (5×15 ml) and brine (20 ml), dried over sodium sulfate, filtered, and concentrated in vacuo. The crude product was purified by flash chromatography [silica gel 60 (40 g, 30 μm) dichloromethane/methanol (98:2 to 97:3)]. The title compound (70 mg, 38%) was isolated in form of an orange-coloured solid. 1H-NMR (400 MHz, CDCl₃): d [ppm]=8.81 (s, 1H), 7.37 (dd, 2H), 7.02 (t, 2H), 6.92 (s, 1H), 5.80 (br d, 1H), 5.57 (quin, 1H), 4.58-4.47 (m, 2H), 3.77-3.69 (m, 2H), 3.41 (s, 3H), 2.58 (s, 3H), 1.64 (d, 3H).

### EXAMPLE 11

### N-[1R)-1-(4-fluorophenypethyl]-2-methyl-6-(tetrahydro-2H-pyran-4-ylmethoxy)-pyrido[3,4-d]pyrimidin-4-amine

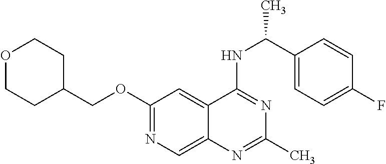 [SMILES: Cc1nc(N[C@H](C)c2ccc(F)cc2)c2cc(OCC3CCOCC3)ncc2n1]

A microwave vial was charged with sodium hydride (60% dispersion in mineral oil, 32 mg, 0.80 mmol) under a flow of argon. Tetrahydropyran-4-methanol (93 mg, 0.80 mmol, commercially available) in N,N-dimethylformamid (3 ml) was added and the mixture was stirred at room temperature for 20 min. 6-fluoro-N-[(1R)-1-(4-fluorophenyl)ethyl]-2-methylpyrido[3,4-d]pyrimidin-4-amine (150 mg, 0.50 mmol, described in example 9) was added and the mixture was heated to 170° C. for 3000 s using microwave irradiation. The course of the reaction was monitored by LC/MS. Conversion was observed. The mixture was poured into ethyl acetate (100 ml) and washed with water (3×50 ml). The organic layer was dried over sodium sulfate, filtered, and concentrated in vacuo. The crude product was purified by flash chromatography [silica gel 60 (40 g, 30 μm) dichloromethane/methanol (97:3 to 95:5)]. The title compound (133 mg, 67%) was isolated in form of an orange-coloured solid. 1H-NMR (400 MHz, CDCl₃): d [ppm]=8.84 (s, 1H), 7.45-7.34 (m, 2H), 7.03 (t, 2H), 6.83 (s, 1H), 5.75 (br s, 1H), 5.64-5.52 (m, 1H), 4.23 (d, 2H), 4.00 (dd, 2H), 3.58-3.35 (m, 2H), 2.59 (s, 3H), 2.14-1.97 (m, 1H), 1.74 (br dd, 2H), 1.66 (d, 3H), 1.53-1.41 (m, 2H).

### EXAMPLE 12

### N-[(1R)-1-(3-cyclopropyl-4-fluorophenypethyl]-6-methoxy-2-methylpyrido[3,4-cl]pyrimidin-4-amine

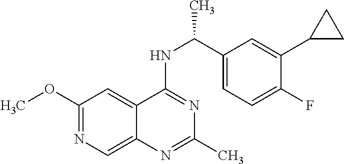 [SMILES: COc1cc2c(N[C@H](C)c3ccc(F)c(C4CC4)c3)nc(C)nc2cn1]

### Step a

### 1-(3-Cyclopropyl-4-fluorophenyl)ethanone

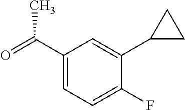

A mixture of 3-bromo-4′-fluoroacetophenone (842 mg, 3.88 mmol, commercially available), cyclopropylboronic acid (1 g, 11.64 mmol), bis(diphenylphosphino)ferrocene-dichloropalladium(II) complex in dichloromethane (317 mg, 0.388 mmol), and cesium carbonate (3.80 g, 11.64 mmol) was suspended in a microwave vessel under argon in 1,4-dioxane (30 ml). The vessel was degassed three times and refilled with argon. The mixture was stirred for 30 min. at 100° C. The course of the reaction was monitored by LC/MS. The reaction solution was concentrated in vacuo to dryness. The residue was taken up in dichloromethane (10 ml) and washed three times with water (10 ml). The organic phase was dried over sodium sulfate and concentrated in vacuo to dryness. The crude product was purified by flash chromatography [silica gel 60 (40 g, 30 μm); dichloromethane/methanol (96 : 4)]. The title compound (570 mg, 82%) was isolated as a solid. ¹1-I-NMR (400 MHz, CDCl₃): δ [ppm]=0.78-0.81 (m, 2H), 1.00-1.05 (m, 2H), 2.10-2.13 (m, 1H), 2.55 (s, 3H), 7.04-7.08 (m, 1H), 7.54-7.57 (m, 1H), 7.71-7.75 (m, 1H). LC-MS (method 1): m/z: [M+H]±=179.2, Rₜ=3.49 min. Step b

### 1-(3-Cyclopropyl-4-fluorophenyl)ethylamine

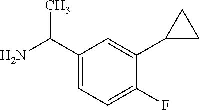

A mixture of 1-(3-cyclopropyl-4-fluorophenyl)ethanone (570 g, 3.2 mmol, described in example 12, step a), titan(IV)isopropylate (1.818 mg, 6.39 mmol) and ammonia in ethyl alcohol (2M, 7.99 ml, 15.993 mmol) was stirred under argon in a capped flask at ambient temperature for 6 h. Sodium borohydride (181.5 mg, 4.798 mmol) was then added and the resulting mixture was stirred at room temperature for additional 3 h. The reaction was then quenched by pouring into ammonium hydroxide (2M, 12 ml), the resulting inorganic precipitate was filtered off, and washed with ethyl acetate (2×10 ml). The organic layer was separated and the remaining aqueous layer was extracted with ethyl acetate (2×12 ml). The combined organic extracts were extracted with hydrochloric acid (1M, 15 ml) to separate the neutral materials. The acidic aqueous extracts were washed with ethyl acetate (25 ml), then adjusted with aqueous sodium hydroxide (2M) to pH 10-12, and extracted with ethyl acetate (3×25 ml). The combined organic extracts were washed with brine (25 ml), dried over sodium sulfate, and concentrated in vacuo to afford the title compound (290 mg, 51%). ¹H-NMR (400 MHz, CDCl₃): δ [ppm]=0.72-0.76 (m, 2H), 0.94-0.99 (m, 2H), 1.33-1.35 (m, 3H), 1.67 (s, 2H), 2.05-2.08 (m, 1H), 4.03-4.08 (m, 1H), 6.87-6.96 (m, 2H), 7.06-7.10 (m, 1H).

### Step c

### N-[(1R)-1-(3-cyclopropyl-4-fluorophenyl)ethyl]-6-methoxy-2-methylpyrido[3,4-d]pyrimidin-4-amine

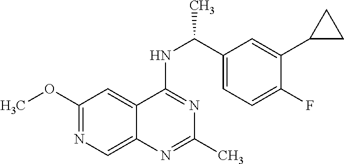 [SMILES: COc1cc2c(N[C@H](C)c3ccc(F)c(C4CC4)c3)nc(C)nc2cn1]

1-(3-Cyclopropyl-4-fluorophenyl)ethylamine (290 mg, 1.618 mmol, described in example 12, step b) was added to the reaction mixture of 2,4,6-trisopropylbenzenesulfonic acid 6-methoxy-2-methyl-pyrido-[3,4d]pyrimidin-4-yl ester (493 mg, 1.078 mmol, described in example 2, step b) in dimethylformamide (2 ml) and stirred at room temperature overnight. The progress of the reaction was monitored by LC/MS. The reaction mixture was concentrated under reduced pressure and the residue was partitioned between dichloromethane (20 ml) and water (10 ml). The organic phase was dried over sodium sulfate, filtered, and concentrated under reduced pressure. The crude product was purifed by flash chromatography [silica gel 60 (25 g, 30 μm); chloroform/methanol 98:2]. 123 mg (29% yield) of the title compound was isolated in form of a white solid. ¹H-NMR (400 MHz, CDCl₃): δ [ppm]=0.71-0.75 (m, 2H), 0.97-0.99 (m, 2H), 1.64-1.66 (m, 3H), 2.07 (s, 1H), 2.61 (s, 3H), 3.99 (s, 3H), 5.51-5.93 (m, 2H), 6.87 (s, 1H), 6.95-7.02 (m, 2H), 7.18-7.19 (m, 1H), 8.86 (s, 1H). LC-MS (method 1): m/z: [M+H]⁺=353.2, Rₜ=3.53 min.

### EXAMPLE 13

### N-[(1R)-1-(4-bromophenyl)ethyl]-6-ethoxy-2-methylpyrido[3,4-d]pyrimidin-4-amine

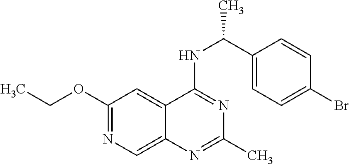 [SMILES: CCOc1cc2c(N[C@H](C)c3ccc(Br)cc3)nc(C)nc2cn1]

(R)-4-bromo-alpha-methylbenzylamine (293 mg, 1.463 mmol, commercially available) was added to the reaction mixture of 6-ethoxy-2-methylpyrido[3,4-d]pyrimidin-4-yl 2,4,6-triisopropyl¬benzene¬sulfonate (459 mg, 0.975 mmol, described in example 1, step b) and stirred overnight. The progress of the reaction was monitored by LC/MS. The reaction mixture was concentrated under pressure and the residue was partitioned between dichloro-methane (20 ml) and water (10 ml). The organic phase was dried over sodium sulfate, filtered and concentrated under reduced pressure. The crude product was purifed by flash chromatography [silica gel 60 (40 g, 30 μm); chloroform/methanol (98:2)]. 110 mg (29% yield) of the title compound was isolated in form of a orange solid. 1H-NMR (400 MHz, CDCl₃): d [ppm]=8.87 (s, 1H), 7.52-7.42 (m, 2H), 7.31 (d, 2H), 6.87 (br s, 1H), 5.57 (quin, 1H), 4.41 (q, 2H), 2.59 (s, 3H), 1.67 (d, 3H), 1.39 (t, 3H).

### EXAMPLE 14

### 6-methoxy-2-methyl-N-[1-(1-methyl-1H-indazol-4-yl)ethyl]pyrido[3,4-d]pyrimidin-4-amine

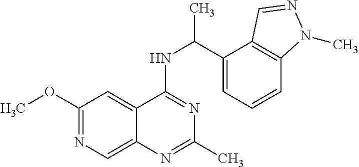

### Step a

### 1-methyl-4-(1-nitroethyl)-1H-indazole

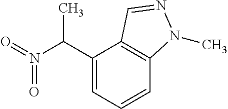

A microwave vial was charged with 4-bromo-1-methyl-1H-indazol (200 mg, 0.948 mmol, commercially available), nitroethane (711 mg, 9.47 mmol), tris(dibenzylidene-acetone)dipalladium (43 mg, 0.047 mmol), 2-dicyclohexylphosphino-2′,4′,6′-triisopropylbiphenyl (54 mg, 0.114 mmol), and tripotassium phosphate (241 mg, 1.137 mmol). The vial was sealed with a teflon cap and the reaction mixture was dissolved in 3 ml of dry 1,4-dioxane. The vial was degassed three times, refilled with argon and then stirred at 110° C. for 4 h. The course of the reaction was monitored by LC/MS. Conversion and violent decomposition was observed. The mixture was cooled to room temperature, diluted with 50 ml dichloromethane, washed with 15 ml of 1M aqueous hydrochloric acid, which was then extracted three times with 5 ml of dichloromethane. The combined organic layers were dried over sodium sulfate, filtered, and concentrated in vacuo. The crude product was purifed by flash chromatography [silica gel 60 (25 g, 30 pm); dichloromethane/methanol (99 : 1 to 97:3)]. 297 mg (100% yield) of the title compound was isolated in form of an orange-coloured liquid. ¹H-NMR (400 MHz, CDCl₃): δ [ppm]=2.04-2.10 (m, 3H), 4.10 (s, 3H), 5.98-6.03 (m, 1H), 7.24-7.26 (m, 1H), 7.39-7.47 (m, 2H), 8.14 (s, 1H). LC-MS (method 1): m/z: [M+H]⁺=206.2, Rₜ=2.99 min.

### Step b

### 1-(1-Methyl-1H-indazol-4-yl)ethylamine

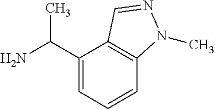

A round-bottomed flask was charged with 1-methyl-4-(1-nitroethyl)-1H-indazole (140 mg, 0.682 mmol, described in example 14, step a), acetic acid (10 ml) and zinc (312 mg, 4.775 mmol) under a flow of argon. The reaction mixture was then stirred at room temperature for 15 minutes. The course of the reaction was monitored by LC/MS. The mixture was filtered and concentrated in vacuo. The residue was treated with aqueous sodium hydroxide (2M) to pH 10-12, and extracted with dichloromethane (3×25 ml). The organic phase was dried over sodium sulfate, filtered and concentrated under reduced pressure. 110 mg (92% yield) of the title compound was isolated. ¹H-NMR (400 MHz, CDCl₃): δ [ppm]=1.52-1.54 (m, 3H), 4.07 (s, 3H), 4.56-4.57 (m, 1H), 7.14-7.16 (m, 1H), 7.26-7.28 (m, 1H), 7.34-7.37 (m, 1H), 8.13 (s, 1H.

### Step c

### 6-methoxy-2-methyl-N-[1-(1-methyl-1H-indazol-4-yl)ethyl]pyrido[3,4-d]pyrimidin-4-amine

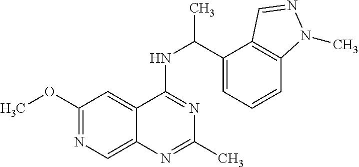

1-(1-Methyl-1H-indazol-4-yeethylamine (110 mg, 0.626 mmol, described in example 14, step b) was added to the reaction mixture of 2,4,6-trisopropylbenzenesulfonic acid 6-methoxy-2-methylpyrido-[3,4d]pyrimidin-4-yl ester (191 mg, 0.417 mmol, described in example 2, step b) in dimethylformamide (1 ml) and stirred at room temperature overnight. The progress of the reaction was monitored by LC/MS. The reaction mixture was concentrated under pressure and the residue was partitioned between dichlormethane (20 ml) and water (10 ml). The organic phase was dried over sodium sulfate, filtered and concentrated under reduced pressure. The crude product was purifed by flash chromatography [silica gel 60 (40 g, 30 μm); chloroform/methanol 98:2]. 45 mg (31% yield) of the title compound were isolated in form of a white solid. 1H-NMR (400 MHz, CDCl₃): δ [ppm]=1.80-1.82 (m, 3H), 2.60 (s, 3H), 3.97 (s, 3H), 4.07 (s, 3H), 6.03-6.08 (m, 2H), 6.82 (s, 1H), 7.18-7.19 (m, 1H), 7.31-7.38 (m, 2H), 8.13 (s, 1H), 8.85 (s, 1H).

### EXAMPLE 15

### N-[(1R)-1-(4-fluorophenypethyl]-2-methyl-6-(propan-2-yloxy)pyrido[3,4-d]pyrimidin-4-amine

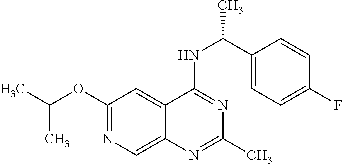 [SMILES: Cc1nc(N[C@H](C)c2ccc(F)cc2)c2cc(OC(C)C)ncc2n1]

A microwave vial was charged with sodium hydride (60% dispersion in mineral oil, 32 mg, 0.80 mmol) under a flow of argon. 2-propanol (48 mg, 0.80 mmol) in N,N-dimethylformamid (3 ml) was added and the mixture was stirred at room temperature for 20 min. 6-fluoro-N-[(1R)-1-(4-fluorophenyl)ethyl]-2-methylpyrido[3,4-d]pyrimidin-4-amine (150 mg, 0.50 mmol, described in example 9) was added and the mixture was heated to 170° C. for 4000 s using microwave irradiation. The course of the reaction was monitored by LC/MS. Half conversion was observed but the reaction stand still. A solution of sodium hydride (60% dispersion in mineral oil, 32 mg, 0.80 mmol) and 2-Propanol (48 mg, 0.80 mmol) in N,N-dimethylformamid (3 ml) was stirred for 20 min. and was added to the reaction mixture and they was heated to 170° C. for 3000 s using microwave irradiation. The mixture was poured into ethyl acetate (100 ml) and washed with water (3×50 ml). The organic layer was dried over sodium sulfate, filtered, and concentrated in vacuo. The crude product was purified by flash chromatography [silica gel 60 (40 g, 30 μm) dichloromethane/methanol (97:3)]. The title compound (82 mg, 42%) was isolated in form of an orange-coloured solid. 1H-NMR (400 MHz, CDCl₃): d [ppm]=8.85 (s, 1H), 7.39 (dd, 2H), 7.03 (t, 2H), 6.76 (s, 1H), 5.78-5.51 (m, 2H), 5.37 (dt, 1H), 2.59 (s, 3H), 1.65 (d, 3H), 1.36 (dd, 6H).

### EXAMPLE 16

### N-[(1R)-1-(4-fluorophenyl)ethyl]-2-methyl-6-(methylsulfanyl)pyrido[3,4-d]pyrimidin-4-amine

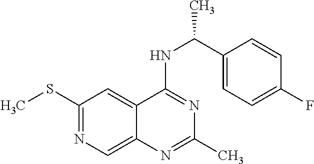 [SMILES: CSc1cc2c(N[C@H](C)c3ccc(F)cc3)nc(C)nc2cn1]

A microwave vial was charged with sodium methanethiolate (56 mg, 0.80 mmol) and 6-fluoro-N-[(1R)-1-(4-fluorophenyl)ethyl]-2-methylpyrido [3 ,4-d]pyrimidin-4-amine (150 mg, 0.50 mmol, described in example 9). N,N-dimethylformamide (3 ml) was added and the mixture was heated to 170° C. for 4000 s using microwave irradiation. The course of the reaction was monitored by LC/MS. Conversion was observed. The mixture was poured into ethyl acetate (100 ml) and washed with water (3×50 ml). The organic layer was dried over sodium sulfate, filtered, and concentrated in vacuo. The crude product was purified by flash chromatography [2 times, silica gel 60 (40 g, 30 μm) dichloromethane/methanol (98:2)]. The title compound (85 mg, 52%) was isolated in form of an orange-colored solid. 1H-NMR (400 MHz, DMSO-d6): d [ppm]=8.87 (d, 1H), 8.62 (d, 1H), 8.12 (d, 1H), 7.54-7.40 (m, 2H), 7.15 (t, 2H), 5.60 (quin, 1H), 2.61 (s, 3H), 2.41 (s, 3H), 1.58 (d, 3H). LCMS (method 7): m/z [M+H]⁺=329.1, Rₜ=0.80 min.

### EXAMPLE 17

### 6-methoxy-2-methyl-N-[1-(2-methyl-2H-indazol-4-yl)ethyl]pyrido[3,4-d]pyrimidin-4-amine

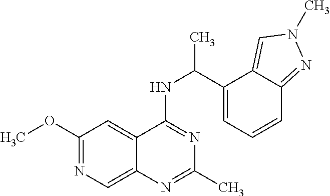

### Step a

### 2-Methyl-4-(1-nitroethyl)-2H-indazole

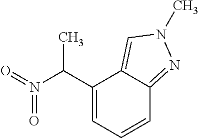

A microwave vial was charged with 4-bromo-2-methyl-1H-indazole (200 mg, 0.948 mmol, commercially available), nitroethane (711 mg, 9.47 mmol), tris(dibenzylideneacetone)-dipalladium (43 mg, 0.047 mmol), 2-dicyclohexylphosphino-2′,4′,6′-triisopropylbiphenyl (54 mg, 0.114 mmol), and tripotassium phosphate (241 mg, 1.137 mmol). The vial was sealed with a teflon cap and the reaction mixture was dissolved in 3 ml of dry 1,4-dioxane. The vial was degassed three times, refilled with argon and then stirred at 110° C. for 4 h. The course of the reaction was monitored by LC/MS. Conversion and violent decomposition were observed. The mixture was cooled to room temperature, diluted with dichloromethane (50 nal), washed with 1M aqueous hydrochloric acid (15 ml), which was then extracted three times with dichloromethane (5 ml). The combined organic layers were dried over sodium sulfate, filtered, and concentrated in vacuo. The crude product was purified by flash chromatography [silica gel 60 (25 g, 30 pm); dichloromethane/methanol (99:1 to 97:3)]. 188 mg (97% yield) of the title compound was isolated in form of an orange-colored liquid contaminated with a by-product. The product was used without further purification in the next step.

### Step b

### 1-(2-Methyl-2H-indazol-4-yl)ethylamine

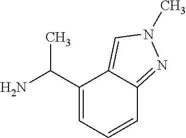

A round-bottomed flask was charged with 2-methyl-4-(1-nitroethyl)-2H-indazole (188 mg, 0.916 mmol, described in example 17, step a), acetic acid (10 ml) and zinc (419 mg, 6.413 mmol) under a flow of argon. The reaction mixture was then stirred at room temperature for 15 minutes. The course of the reaction was monitored by LC/MS. The mixture was filtered and concentrated in vacuo. The residue was then adjusted with aqueous sodium hydroxide (2M) to pH 10-12 and extracted with dichloromethane (3×25 ml). The organic phase was dried over sodium sulfate, filtered, and concentrated under reduced pressure. 160 mg (100% yield) of the title compound were isolated. ¹H-NMR (400 MHz, CDCl₃): δ [ppm]=1.50-1.52 (m, 3H), 4.22 (s, 3H), 4.40-4.43 (m, 1H), 7.19-7.27 (m, 2H), 7.57-7.59 (m, 1H), 8.12 (s, 1H).

### Step c

### (6-Methoxy-2-methylpyrido[3,4-d]pyrimidin-4-y0-[1-(1-methyl-1H-indazol-4-yl)ethyl]amine

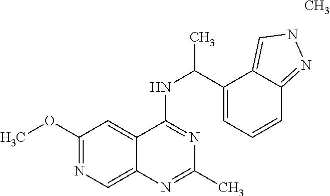

1-(2-Methyl-2H-indazol-4-yl)ethylamine (160 mg, 0.913 mmol, described in example 17, step b) was added to the reaction mixture of 2,4,6-trisopropylbenzenesulfonic acid 6-methoxy-2-methylpyrido-[3,4d]pyrimidin-4-yl ester (278 mg, 0.609 mmol, described in example 2, step b) in dimethylformamide (1 ml) and stirred at room temperature overnight. The progress of the reaction was monitored by LC/MS. The reaction mixture was concentrated under reduced pressure and the residue was partitioned between dichlormethane (20 ml) and water (10 ml). The organic phase was dried over sodium sulfate, filtered and concentrated under reduced pressure. The crude product was purified by flash chromatography [silica gel 60 (40 g, 30 ium); chloroform/methanol 98:2]. 39 mg (17% yield) of the title compound was isolated in form of a white solid. ¹H-NMR (400 MHz, CDCl₃): δ [ppm]=1.78-1.80 (m, 3H), 2.66 (s, 3H), 3.96 (s, 3H), 4.16 (s, 3H), 5.98-6.03 (m, 1H), 6.82 (s, 1H), 7.12-7.14 (m, 1H), 7.24-7.28 (m, 2H), 7.62-7.64 (m, 1H), 7.97 (s, 1H), 8.87 (s, 1H). LC-MS (method 1): m/z: [M+H]⁺=349.2, Rₜ=3.02 min.

### EXAMPLE 18

### 4-{(1R)-1-[(6-ethoxy-2-methylpyrido[3,4-d]pyrimidin-4-yl)amino]ethyl}benzonitrile

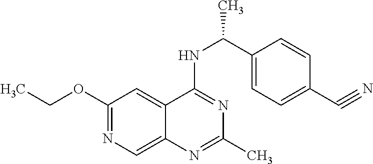 [SMILES: CCOc1cc2c(N[C@H](C)c3ccc(C#N)cc3)nc(C)nc2cn1]

(R)-4-(1-aminoethyl)benzonitrile hydrochloride (110 mg, 0.626 mmol, commercially available) and N,N-diisopropylethylamine (252 mg, 1.951 mmol) were added to the reaction mixture of 6-ethoxy-2-methylpyrido[3,4-d]pyrimidin-4-yl 2,4,6-triisopropyl¬benzene¬sulfonate (460 mg, 0.975 mmol, described in example 1, step b) and stirred overnight. The progress of the reaction was monitored by LC/MS. The reaction mixture was concentrated under pressure and the residue was partitioned between dichlormethane (20 ml) and water (10 ml). The organic phase was dried over sodium sulfate, filtered and concentrated under reduced pressure. The crude product was purifed by flash chromatography (silica gel 60 (40 g, 30 μm); chloroform/methanol (98:2)). 102 mg (31% yield) of the title compound was isolated in form of a white solid. 1H-NMR (400 MHz, CDCl₃): d [ppm]=8.82 (s, 1H), 7.64-7.55 (m, 2H), 7.53-7.44 (m, 2H), 6.94 (s, 1H), 6.06 (br d, 1H), 5.60 (quin, 1H), 4.40 (q, 2H), 2.51 (s, 3H), 1.66 (d, 3H), 1.38 (t, 3H).

### EXAMPLE 19

### 6-ethoxy-2-methyl-N-[(1R)-1-phenylethyl]pyrido[3,4-d]pyrimidin-4-amine

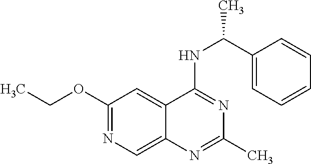 [SMILES: CCOc1cc2c(N[C@H](C)c3ccccc3)nc(C)nc2cn1]

(R)-(+)-alpha-methylbenzylamine (177 mg, 1.463 mmol, commercially available) was added to the reaction mixture of 6-ethoxy-2-methylpyrido[3,4-d]pyrimidin-4-yl 2,4,6-triisopropyl¬benzene¬isulfonate (460 mg, 0.975 mmol, described in example 1, step b) and stirred overnight. The progress of the reaction was monitored by LC/MS. The reaction mixture was concentrated under pressure and the residue was partitioned between dichlormethane (20 ml) and water (10 ml). The organic phase was dried over sodium sulfate, filtered and concentrated under reduced pressure. The crude product was purifed by flash chromatography [silica gel 60 (40 g, 30 μm); chloroform/methanol (98:2)]. 147 mg (46% yield) of the title compound was isolated in form of a white solid. 1H-NMR (400 MHz, CDCl₃): d [ppm]=8.81 (d, 1H), 7.43-7.38 (m, 2H), 7.35-7.29 (m, 2H), 7.28-7.24 (m, 1H), 6.85 (d, 1H), 5.95 (br d, 1H), 5.62 (quin, 1H), 4.37 (q, 2H), 2.58 (s, 3H), 1.65 (d, 3H), 1.37 (t, 3H).

### EXAMPLE 20

### 6-(benzyloxy)-N-[(1R)-1-(3-bromophenyl)ethyl]-2-methylpyrimido[5,4-d]pyrimidin-4-amine

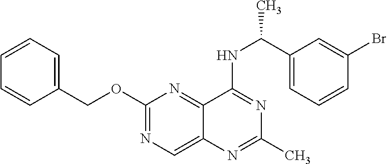 [SMILES: Cc1nc(N[C@H](C)c2cccc(Br)c2)c2nc(OCc3ccccc3)ncc2n1]

### Step a

### 6-(Benzyloxy)-2-methylpyrimido[5,4-d]pyrimidin-4(3H)-one

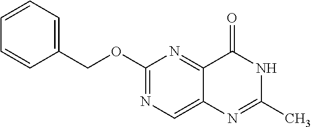 [SMILES: Cc1nc2cnc(OCc3ccccc3)nc2c(=O)[nH]1]

This compound was synthesized by the same method as described in example 24 (step c) to give 141.00 mg (62%) of product as a yellow solid. MS (ESIpos): m/z=269 (M+H)⁺; LC-MS (Method 4, Acetonitrile-Water-0.05%TFA-5%B): Rₜ=0.77 min.

### Step b

### 6-(Benzyloxy)-4-chloro-2-methylpyrimido[5,4-d]pyrimidine

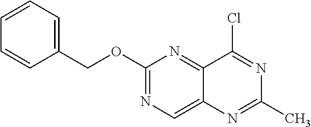 [SMILES: Cc1nc(Cl)c2nc(OCc3ccccc3)ncc2n1]

This compound was synthesized by the same method as described in example 24 (step d) to give 150.00 mg (crude) of the product as a yellow solid and the product was used directly for next step without further purification.

### Step c

### (R)-6-(benzyloxy)-N-(1-(3-bromophenypethyl)-2-methylpyrimido[5,4-d]pyrimidin-4-amine

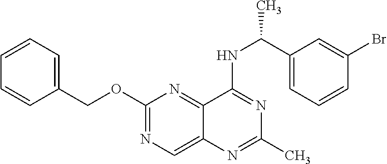 [SMILES: Cc1nc(N[C@H](C)c2cccc(Br)c2)c2nc(OCc3ccccc3)ncc2n1]

This compound was synthesized by the same method as described in example 24 (step e) to give 58.20 mg (24%) of the product as a yellow solid. MS (ESIpos): m/z=450 (M+H)⁺; LC-MS (Acetonitrile-Water-0.05%TFA-5%B): Rₜ=1.28 min. ¹H-NMR (400 MHz, DMSO-d₆): δ [ppm]=1.62-1.64 (d, 3H), 2.44 (s, 3H), 5.54-5.66 (m, 3H), 7.29-7.55 (m, 6H), 7.57-7.58 (d, 2H), 7.72-7.73 (d, 1H), 8.56-8.58 (d, 1H), 9.16 (s, 1H).

### EXAMPLE 21

### N8-[(1R)-1-(3-bromophenypethyl]-N2,N2,6-trimethylpyrimido[5,4-d]pyrimidine-2,8-diamine

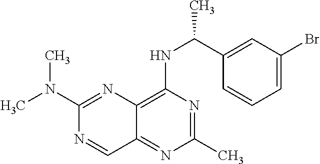 [SMILES: Cc1nc(N[C@H](C)c2cccc(Br)c2)c2nc(N(C)C)ncc2n1]

### Step a

### 6-(Dimethylamino)-2-methylpyrimido [5,4-d]pyrimidin-4(3H)-one

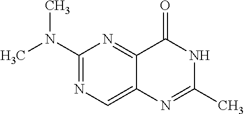

2-Methyl-6-(methylsulfonyl)pyrimido[5,4-d]pyrimidin-4(3H)-one, 200 mg (0.8 mmol, described in example 24), and dimethylamine (4.2 mL, 2M in tetrahydrofuran) were dissolved in 5 mL of acetonitrile and the resulting mixture was stirred at 100° C. for 12 hours. After evaporation in vacuo, the residue was purified with silica gel column chromatography to give 130 mg (76%) of the product as a yellow solid. MS (ESIpos): m/z=206 (M+H)⁺; LC-MS (Method 4, Acetonitrile-Water-0.05%TFA-5%B): Rₜ=0.97 min.

### Step b

### 8-Chloro-N,N,6-trimethylpyrimido[5,4-d]pyrimidin-2-amine

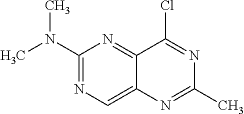

This compound was synthesized by the same method as described in example 24 (step d) to give 140.00 mg (crude) of the product as a yellow solid and the product was used directly for next step without further purification.

### Step c

### (R)-N⁸-(1-(3-bromophenypethyl)-N²,N²,6-trimethylpyrimido[5,4-d]pyrimidine-2,8-diamine

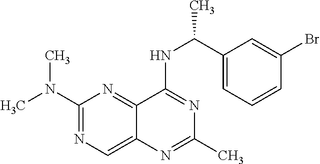 [SMILES: Cc1nc(N[C@H](C)c2cccc(Br)c2)c2nc(N(C)C)ncc2n1]

This compound was synthesized by the same method as described in example 24 (step e) to give 37.10 mg (12%) of the product as a yellow solid. MS (ESIpos): m/z=387 (M+H)⁺; LC-MS (Method 4, Acetonitrile-Water-0.05%TFA-5%B): Rₜ=1.10 min. ¹H-NMR (400 MHz, DMSO-d₆): δ[ppm]=1.60-1.62 (d, 3H), 2.37 (s, 3H), 3.27 (s, 6H), 5.49-5.53 (t, 1H), 7.28-7.32 (t, 1H), 7.42-7.48 (m, 2H), 7.68-7.69 (t, 1H), 8.05-8.07 (d, 1H), 8.90 (s, 1H).

### EXAMPLE 22

### N-[(1R)-1-(3-bromophenyl)ethyl]-2-methyl-6-(morpholin-4-yl)pyrimido[5,4-d]pyrimidin-4-amine

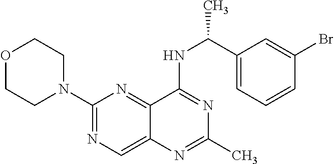 [SMILES: Cc1nc(N[C@H](C)c2cccc(Br)c2)c2nc(N3CCOCC3)ncc2n1]

### Step a

### 2-Methyl-6-morpholinopyrimido[5,4-d]pyrimidin-4(3H)-one

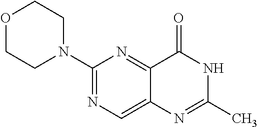 [SMILES: Cc1nc2cnc(N3CCOCC3)nc2c(=O)[nH]1]

This compound was synthesized by the same method as described in example 21 (step a) to give 170 mg (81%) of the product, as a yellow solid. MS (ESIpos): m/z=248 (M+H)⁺; LC-MS (Method 4, Acetonitrile-Water-0.05%TFA-5%B): Rₜ=0.69 min.

### Step b

### 4-(8-Chloro-6-methylpyrimido[5,4-d]pyrimidin-2-yl)morpholine

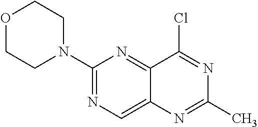 [SMILES: Cc1nc(Cl)c2nc(N3CCOCC3)ncc2n1]

This compound was synthesized by the same method as described in example 24 (step d) to give 105.00 mg (crude) of the product as a yellow solid and the product was used directly for next step without further purification.

### Step c

### (R)-N-(1-(3-bromophenypethyl)-2-methyl-6-morpholinopyrimido[5,4-d]pyrimidin-4-amine

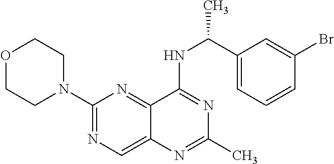 [SMILES: Cc1nc(N[C@H](C)c2cccc(Br)c2)c2nc(N3CCOCC3)ncc2n1]

This compound was synthesized by the same method as described in example 24 (step e) to give 16.70 mg (12%) of the product as a yellow solid. MS (ESIpos): m/z=429 (M+H)⁺. LC-MS (Method 4, Acetonitrile-Water-0.05%TFA-5%B): Rₜ=1.07 min. ¹H-NMR (400 MHz, DMSO-d₆): δ[ppm]=1.53-1.55 (d, 3H), 2.31 (s, 3H), 3.64-3.67 (t, 4H), 3.81-3.83 (t, 4H), 5.44-5.48 (t, 1H), 7.21-7.25 (t, 1H), 7.35-7.41 (m, 2H), 7.60-7.61 (d, 1H), 8.14-8.16 (d, 1H), 8.87 (s, 1H).

### EXAMPLE 23

### N-[(1R)-1-(3-bromophenyl)ethyl]-6-ethoxy-2-methylpyrimido[5,4-d]pyrimidin-4-amine

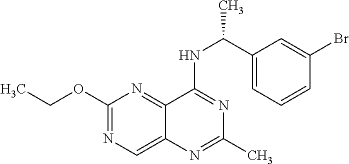 [SMILES: CCOc1ncc2nc(C)nc(N[C@H](C)c3cccc(Br)c3)c2n1]

### Step a

### 6-Ethoxy-2-methylpyrimido[5,4-d]pyrimidin-4(3H)-one

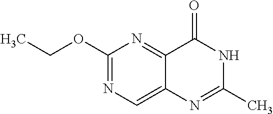

This compound was synthesized by the same method as described in example 24 (step c) to give 160.00 mg (88%) of the product as a yellow solid. MS (ESIpos): m/z=207 (M+H)⁺; LC-MS (Method 4, Acetonitrile-Water-0.05%TFA-5%B): Rₜ=1.22 min.

### Step b

### 4-Chloro-6-ethoxy-2-methylpyrimido[5,4-d]pyrimidine

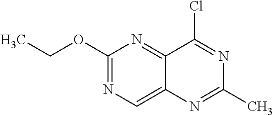

This compound was synthesized by the same method as described in example 24 (step d) to give 170.00 mg (crude) of the product as a yellow solid and the product was used directly for next step without further purification.

### Step c

### (R)-N-(1-(3-bromophenypethyl)-6-ethoxy-2-methylpyrimido[5,4-d]pyrimidin-4-amine

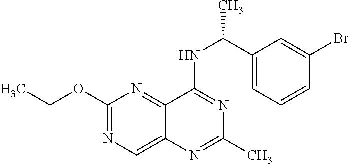 [SMILES: CCOc1ncc2nc(C)nc(N[C@H](C)c3cccc(Br)c3)c2n1]

This compound was synthesized by the same method as described in example 24 (step e) to give 20.90 mg (7%) of the product as a yellow semi-solid. MS (ESIpos): m/z=388 (M+H)⁺; LC-MS (Method 4, Acetonitrile-Water-0.05%TFA-5%B): Rₜ=1.12 min. ¹H-NMR (400 MHz, DMSO-d₆): δ [ppm]=1.38-1.42 (t, 3H), 1.61-1.62 (d, 3H), 2.43 (s, 3H), 4.54-4.57 (m, 2H), 5.55 (m, 1H), 7.28-7.32 (t, 1H), 7.42-7.44 (d, 1H), 7.48-7.50 (d, 1H), 7.71 (s, 1H), 9.13 (s, 1H).

### EXAMPLE 24

### N-[(1R)-1-(3-bromophenyl)ethyl]-6-methoxy-2-methylpyrimido[5,4-d]pyrimidin-4-amine

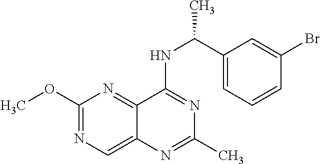 [SMILES: COc1ncc2nc(C)nc(N[C@H](C)c3cccc(Br)c3)c2n1]

### Step a

### 2-Methyl-6-(methylthio)pyrimido[5,4-d]pyrimidin-4(3H)-one

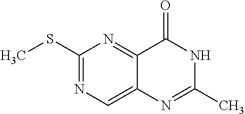

A three-necked round bottom flask was charged with a magnetic stirrer, evacuated and back-filled with nitrogen. 5-Bromo-2-(methylthio)pyrimidine-4-carboxylic acid, 15.00 g (60.2 mmol, commercially available), and acetimidamide hydrochloride, 8.49 g (90.3 mmol), in N,N-dimethylformamide (150 mL) were added under nitrogen and the resulting solution was stirred at room temparature for 2 hours. Then cesium carbonate, 39.24 g (120.4 mmol), was added to the flask and stirred at room temperature for 2.5 hours. Copper(I) iodide, 2.29 g (4.1 mmol), was added to the above mixture at room temperature and the resulting slurry was stirred at 80° C. for 12 hours under nitrogen. The mixture was filtered and the filtrate was concentrated under vacuum. The residue was purified by silica gel column chromatography to give 3.76 g (30%) of product as a yellow solid. MS (ESIpos): m/z=209 (M+H)⁺; LC-MS (Acetonitrile-Water-0.05%TFA-5%B): Rₜ=0.99 min.

### Step b

### 2-Methyl-6-(methylsulfonyl)pyrimido[5,4-d]pyrimidin-4(3H)-one

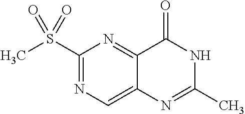

2-Methyl-6-(methylthio)pyrimido[5,4-d]pyrimidin-4(3H)-one, 2.36 g (11.3 mmol), was dissolved in 60 mL of dichloromethane, then meta-chloroperoxybenzoic acid, 5.87 g (34.0 mmol) was added in several batches. The resulting mixture was stirred at room temperature for 8 hours. After evaporation in vacuo, the residue was purified by silica gel column chromatography to give 2.32 g (84%) of the product as a yellow solid. MS (ESIpos): m/z=241 (M+H)⁺; LC-MS (Method 4, Acetonitrile-Water-0.05%TFA-5%B): Rₜ=0.40 min. See reference Angew. Chem. Int. Ed. 2009, 48, 348.

### Step c

### 6-Methoxy-2-methylpyrimido[5,4-d]pyrimidin-4(3H)-one

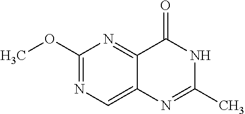

2-Methyl-6-(methylsulfonyl)pyrimido[5,4-d]pyrimidin-4(3H)-one, 200.00 mg (0.8 mmol), and sodium methoxide, 899.49 mg (16.7 mmol) were dissolved in 10 mL of methanol and the resulting mixture was stirred at room temperature for 4 hours. The precipitated solid was filtered out and the filtration was concentrated in vacuo. The residue was purified by silica gel column chromatography to give 145.00 mg (88%) of the product as an off-white solid. MS (ESIpos): m/z=193 (M+H)⁺; LC-MS (Method 4, Acetonitrile-Water-0.05%TFA-5%B): Rₜ=1.11 min.

### Step d

### 4-Chloro-6-methoxy-2-methylpyrimido[5,4-d]pyrimidine

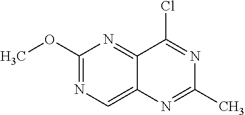

6-Methoxy-2-methylpyrimidol5,4-dlpyrimidin-4(3H)-one, 170.00 mg (0.9 mmol), was dissolved in 5 mL of thionyl chloride and the resulting mixture was stirred at 90° C. for 4 hours. The solvent was removed in vacuo to give 185.00 mg (crude) of the product as a yellow solid and the product was used directly for next step without further purification.

### Step e

### (R)-N-(1-(3-bromophenypethyl)-6-methoxy-2-methylpyrimido[5,4-d]pyrimidin-4-amine

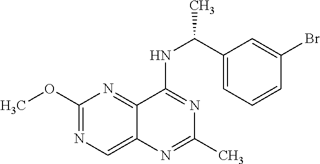 [SMILES: COc1ncc2nc(C)nc(N[C@H](C)c3cccc(Br)c3)c2n1]

4-Chloro-6-methoxy-2-methylpyrimido[5,4-d]pyrimidine, 185.00 mg (0.9 mmol), was treated with a solution of (R)-1-(3-bromophenyl)ethanamine, 263.61 mg (1.3 mmol, commercially available), in 2-propanol (5 mL) and the resulting mixture was stirred at 110° C. for 20 min. After evaporation in vacuo, the residue was purified by Prep-HPLC to give 100.80 mg (30%) of the product as a yellow semi-solid. MS (ESIpos): m/z=374 (M+H)⁺; LC-MS (Method 4, Acetonitrile-Water-0.05%TFA-5%B): Rₜ=1.06 min. ¹H-NMR (400 MHz, CD₃OD): δ [ppm]=1.69-1.71 (d, 3H), 2.53 (s, 3H), 4.18 (s, 3H), 5.55-5.60 (m, 1H), 7.24-7.28 (t, 1H), 7.40-7.42 (d, 1H), 7.46-7.48 (d, 1H), 7.67 (s, 1H), 9.04 (s, 1H).

### EXAMPLE 25

### N-[(1R)-1-(3-bromophenyl)ethyl]-2-methyl-6-phenoxypyrimido[5,4-d]pyrimidin-4-amine

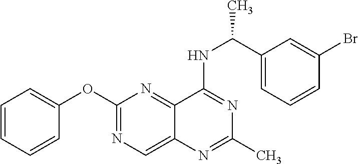 [SMILES: Cc1nc(N[C@H](C)c2cccc(Br)c2)c2nc(Oc3ccccc3)ncc2n1]

### Step a

### 2-Methyl-6-phenoxypyrimido[5,4-d]pyrimidin-4(31/)-one

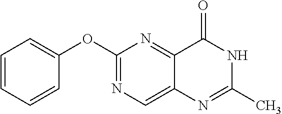 [SMILES: Cc1nc2cnc(Oc3ccccc3)nc2c(=O)[nH]1]

2-Methyl-6-(methylsulfonyl)pyrimido[5,4-d]pyrimidin-4(3H)-one, 200.00 mg (0.8 mmol, described in example 24), potassium carbonate, 345.16 mg (2.5 mmol), and phenol, 117.52 mg (1.3 mmol), were dissolved in 19 mL of NN-dimethylformamide and the resulting mixture was stirred at 70° C. for 4 hours. The solids were filtered out and the filtrate was concentrated in vacuo. The residue was purified with silica gel column chromatography to give 180.00 mg (82%) of the product as a light yellow solid. MS (ESIpos): m/z=255 (M+H)⁺; LC-MS (Method 4, Acetonitrile-Water-0.05%TFA-5%B): Rₜ=1.33 min.

### Step b

### 4-Chloro-2-methyl-6-phenoxypyrimido[5,4-d]pyrimidine

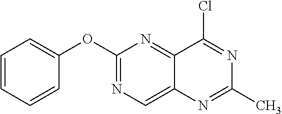 [SMILES: Cc1nc(Cl)c2nc(Oc3ccccc3)ncc2n1]

This compound was synthesized by the same method as described in example 24 (step d) to give 190.00 mg (crude) of the product as a yellow solid and the product was used directly for next step without further purification.

### Step c

### (R)-N-(1-(3-bromophenypethyl)-2-methyl-6-phenoxypyrimido[5,4-d]pyrimidin-4-amine

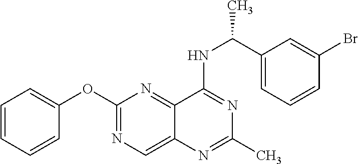 [SMILES: Cc1nc(N[C@H](C)c2cccc(Br)c2)c2nc(Oc3ccccc3)ncc2n1]

This compound was synthesized by the same method as described in example 24 (step e) to give 37.10 mg (12%) of the product as a yellow solid. MS (ESIpos): m/z=436 (M+H)⁺. LC-MS (Method 4, Acetonitrile-Water-0.05%TFA-5%B): Rₜ=1.27 min. ¹H-NMR (400 MHz, CD₃OD): δ [ppm]=1.60-1.62 (d, 3H), 2.53 (s, 3H), 5.47-5.52 (m, 1H), 7.23-7.33 (m, 4H), 7.39-7.41 (t, 2H), 7.47-7.51 (m, 2H), 7.60 (s, 1H), 9.04 (s, 1H).

### EXAMPLE 26

### N-[(1R)-1-(3-bromophenyl)ethyl]-6-(2-methoxyethoxy)-2-methylpyrimido[5,4-d]pyrimidin-4-amine

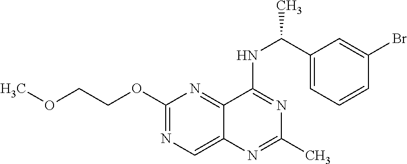 [SMILES: COCCOc1ncc2nc(C)nc(N[C@H](C)c3cccc(Br)c3)c2n1]

### Step a

### 6-(2-Methoxyethoxy)-2-methylpyrimido[5,4-d]pyrimidin-4(3H)-one

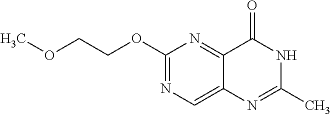

2-Methyl-6-(methylsulfonyl)pyrimido[5,4-d]pyrimidin-4(3H)-one, 200 mg (0.8 mmol, described in example 24), and 2-methoxyethanol, 95.02 mg (1.3 mmol), were dissolved in 8 mL of N,N-dimethylformamide This was followed by the addition of sodium hydride, 66.59 mg (1.7 mmol), at 0° C. and the resulting mixture was stirred at room temperature for 4 hours.

The reaction was then quenched by the addition of saturated aqueous ammonium chloride and the resulting solution was extracted with ethyl acetate. The combined organic layers were dried over anhydrous sodium sulfate and the solvent was removed under vacuum. The residue was purified with silica gel column chromatography to give 175.00 mg (85%) of the product as a light yellow solid. MS (ESIpos): m/z=237 (M+H)⁺; LC-MS (Method 4, Acetonitrile-Water-0.05%TFA-5%B): Rₜ=1.19 min.

### Step b

### 4-Chloro-6-(2-methoxyethoxy)-2-methylpyrimido[5,4-d]pyrimidine

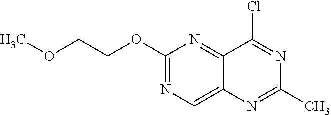

This compound was synthesized by the same method as described in example 24 (step d) to give 185.00 mg (crude) of the product as a yellow solid and the product was used directly for next step without further purification.

### Step c

### (R)-N-(1-(3-bromophenypethyl)-6-(2-methoxyethoxy)-2-methylpyrimido[5,4-d]pyrimidin-4-amine

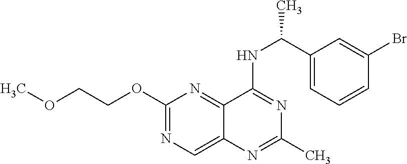 [SMILES: COCCOc1ncc2nc(C)nc(N[C@H](C)c3cccc(Br)c3)c2n1]

This compound was synthesized by the same method as described in example 24 (step e) to give 69.80 mg (23%) of the product as a yellow semi-solid. MS (ESIpos): m/z=418 (M+H)⁺; LC-MS (Method 4, Acetonitrile-Water-0.05%TFA-5%B): Rₜ=1.08 min. ¹H-NMR (400 MHz, CD₃OD): δ [ppm]=1.69-1.70 (d, 3H), 2.53 (s, 3H), 3.46 (s, 3H), 3.83-3.86 (t, 2H), 4.71-4.74 (m, 2H), 5.56-5.61 (m, 1H), 7.25-7.29 (t, 1H), 7.40-7.42 (t, 1H), 7.46-7.48 (d, 1H), 7.67 (s, 1H), 9.05 (s, 1H).

### EXAMPLE 27

### N-[(1R)-1-(3-bromophenyl)ethyl]-2-methyl-6-(morpholin-4-yl)pyrido[3,2-d]pyrimidin-4-amine

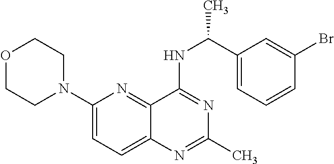 [SMILES: Cc1nc(N[C@H](C)c2cccc(Br)c2)c2nc(N3CCOCC3)ccc2n1]

This compound was synthesized by the same method as described in example 31 to give 37.60 mg (55%) of the product as a light yellow solid. MS (ESIpos): m/z=428 (M+H)⁺; LC-MS (Method 4, Acetonitrile-Water-0.05%TFA-5%B): Rₜ=1.74 min. ¹H-NMR (400 MHz, DMSO-d₆): δ [ppm]=1.60-1.61 (d, 3H), 2.36 (s, 3H), 3.67-3.70 (m, 4H), 3.72-3.77 (m, 4H), 5.51-5.54 (m, 1H), 7.27-7.31 (t, 1H), 7.41-7.48 (m, 3H), 7.67-7.68 (m, 1H), 7.76-7.80 (m, 2H).

### EXAMPLE 28

### N-[(1R)-1-(3-bromophenyl)ethyl]-6-methoxy-2-methylpyrido[3,2-d]pyrimidin-4-amine

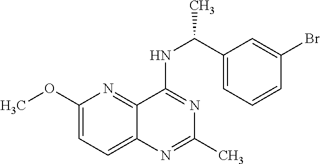 [SMILES: COc1ccc2nc(C)nc(N[C@H](C)c3cccc(Br)c3)c2n1]

### Step a

### 6-Chloro-3-nitropicolinamide

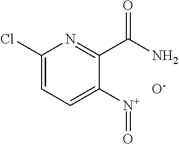

6-Chloro-3-nitropicolinonitrile, 5.00 g (27.2 mmol, commercially available), was dissolved in 75 mL of 95% sulfuric acid and the resulting mixture was stirred at 70° C. for 10 hours. The mixture was poured into ice water and extracted with ethyl acetate. The combined organic phase was dried over anhydrous sodium sulfate and the solvent was removed in vacuo to give 4.80 g (86%) of the final product as a light yellow solid. MS (ESIpos): m/z=202 (M+H)⁺; LC-MS (Method 4, Acetonitrile-Water-0.1%FA-10%B): Rₜ=0.50 min.

### Step b

### 3-Amino-6-chloropicolinamide

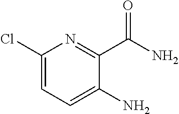 [SMILES: NC(=O)c1nc(Cl)ccc1N]

6-Chloro-3-nitropicolinamide, 4.80 g (23.8 mmol), and palladium carbon (10%), 0.67 g (6.3 mmol), were added into 60 mL of ethyl acetate. The resulting mixture was stirred under hydrogen atmosphere (2 atm) for 3 hours at room temperature. Then palladium on carbon was filtered out and washed with ethyl acetate. The solvent was removed in vacuo to give 3.79 g (89%) of the product as a yellow solid. MS (ESIpos): m/z=172 (M+H)⁺; LC-MS (Method 4, Acetonitrile-Water-0.1% FA-10%B): Rₜ=0.59 min.

### Step c

### 6-Chloro-2-methylpyrido[3,2-d]pyrimidin-4(3H)-one

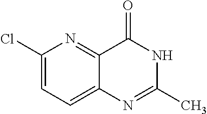

3-Amino-6-chloropicolinamide, 3.79 g (22.1 mmol), was added into 30 mL of triethyl orthoacetate and the resulting mixture was stirred at 120° C. for 10 hours under nitrogen. The precipitated solid was collected by filtration and washed with petroleum ether, dried in oven to give 3.27 g (73%) of product as a light grey solid. MS (ESIpos): m/z=196 (M+H)⁺; LC-MS (Method 4, Acetonitrile-Water-0.05% TFA-5%B): Rₜ=0.53 min.

### Step d

### 6-Chloro-2-methylpyrido[3,2-d]pyrimidin-4(3H)-one

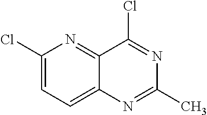

6-Chloro-2-methylpyrido[3,2-d]pyrimidin-4(3H)-one, 0.40 g (2.0 mmol), was dissolved in 20 mL of thionyl chloride and the resulting mixture was stirred at 90° C. for 10 hours. The solvent was removed in vacuo to give 0.43 g (crude) of the product as a yellow solid and the product was used directly for next step without further purification.

### Step e

### (R)-N-(1-(3-bromophenypethyl)-6-chloro-2-methylpyrido [3,2-d]pyrimidin-4-amine

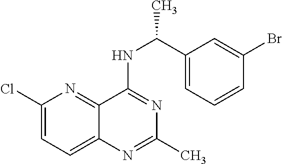 [SMILES: Cc1nc(N[C@H](C)c2cccc(Br)c2)c2nc(Cl)ccc2n1]

2-Propanol (10 mL) solution of (R)-1-(3-bromophenyl)ethanamine, 0.60 g (3.0 mmol) was added into 6-chloro-2-methylpyrido[3,2-d]pyrimidin-4(3H)-one, 0.43 g (2.0 mmol), and the resulting mixture was stirred at 110° C. for 20 min. After evaporation in vacuo, the residue was purified with silica gel column chromatography to give 0.41 g (53%) of the product as a light yellow solid. MS (ESIpos): m/z=377 (M+H)⁺; LC-MS (Method 4, Acetonitrile-Water-0.05% TFA-5%B): Rₜ=1.51 min.

### Step f

### (R)-N-(1-(3-bromophenypethyl)-6-methoxy-2-methylpyrido[3,2-d]pyrimidin-4-amine

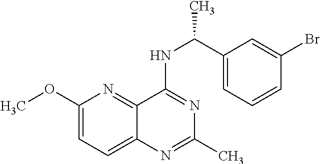 [SMILES: COc1ccc2nc(C)nc(N[C@H](C)c3cccc(Br)c3)c2n1]

(R)-N-(1-(3-bromophenyeethyl)-6-chloro-2-methylpyrido[3,2-d]pyrimidin-4-amine, 60.00 mg (0.2 mmol), and sodium methoxide, 171.65 mg (3.2 mmol), were dissolved in 4 mL of methanol and the resulting mixture was stirred at 70° C. for 6 hours. The precipitated solid was filtered out and the filtrate was concentrated in vacuo. The residue was purified with Prep-HPLC to give 31.30 mg (52%) of the product as a light yellow semi-solid. MS (ESIpos): m/z=373 (M+H)⁺; LC-MS (Method 4, Acetonitrile-Water-0.05% TFA-5%B): Rₜ=1.11 min. ¹H-NMR (400 MHz, CD₃OD): δ [ppm]=1.67-1.69 (d, 3H), 2.50 (s, 3H), 4.08 (s, 3H), 5.51-5.56 (m, 1H), 7.15-7.17 (d, 1H), 7.22-7.26 (t, 1H), 7.37-7.39 (d, 1H), 7.44-7.46 (d, 1H), 7.65 (s, 1H), 7.81-7.83 (d, 1H).

### EXAMPLE 29

### N-[(1R)-1-(3-bromophenyl)ethyl]-6-ethoxy-2-methylpyrido[3,2-d]pyrimidin-4-amine

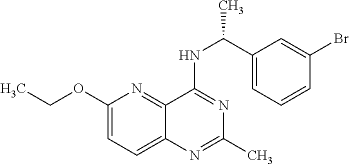 [SMILES: CCOc1ccc2nc(C)nc(N[C@H](C)c3cccc(Br)c3)c2n1]

This compound was synthesized by the same method as described in example 28 (step f) to give 23.30 mg (37%) of the product as a light yellow semi-solid. MS (ESIpos): m/z=387 (M+H)⁺. LC-MS (Method 4, Acetonitrile-Water-0.05%TFA-5%B): Rₜ=1.94 min. LC-MS (Acetonitrile-Water-0.05%NH₄HCO₃-10%B): Rₜ=1.94 min. ¹H-NMR (400 MHz, CD₃OD): δ [ppm]=1.43-1.46 (t, 3H), 1.66-1.68 (d, 3H), 2.49 (s, 3H), 4.49-4.55 (m, 2H), 5.50-5.56 (m, 1H), 7.13-7.15 (d, 1H), 7.22-7.26 (t, 1H), 7.37-7.39 (m, 1H), 7.43-7.45 (d, 1H), 7.64-7.65 (t, 1H), 7.81-7.83 (d, 1H).

### EXAMPLE 30

### N-[(1R)-1-(3-bromophenyl)ethyl]-6-(2-methoxyethoxy)-2-methylpyrido[3,2-d]pyrimidin-4-amine

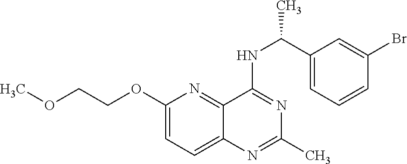 [SMILES: COCCOc1ccc2nc(C)nc(N[C@H](C)c3cccc(Br)c3)c2n1]

(R)-N-(1-(3-bromophenyeethyl)-6-chloro-2-methylpyrido[3,2-d]pyrimidin-4-amine, 60.00 mg (0.2 mmol, described in example 28 (step e), and 2-methoxyethanol, 18.13 mg (0.3 mmol) were dissolved in 4 mL of N,N-dimethylformamide Sodium hydride, 9.53 mg (0.3 mmol), was added at 0° C. and the resulting mixture was stirred at 60° C. for 4 hours. After evaporation in vacuo, the residue was purified by Prep-HPLC to give 42.10 mg (61%) of the product as a light yellow solid. MS (ESIpos): m/z=417 (M+H)⁺. LC-MS (Acetonitrile-Water-0.05%NH₄HCO₃-10%B): Rₜ=1.75 min. ¹H-NMR (400 MHz, CD₃OD): δ [ppm]=1.68-1.70 (d, 3H), 2.51 (s, 3H), 3.46 (s, 3H), 3.82-3.84 (t, 2H), 4.63-4.72 (m, 2H), 5.55-5.60 (m, 1H), 7.24-7.28 (m, 2H), 7.40-7.42 (d, 1H), 7.45-7.47 (d, 1H), 7.66 (s, 1H), 7.89-7.91 (d, 1H).

### EXAMPLE 31

N-[(1R)-1-(3-bromophenyl)ethyl]-6-(2-methoxyethoxy)-2-methylpyrido[3,2-d]pyrimidin-4-amine

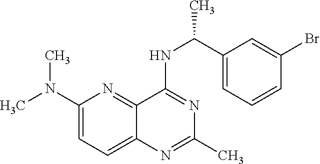 [SMILES: Cc1nc(N[C@H](C)c2cccc(Br)c2)c2nc(N(C)C)ccc2n1]

(R)-N-(1-(3-bromophenyl)ethyl)-6-chloro-2-methylpyrido[3,2-d]pyrimidin-4-amine, 60 mg (0.2 mmol, described in example 28 (step e)), and dimethylamine (1.6 mL, 2M in tetrahydrofuran), were dissolved into 4 mL of N,N-dimethylformamide and the resulting mixture was stirred at 80° C. for 4 hours. The resulting mixture was purified by Prep-HPLC to give 23.20 mg (38%) of the product as a light yellow solid. MS (ESIpos): m/z=386 (M+H)⁺; LC-MS (Method 4, Acetonitrile-Water-0.05% TFA-5%B): Rₜ=1.80 min. ¹H-NMR (400 MHz, CD₃OD): δ [ppm]=1.67-1.68 (d, 3H), 2.47 (s, 3H), 3.24 (s, 6H), 5.47-5.53 (m, 1H), 7.25-7.28 (m, 2H), 7.40-7.45 (m, 2H), 7.63 (s, 1H), 7.73-7.75 (d, 1H).

### EXAMPLE 32

### N-[(1R)-1-(3-bromophenyl)ethyl]-2-methyl-6-phenoxypyrido[3,2-d]pyrimidin-4-amine

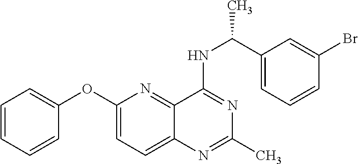 [SMILES: Cc1nc(N[C@H](C)c2cccc(Br)c2)c2nc(Oc3ccccc3)ccc2n1]

(R)-N-(1-(3-bromophenyeethyl)-6-chloro-2-methylpyrido[3,2-d]pyrimidin-4-amine, 100 mg (0.3 mmol, described in example 28 (step e)), potassium carbonate, 105.40 mg (0.8 mmol), and phenol, 35.88 mg (0.5 mmol), were added in 4 mL of N,N-dimethylformamide and the resulting mixture was stirred at 90° C. for 16 hours. The solid was filtered out and the filtrate was purified with Prep-HPLC to give 69.60 mg (62%) of the product as a light yellow semi-solid. MS (ESIpos): m/z=435 (M+H)⁺; LC-MS (Method 4, Acetonitrile-Water-0.05%NH₄HCO₃-10%B): Rₜ=2.04 min. ¹H-NMR (400 MHz, CD₃OD): δ [ppm]=1.52-1.54 (d, 3H), 2.52 (s, 3H), 5.39-5.44 (m, 1H), 7.23-7.35 (m, 5H), 7.40-7.45 (m, 2H), 7.49-7.54 (m, 3H), 8.03-8.05 (d, 1H).

### EXAMPLE 33

### 6-(benzyloxy)-N-[(1R)-1-(3-bromophenyl)ethyl]-2-methylpyrido[3,2-d]pyrimidin-4-amine

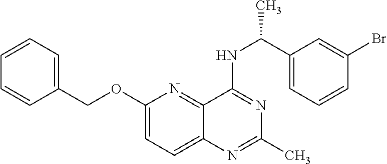 [SMILES: Cc1nc(N[C@H](C)c2cccc(Br)c2)c2nc(OCc3ccccc3)ccc2n1]

This compound was synthesized by the same method as described in example 32 to give 57.80 mg (81%) of the product as a light yellow semi-solid. MS (ESIpos): m/z=449 (M+H)⁺; LC-MS (Method 4, Acetonitrile-Water-0.05% NH₄HCO₃-10%B): Rₜ=1.59 min. ¹H-NMR (400 MHz, CD₃OD): δ [ppm]=1.68-1.70 (d, 3H), 2.50 (s, 3H), 5.50-5.63 (m, 3H), 7.25-7.44 (m, 7H), 7.51-7.53 (m, 2H), 7.63-7.64 (m, 1H), 7.89-7.91 (d, 1H).

### EXAMPLE 34

### N-[(1R)-1-(3-bromophenyl)ethyl]-6-fluoro-2-methylpyrido[3,4-d]pyrimidin-4-amine

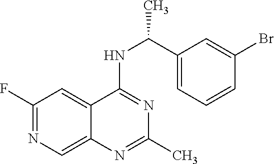 [SMILES: Cc1nc(N[C@H](C)c2cccc(Br)c2)c2cc(F)ncc2n1]

The the crude reaction mixture of 6-fluoro-2-methylpyrido[3,4-d]pyrimidin-4-yl 2,4,6-tri(propan-2-yl)benzene-sulfonate (as described in example 35, step b) obtained from 6-fluoro-2-methylpyrido[3,4-d]pyrimidin-4-ol (500 mg) in DMF (25 ml) was added (R)-1-(3-bromophenyl)ethylamine (670 mg, 3.35 mmol, commercially available) and the reaction mixture was stirred at ambient temperature overnight. The solvent was removed under reduced pressure and the residue was dissolved in ethyl acetate (100 ml) and water (30 ml). The layers were separated and the aqueous layer was extracted with ethyl acetate (5×). The combined organic layers were washed with brine, dried over sodium sulfate and the solvent was removed under reduced pressure. The title compound was obtained after Isolera flash chromatography (eluent: dichloromethane/MeOH), 100 g silica column) in 5% yield (46 mg). 1H-NMR (400 MHz, DMSO-d6): d [ppm]=8.74-8.68 (m, 2H), 8.10 (d, 1H), 7.66 (t, 1H), 7.49-7.40 (m, 2H), 7.34-7.24 (m, 1H), 5.56 (quin, 1H), 2.43 (s, 3H), 1.58 (d, 3H). LC-MS (Method 7): m/z: [M+H]⁺=363, Rₜ=0.93 min.

### EXAMPLE 35

### N-[1-(5-bromothiophen-2-ypethyl]-6-fluoro-2-methylpyrido[3,4-d]pyrimidin-4-amine

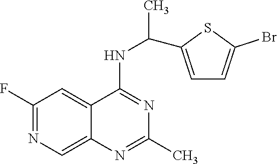 [SMILES: Cc1nc(NC(C)c2ccc(Br)s2)c2cc(F)ncc2n1]

### Step a

### 6-fluoro-2-methylpyrido[3,4-d]pyrimidin-4-ol

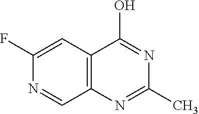

A round-bottom flask was charged with 5.00 g (32.0 mmol, commercially available) 5-Amino-2-fluoro-4-pyridinecarboxylic acid, 7.57 g (80 mmol, commercially available) acetamidine hydrochloride , and 6.56 g (80 mmol) anhydrous sodium acetate. The mixture was suspended in 50.0 ml of 2-methoxyethanol, and then the mixture was stirred at 130° C. for 16 h. The course of the reaction was monitored by LC/MS. Complete conversion was observed. The resulting mixture was poured into cold water and stirred for 30 min. The precipitate was filtered off and dried in vacuo. 5.95 g (98% d. Th.) of the title compound was obtained in form of a beige-coloured solid. 1H-NMR (400 MHz, <dmso>): d [ppm]=13.14-11.96 (br s, 1H), 8.66 (s, 1H), 7.59 (d, 1H), 2.37 (s, 3H).

### Step b:

### 6-fluoro-2-methylpyrido[3,4-d]pyrimidin-4-yl 2,4,6-tri(propan-2-yl)benzenesulfonate

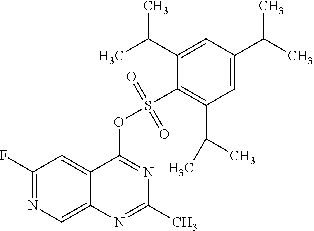 [SMILES: Cc1nc(OS(=O)(=O)c2c(C(C)C)cc(C(C)C)cc2C(C)C)c2cc(F)ncc2n1]

A solution of 6-fluoro-2-methylpyrido[3,4-d]pyrimidin-4-ol (12.1 g, 67.5 mmol, described in example 35, step a), 2,4,6-tri(propan-2-yl)benzenesulfonyl chloride (12.0 g, 39.7 mmol, commercially available) and triethylamine (19 mL, 140 mmol) in DMF (250 mL) was stirred at room temperature for 1 hour (complete conversion) and then used directly in the next step. LC-MS (Method 10): m/z: [M+H]⁺446, Rₜ=1.71 min.

### Step c

### N-[1-(5-bromothiophen-2-yl)ethyl]-6-fluoro-2-methylpyrido[3,4-d]pyrimidin-4-amine

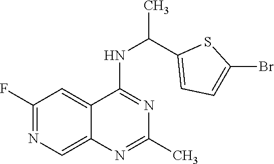 [SMILES: Cc1nc(NC(C)c2ccc(Br)s2)c2cc(F)ncc2n1]

To a solution of 6-fluoro-2-methylpyrido[3,4-d]pyrimidin-4-yl 2,4,6-tri(propan-2-yl)benzenesulfonate in DMF (250 mL) obtained in step a was added 1-(5-bromothiophen-2-yl)ethanamine (9.00 g, 43.7 mmol) and the reaction mixture stirred at room temperature overnight. The solvent was removed in vacuo and the obtained residue taken up in MTBE. The precipitate was filtered out and the filtrate evaporated to dryness, then purified by column chromatography (silica gel, EtOAc/hexane 20-100%) to give the title compound (5.07 g, 35% over two steps). LC-MS (Method 10): m/z: [M+H]⁺=367, Rₜ=1.30 min.

### EXAMPLE 36

### 6-fluoro-2-methyl-N-[1-(5-{2-[(methylamino)methyl]phenyl}thiophen-2-yl)ethyl]pyrido[3,4-d]pyrimidin-4-amine

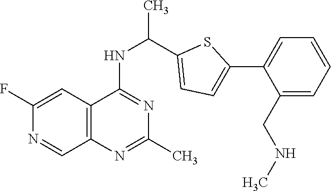 [SMILES: CNCc1ccccc1-c1ccc(C(C)Nc2nc(C)nc3cnc(F)cc23)s1]

To a solution of 2-(5-{1-[(6-fluoro-2-methylpyrido[3,4-d]pyrimidin-4-yl)amino]-ethyl}thiophen-2-yl)benzaldehyde (530 mg, 1.35 mmol, described in example 93, step a) and methylamine (2M in THF, 1.35 ml, 2.70 mmol) in 1,2-dichloroethane (11.7 ml) were added acetic acid (0.155 ml, 2.70 mmol) and sodium triacetoxyborohydride (572 mg, 2.70 mmol) and the reaction was stirred at ambient temperature overnight. The reaction was diluted with aqueous NaOH (1M, 50 ml) and extracted with dichloromethane (3×30 ml). The combined organic layers were washed with brine, dried over sodium sulfate and the solvent was removed under reduced pressure. The residue was purified via Isolera flash chromatography (SNAP NH column 110 g; eluent hexanes/ethyl acetate gradient 10-100%) to yield the title compound (414 mg, 75%). 1H-NMR (400 MHz, DMSO-d6): d [ppm]=8.86 (d, 1H), 8.76 (s, 1H), 8.06 (d, 1H), 7.48 (dd, 1H), 7.36-7.23 (m, 3H), 7.18-7.15 (m, 1H), 7.12 (dd, 1H), 5.91 (quin, 1H), 3.64 (s, 2H), 2.52 (s, 3H), 2.23 (s, 3H), 1.73 (d, 3H). LC-MS (Method 7): m/z: [M+H]⁺=408.2, Rₜ=0.61 min.

### EXAMPLE 37

### 4-{[(2-methyl-4-{[1-(5-{2-[(methylamino)methyl]phenyl}thiophen-2-yl)ethyl]amino}pyrido[3,4-d]pyrimidin-6-yl)oxy]methyl}piperidin-2-one

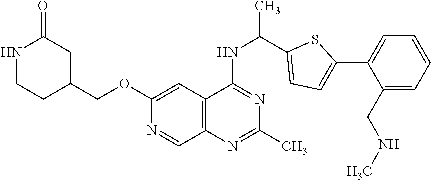 [SMILES: CNCc1ccccc1-c1ccc(C(C)Nc2nc(C)nc3cnc(OCC4CCNC(=O)C4)cc23)s1]

To a mixture of 2-oxopiperidine-4-methanol (55 mg, 0.43 mmol, commercially available) in DMF (2 ml) was added under argon NaH (34 mg, 0.83 mmol, 60% in mineral oil) and the reaction was stirred at ambient temperature for 20 minutes. 6-fluoro-2-methyl-N-[1-(5-{2-[(methylamino)methyl]phenyl}thiophen-2-yl)ethyl]pyrido[3,4-d]pyrimidin-4-amine (87 mg, 0.21 mmol, described in example 36) in DMF (1 ml) was added and the reaction was stirred at 180° C. in a microwave oven for five hours. The reaction was allowed to cool to ambient temperature, diluted with water (50 ml) and extracted with ethyl acetate (3×). The combined organic layers were washed with brine (1×), dried over sodium sulfate and the solvent was removed under reduced pressure. The residue was purified via HPLC chromatography (actonitrile 30-70%; basic) to yield the title compound (9 mg, 8%). 1H-NMR (400 MHz, DMSO-d6): d [ppm]=8.72 (s, 1H), 8.66 (d, 1H), 7.69 (s, 1H), 7.53-7.47 (m, 2H), 7.35-7.23 (m, 3H), 7.15 (d, 1H), 7.10 (dd, 1H), 5.90 (quin, 1H), 4.20 (d, 2H), 3.66 (s, 2H), 3.24-3.10 (m, 2H), 2.38-2.27 (m, 2H), 2.24 (s, 3H), 2.09-2.00 (m, 1H), 1.92 (br d, 1H), 1.72 (d, 3H), 1.69-1.44 (m, 1H). LC-MS (Method 7): m/z: [M+H]⁺=517.2, Rₜ=0.55 min.

### EXAMPLE 38

### 1-{4-[(2-methyl-4-{[1-(5-{2-[(methylamino)methyl]phenyl}thiophen-2-yl)ethyl]amino}pyrido[3,4-d]pyrimidin-6-yl)oxy]phenyl}pyrrolidin-2-one

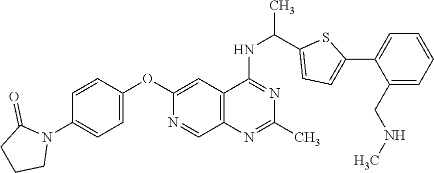 [SMILES: CNCc1ccccc1-c1ccc(C(C)Nc2nc(C)nc3cnc(Oc4ccc(N5CCCC5=O)cc4)cc23)s1]

To a mixture of 1-(4-hydroxyphenyl)pyrrolidin-2-one (100 mg, 0.57 mmol, commercially available) in 1-methyl-2-pyrrolidon (4 ml) was added under argon NaH (23 mg, 0.57 mmol, 60% in mineral oil) and the reaction was stirred at ambient temperature for 20 minutes. 6-fluoro-2-methyl-N-[1-(5-{2-[(methylaminonnethyl]phenyl}thiophen-2-yl)ethyl]pyrido [3,4-d]pyrimidin-4-amine (77 mg, 0.19 mmol, described in example 36) in 1-methyl-2-pyrrolidon (2 ml)was added and the reaction was stirred at 170° C. for two hours. The reaction was allowed to cool to ambient temperature, diluted with water (50 ml) and extracted with ethyl acetate (3×). The combined organic layers were washed with brine (1×), dried over sodium sulfate and the solvent was removed under reduced pressure. The residue was purified via HPLC chromatography (actonitrile 30-70%; basic) to yield the title compound (37 mg, 32%). 1H-NMR (400 MHz, DMSO-d6): d [ppm]=8.79 (d, 1H), 8.72 (s, 1H), 7.93 (s, 1H), 7.70-7.62 (m, 2H), 7.51-7.46 (m, 1H), 7.36-7.25 (m, 3H), 7.18-7.08 (m, 4H), 5.93 (quin, 1H), 3.83 (t, 2H), 3.64 (s, 2H), 2.24 (s, 3H), 2.06 (quin, 2H), 1.76-1.68 (m, 3H). LC-MS (Method 7): m/z: [M+H]⁺=565.2, Rₜ=0.57 min.

### EXAMPLE 39

### N4-[(1R)-1-(3-bromophenypethyl]-2-methyl-N6-[2-(morpholin-4-yl)ethyl]pyrido[3,4-d]pyrimidine-4,6-diamine

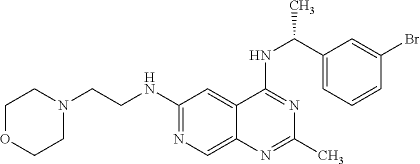 [SMILES: Cc1nc(N[C@H](C)c2cccc(Br)c2)c2cc(NCCN3CCOCC3)ncc2n1]

A solution of N-[(1R)-1-(3-bromophenyl)ethyl]-6-fluoro-2-methylpyrido[3,4-d]pyrimidin-4-amine (100 mg, 0.28 mmol, described in example 34), triethylamine (0.12 ml, 0.83 mmol) and 4-(2-Aminoethyl)morpholine (109 mg, 0.83 mmol, commercially available) in DMSO (10 ml) was stirred at 150° C. over the weekend. The reaction mixture was allowed to cool to ambient temperature, poured on ethyl acetate and extracted with aqueous NaOH (2×2N) and water (2×). The aqueous layers were re-extracted with ethyl acetate (2×) The combined organic layers were dried over sodium sulfate and the solvent was removed under reduced pressure. The crude product was purified by HPLC chromatography to yield 6% (9 mg) of the title compound. 1H-NMR (400 MHz, DMSO-d6): d [ppm]=8.54 (s, 1H), 8.25 (d, 1H), 7.63 (t, 1H), 7.47-7.40 (m, 2H), 7.32-7.26 (m, 1H), 7.03 (s, 1H), 6.18 (t, 1H), 5.56 (quin, 1H), 3.59 (t, 4H), 3.40-3.34 (m, 2H), 2.57 (t, 2H), 2.45 (br s, 4H), 2.33 (s, 3H), 1.57 (d, 3H). LC-MS (Method 9): m/z: [M+H]⁺=473.3, Rₜ=0.67 min.

### EXAMPLE 40

### 3-[(4-{[(1R)-1-(3-bromophenyl)ethyl]amino}-2-methylpyrido[3,4-d]pyrimidin-6-yl)oxy]phenol

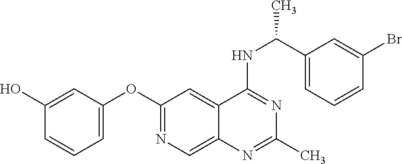 [SMILES: Cc1nc(N[C@H](C)c2cccc(Br)c2)c2cc(Oc3cccc(O)c3)ncc2n1]

In a microwave vial under argon a mixture of Resorcinol (154 mg, 1.38 mmol, commercially available), sodium hydride (18 mg, 0.44 mmol, 60% in mineral oil) in DMF (10 ml) were stirred at ambient temperature for 10 minutes. N-[(1R)-1-(3-bromophenyeethyl]-6-fluoro-2-methylpyrido[3,4-d]pyrimidin-4-amine (100 mg, 0.27 mmol, described in example 34) were added and the reaction was stirred at 170° C. overnight. The reaction mixture was allowed to cool to ambient temperature, poured into ethyl acetate and washed with water (3×). The organic layer was dried over sodium sulfate and the solvent was removed under reduced pressure. The residue was purified via HPCL chromatography to yield the title compound (23 mg, 18%). 1H-NMR (400 MHz, DMSO-d6): d [ppm]=9.61 (br s, 1H), 8.73 (d, 1H), 8.64 (d, 1H), 7.99 (s, 1H), 7.66 (t, 1H), 7.50-7.39 (m, 2H), 7.34-7.26 (m, 1H), 7.17 (t, 1H), 6.58 (ddd, 1H), 6.53-6.44 (m, 2H), 5.57 (quin, 1H), 2.42 (s, 3H), 1.57 (d, 3H). LC-MS (Method 9): m/z: [M+H]⁺=453.3, Rₜ=0.64 min.

### EXAMPLE 41

### N-[(1R)-1-(3-bromophenyl)ethyl]-2-methyl-6-[3-(1-methyl-4,5-dihydro-1H-imidazol-2-yl)phenoxy]pyrido[3,4-d]pyrimidin-4-amine

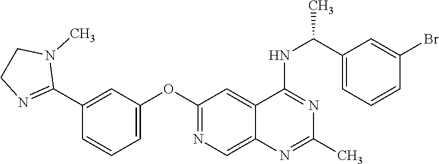 [SMILES: Cc1nc(N[C@H](C)c2cccc(Br)c2)c2cc(Oc3cccc(C4=NCCN4C)c3)ncc2n1]

In a microwave vial under argon a mixture of 3-(1-methyl-4,5-dihydro-1H-imidazole-2-YL)phenol (312 mg, 1.76 mmol, commercially available), sodium hydride (72 mg, 1.76 mmol, 60% in mineral oil) in DMF (10 ml) were stirred at ambient temperature for 10 minutes. N-[(1R)-1-(3-bromophenyeethyl]-6-fluoro-2-methylpyrido[3,4-d]pyrimidin-4-amine (100 mg, 0.27 mmol, described in example 34) were added and the reaction was stirred at 170° C. overnight. The reaction mixture was allowed to cool to ambient temperature, poured into ethyl acetate and washed with water (3×). The organic layer was dried over sodium sulfate and the solvent was removed under reduced pressure. The residue was purified via HPCL chromatography to yield the title compound (18 mg, 12%). 1H-NMR (400 MHz, DMSO-d6): d [ppm]=8.73 (s, 1H), 8.70 (d, 1H), 8.28 (s, 1H), 8.08 (s, 1H), 7.66 (t, 1H), 7.56-7.50 (m, 1H), 7.48-7.41 (m, 2H), 7.38-7.34 (m, 1H), 7.32-7.27 (m, 2H), 7.26-7.24 (m, 1H), 5.57 (quin, 1H), 3.76-3.69 (m, 2H), 3.52-3.46 (m, 2H), 2.79 (s, 3H), 2.43 (s, 3H), 1.57 (d, 3H). LC-MS (Method 7): m/z: [M+H]⁺=519.1, Rₜ=0.74 min.

### EXAMPLE 42

### N-[1-(4-{[(1R)-1-(3-bromophenyl)ethyl]amino}-2-methylpyrido[3,4-d]pyrimidin-6-yl)pyrrolidin-3-yl]acetamide

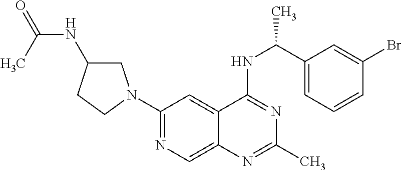 [SMILES: CC(=O)NC1CCN(c2cc3c(N[C@H](C)c4cccc(Br)c4)nc(C)nc3cn2)C1]

A solution of N-[(1R)-1-(3-bromophenyl)ethyl]-6-fluoro-2-methylpyrido[3,4-d]pyrimidin-4-amine (100 mg, 0.28 mmol, described in example 34), triethylamine (0.12 ml, 0.83 mmol) and (rac)-3-acetamidopyrrolidine (109 mg, 0.83 mmol, commercially available) in DMSO (10 ml) was stirred at 120° C. overnight. The reaction mixture was allowed to cool to ambient temperature, poured on ethyl acetate and extracted with aqueous NaOH (2×2N) and water (2×). The aqueous layers were re-extracted with ethyl acetate (2×). The combined organic layers were dried over sodium sulfate and the solvent was removed under reduced pressure. The crude product was purified by HPLC chromatography to yield 11% (14 mg) of the title compound as a mixture of diastereomers. 1H-NMR (400 MHz, DMSO-d6): d [ppm]=8.64 (s, 1H), 8.32 (d, 1H), 8.23-8.16 (m, 1H), 7.62 (s, 1H), 7.46-7.39 (m, 2H), 7.34-7.27 (m, 1H), 7.03 (d, 1H), 5.58 (br t, 1H), 4.46-4.35 (m, 1H), 3.73-3.49 (m, 3H), 2.33 (s, 3H), 2.20 (dq, 1H), 1.99-1.88 (m, 1H), 1.82 (s, 3H), 1.58 (d, 3H). LC-MS (Method 7): m/z: [M+H]⁺=471.1, Rₜ=0.78 min.

### EXAMPLE 43

### N-[(1R)-1-(3-bromophenyl)ethyl]-2-methyl-6-[4-(pyridin-3-ylmethyl)piperazin-1-yl]pyrido[3,4-d]pyrimidin-4-amine

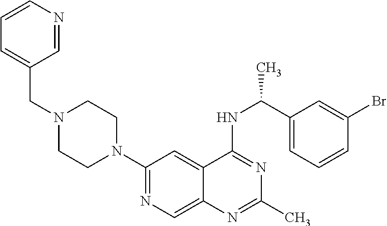 [SMILES: Cc1nc(N[C@H](C)c2cccc(Br)c2)c2cc(N3CCN(Cc4cccnc4)CC3)ncc2n1]

A solution of N-[(1R)-1-(3-bromophenyl)ethyl]-6-fluoro-2-methylpyrido[3,4-d]pyrimidin-4-amine (100 mg, 0.28 mmol, described in example 34), triethylamine (0.12 ml, 0.83 mmol) and 1-(3-Pyridylmethyl)piperazine (620 mg, 3.3 mmol, commercially available) in DMSO (10 ml) was stirred at 120° C. for two days. The reaction mixture was allowed to cool to ambient temperature, poured on ethyl acetate and extracted with aqueous NaOH (2×2N) and water (2×). The aqueous layers were re-extracted with ethyl acetate (2×) The combined organic layers were dried over sodium sulfate and the solvent was removed under reduced pressure. The crude product was purified by HPLC chromatography to yield 12% (17 mg) of the title compound. 1H-NMR (400 MHz, DMSO-d6): d [ppm]=8.66 (s, 1H), 8.55 (d, 1H), 8.49 (dd, 1H), 8.35 (d, 1H), 7.77 (dt, 1H), 7.62 (t, 1H), 7.46-7.36 (m, 4H), 7.32-7.26 (m, 1H), 5.58 (quin, 1H), 3.59-3.55 (m, 4H), 2.58-2.53 (m, 4H), 2.35 (s, 3H), 1.58 (d, 3H). LC-MS (Method 7): m/z: [M+H]⁺=520.2, Rₜ=0.63 min.

### EXAMPLE 44

### N-{2-[(4-{[(1R)-1-(3-bromophenyl)ethyl]amino}-2-methylpyrido[3,4-d]pyrimidin-6-yl)amino]ethyl}acetamide

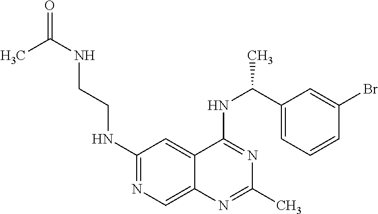 [SMILES: CC(=O)NCCNc1cc2c(N[C@H](C)c3cccc(Br)c3)nc(C)nc2cn1]

A solution of N-[(1R)-1-(3-bromophenyl)ethyl]-6-fluoro-2-methylpyrido[3,4-d]pyrimidin-4-amine (100 mg, 0.28 mmol, described in example 34), triethylamine (0.12 ml, 0.83 mmol) and N-acetylethylendiamine (360 mg, 3.2 mmol, commercially available) in DMSO (10 ml) was stirred at 120° C. for two days. The reaction mixture was allowed to cool to ambient temperature, poured on ethyl acetate and extracted with aqueous NaOH (2×2N) and water (2×). The aqueous layers were re-extracted with ethyl acetate (2×) The combined organic layers were dried over sodium sulfate and the solvent was removed under reduced pressure. The crude product was purified by HPLC chromatography to yield 2% (3 mg) of the title compound. 1H-NMR (400 MHz, DMSO-d6): d [ppm]=8.55 (s, 1H), 8.28 (br d, 1H), 8.07 (br s, 1H), 7.64 (t, 1H), 7.46-7.39 (m, 2H), 7.32-7.24 (m, 1H), 7.06 (s, 1H), 6.47 (s, 1H), 5.55 (quin, 1H), 2.35-2.30 (m, 5H), 1.82 (s, 3H), 1.57 (d, 3H). LC-MS (Method 7): m/z: [M+H]⁺=443.1, Rₜ=0.76 min.

### EXAMPLE 45

### 4-(4-{[(1R)-1-(3-bromophenypethyl]amino}-2-methylpyrido[3,4-d]pyrimidin-6-yl)piperazin-2-one

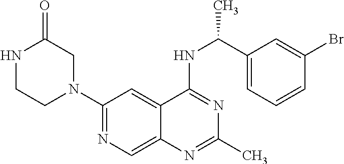 [SMILES: Cc1nc(N[C@H](C)c2cccc(Br)c2)c2cc(N3CCNC(=O)C3)ncc2n1]

A solution of N-[(1R)-1-(3-bromophenyl)ethyl]-6-fluoro-2-methylpyrido[3,4-d]pyrimidin-4-amine (100 mg, 0.28 mmol, described in example 34), triethylamine (0.12 ml, 0.83 mmol) and 2-Oxopiperazine (166 mg, 1.64 mmol, commercially available) in 1-Methyl-2-pyrrolidon (10 ml) was stirred at 150° C. over the weekend. The reaction mixture was allowed to cool to ambient temperature, poured on ethyl acetate and extracted with aqueous NaOH (2×2N) and water (2×). The aqueous layers were re-extracted with ethyl acetate (2×) The combined organic layers were dried over sodium sulfate and the solvent was removed under reduced pressure. The crude product was purified by HPLC chromatography to yield 7% (9 mg) of the title compound. 1H-NMR (400 MHz, DMSO-d6): d [ppm]=8.69 (s, 1H), 8.41 (d, 1H), 8.17 (br s, 1H), 7.63 (t, 1H), 7.43 (dtd, 2H), 7.37 (s, 1H), 7.33-7.27 (m, 1H), 5.59 (quin, 1H), 4.10-3.97 (m, 2H), 3.88-3.83 (m, 2H), 2.36 (s, 3H), 1.59 (d, 3H). LC-MS (Method 7): m/z: [M+H]⁺=441.1, Rₜ=0.75 min.

### EXAMPLE 46

### N4-[(1R)-1-(3-bromophenypethyl]-2-methyl-N6-(tetrahydro-2H-pyran-4-yOpyrido[3,4-d]pyrimidine-4,6-diamine

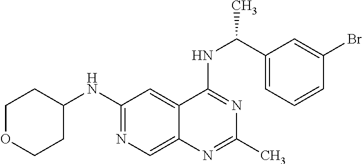 [SMILES: Cc1nc(N[C@H](C)c2cccc(Br)c2)c2cc(NC3CCOCC3)ncc2n1]

A solution of N-[(1R)-1-(3-bromophenyl)ethyl]-6-fluoro-2-methylpyrido[3,4-d]pyrimidin-4-amine (100 mg, 0.28 mmol, described in example 34), triethylamine (0.12 ml, 0.83 mmol) and 4-aminotetrahydropyran (140 mg, 1.38 mmol, commercially available) in DMSO (10 ml) was stirred at 120° C. overnight. The reaction mixture was allowed to cool to ambient temperature, poured on ethyl acetate and extracted with aqueous NaOH (2×2N) and water (2×). The aqueous layers were re-extracted with ethyl acetate (2×) The combined organic layers were dried over sodium sulfate and the solvent was removed under reduced pressure. The crude product was purified by HPLC chromatography to yield 6% (8 mg) of the title compound. 1H-NMR (400 MHz, DMSO-d6): d [ppm]=8.56 (s, 1H), 8.20 (d, 1H), 7.64 (t, 1H), 7.47-7.39 (m, 2H), 7.33-7.25 (m, 1H), 7.00 (s, 1H), 6.45 (d, 1H), 5.56 (t, 1H), 3.95-3.80 (m, 4H), 3.44 (br t, 2H), 2.32 (s, 3H), 1.90 (br d, 2H), 1.63-1.45 (m, 6H). LC-MS (Method 7): m/z: [M+H]⁺=442.1, Rₜ=0.82 min.

### EXAMPLE 47

### N-[1-(4-{[1-(5-{2-[(dimethylamino)methyl]phenyl}thiophen-2-yl)ethyl]amino}-2-methylpyrido[3,4-d]pyrimidin-6-yl)pyrrolidin-3-yl]acetamide

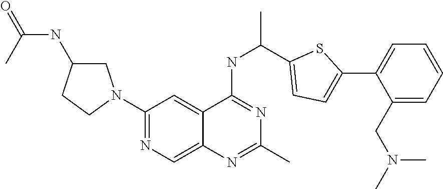 [SMILES: CC(=O)NC1CCN(c2cc3c(NC(C)c4ccc(-c5ccccc5CN(C)C)s4)nc(C)nc3cn2)C1]

A mixture of N-[1-(5-{2-[(dimethylamino)methyl]phenyl}thiophen-2-yl)ethyl]-6-fluoro-2-methylpyrido[3,4-d]pyrimidin-4-amine (100 mg, 0.24 mmol, described in example 93, step b), (rac)-3-acetamidopyrrolidine (94 mg, 0.71 mmol, commercially available) and triethylamine (99 μl, 0.71 mmol) in DMSO (5 ml) were stirred at 140° C. overnight. The solvent was removed under reduced pressure and the residue was purified via HPLC chromatography to yield the title compound as a mixture of diastereomers and enantiomers (72 mg, 56%). 1H-NMR (400 MHz, DMSO-d6): d [ppm]=8.66 (s, 1H), 8.47 (d, 1H), 8.18 (d, 1H), 7.45-7.40 (m, 1H), 7.39-7.33 (m, 1H), 7.32-7.26 (m, 2H), 7.18 (dd, 1H), 7.08 (dd, 1H), 7.00 (s, 1H), 5.93 (quin, 1H), 4.44-4.32 (m, 1H), 3.69-3.47 (m, 3H), 3.35 (s, 2H), 3.29 (dd, 1H), 2.42 (s, 3H), 2.24-2.13 (m, 1H), 2.10 (d, 6H), 1.98-1.88 (m, 1H), 1.81 (d, 3H), 1.72 (d, 3H). LC-MS (Method 7): m/z: [M+H]⁺=530.3, Rₜ=0.52 min.

### EXAMPLE 48

### N4-[1-(5-{2-[(dimethylamino)methyl]phenyl}-2-thienypethyl]-2-methyl-N6-[2-(morpholin-4-yl)ethyl]pyrido[3,4-d]pyrimidine-4,6-diamine

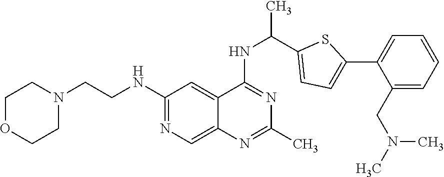 [SMILES: Cc1nc(NC(C)c2ccc(-c3ccccc3CN(C)C)s2)c2cc(NCCN3CCOCC3)ncc2n1]

A mixture of N-[1-(5-{2-[(dimethylamino)methyl]phenyl}thiophen-2-yl)ethyl]-6-fluoro-2-methylpyrido[3,4-d]pyrimidin-4-amine (100 mg, 0.24 mmol, described in example 93, step b), 4-(2-Aminoethyl)morpholine (96 mg, 0.71 mmol, commercially available) and triethylamine (99 μl, 0.71 mmol) in DMSO (5 ml) were stirred at 150° C. for two days. The solvent was removed under reduced pressure and the residue was purified via HPLC chromatography to yield the title compound (46 mg, 36%). 1H-NMR (400 MHz, DMSO-d6): d [ppm]=8.56 (s, 1H), 8.40 (d, 1H), 7.42 (dd, 1H), 7.39-7.34 (m, 1H), 7.33-7.26 (m, 2H), 7.18 (d, 1H), 7.08 (dd, 1H), 7.00 (s, 1H), 6.18 (t, 1H), 5.91 (t, 1H), 3.57 (t, 4H), 2.55 (t, 2H), 2.41 (s, 7H), 2.10 (s, 6H), 1.71 (d, 3H). LC-MS (Method 7): m/z: [M+H]⁺=532.3, Rₜ=0.40 min.

### EXAMPLE 49

### N4-[(1R)-1-(3-bromophenl)pethyl]-2-methyl-N6-[2-(1H-pyrazol-1-yl)ethyl]pyrido[3,4-d]pyrimidine-4,6-diamine

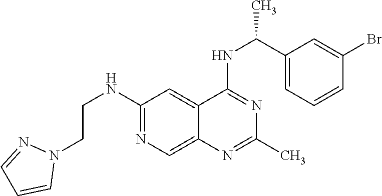 [SMILES: Cc1nc(N[C@H](C)c2cccc(Br)c2)c2cc(NCCn3cccn3)ncc2n1]

A solution of N-[(1R)-1-(3-bromophenyl)ethyl]-6-fluoro-2-methylpyrido[3,4-d]pyrimidin-4-amine (100 mg, 0.28 mmol, described in example 34), triethylamine (0.12 ml, 0.83 mmol) and 2-pyrazol-1-yl-ethylamine (154 mg, 0.13 mmol, commercially available) in DMSO (10 ml) was stirred at 120° C. overnight. The reaction mixture was allowed to cool to ambient temperature, poured on ethyl acetate and extracted with aqueous NaOH (2×2N) and water (2×). The aqueous layers were re-extracted with ethyl acetate (2×) The combined organic layers were dried over sodium sulfate and the solvent was removed under reduced pressure. The crude product was purified by HPLC chromatography to yield 8% (10 mg) of the title compound. 1H-NMR (400 MHz, DMSO-d6): d [ppm]=8.56 (s, 1H), 8.27 (d, 1H), 7.76-7.73 (m, 1H), 7.63 (t, 1H), 7.48-7.40 (m, 3H), 7.33-7.25 (m, 1H), 7.03 (s, 1H), 6.55 (t, 1H), 6.25-6.19 (m, 1H), 5.55 (quin, 1H), 4.38 (t, 2H), 3.66 (q, 2H), 2.33 (s, 3H), 1.56 (d, 3H). LC-MS (Method 7): m/z: [M+H]⁺=454.1, Rₜ=0.89 min.

### EXAMPLE 50

### 4-(4-{[1-(5-{2-[(dimethylamino)methyl]phenyl}thiophen-2-yl)ethyl]amino}-2-methylpyrido[3,4-d]pyrimidin-6-yl)piperazin-2-one

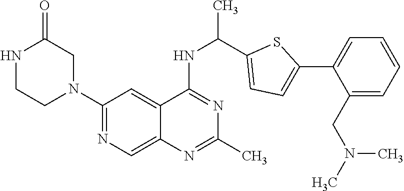 [SMILES: Cc1nc(NC(C)c2ccc(-c3ccccc3CN(C)C)s2)c2cc(N3CCNC(=O)C3)ncc2n1]

In a sealed tube a mixture of N-[1-(5-{2-[(dimethylamino)methyl]phenyl}thiophen-2-yl)ethyl]-6-fluoro-2-methylpyrido[3,4-d]pyrimidin-4-amine (120 mg, 0.28 mmol, described in example 93, step b), 2-Oxopiperazine (88 mg, 0.85 mmol, commercially available) and triethylamine (119 μl, 0.85 mmol) in DMSO (6 ml) were stirred at 140° C. overnight. NaH (23 mg, 0.57 mmol, 60% in mineral oil) was added and the reaction was stirred at ambient temperature for 30 minutes. After releasing pressure the sealed tube was heated to 150° C. and stirred overnight. The reaction mixture was allowed to cool to ambient temperature and separated between ethyl acetate and water. The aqueous layer was extracted with ethyl acetate (1×) and the combined organic layers were washed with brine, dried over sodium sulfate and the solvent was removed under reduced pressure. The residue was purified via HPLC chromatography to yield the title compound (20 mg, 13%). 1H-NMR (400 MHz, DMSO-d6): d [ppm]=8.72 (s, 1H), 8.56 (d, 1H), 8.15 (s, 1H), 7.44-7.39 (m, 1H), 7.39-7.26 (m, 4H), 7.19 (d, 1H), 7.10 (dd, 1H), 5.94 (quin, 1H), 4.00 (s, 2H), 3.87-3.80 (m, 2H), 3.35 (s, 3H), 2.44 (s, 3H), 2.10 (s, 6H), 1.73 (d, 3H). LC-MS (Method 7): m/z: [M+H]⁺=502.2, Rₜ=0.52 min.

### EXAMPLE 51

### N-{2-[(4-{[1-(5-{2-[(dimethylamino)methyl]phenyl}thiophen-2-yl)ethyl]amino}-2-methylpyrido[3,4-d]pyrimidin-6-yl)amino]ethyl}acetamide

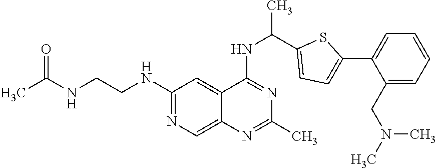 [SMILES: CC(=O)NCCNc1cc2c(NC(C)c3ccc(-c4ccccc4CN(C)C)s3)nc(C)nc2cn1]

A mixture of N-[1-(5-{2-[(dimethylamino)methyl]phenyl}thiophen-2-yl)ethyl]-6-fluoro-2-methylpyrido[3,4-d]pyrimidin-4-amine (107 mg, 0.25 mmol, described in example 93, step b), N-(2-aminoethyl)acetamide (91 mg, 0.89 mmol, commercially available) and triethylamine (124 μl, 0.89 mmol) in 1-Methyl-2-pyrrolidon (4 ml) were stirred in a microwave oven at 250° C. for three hours. The reaction mixture was allowed to cool to ambient temperature and separated between ethyl acetate and water. The aqueous layer was extracted with ethyl acetate (1×) and the combined organic layers were washed with brine, dried over sodium sulfate and the solvent was removed under reduced pressure. The residue was purified via HPLC chromatography to yield the title compound (40 mg, 30%). 1H-NMR (400 MHz, DMSO-d6): d [ppm]=8.57 (s, 1H), 8.44-8.37 (m, 1H), 8.07-7.94 (m, 1H), 7.44-7.41 (m, 1H), 7.39-7.35 (m, 1H), 7.33-7.28 (m, 2H), 7.18 (d, 1H), 7.08 (dd, 1H), 7.00 (s, 1H), 6.49-6.41 (m, 1H), 5.91 (quin, 1H), 3.35 (s, 3H), 3.30-3.25 (m, 4H), 2.41 (s, 3H), 2.10 (s, 6H), 1.81 (s, 3H), 1.71 (d, 3H). LC-MS (Method 7): m/z: [M+H]⁺=504.3, Rₜ=0.54 min.

### EXAMPLE 52

### 3-[(4-{[1-(5-{2-[(dimethylamino)methyl]phenyl}thiophen-2-yl)ethyl]amino}-2-methylpyrido[3,4-d]pyrimidin-6-yl)oxy]phenol

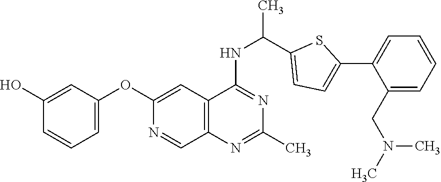 [SMILES: Cc1nc(NC(C)c2ccc(-c3ccccc3CN(C)C)s2)c2cc(Oc3cccc(O)c3)ncc2n1]

Under argon a mixture of resorcinol (78 mg, 0.71 mmol, commercially available), sodium hydride (28 mg, 0.71 mmol) in 1-Methyl-2-pyrrolidon (5 ml) were stirred at ambient temperature for 20 minutes. N-[1-(5-{2-[(dimethylaminonnethyl]phenyl}thiophen-2-yl)ethyl]-6-fluoro-2-methylpyrido[3,4-d]pyrimidin-4-amine (100 mg, 0.24 mmol, described in example 93, step b) in 1-Methyl-2-pyrrolidon (2 ml) were added and the reaction was stirred in a microwave oven at 170° C. for two hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (8 mg, 6%). 1H-NMR (400 MHz, DMSO-d6): d [ppm]=8.80 (br d, 1H), 8.75 (s, 1H), 7.93 (s, 1H), 7.44-7.40 (m, 1H), 7.39-7.35 (m, 1H), 7.34-7.27 (m, 2H), 7.20-7.12 (m, 2H), 7.10 (dd, 1H), 6.56 (ddd, 1H), 6.50-6.43 (m, 2H), 5.92 (br t, 1H), 3.35 (br s, 2H), 2.10 (s, 6H), 1.72 (d, 3H). LC-MS (Method 8): m/z: [M+H]⁺=512, Rₜ=0.77 min.

### EXAMPLE 53

### N4-[1-(5-{2-[(dimethylamino)methyl]phenyl}-2-thienyl)ethyl]-2-methyl-N6-(tetrahydro-2H-pyran-4-yl)pyrido[3,4-d]pyrimidine-4,6-diamine

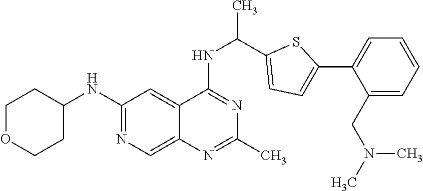 [SMILES: Cc1nc(NC(C)c2ccc(-c3ccccc3CN(C)C)s2)c2cc(NC3CCOCC3)ncc2n1]

A mixture of N-[1-(5-{2-[(dimethylamino)methyl]phenyl}thiophen-2-yl)ethyl]-6-fluoro-2-methylpyrido[3,4-d]pyrimidin-4-amine (100 mg, 0.27 mmol, described in example 93, step b), 4-aminotetrahydropyrane(84 mg, 0.83 mmol, commercially available) and triethylamine (116 μl, 0.83 mmol) in 1-Methyl-2-pyrrolidon (4 ml) were stirred in a microwave oven at 250° C. for six hours and at 200° C. overnight. The reaction mixture was allowed to cool to ambient temperature and separated between ethyl acetate and water. The aqueous layer was extracted with ethyl acetate (1×) and the combined organic layers were washed with brine, dried over sodium sulfate and the solvent was removed under reduced pressure. The residue was purified via HPLC chromatography to yield the title compound (22 mg, 18%). 1H-NMR (400 MHz, DMSO-d6): d [ppm]=8.57 (s, 1H), 8.36 (d, 1H), 7.46-7.39 (m, 1H), 7.38-7.33 (m, 1H), 7.30 (ddd, 2H), 7.18 (d, 1H), 7.08 (dd, 1H), 6.95 (s, 1H), 6.43 (d, 1H), 5.92 (quin, 1H), 3.94-3.85 (m, 2H), 3.81-3.71 (m, 1H), 3.42 (br t, 2H), 3.35 (s, 2H), 2.41 (s, 3H), 2.10 (s, 6H), 1.88 (br d, 2H), 1.72 (d, 3H), 1.59-1.41 (m, 2H). LC-MS (Method 7): m/z: [M+H]⁺=503.3, Rₜ=0.57 min.

### EXAMPLE 54

### N4-[1-(5-{2-[(dimethylamino)methyl]phenyl}-2-thienyl)ethyl]-2-methyl-N6-[2-(1H-pyrazol-1-yl)ethyl]pyrido[3,4-d]pyrimidine-4,6-diamine

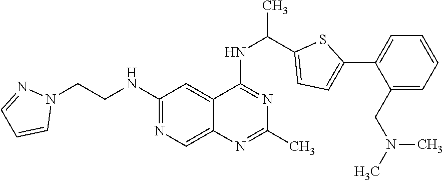 [SMILES: Cc1nc(NC(C)c2ccc(-c3ccccc3CN(C)C)s2)c2cc(NCCn3cccn3)ncc2n1]

A mixture of N-[1-(5-{2-[(dimethylamino)methyl]phenyl}thiophen-2-yl)ethyl]-6-fluoro-2-methylpyrido[3,4-d]pyrimidin-4-amine (116 mg, 0.27 mmol, described in example 93, step b), 2-(1H-pyrazol-1-yl)ethanamine (92 mg, 0.83 mmol, commercially available) and triethylamine (134 μl, 0.96 mmol) in 1-Methyl-2-pyrrolidon (3.5 ml) were stirred in a microwave oven at 250° C. for three hours and at 200° C. overnight. The reaction mixture was allowed to cool to ambient temperature and separated between ethyl acetate and water. The aqueous layer was extracted with ethyl acetate (1×) and the combined organic layers were washed with brine, dried over sodium sulfate and the solvent was removed under reduced pressure. The residue was purified via HPLC chromatography to yield the title compound (60 mg, 40%). 1H-NMR (400 MHz, DMSO-d6): d [ppm]=8.58 (s, 1H), 8.42 (d, 1H), 7.73 (d, 1H), 7.47-7.39 (m, 2H), 7.39-7.34 (m, 1H), 7.33-7.26 (m, 2H), 7.18 (d, 1H), 7.08 (dd, 1H), 7.00 (s, 1H), 6.53 (t, 1H), 6.20 (t, 1H), 5.91 (quin, 1H), 4.36 (t, 2H), 3.62 (q, 2H), 3.35 (s, 2H), 2.42 (s, 3H), 2.10 (s, 6H), 1.71 (d, 3H). LC-MS (Method 7): m/z: [M+H]⁺=513.3, Rₜ=0.59 min.

### EXAMPLE 55

### N-[1-(5-{2-[(dimethylamino)methyl]phenyl}thiophen-2-yl)ethyl]-2-methyl-6-[4-(pyridin-3-ylmethyl)piperazin-1-yl]pyrido[3,4-d]pyrimidin-4-amine

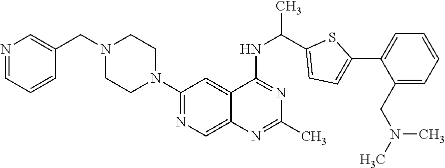 [SMILES: Cc1nc(NC(C)c2ccc(-c3ccccc3CN(C)C)s2)c2cc(N3CCN(Cc4cccnc4)CC3)ncc2n1]

A mixture of N-[1-(5-{2-[(dimethylamino)methyl]phenyl}thiophen-2-yl)ethyl]-6-fluoro-2-methylpyrido[3,4-d]pyrimidin-4-amine (120 mg, 0.28 mmol, described in example 93, step b), 1-(pyridin-3-ylmethyl)piperazine (177 mg, 1.00 mmol, commercially available) and triethylamine (139 μl, 1.00 mmol) in 1-Methyl-2-pyrrolidon (4 ml) was stirred in a microwave oven at 250° C. overnight. The reaction mixture was allowed to cool to ambient temperature and separated between ethyl acetate and water. The aqueous layer was extracted with ethyl acetate (1×) and the combined organic layers were washed with brine, dried over sodium sulfate and the solvent was removed under reduced pressure. The residue was purified via HPLC chromatography to yield the title compound (41 mg, 24%). 1H-NMR (400 MHz, DMSO-d6): d [ppm]=8.68 (s, 1H), 8.54 (d, 1H), 8.51-8.46 (m, 2H), 7.76 (dt, 1H), 7.45-7.34 (m, 4H), 7.32-7.27 (m, 2H), 7.19 (d, 1H), 7.09 (dd, 1H), 5.93 (quin, 1H), 3.61-3.52 (m, 6H), 3.35 (s, 2H), 2.44 (s, 3H), 2.10 (s, 6H), 1.72 (d, 3H). LC-MS (Method 7): m/z: [M+H]⁺=579.3, Rₜ=0.47 min.

### EXAMPLE 56

### N4-[1-(5-{2-[(dimethylamino)methyl]phenyl}-2-thienyl)ethyl]-N6-[2-(1H-imidazol-1-yl)ethyl]-2-methylpyrido[3,4-d]pyrimidine-4,6-diamine

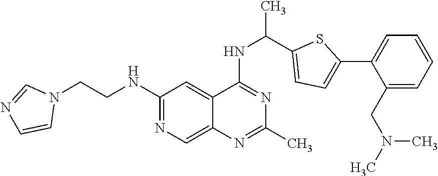 [SMILES: Cc1nc(NC(C)c2ccc(-c3ccccc3CN(C)C)s2)c2cc(NCCn3ccnc3)ncc2n1]

A mixture of N-[1-(5-{2-[(dimethylamino)methyl]phenyl}thiophen-2-yl)ethyl]-6-fluoro-2-methylpyrido[3,4-d]pyrimidin-4-amine (100 mg, 0.24 mmol, described in example 93, step b), 2-(1H-Imidazol-1-yl)ethanamine (79 mg, 0.71 mmol, commercially available) and triethylamine (116 μl, 0.83 mmol) in 1-Methyl-2-pyrrolidon (3.6 ml) was stirred at 150° C. overnight. The reaction mixture was allowed to cool to ambient temperature and separated between ethyl acetate and water. The aqueous layer was extracted with ethyl acetate (1×) and the combined organic layers were washed with brine, dried over sodium sulfate and the solvent was removed under reduced pressure. The residue was purified via HPLC chromatography to yield the title compound (36 mg, 27%). 1H-NMR (400 MHz, DMSO-d6): d [ppm]=8.58 (s, 1H), 8.40 (d, 1H), 7.62 (s, 1H), 7.42 (dd, 1H), 7.38-7.33 (m, 1H), 7.33-7.25 (m, 3H), 7.21-7.15 (m, 2H), 7.08 (dd, 1H), 6.98 (s, 1H), 6.85 (t, 1H), 6.63 (t, 1H), 5.91 (br t, 1H), 4.21 (t, 2H), 3.55 (q, 2H), 3.35 (s, 2H), 2.42 (s, 3H), 2.10 (s, 6H), 1.71 (d, 3H). LC-MS (Method 7): m/z: [M+H]⁺=513.3, Rₜ=0.42 min.

### EXAMPLE 57

### N-[(1R)-1-(3-bromophenyl)ethyl]-6-methoxy-2-methylpyrido[3,4-d]pyrimidin-4-amine

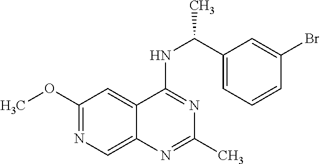 [SMILES: COc1cc2c(N[C@H](C)c3cccc(Br)c3)nc(C)nc2cn1]

Under argon a mixture of methanol (45 mg, 1.38 mmol, commercially available), sodium hydride (18 mg, 0.44 mmol, 60% in mineral oil) in DMF (5 ml) were stirred at ambient temperature for 10 minutes. N-[(1R)-1-(3-bromophenyl)ethyl]-6-fluoro-2-methylpyrido[3,4-d]pyrimidin-4-amine (100 mg, 0.27 mmol, described in example 34) were added and the reaction was stirred at 170° C. overnight. The reaction mixture was allowed to cool to ambient temperature, poured into ethyl acetate and washed with water (3×). The organic layer was dried over sodium sulfate and the solvent was removed under reduced pressure. The residue was purified via HPCL chromatography to yield the title compound (17 mg, 16%). 1H-NMR (400 MHz, DMSO-d6): d [ppm]=8.71 (s, 1H), 8.53 (d, 1H), 7.70 (d, 1H), 7.64 (t, 1H), 7.48-7.40 (m, 2H), 7.33-7.24 (m, 1H), 5.56 (quin, 1H), 3.95 (s, 3H), 2.39 (s, 3H), 1.57 (d, 3H). LC-MS (Method 7): m/z: [M+H]⁺=373, Rₜ=0.83 min.

### EXAMPLE 58

### N-[1-(5-{2-[(dimethylamino)methyl]phenyl}thiophen-2-yl)ethyl]-2-methyl-6-[3-(1-methyl-4,5-dihydro-1H-imidazol-2-yl)phenoxy]pyrido[3,4-d]pyrimidin-4-amine

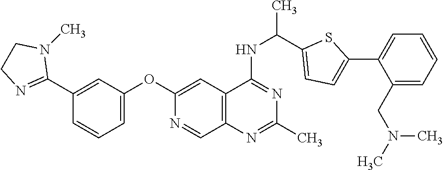 [SMILES: Cc1nc(NC(C)c2ccc(-c3ccccc3CN(C)C)s2)c2cc(Oc3cccc(C4=NCCN4C)c3)ncc2n1]

Under argon a mixture of 3-(1-methyl-4,5-dihydro-1H-imidazol-2-yl)phenol (125 mg, 0.71 mmol, commercially available), sodium hydride (28 mg, 0.71 mmol) in 1-Methyl-2-pyrrolidon (5 ml) were stirred at ambient temperature for 20 minutes. N-[1-(5-{2-[(dimethylamino)methyl]phenyl}thiophen-2-yl)ethyl]-6-fluoro-2-methylpyrido [3,4-d]pyrimidin-4-amine (100 mg, 0.24 mmol, described in example 93, step b) in 1-Methyl-2-pyrrolidon (2 ml) were added and the reaction was stirred at 170° C. for two hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (32 mg, 21%). 1H-NMR (400 MHz, DMSO-d6): d [ppm]=8.81 (br d, 1H), 8.75 (s, 1H), 8.02 (s, 1H), 7.53-7.46 (m, 1H), 7.42 (dd, 1H), 7.39-7.36 (m, 1H), 7.35-7.28 (m, 3H), 7.26-7.22 (m, 1H), 7.19 (s, 1H), 7.11 (dd, 1H), 5.93 (quin, 1H), 3.72-3.64 (m, 1H), 2.72 (s, 2H), 2.10 (s, 6H), 1.72 (d, 3H). LC-MS (Method 7): m/z: [M+H]⁺=578.3, Rₜ=0.55 min.

### EXAMPLE 59

### N-[1-(5-{2-[(dimethylamino)methyl]phenyl}thiophen-2-yl)ethyl]-2-methyl-6-(methylsulfanyl)pyrido[3,4-d]pyrimidin-4-amine

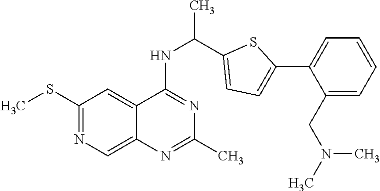 [SMILES: CSc1cc2c(NC(C)c3ccc(-c4ccccc4CN(C)C)s3)nc(C)nc2cn1]

A mixture of N-[1-(5-{2-[(dimethylamino)methyl]phenyl}thiophen-2-yl)ethyl]-6-fluoro-2-methylpyrido[3,4-d]pyrimidin-4-amine (150 mg, 0.36 mmol, described in example 93, step b), sodium methanethiolate (75 mg, 1.07 mmol, commercially available) and triethylamine (149 μl, 1.07 mmol) in DMSO (6 ml) were stirred at 150° C. overnight. The rection mixture was allowed to cool to ambient temperature and separated between ethyl acetate and water. The aqueous layer was extracted with ethyl acetate (2×) and the combined organic layers were washed with brine (1×), dried over sodium sulfate and the solvent was removed under reduced pressure. The residue was purified via HPLC chromatography to yield the title compound (26 mg, 19%). 1H-NMR (400 MHz, DMSO-d6): d [ppm]=8.90 (s, 1H), 8.80 (d, 1H), 8.06 (d, 1H), 7.42 (dd, 1H), 7.39-7.35 (m, 1H), 7.34-7.25 (m, 2H), 7.19 (d, 1H), 7.11 (dd, 1H), 5.94 (quin, 1H), 3.35 (s, 2H), 2.60 (s, 3H), 2.10 (s, 6H), 1.73 (d, 3H). LC-MS (Method 8): m/z: [M+H]⁺=450, Rₜ=0.75 min.

### EXAMPLE 60

### N-[(3R)-1-(4-{[1-(5-{2-[(dimethylamino)methyl]phenyl}thiophen-2-yl)ethyl]amino}-2-methylpyrido[3,4-d]pyrimidin-6-yl)pyrrolidin-3-yl]acetamide

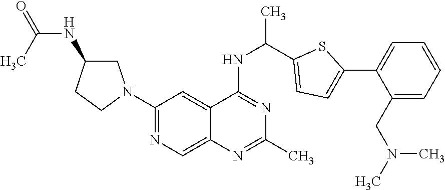 [SMILES: CC(=O)N[C@@H]1CCN(c2cc3c(NC(C)c4ccc(-c5ccccc5CN(C)C)s4)nc(C)nc3cn2)C1]

A mixture of N-[1-(5-{2-[(dimethylamino)methyl]phenyl}thiophen-2-yl)ethyl]-6-fluoro-2-methylpyrido[3,4-d]pyrimidin-4-amine (150 mg, 0.36 mmol, described in example 93, step b), (3R)-(+)-3-acetamidopyrrolidine (140 mg, 1.07 mmol, commercially available) and triethylamine (149 μl, 1.07 mmol) in 1-Methyl-2-pyrrolidon (7.5 ml) were stirred at 170° C. overnight. The rection mixture was allowed to cool to ambient temperature and separated between ethyl acetate and water. The aqueous layer was extracted with ethyl acetate (2×) and the combined organic layers were washed with brine (1×), dried over sodium sulfate and the solvent was removed under reduced pressure. The residue was purified via HPLC chromatography to yield the title compound as mixture of diastereomers (87 mg, 45%). 1H-NMR (400 MHz, DMSO-d6): d [ppm]=8.66 (s, 1H), 8.47 (d, 1H), 8.18 (d, 1H), 7.44-7.40 (m, 1H), 7.39-7.35 (m, 1H), 7.34-7.26 (m, 2H), 7.19 (dd, 1H), 7.08 (dd, 1H), 7.00 (s, 1H), 5.93 (quin, 1H), 4.44-4.33 (m, 1H), 3.71-3.46 (m, 3H), 3.35 (s, 2H), 3.30 (br d, 1H), 2.42 (s, 3H), 2.18 (dd, 1H), 2.10 (d, 6H), 1.99-1.88 (m, 1H), 1.81 (d, 3H), 1.72 (d, 3H). LC-MS (Method 7): m/z: [M+H]⁺=530.3, Rₜ=0.54 min.

### EXAMPLE 61

### N-[(3S)-1-(4-{[1-(5-{2-[(dimethylamino)methyl]phenyl}thiophen-2-yl)ethyl]amino}-2-methylpyrido[3,4-d]pyrimidin-6-yl)pyrrolidin-3-yl]acetamide

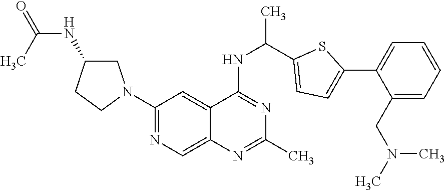 [SMILES: CC(=O)N[C@H]1CCN(c2cc3c(NC(C)c4ccc(-c5ccccc5CN(C)C)s4)nc(C)nc3cn2)C1]

A mixture of N-[1-(5-{2-[(dimethylamino)methyl]phenyl}thiophen-2-yl)ethyl]-6-fluoro-2-methylpyrido[3,4-d]pyrimidin-4-amine (150 mg, 0.36 mmol, described in example 93, step b), (3S)-(−)-3-acetamidopyrrolidine (140 mg, 1.07 mmol, commercially available) and triethylamine (149 μl, 1.07 mmol) in 1-Methyl-2-pyrrolidon (7.5 ml) were stirred at 170° C. overnight. The rection mixture was allowed to cool to ambient temperature and separated between ethyl acetate and water. The aqueous layer was extracted with ethyl acetate (2×) and the combined organic layers were washed with brine (1×), dried over sodium sulfate and the solvent was removed under reduced pressure. The residue was purified via HPLC chromatography to yield the title compound as mixture of diastereomers (88 mg, 46%). 1H-NMR (400 MHz, DMSO-d6): d [ppm]=8.66 (s, 1H), 8.47 (d, 1H), 8.18 (d, 1H), 7.42 (br d, 1H), 7.39-7.35 (m, 1H), 7.34-7.25 (m, 2H), 7.19 (dd, 1H), 7.08 (d, 1H), 7.00 (s, 1H), 5.94 (quin, 1H), 4.44-4.31 (m, 1H), 3.73-3.45 (m, 3H), 3.35 (s, 2H), 3.29 (br dd, 1H), 2.42 (s, 3H), 2.26-2.14 (m, 1H), 2.10 (s, 6H), 1.98-1.88 (m, 1H), 1.81 (d, 3H), 1.72 (d, 3H). LC-MS (Method 7): m/z: [M+H]⁺=530.3, Rₜ=0.54 min.

### EXAMPLE 62

### N-{2-[(4-{[(1R)-1-(3-bromophenyl)ethyl]amino}-2-methylpyrido[3,4-d]pyrimidin-6-yl)oxy]ethyl}acetamide

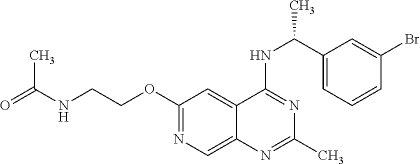 [SMILES: CC(=O)NCCOc1cc2c(N[C@H](C)c3cccc(Br)c3)nc(C)nc2cn1]

Under argon a mixture of N-Acetylethanolamine (150 mg, 1.38 mmol, commercially available), sodium hydride (18 mg, 0.44 mmol, 60% in mineral oil) in DMF (6 ml) were stirred at ambient temperature for 10 minutes. N-[(1R)-1-(3-bromophenyeethyl]-6-fluoro-2-methylpyrido[3,4-d]pyrimidin-4-amine (100 mg, 0.27 mmol, described in example 34) were added and the reaction was stirred at 170° C. overnight. The reaction mixture was allowed to cool to ambient temperature, poured into ethyl acetate and washed with water (3×). The organic layer was dried over sodium sulfate and the solvent was removed under reduced pressure. The residue was purified via HPCL chromatography to yield the title compound (4 mg, 3%). 1H-NMR (400 MHz, DMSO-d6): d [ppm]=8.70 (s, 1H), 8.53 (d, 1H), 8.10 (br t, 1H), 7.73 (s, 1H), 7.64 (t, 1H), 7.47-7.38 (m, 2H), 7.32-7.24 (m, 1H), 5.55 (quin, 1H), 4.32 (t, 2H), 3.46 (q, 2H), 2.39 (s, 3H), 1.83 (s, 3H), 1.56 (d, 3H). LC-MS (Method 7): m/z: [M+H]⁺=444 & 446, Rₜ=0.77 min.

### EXAMPLE 63

### N-[1-(5-{2-[(dimethylamino)methyl]phenyl}thiophen-2-yl)ethyl]-6-ethoxy-2-methylpyrido[3,4-d]pyrimidin-4-amine

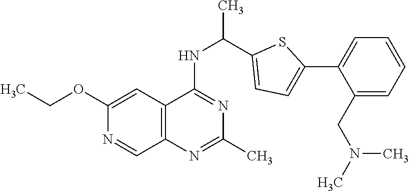 [SMILES: CCOc1cc2c(NC(C)c3ccc(-c4ccccc4CN(C)C)s3)nc(C)nc2cn1]

Under argon a mixture of ethanol (54 mg, 1.19 mmol, commercially available), sodium hydride (58 mg, 1.4 mmol) in 1-Methyl-2-pyrrolidon (4.5 ml) were stirred at ambient temperature for 20 minutes. N-[1-(5-{2-[(dimethylamino)methyl]phenyl}thiophen-2-yl)ethyl]-6-fluoro-2-methylpyrido[3,4-d]pyrimidin-4-amine (100 mg, 0.24 mmol, described in example 93, step b) were added and the reaction was stirred at 170° C. for three hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (75 mg, 71%). 1H-NMR (400 MHz, DMSO-d6): d [ppm]=8.72 (d, 1H), 8.63 (d, 1H), 7.65 (d, 1H), 7.44-7.40 (m, 1H), 7.39-7.35 (m, 1H), 7.32-7.27 (m, 2H), 7.18 (d, 1H), 7.09 (dd, 1H), 5.91 (quin, 1H), 4.34 (q, 2H), 3.35 (s, 2H), 2.47 (s, 3H), 2.09 (s, 6H), 1.72 (d, 3H), 1.35 (t, 3H). LC-MS (Method 7): m/z: [M+H]⁺=448.2, Rₜ=0.60 min.

### EXAMPLE 64

### N-[1-(5-{2-[(dimethylamino)methyl]phenyl}thiophen-2-yl)ethyl]-6-methoxy-2-methylpyrido[3,4-d]pyrimidin-4-amine

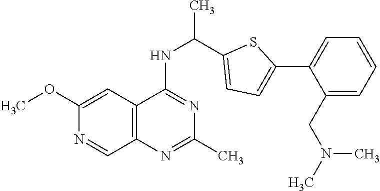 [SMILES: COc1cc2c(NC(C)c3ccc(-c4ccccc4CN(C)C)s3)nc(C)nc2cn1]

Under argon a mixture of methanol (38 mg, 1.19 mmol, commercially available), sodium hydride (58 mg, 1.4 mmol) in 1-Methyl-2-pyrrolidon (4.5 ml) were stirred at ambient temperature for 20 minutes. N-[1-(5-{2-[(dimethylamino)methyl]phenyl}thiophen-2-yl)ethyl]-6-fluoro-2-methylpyrido[3,4-d]pyrimidin-4-amine (100 mg, 0.24 mmol, described in example 93, step b) were added and the reaction was stirred at 170° C. for three hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (73 mg, 67%). 1H-NMR (400 MHz, DMSO-d6): d [ppm]=8.74 (s, 1H), 8.68 (d, 1H), 7.66 (s, 1H), 7.42 (dd, 1H), 7.39-7.35 (m, 1H), 7.33-7.26 (m, 2H), 7.18 (d, 1H), 7.10 (dd, 1H), 5.92 (quin, 1H), 3.93 (s, 3H), 3.35 (s, 2H), 2.47 (s, 3H), 2.09 (s, 6H), 1.72 (d, 3H). LC-MS (Method 7): m/z: [M+H]⁺=434.2, Rₜ=0.56 min.

### EXAMPLE 65

### N-[1-(5-{2-[(dimethylamino)methyl]phenyl}thiophen-2-yl)ethyl]-2-methyl-6-propoxypyrido[3,4-d]pyrimidin-4-amine

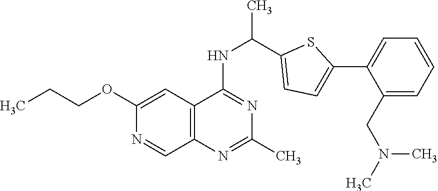 [SMILES: CCCOc1cc2c(NC(C)c3ccc(-c4ccccc4CN(C)C)s3)nc(C)nc2cn1]

Under argon a mixture of 1-Propanol (71 mg, 1.19 mmol, commercially available), sodium hydride (58 mg, 1.4 mmol) in 1-Methyl-2-pyrrolidon (4.5 ml) were stirred at ambient temperature for 20 minutes. N-[1-(5-{2-[(dimethylamino)methyl]phenyl}thiophen-2-yl)ethyl]-6-fluoro-2-methylpyrido[3,4-d]pyrimidin-4-amine (100 mg, 0.24 mmol, described in example 93, step b) were added and the reaction was stirred at 170° C. for three hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (62 mg, 54%). 1H-NMR (400 MHz, DMSO-d6): d [ppm]=8.72 (d, 1H), 8.64 (d, 1H), 7.66 (s, 1H), 7.42 (dd, 1H), 7.39-7.34 (m, 1H), 7.33-7.25 (m, 2H), 7.18 (d, 1H), 5.91 (quin, 1H), 4.24 (t, 2H), 3.35 (s, 2H), 2.47 (s, 3H), 2.09 (s, 6H), 1.83-1.67 (m, 5H), 1.00 (t, 3H). . LC-MS (Method 7): m/z: [M+H]⁺=462.2, Rₜ=0.66 min.

### EXAMPLE 66

### 6-[2-(dimethylamino)ethoxy]-N-[1-(5-{2-[(dimethylamino)methyl]phenyl}thiophen-2-yl)ethyl]-2-methylpyrido[3,4-d]pyrimidin-4-amine

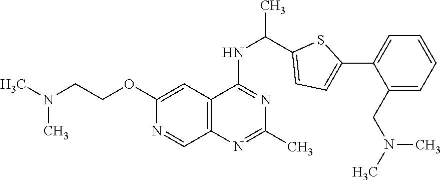 [SMILES: Cc1nc(NC(C)c2ccc(-c3ccccc3CN(C)C)s2)c2cc(OCCN(C)C)ncc2n1]

Under argon a mixture of 2-(dimethylamino)-ethanol (105 mg, 1.19 mmol, commercially available), sodium hydride (58 mg, 1.4 mmol) in 1-Methyl-2-pyrrolidon (4.5 ml) were stirred at ambient temperature for 20 minutes. N-[1-(5-{2-[(dimethylamino)methyl]phenyl}thiophen-2-yl)ethyl]-6-fluoro-2-methylpyrido[3,4-d]pyrimidin-4-amine (100 mg, 0.24 mmol, described in example 93, step b) were added and the reaction was stirred at 170° C. for three hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (63 mg, 51%). 1H-NMR (400 MHz, DMSO-d6): d [ppm]=8.72 (s, 1H), 8.63 (d, 1H), 7.67 (s, 1H), 7.41 (dd, 1H), 7.39-7.33 (m, 1H), 7.32-7.26 (m, 2H), 7.18 (d, 1H), 7.09 (dd, 1H), 5.90 (quin, 1H), 4.37 (t, 2H), 2.64 (t, 2H), 2.47 (s, 4H), 2.22 (s, 6H), 2.09 (s, 6H), 1.71 (d, 3H). LC-MS (Method 7): m/z: [M+H]⁺=491.3, Rₜ=0.43 min.

### EXAMPLE 67

### N-[1-(5-{2-[(dimethylamino)methyl]phenyl}thiophen-2-yl)ethyl]-6-[(1,1-dioxidotetrahydro-2H-thiopyran-4-yl)oxy]-2-methylpyrido[3,4-d]pyrimidin-4-amine

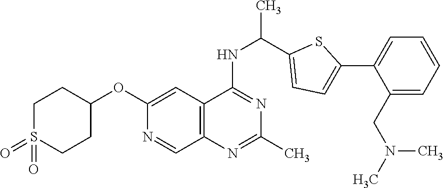 [SMILES: Cc1nc(NC(C)c2ccc(-c3ccccc3CN(C)C)s2)c2cc(OC3CCS(=O)(=O)CC3)ncc2n1]

Under argon a mixture of 4-hydroxytetrahydro-2H-thiopyrane 1,1-dioxide (178 mg, 1.19 mmol, commercially available), sodium hydride (58 mg, 1.4 mmol) in 1-Methyl-2-pyrrolidon (4.5 ml) were stirred at ambient temperature for 20 minutes. N-[1-(5-{2-[(dimethylamino)-methyl]phenyl}thiophen-2-yl)ethyl]-6-fluoro-2-methylpyrido [3,4-d]pyrimidin-4-amine (100 mg, 0.24 mmol, described in example 93, step b) in 1-Methyl-2-pyrrolidon (2ml) were added and the reaction was stirred at 170° C. for two hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (70 mg, 51%). 1H-NMR (400 MHz, DMSO-d6): d [ppm]=8.74 (s, 1H), 8.67 (d, 1H), 7.75 (s, 1H), 7.44-7.40 (m, 1H), 7.39-7.34 (m, 1H), 7.33-7.26 (m, 2H), 7.18 (d, 1H), 7.10 (dd, 1H), 5.89 (quin, 1H), 5.35 (tt, 1H), 3.33 (s, 6H), 3.29-3.14 (m, 4H), 2.48 (s, 3H), 2.36-2.22 (m, 4H), 2.10 (s, 6H), 1.71 (d, 3H). LC-MS (Method 7): m/z: [M+H]⁺=552.2, Rₜ=0.58 min.

### EXAMPLE 68

### [2-(5-{1-[(6-fluoro-2-methylpyrido[3,4-d]pyrimidin-4-yl)amino]ethyl}thiophen-2-yl)phenyl]methanol

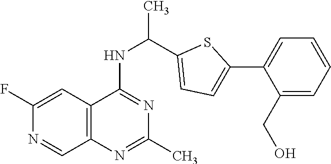 [SMILES: Cc1nc(NC(C)c2ccc(-c3ccccc3CO)s2)c2cc(F)ncc2n1]

A mixture of 2-(5-{1-[(6-fluoro-2-methylpyrido[3,4-d]pyrimidin-4-yl)amino]¬ethyl}thiophen-2-yl)benzaldehyde (5.59 g, 14.24 mmol, described in example 93, step a), sodium triacetoxyborohydride (6.038 g, 28.49 mmol), acetic acid (2.038 ml, 35.61 mmol) in 1,2-dichloroethane (124 ml) was stirred at ambient temperature overnight. The reaction mixture was stopped by the addition of NaOH solution (1M in water) and extracted with dichloromethane (3×). The combined organic layers were washed with brine (1×), dried over sodium sulfate and the solvent was removed under reduced pressure. The residue was purified via Isolera chromatography (375 g SNAP-NH column) using a hexanes/ethyl acetate 0-60% gradient. The title compound was obtained in 7% yield (410 mg). 1H-NMR (400 MHz, DMSO-d6): d [ppm]=8.86 (d, 1H), 8.76 (s, 1H), 8.06 (d, 1H), 7.57-7.51 (m, 1H), 7.40-7.32 (m, 2H), 7.30-7.24 (m, 1H), 7.17-7.10 (m, 2H), 5.91 (quin, 1H), 5.24 (t, 1H), 4.53 (d, 2H), 2.52 (s, 3H), 1.73 (d, 3H). LC-MS (Method 7): m/z: [M+H]⁺=395.1, Rₜ=0.86 min.

### EXAMPLE 69

### 3-(4-{[1-(5-{2-[(dimethylamino)methyl]phenyl}thiophen-2-yl)ethyl]amino}-2-methylpyrido[3,4-d]pyrimidin-6-yl)-1-methylpyrrolidin-2-one

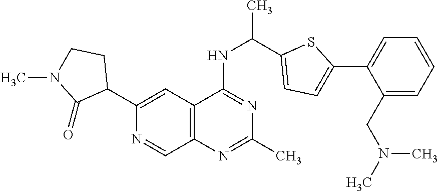 [SMILES: Cc1nc(NC(C)c2ccc(-c3ccccc3CN(C)C)s2)c2cc(C3CCN(C)C3=O)ncc2n1]

To a solution of N-[1-(5-{2-[(dimethylaminomethyl]phenyl}¬thiophen-2-yl)ethyl]-6-fluoro-2-methylpyrido[3,4-d]pyrimidin-4-amine (100 mg, 0.23 mmol, described in example 93, step b) in 1-methyl-2-pyrrolidinone (3.2 ml) was added NaH (95 mg, 2.37 mmol) at ambient temperature under argon and the reaction was stirred at ambient temperature for twenty minutes and at 170° C. for three hours. The reaction was allowed to cool to ambient temperature and diluted with water/ethyl acetate. The layers were separated and the aqueous layer was extracted with ethyl acetate (2×). The combined organic layers were washed with brine (1×), dried over sodium sulfate and the solvent was removed under reduced pressure. The title comound was obtained after HPCL chromatography (14 mg, 11%). 1H-NMR (400 MHz, DMSO-d6): d [ppm]=8.96 (s, 1H), 8.86 (dd, 1H), 8.13 (s, 1H), 7.42 (dd, 1H), 7.39-7.35 (m, 1H), 7.33-7.26 (m, 2H), 7.21-7.17 (m, 1H), 7.11 (br d, 1H), 5.94 (td, 1H), 3.81 (t, 1H), 3.57-3.49 (m, 1H), 3.44 (q, 1H), 3.35 (s, 2H), 2.79 (d, 3H), 2.46-2.35 (m, 2H), 2.09 (s, 6H), 1.73 (dd, 3H). LC-MS (Method 7): m/z: [M+H]⁺=501.2 & 501.2, Rₜ=0.55 & 0.57 min. (mixture of diastereoisomers).

### EXAMPLE 70

### N-[(1R)-1-(3-bromophenyl)ethyl]-6-[3-(dimethylamino)propoxy]-2-methylpyrido[3,4-d]pyrimidin-4-amine

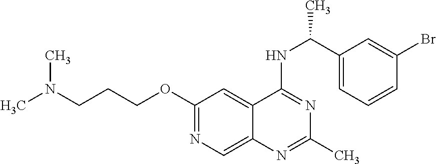 [SMILES: Cc1nc(N[C@H](C)c2cccc(Br)c2)c2cc(OCCCN(C)C)ncc2n1]

Under argon a mixture of 3-Dimethylamino-1-propanol (144 mg, 1.38 mmol, commercially available), sodium hydride (18 mg, 0.44 mmol, 60% in mineral oil) in DMF (6 ml) were stirred at ambient temperature for 10 minutes. N-[(1R)-1-(3-bromophenyeethyl]-6-fluoro-2-methylpyrido[3,4-d]pyrimidin-4-amine (100 mg, 0.27 mmol, described in example 34) were added and the reaction was stirred at 170° C. overnight. The reaction mixture was allowed to cool to ambient temperature, poured into ethyl acetate and washed with water (3×). The organic layer was dried over sodium sulfate and the solvent was removed under reduced pressure. The residue was purified via HPCL chromatography to yield the title compound (8 mg, 6%). 1H-NMR (400 MHz, CD₃OD): d [ppm]=8.65 (d, 1H), 7.61 (t, 1H), 7.52 (d, 1H), 7.44-7.34 (m, 2H), 7.26-7.17 (m, 1H), 5.59 (q, 1H), 4.37 (t, 2H), 2.59-2.51 (m, 2H), 2.46 (s, 3H), 2.28 (s, 6H), 2.09-1.93 (m, 2H), 1.63 (d, 3H). LC-MS (Method 7): m/z: [M+H]⁺=446.1, Rₜ=0.62 min.

### EXAMPLE 71

### 6-(azetidin-1-yl)-N-[1-(5-{2-[(dimethylamino)methyl]phenyl}thiophen-2-yl)ethyl]-2-methylpyrido[3,4-d]pyrimidin-4-amine

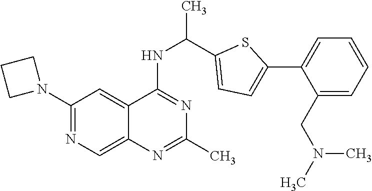 [SMILES: Cc1nc(NC(C)c2ccc(-c3ccccc3CN(C)C)s2)c2cc(N3CCC3)ncc2n1]

A solution of azetidine (52 mg, 0.91 mmol, commercially available), N-[1-(5-{2-[(dimethylamino)¬methyl]phenyl}¬thiophen-2-yl)ethyl]-6-fluoro-2-methylpyrido [3,4-d]pyrimidin-4-amine (110 mg, 0.26 mmol, described in example 93, step b) and triethylamine (127 μl, 0.91 mmol) in 1-Methyl-2-pyrrolidon (4 ml) was heated in a microwave oven at 250° C. for three hours. The reaction was allowed to cool to ambient temperature, diluted with ethyl acetate and water. The layers were separated and the aqueous layer was extracted with ethyl acetate (2×). The combined organic layers were washed with brine (1×), dried over sodium sulfate and the solvent was removed under reduced pressure. After HPLC chromatography the title compound was obtained in 10% yield (12 mg). 1H-NMR (400 MHz, DMSO-d6): d [ppm]=8.63 (s, 1H), 8.48 (d, 1H), 7.42 (dd, 1H), 7.38-7.35 (m, 1H), 7.33-7.25 (m, 2H), 7.18 (d, 1H), 7.09 (dd, 1H), 7.01 (s, 1H), 5.92 (quin, 1H), 3.99 (t, 4H), 3.35 (s, 2H), 2.43 (s, 3H), 2.39-2.31 (m, 3H), 2.10 (s, 6H), 1.72 (d, 3H). LC-MS (Method 7): m/z: [M+H]⁺=459.2, Rₜ=0.57 min.

### EXAMPLE 72

### N-[1-(5-{2-[(dimethylamino)methyl]phenyl}thiophen-2-yl)ethyl]-6-[3-(dimethylamino)propoxy]-2-methylpyrido[3,4-d]pyrimidin-4-amine

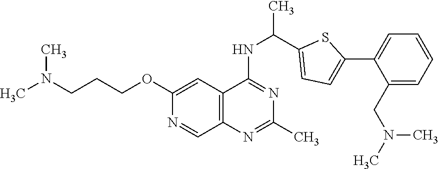 [SMILES: Cc1nc(NC(C)c2ccc(-c3ccccc3CN(C)C)s2)c2cc(OCCCN(C)C)ncc2n1]

Under argon a mixture of 3-dimethylamino-1-propanol (122 mg, 1.19 mmol, commercially available), sodium hydride (58 mg, 1.4 mmol) in 1-Methyl-2-pyrrolidon (4.5 ml) were stirred at ambient temperature for 20 minutes. N-[1-(5-{2-[(dimethylamino)methyl]phenyl}thiophen-2-yl)ethyl]-6-fluoro-2-methylpyrido[3,4-d]pyrimidin-4-amine (100 mg, 0.24 mmol, described in example 93, step b) were added and the reaction was stirred at 170° C. for six hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (49 mg, 39%). 1H-NMR (400 MHz, DMSO-d6): d [ppm]=8.71 (s, 1H), 8.65 (d, 1H), 7.65 (s, 1H), 7.44-7.39 (m, 1H), 7.39-7.35 (m, 1H), 7.33-7.26 (m, 2H), 7.18 (d, 1H), 7.09 (dd, 1H), 5.91 (quin, 1H), 4.30 (t, 2H), 3.35 (s, 2H), 2.47 (s, 3H), 2.36 (t, 2H), 2.13 (s, 6H), 2.09 (s, 6H), 1.87 (quin, 2H), 1.71 (d, 3H). LC-MS (Method 7): m/z: [M+H]⁺=505.3, Rₜ=0.44 min.

### EXAMPLE 73

### 6-(cyclopropylmethoxy)-N-[1-(5-{2-[(dimethylamino)methyl]phenyl}thiophen-2-yl)ethyl]-2-methylpyrido[3,4-d]pyrimidin-4-amine

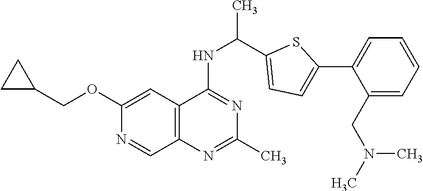 [SMILES: Cc1nc(NC(C)c2ccc(-c3ccccc3CN(C)C)s2)c2cc(OCC3CC3)ncc2n1]

Under argon a mixture of cyclopropanemethanol (86 mg, 1.19 mmol, commercially available), sodium hydride (28 mg, 0.71 mmol) in 1-Methyl-2-pyrrolidon (4.5 ml) were stirred at ambient temperature for 20 minutes. N-[1-(5-{2-[(dimethylamino)methyl]phenyl}-thiophen-2-yl)ethyl]-6-fluoro-2-methylpyrido[3,4-d]pyrimidin-4-amine (100 mg, 0.24 mmol, described in example 93, step b) were added and the reaction was stirred at 170° C. for six hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (57 mg, 48%). 1H-NMR (400 MHz, DMSO-d6): d [ppm]=8.71 (s, 1H), 8.62 (d, 1H), 7.66 (s, 1H), 7.42 (dd, 1H), 7.39-7.34 (m, 1H), 7.33-7.25 (m, 2H), 7.18 (d, 1H), 7.09 (dd, 1H), 5.91 (quin, 1H), 4.14 (d, 2H), 3.35 (s, 2H), 2.47 (s, 3H), 2.09 (s, 6H), 1.72 (d, 3H), 1.32-1.20 (m, 1H), 0.61-0.53 (m, 2H), 0.39-0.29 (m, 2H). LC-MS (Method 7): m/z: [M+H]⁺=474.2, Rₜ=0.67 min.

### EXAMPLE 74

### 2-[(4-{[1-(5-{2-[(dimethylamino)methyl]phenyl}thiophen-2-yl)ethyl]amino}-2-methylpyrido[3,4-d]pyrimidin-6-yl)oxy]ethanol

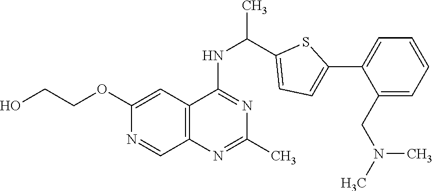 [SMILES: Cc1nc(NC(C)c2ccc(-c3ccccc3CN(C)C)s2)c2cc(OCCO)ncc2n1]

Under argon a mixture of ethylene glycol (74 mg, 1.19 mmol, commercially available), sodium hydride (28 mg, 0.71 mmol) in 1-Methyl-2-pyrrolidon (3.5 ml) were stirred at ambient temperature for 10 minutes. N-[1-(5-{2-[(dimethylamino)methyl]phenyl}thiophen-2-yl)ethyl]-6-fluoro-2-methylpyrido[3,4-d]pyrimidin-4-amine (100 mg, 0.24 mmol, described in example 93, step b) were added and the reaction was stirred in a microwave oven at 170° C. for five hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (19 mg, 17%). 1H-NMR (400 MHz, DMSO-d6): d [ppm]=8.72 (s, 1H), 8.66 (d, 1H), 7.68 (s, 1H), 7.42 (dd, 1H), 7.39-7.34 (m, 1H), 7.34-7.23 (m, 2H), 7.18 (d, 1H), 7.09 (dd, 1H), 5.91 (quin, 1H), 4.86 (t, 1H), 4.31 (t, 2H), 3.74 (q, 2H), 3.35 (s, 2H), 2.47 (s, 3H), 2.10 (s, 6H), 1.72 (d, 3H). LC-MS (Method 7): m/z: [M+H]⁺=464.2, Rₜ=0.52 min.

### EXAMPLE 75

### N-[(1R)-1-(3-bromophenyl)ethyl]-6-(cyclopropylmethoxy)-2-methylpyrido[3,4-d]pyrimidin-4-amine

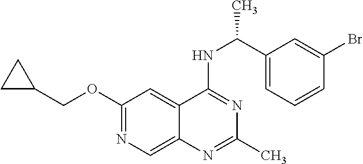 [SMILES: Cc1nc(N[C@H](C)c2cccc(Br)c2)c2cc(OCC3CC3)ncc2n1]

Under argon a mixture of (Hydroxymethyl)-cyclopropan (102 mg, 1.38 mmol, commercially available), sodium hydride (18 mg, 0.44 mmol, 60% in mineral oil) in DMF (6 ml) were stirred at ambient temperature for 10 minutes. N-[(1R)-1-(3-bromophenyeethyl]-6-fluoro-2-methylpyrido[3,4-d]pyrimidin-4-amine (100 mg, 0.27 mmol, described in example 34) were added and the reaction was stirred at 170° C. overnight. The reaction mixture was allowed to cool to ambient temperature, poured into ethyl acetate and washed with water (3×). The organic layer was dried over sodium sulfate and the solvent was removed under reduced pressure. The residue was purified via HPCL chromatography to yield the title compound (8 mg, 7%). 1H-NMR (400 MHz, CD3OD): d [ppm]=8.64 (d, 1H), 7.61 (t, 1H), 7.51 (d, 1H), 7.44-7.32 (m, 2H), 7.26-7.17 (m, 1H), 5.59 (q, 1H), 4.17 (d, 2H), 2.45 (s, 3H), 1.63 (d, 3H), 1.38-1.23 (m, 1H), 0.67-0.57 (m, 2H), 0.42-0.30 (m, 2H). LC-MS (Method 7): m/z: [M+H]⁺=413.1, Rₜ=0.95 min.

### EXAMPLE 76

### 3-[(4-{[1-(5-{2-[(dimethylamino)methyl]phenyl}thiophen-2-yl)ethyl]amino}-2-methylpyrido[3,4-d]pyrimidin-6-yl)oxy]propan-1-ol

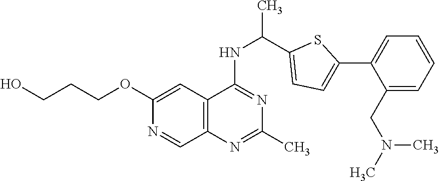 [SMILES: Cc1nc(NC(C)c2ccc(-c3ccccc3CN(C)C)s2)c2cc(OCCCO)ncc2n1]

Under argon a mixture of 1,3-propanediole (90 mg, 1.19 mmol, commercially available), sodium hydride (28 mg, 0.71 mmol) in 1-Methyl-2-pyrrolidon (3.5 ml) were stirred at ambient temperature for 10 minutes. N-[1-(5-{2-[(dimethylaminolmethyl]phenyl}thiophen-2-yl)ethyl]-6-fluoro-2-methylpyrido[3,4-d]pyrimidin-4-amine (100 mg, 0.24 mmol, described in example 93, step b) were added and the reaction was stirred at 170° C. for 2.5 hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (5 mg, 4%). 1H-NMR (400 MHz, DMSO-d6): d [ppm]=8.72 (s, 1H), 8.65 (d, 1H), 7.66 (s, 1H), 7.42 (dd, 1H), 7.39-7.34 (m, 1H), 7.33-7.25 (m, 2H), 7.18 (d, 1H), 7.09 (dd, 1H), 5.91 (quin, 1H), 4.57 (t, 1H), 4.34 (t, 2H), 3.61-3.54 (m, 2H), 3.35 (s, 2H), 2.47 (s, 3H), 2.10 (s, 6H), 1.89 (quin, 2H), 1.72 (d, 3H). LC-MS (Method 7): m/z: [M+H]⁺=478.2, Rₜ=0.54 min.

### EXAMPLE 77

### N-[1-(5-{2-[(dimethylamino)methyl]phenyl}-2-thienyl)ethyl]-6-[(1-imino-1-oxidohexahydro-1lambda4-thiopyran-4-yl)oxy]-2-methylpyrido[3,4-d]pyrimidin-4-amine

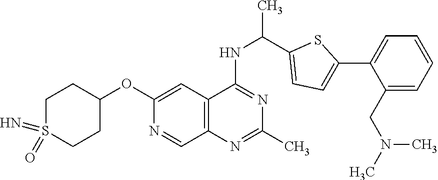 [SMILES: Cc1nc(NC(C)c2ccc(-c3ccccc3CN(C)C)s2)c2cc(OC3CCS(=N)(=O)CC3)ncc2n1]

### Step a

### 2,2,2-trifluoro-N-(4-hydroxy-1-oxo-thian-1-ylidene)acetamide

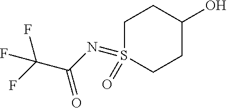 [SMILES: O=C(N=S1(=O)CCC(O)CC1)C(F)(F)F]

A mixture of tetrahydrothiopyran-4-ol (98.00 g, 829.10 mmol, 1.00 eq), 2,2,2-trifluoroacetamide (141.00 g, 1.25 mol, 1.50 eq), PhI(OAc)₂ (401.00 g, 1.24 mol, 1.50 eq), MgO (134.00 g, 3.33 mol, 4.01 eq) and Rh2(OAc)4 (11.00 g, 24.89 mmol, 0.03 eq) in DCM (1.50 L) was stirred at 20° C. for 18 hrs. TLC (DCM/MeOH=20/1, Rf=0.35) showed source material was consumed completely and three new spots were found. The mixture was filtered through a pad of Celite and the filter cake was washed with DCM (400 mL*2). The combined filtrates were concentrated under vacuum. The residue was purified by silica gel column (DCM/MeOH=40/1 to 20/1) to give the title compound (83.00 g, 270.78 mmol, 32.66% yield, 80% purity) as light yellow solid. LCMS (method 2): Rt=0.655 min., m/z=122.1 (M+H)+.

### Step b

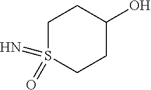

### 1-imino-1-oxo-thian-4-ol

To a solution of 2,2,2-trifluoro-N-(4-hydroxy-1-oxo-thian-1-ylidene)acetamide (83.00 g, crude, described in example 77, step a) in MeOH (1.00 L) was added K₂CO₃ (75.00 g, 542.65 mmol, 2.00 eq). The mixture was stirred at 15° C. for 18 hrs. TLC (DCM/MeOH=10/1, Rf=0.3) showed the reaction was complete. The mixture was filtered and the filtrate was concentrated under vacuum. The residue was purified by silica gel column (DCM/MeOH=40/1 to 20/1) to give the title compound (20.12 g, 134.84 mmol) as white solid. LCMS (method 2): Rt=0.315 min., m/z=150.1 (M+H)+.1H NMR (400 MHz, DMSO-d6): 4.89 (d, J=4.0 Hz, 1H), 3.79-3.76 (m, 1H), 3.53 (br, 1H), 3.04-2.89 (m, 4H), 1.97-1.86 (m, 4H).

### Step c

### N-[1-(5-{2-[(dimethylamino)methyl]phenyl}-2-thienyl)ethyl]-6-[(1-imino-1-oxidohexahydro-1lambda4-thiopyran-4-yl)oxy]-2-methylpyrido[3,4-d]pyrimidin-4-amine

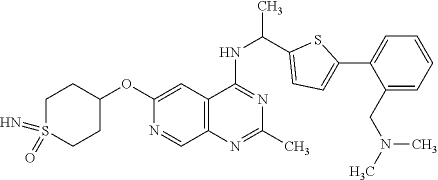 [SMILES: Cc1nc(NC(C)c2ccc(-c3ccccc3CN(C)C)s2)c2cc(OC3CCS(=N)(=O)CC3)ncc2n1]

Under argon a mixture of 1-imino-1-oxo-thian-4-ol (177 mg, 1.19 mmol, described in example 77, step b), sodium hydride (28 mg, 0.71 mmol) in 1-Methyl-2-pyrrolidon (3.5 ml) was stirred at ambient temperature for 20 minutes. N-[1-(5-{2-[(dimethylamino)methyl]phenyl}thiophen-2-yl)ethyl]-6-fluoro-2-methylpyrido [3,4-d]pyrimidin-4-amine (100 mg, 0.24 mmol, described in example 93, step b) were added and the reaction was stirred at 170° C. for six hours. The reaction mixture was allowed to cool to ambient temperature, poured into water and extracted with ethyl acetate (2×). The combined organic layers were washed with brine (1×), dried over sodium sulfate and the solvent was removed under reduced pressure. The residue was purified via HPCL chromatography to yield the title compound (9 mg, 6%). 1H-NMR (400 MHz, DMSO-d6): d [ppm]=8.74 (s, 1H), 8.65 (d, 1H), 7.73 (s, 1H), 7.42 (dd, 1H), 7.39-7.35 (m, 1H), 7.34-7.26 (m, 2H), 7.18 (d, 1H), 7.10 (dd, 1H), 5.89 (quin, 1H), 5.32 (tt, 1H), 3.75 (s, 1H), 3.35 (s, 2H), 3.11 (br s, 4H), 2.47 (s, 3H), 2.22 (br dd, 4H), 2.10 (s, 6H), 1.71 (d, 3H). LC-MS (Method 7): m/z: [M+H]+=551.2, Rₜ=0.53 min.

### EXAMPLE 78

### N-[(1R)-1-(3-bromophenyl)ethyl]-6-[2-(dimethylamino)ethoxy]-2-methylpyrido[3,4-d]pyrimidin-4-amine

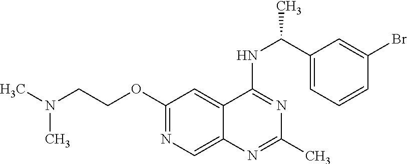 [SMILES: Cc1nc(N[C@H](C)c2cccc(Br)c2)c2cc(OCCN(C)C)ncc2n1]

In a sealed tube N-[(1R)-1-(3-bromophenyeethyl]-6-fluoro-2-methylpyrido[3,4-d]pyrimidin-4-amine (100 mg, 0.27 mmol, described in example 34), 2-Dimethylaminoaethanol (125 mg, 1.38 mmol, commercially available) and sodium hydride (18 mg, 0.44 mmol) in DMF (7 ml) were stirred at 170° C. overnight. The reaction mixture was cooled to ambient temperature and poured into ethyl acetate (100 ml). The organic layer was washed with water (3×50 ml), dried over sodium sulfate and the solvent was removed under reduced pressure. The residue was purified via HPLC chromatography to yield the title compound (1.2 mg, 0.9%). 1H-NMR (400 MHz, CD₃OD): d [ppm]=8.67 (s, 1H), 7.61 (t, 1H), 7.55 (s, 1H), 7.44-7.33 (m, 2H), 7.27-7.19 (m, 1H), 5.67-5.53 (m, 1H), 4.52 (t, 2H), 2.89 (t, 2H), 2.46 (s, 3H), 2.43 (s, 6H), 1.64 (d, 3H). LC-MS (Method 7): m/z: [M+H]⁺=430.1, Rₜ=0.60 min.

### EXAMPLE 79

### N-[(3R)-1-(4-{[1-(5-{2-[(dimethylamino)methyl]phenyl}thiophen-2-yl)ethyl]amino}-2-methylpyrido[3,4-d]pyrimidin-6-yl)pyrrolidin-3-yl]acetamide (diastereomer 1)

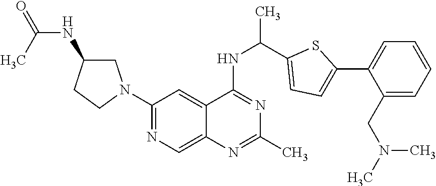 [SMILES: CC(=O)N[C@@H]1CCN(c2cc3c(NC(C)c4ccc(-c5ccccc5CN(C)C)s4)nc(C)nc3cn2)C1]

The diastereomeric mixture of N-[(3R)-1-(4-{[1-(5-{2-[(dimethylamino)methyl]phenyl}-thiophen-2-yl)ethyl]amino}-2-methylpyrido [3 ,4-d]pyrimidin-6-yl)pyrrolidin-3-yl]acetamide was prepared as described in example 60. The diastereomers were separated via preparative HPLC: Instrument: Labomatic HD5000, Labocord-5000; Gilson GX-241, Labcol Vario 4000; Column: Chiralpak ID 5 μm 250×30 mm; Eluent A: MTBE, Eluent B: Ethanol; isocratic 90% A +10% B +0.1 vol-% diethylamine (99%); flow 40.0 mL/min; UV 280 nm. Retention time of title compound: 6.9-8.8 minutes (>99.9% ee). 1H-NMR (400 MHz, DMSO-d6): d [ppm]=8.66 (s, 1H), 8.47 (d, 1H), 8.18 (d, 1H), 7.48-7.40 (m, 1H), 7.38-7.33 (m, 1H), 7.33-7.26 (m, 2H), 7.19 (d, 1H), 7.08 (dd, 1H), 7.00 (s, 1H), 5.94 (quin, 1H), 4.46-4.32 (m, 1H), 3.73-3.55 (m, 2H), 3.54-3.47 (m, 1H), 3.36 (s, 2H), 3.29 (dd, 1H), 2.42 (s, 3H), 2.24-2.15 (m, 1H), 2.11 (s, 6H), 1.97-1.87 (m, 1H), 1.81 (s, 2H), 1.72 (d, 3H). LC-MS (Method 7): m/z: [M+H]⁺=530.4, Rₜ=0.53 min. [□]_{D}=−284.2°+/−0.20° (MeOH).

### EXAMPLE 80

### N-[(3R)-1-(4-{[1-(5-{2-[(dimethylamino)methyl]phenyl}thiophen-2-yl)ethyl]amino}-2-methylpyrido[3,4-d]pyrimidin-6-yl)pyrrolidin-3-yl]acetamide (diastereomer 2)

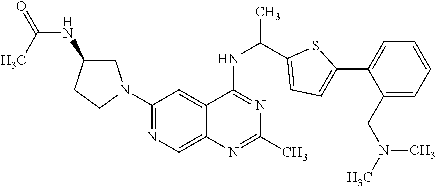 [SMILES: CC(=O)N[C@@H]1CCN(c2cc3c(NC(C)c4ccc(-c5ccccc5CN(C)C)s4)nc(C)nc3cn2)C1]

The diastereomeric mixture of N-[(3R)-1-(4-{[1-(5-{2-[(dimethylamino)methyl]phenyl}-thiophen-2-yl)ethyl]amino}-2-methylpyrido [3 ,4-d]pyrimidin-6-yl)pyrrolidin-3-yl]acetamide was prepared as described in example 60. The diastereomers were separated via preparative HPLC: Instrument: Labomatic HD5000, Labocord-5000; Gilson GX-241, Labcol Vario 4000; Column: Chiralpak ID 5 μm 250×30 mm; Eluent A: MTBE, Eluent B: Ethanol; isocratic 90% A +10% B +0.1 vol-% diethylamine (99%); flow 40.0 mL/min; UV 280 nm. Retention time of title compound: 10.3-12.7 minutes (99.5% ee). 1H-NMR (400 MHz, DMSO-d6): d [ppm]=8.66 (s, 1H), 8.47 (d, 1H), 8.18 (d, 1H), 7.42 (dd, 1H), 7.38-7.34 (m, 1H), 7.33-7.26 (m, 2H), 7.18 (d, 1H), 7.08 (dd, 1H), 7.00 (s, 1H), 5.93 (quin, 1H), 4.47-4.28 (m, 1H), 3.70-3.47 (m, 3H), 3.36 (s, 2H), 3.29 (dd, 1H), 2.42 (s, 3H), 2.24-2.13 (m, 1H), 2.10 (s, 6H), 1.98-1.87 (m, 1H), 1.81 (s, 3H), 1.72 (d, 3H). LC-MS (Method 7): m/z: [M+H]⁺=530.4, Rₜ=0.53 min [□]_{D}=+305.1°+/−2.22° (MeOH).

### EXAMPLE 81

### N-[(3S)-1-(4-{[1-(5-{2-[(dimethylamino)methyl]phenyl}thiophen-2-yl)ethyl]amino}-2-methylpyrido[3,4-d]pyrimidin-6-yl)pyrrolidin-3-yl]acetamide (diastereomer 1)

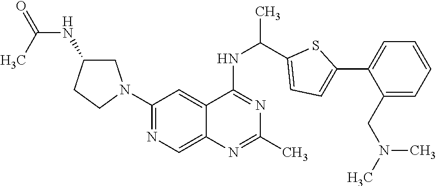 [SMILES: CC(=O)N[C@H]1CCN(c2cc3c(NC(C)c4ccc(-c5ccccc5CN(C)C)s4)nc(C)nc3cn2)C1]

The diastereomeric mixture of N-[(3S)-1-(4-{[1-(5-{2-[(dimethylamino)methyl]phenyl}-thiophen-2-yl)ethyl]amino}-2-methylpyrido[3 ,4-d]pyrimidin-6-yl)pyrrolidin-3-yl]acetamide was prepared as described in example 61. The diastereomers were separated via preparative HPLC: Instrument: Labomatic HD5000, Labocord-5000; Gilson GX-241, Labcol Vario 4000; Column: Chiralpak IF 5 μm 250×20 mm; Eluent A: Hexanes, Eluent B: Ethanol; gradient 5-50% B in 20 minutes +0.1 vol-% diethylamine (99%); flow 15.0 mL/min; UV 254 nm. Retention time of title compound: 14.9-15.9 minutes (99.6% ee). 1H-NMR (400 MHz, DMSO-d6): d [ppm]=8.66 (s, 1H), 8.57-8.45 (m, 1H), 8.18 (d, 2H), 7.49 (br s, 1H), 7.38 (br s, 3H), 7.18-7.06 (m, 2H), 7.00 (s, 1H), 5.94 (t, 1H), 4.44-4.30 (m, 1H), 3.71-3.45 (m, 4H), 3.30 (br d, 2H), 2.93 (br s, 1H), 2.42 (s, 3H), 2.27-2.11 (m, 4H), 2.01-1.90 (m, 1H), 1.81 (s, 3H), 1.73 (d, 3H), 1.15 (t, 2H). LC-MS (Method 7): m/z: [M+H]⁺=530.4, Rₜ=0.53 min. [□]_{D}=−287.9°+/−0.18° (MeOH).

### EXAMPLE 82

### N-[(3S)-1-(4-{[1-(5-{2-[(dimethylamino)methyl]phenyl}thiophen-2-yl)ethyl]amino}-2-methylpyrido[3,4-d]pyrimidin-6-yl)pyrrolidin-3-yl]acetamide (diastereomer 2)

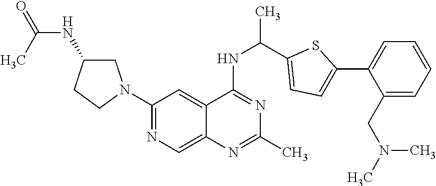 [SMILES: CC(=O)N[C@H]1CCN(c2cc3c(NC(C)c4ccc(-c5ccccc5CN(C)C)s4)nc(C)nc3cn2)C1]

The diastereomeric mixture of N-[(3S)-1-(4-{[1-(5-{2-[(dimethylamino)methyl]phenyl}-thiophen-2-yl)ethyl]amino}-2-methylpyrido [3 ,4-d]pyrimidin-6-yl)pyrrolidin-3-yl]acetamide was prepared as described in example 61. The diastereomers were separated via preparative HPLC: Instrument: Labomatic HD5000, Labocord-5000; Gilson GX-241, Labcol Vario 4000; Column: Chiralpak IF 5 μm 250×20 mm; Eluent A: Hexanes, Eluent B: Ethanol; gradient 5-50% B in 20 minutes +0.1 vol-% diethylamine (99%); flow 15.0 mL/min; UV 254 nm. Retention time of title compound: 16.3-17.4 minutes (94.8% ee). 1H-NMR (400 MHz, DMSO-d6): d [ppm]=8.66 (s, 1H), 8.50 (br d, 1H), 8.18 (d, 1H), 7.48 (br s, 1H), 7.38 (br s, 3H), 7.18-7.08 (m, 2H), 7.01 (s, 1H), 5.94 (quin, 1H), 4.44-4.31 (m, 1H), 3.75-3.57 (m, 2H), 3.55-3.47 (m, 1H), 3.31-3.23 (m, 1H), 2.92 (br s, 1H), 2.42 (s, 3H), 2.25-2.14 (m, 3H), 1.97-1.87 (m, 1H), 1.81 (s, 3H), 1.73 (d, 3H), 1.20-1.11 (m, 1H). LC-MS (Method 7): m/z: [M+H]+=530.4, Rₜ=0.53 min. [0 ]o =+249.1° +/-0.21° (MeOH).

### EXAMPLE 83

### N4-[(1R)-1-(3-bromophenyl)ethyl]-2-methyl-N6-[2-(methylsulfonypethyl]pyrido[3,4-d]-pyrimidine-4,6-diamine

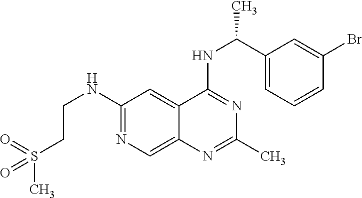 [SMILES: Cc1nc(N[C@H](C)c2cccc(Br)c2)c2cc(NCCS(C)(=O)=O)ncc2n1]

A solution of N-[(1R)-1-(3-bromophenyl)ethyl]-6-fluoro-2-methylpyrido [3 ,4-d]pyrimidin-4-amine (150 mg, 0.41 mmol, described in example 34), triethylamine (0.17 ml, 1.25 mmol) and 2-(Methylsulfonyl)ethanamine (256 mg, 2.08 mmol, commercially available) in 1-Methyl-2-pyrrolidon (6 ml) was stirred at 170° C. for one day. The reaction mixture was allowed to cool to ambient temperature, poured on ethyl acetate and extracted with aqueous NaOH (2×2N) and water (2×). The aqueous layers were re-extracted with ethyl acetate (2×) The combined organic layers were dried over sodium sulfate and the solvent was removed under reduced pressure. The crude product was purified by HPLC chromatography to yield 3% (6 mg) of the title compound. 1H-NMR (400 MHz, DMSO-d6): d [ppm]=8.60 (s, 1H), 8.33 (br d, 1H), 7.64 (s, 1H), 7.47-7.37 (m, 2H), 7.34-7.24 (m, 1H), 7.11 (s, 1H), 6.63 (br s, 1H), 5.56 (quin, 1H), 3.71 (q, 2H), 3.45 (t, 2H), 3.05 (s, 3H), 2.33 (s, 3H), 1.57 (d, 3H). LC-MS (Method 7): m/z: [M+H]⁺=466.1, Rₜ=0.76 min.

### EXAMPLE 84

### N4-[(1R)-1-(3-bromophenyl)ethyl]-N6-[2-(1H-imidazol-1-yl)ethyl]-2-methylpyrido[3,4-d]pyrimidine-4,6-diamine

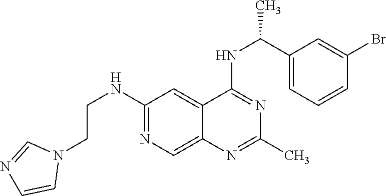 [SMILES: Cc1nc(N[C@H](C)c2cccc(Br)c2)c2cc(NCCn3ccnc3)ncc2n1]

A solution of N-[(1R)-1-(3-bromophenyl)ethyl]-6-fluoro-2-methylpyrido[3,4-d]pyrimidin-4-amine (150 mg, 0.41 mmol, described in example 34), triethylamine (0.17 ml, 1.25 mmol) and 2-(1H-Imidazol-1-yl)ethanamine (243 mg, 2.08 mmol, commercially available) in 1-Methyl-2-pyrrolidon (6 ml) was stirred at 170° C. for one day. The reaction mixture was allowed to cool to ambient temperature, poured on ethyl acetate and extracted with aqueous NaOH (2×2N) and water (2×). The aqueous layers were re-extracted with ethyl acetate (2×) The combined organic layers were dried over sodium sulfate and the solvent was removed under reduced pressure. The crude product was purified by HPLC chromatography to yield 8% (14 mg) of the title compound. 1H-NMR (400 MHz, DMSO-d6): d [ppm]=8.56 (s, 1H), 8.24 (d, 1H), 7.65-7.61 (m, 2H), 7.46-7.38 (m, 2H), 7.33-7.25 (m, 1H), 7.21 (t, 1H), 7.01 (s, 1H), 6.87 (t, 1H), 6.64 (t, 1H), 5.55 (quin, 1H), 4.23 (t, 2H), 3.59 (q, 2H), 2.70-2.64 (m, 1H), 2.33 (s, 4H), 1.56 (d, 3H). LC-MS (Method 7): m/z: [M+H]⁺=454.1, Rₜ=0.58 min.

### EXAMPLE 85

### 3-(4-{[1-(5-{2-[(dimethylamino)methyl]phenyl}thiophen-2-yl)ethyl]amino}-2-methylpyrido[3,4-d]pyrimidin-6-yl)-3-hydroxy-1-methylpyrrolidin-2-one

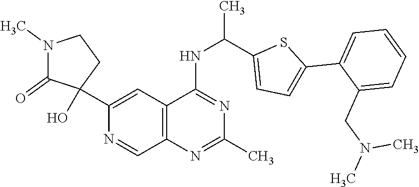 [SMILES: Cc1nc(NC(C)c2ccc(-c3ccccc3CN(C)C)s2)c2cc(C3(O)CCN(C)C3=O)ncc2n1]

The title compound was observed as a byproduct in the following reaction: To a solution of 4-hydroxy-2-piperidinone (55 mg, 0.47 mmol, commercially available) in 1-Methyl-2-pyrrolidon (3.5 ml) was added under argon NaH (38 mg, 0.95 mmol, 60% in mineral oil) and the reaction was stirred at ambient temperature for 20 minutes. N-[1-(5-{2-[(dimethylamino)methyl]phenyl}thiophen-2-yl)ethyl]-6-fluoro-2-methylpyrido[3,4-d]pyrimidin-4-amine (100 mg, 0.24 mmol, described in example 93, step b) were added and the reaction was stirred at 170° C. for six hours. Another portion of NaH (30 mg, 0.75 mmol, 60% in mineral oil) was added and the reaction was stirred at 170° C. overnight. The reaction mixture was allowed to cool to ambient temperature, poured into water and extracted with ethyl acetate (2×). The combined organic layers were washed with brine (1×), dried over sodium sulfate and the solvent was removed under reduced pressure. The title compound was obtained after HPCL chromatography (acetonitrile 30-70%, basic) in 7% yield (9 mg)as a mixture of diastereomers. 1H-NMR (600 MHz, DMSO-d6): d [ppm]=9.15 (t, 1H), 8.90 (s, 1H), 8.51 (d, 1H), 7.42 (ddd, 1H), 7.39-7.35 (m, 1H), 7.34-7.26 (m, 2H), 7.19 (dd, 1H), 7.10 (ddd, 1H), 6.32 (d, 1H), 5.97 (td, 1H), 3.55-3.45 (m, 2H), 3.35 (d, 2H), 2.82 (d, 3H), 2.70-2.63 (m, 1H), 2.52 (s, 3H), 2.24-2.16 (m, 1H), 2.10 (d, 6H), 1.74 (dd, 3H). LC-MS (Method 7): m/z: [M+H]⁺=517.2 & 517.2, Rₜ=0.53 & 0.56 min.

### EXAMPLE 86

### 4-{[(4-{[1-(5-{2-[(dimethylamino)methyl]phenyl}thiophen-2-yl)ethyl]amino}-2-methylpyrido[3,4-d]pyrimidin-6-yl)amino]methyl}piperidin-2-one

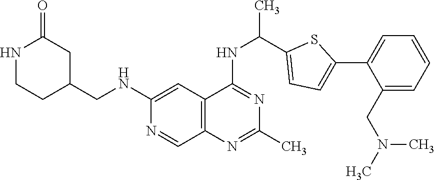 [SMILES: Cc1nc(NC(C)c2ccc(-c3ccccc3CN(C)C)s2)c2cc(NCC3CCNC(=O)C3)ncc2n1]

To a solution of N-[1-(5-{2-[(dimethylamino)methyl]phenyl}thiophen-2-yl)ethyl]-6-fluoro-2-methylpyrido[3,4-d]pyrimidin-4-amine (100 mg, 0.24 mmol, described in example 93, step b) in 1-Methyl-2-pyrrolidon (3 ml) was added under argon triethylamin (248 μl, 1.78 mmol) and 4-aminoethyl-2-piperidone (152 mg, 1.19 mmol, commercially available) and the reaction was stirred at 170° C. overnight. Triethylamin 0 was added and the reaction was stirred for another 9 hours. The reaction was allowed to cool to ambient temperature and extracted with ethyl acetate/water (3×). The combined organic layers were washed with brine, dried over sodium sulfate and the solvent was distilled off under reduced pressure. The crude product was purified via a PrepCon HPCL chromatography (acetonitrile 30-70%, basic conditions) to yield the title compound (10.7 mg, 7.7%). 1H-NMR (400 MHz, DMSO-d6): d [ppm]=8.56 (s, 1H), 8.39 (d, 1H), 7.51-7.46 (m, 1H), 7.44-7.40 (m, 1H), 7.39-7.35 (m, 1H), 7.33-7.28 (m, 2H), 7.18 (d, 1H), 7.08 (dd, 1H), 6.93 (s, 1H), 6.63 (q, 1H), 6.52 (t, 1H), 5.97-5.86 (m, 1H), 4.19 (s, 1H), 3.35 (s, 2H), 3.26-3.14 (m, 2H), 2.91 (dd, 1H), 2.73-2.68 (m, 1H), 2.54 (s, 2H), 2.41 (s, 3H), 2.35-2.23 (m, 2H), 2.20-2.11 (m, 2H), 2.10 (s, 6H), 1.96-1.85 (m, 2H), 1.72 (br d, 3H). LC-MS (Method 7): m/z: [M+H]⁺=530.3, Rₜ=0.52 min.

### EXAMPLE 87

### N-[1-(4-{[1-(5-{2-[(dimethylamino)methyl]phenyl}thiophen-2-yl)ethyl]amino}-2-methylpyrido[3,4-d]pyrimidin-6-yl)azetidin-3-yl]-4-[4,5-dimethyl-2-(pyridin-2-yl)-1H-imidazol-1-yl]butanamide

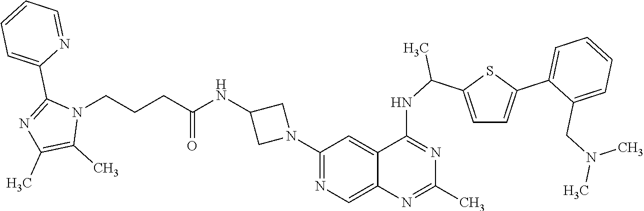 [SMILES: Cc1nc(NC(C)c2ccc(-c3ccccc3CN(C)C)s2)c2cc(N3CC(NC(=O)CCCn4c(-c5ccccn5)nc(C)c4C)C3)ncc2n1]

A solution of 6-(3-aminoazetidin-1-yl)-N-[1-(5-{2-[(dimethylamino)methyl]phenyl}thiophen-2-yl)ethyl]-2-methylpyrido[3,4-d]pyrimidin-4-amine (100 mg, 211 μmol, described in example 93), 4-[4,5-dimethyl-2-(pyridin-2-yl)-1H-imidazol-1-yl]butanoic acid (54.7 mg, 211 ittmol), PyBOP (220 mg, 422 μmol) and N,N-diisopropylethylamine (180 μL, 1.1 mmol) in THF (2.0 mL) was stirred at room temperature overnight. H₂O was added, the mixture extracted with DCM and the solvent removed in vacuo. Purification by preparative HPLC (basic conditions) gave the title compound as a yellow solid (44.7 mg, 28%). ¹H-NMR (400 MHz, DMSO-d₆): δ [ppm]=8.65 (s, 1H), 8.53 (br dd, 1H), 8.47 (t, 2H), 7.99 (d, 1H), 7.79 (td, 1H), 7.44-7.40 (m, 1H), 7.38-7.34 (m, 1H), 7.34-7.28 (m, 2H), 7.25 (ddd, 1H), 7.18 (d, 1H), 7.08 (dd, 1H), 7.07 (s, 1H), 5.92 (quin, 1H), 4.65-4.54 (m, 1H), 4.47 (t, 2H), 4.24 (t, 2H), 3.79-3.73 (m, 2H), 3.34 (br s, 2H), 2.43 (s, 3H), 2.17 (s, 3H), 2.13-2.10 (m, 2H), 2.09 (s, 9H), 1.86 (quin, 2H), 1.71 (d, 3H). LC-MS (Method 10): m/z: [M+H]⁺=715, Rₜ=1.33 min.

### EXAMPLE 88

### N-[1-(4-{[1-(5-{2-[(dimethylamino)methyl]phenyl}thiophen-2-yl)ethyl]amino}-2-methylpyrido[3,4-d]pyrimidin-6-yl)azetidin-3-yl]benzamide

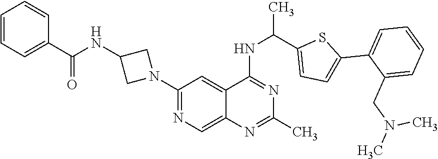 [SMILES: Cc1nc(NC(C)c2ccc(-c3ccccc3CN(C)C)s2)c2cc(N3CC(NC(=O)c4ccccc4)C3)ncc2n1]

A solution of 6-(3-aminoazetidin-1-yl)-N-[1-(5-{2-[(dimethylamino)methyl]phenyl}thiophen-2-yl)ethyl]-2-methylpyrido[3,4-d]pyrimidin-4-amine (100 mg, 211 μmol, described in example 93), benzoic acid (25.8 mg, 211 μmol), PyBOP (220 mg, 422 μmol) and N,N-diisopropylethylamine (180 μL, 1.1 mmol) in THF (2.0 mL) was stirred at room temperature overnight. H₂O was added, the mixture extracted with DCM and the solvent removed in vacuo. Purification by preparative HPLC (basic conditions) gave the title compound as a yellow solid (33.5 mg, 26%). ¹H-NMR (400 MHz, DMSO-d₆): δ [ppm]=9.03 (d, 1H), 8.68 (s, 1H), 8.50 (d, 1H), 7.89 (s, 1H), 7.87 (d, 1H), 7.57-7.51 (m, 1H), 7.50-7.44 (m, 2H), 7.42 (dd, 1H), 7.38-7.35 (m, 1H), 7.34-7.26 (m, 2H), 7.19 (d, 1H), 7.12 (s, 1H), 7.09 (dd, 1H), 5.93 (quin, 1H), 4.90 (sxt, 1H), 4.35 (t, 2H), 4.00 (t, 2H), 3.35 (br s, 2H), 2.44 (s, 3H), 2.10 (s, 6H), 1.72 (d, 3H). LC-MS (Method 10): m/z: [M+H]⁺=578, Rₜ=1.40 min.

### EXAMPLE 89

### N-[1-(4-{[1-(5-{2-[(dimethylamino)methyl]phenyl}thiophen-2-yl)ethyl]amino}-2-methylpyrido[3,4-d]pyrimidin-6-yl)azetidin-3-yl]-2-phenylacetamide

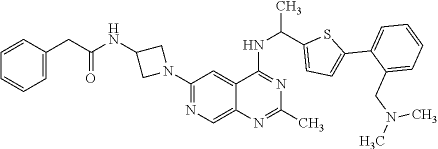 [SMILES: Cc1nc(NC(C)c2ccc(-c3ccccc3CN(C)C)s2)c2cc(N3CC(NC(=O)Cc4ccccc4)C3)ncc2n1]

A solution of 6-(3-aminoazetidin-1-yl)-N-[1-(5-{2-[(dimethylamino)methyl]phenyl}thiophen-2-yl)ethyl]-2-methylpyrido[3,4-d]pyrimidin-4-amine (100 mg, 211 μmol, described in example 93), phenylacetic acid (28.7 mg, 211 μmol), PyBOP (220 mg, 422 μmol) and N,N-diisopropylethylamine (180 μL, 1.1 mmol) in THF (2.0 mL) was stirred at room temperature overnight. H₂O was added, the mixture extracted with DCM and the solvent removed in vacuo. Purification by preparative HPLC (basic conditions) gave the title compound as a yellow solid (37.3 mg, 28%). ¹H-NMR (400 MHz, DMSO-d₆): δ [ppm]=8.77 (d, 1H), 8.66 (s, 1H), 8.46 (d, 1H), 7.44-7.41 (m, 1H), 7.38-7.35 (m, 1H), 7.34-7.28 (m, 3H), 7.28-7.24 (m, 3H), 7.23-7.20 (m, 1H), 7.19 (d, 1H), 7.10-7.06 (m, 2H), 5.92 (quin, 1H), 4.66-4.56 (m, 1H), 4.26 (td, 2H), 3.79 (ddd, 2H), 3.42 (s, 2H), 3.35 (s, 2H), 2.43 (s, 3H), 2.10 (s, 6H), 1.71 (d, 3H). LC-MS (Method 10): m/z: [M+H]⁺=592, Rₜ=1.41 min.

### EXAMPLE 90

### N-[1-(4-{[1-(5-{2-[(dimethylamino)methyl]phenyl}thiophen-2-yl)ethyl]amino}-2-methylpyrido[3,4-d]pyrimidin-6-yl)azetidin-3-yl]cyclohexanecarboxamide

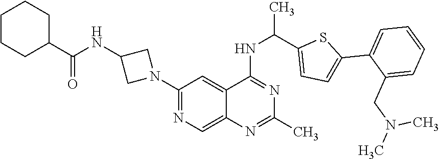 [SMILES: Cc1nc(NC(C)c2ccc(-c3ccccc3CN(C)C)s2)c2cc(N3CC(NC(=O)C4CCCCC4)C3)ncc2n1]

A solution of 6-(3-aminoazetidin-1-yl)-N-[1-(5-{2-[(dimethylamino)methyl]phenyl}thiophen-2-yl)ethyl]-2-methylpyrido[3,4-d]pyrimidin-4-amine (100 mg, 211 μmol, described in example 93), cyclohexanecarboxylic acid (27.1 mg, 211 μmol), PyBOP (220 mg, 422 μmol) and N,N-diisopropylethylamine (180 μL, 1.1 mmol) in THF (2.0 mL) was stirred at room temperature overnight. H₂O was added, the mixture extracted with DCM and the solvent removed in vacuo. Purification by preparative HPLC (basic conditions) gave the title compound as a yellow solid (34.3 mg, 26%). ¹H-NMR (400 MHz, DMSO-d₆): δ [ppm]=8.66 (s, 1H), 8.46 (d, 1H), 8.36 (d, 1H), 7.42 (dd, 1H), 7.38-7.35 (m, 1H), 7.34-7.26 (m, 2H), 7.18 (d, 1H), 7.09 (dd, 1H), 7.07 (s, 1H), 5.92 (quin, 1H), 4.66-4.53 (m, 1H), 4.24 (t, 2H), 3.77 (br t, 2H), 3.35 (s, 2H), 2.43 (s, 3H), 2.14-2.04 (m, 8H), 1.74-1.67 (m, 7H), 1.60 (br d, 1H), 1.39-1.26 (m, 2H), 1.25-1.10 (m, 2H). LC-MS (Method 10): m/z: [M+H]⁺=584, Rₜ=1.46 min.

### EXAMPLE 91

### 2-cyclopropyl-N-[1-(4-{[1-(5-{2-[(dimethylamino)methyl]phenyl}thiophen-2-yl)ethyl]amino}-2-methylpyrido[3,4-d]pyrimidin-6-yl)azetidin-3-yl]acetamide

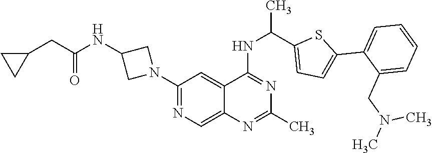 [SMILES: Cc1nc(NC(C)c2ccc(-c3ccccc3CN(C)C)s2)c2cc(N3CC(NC(=O)CC4CC4)C3)ncc2n1]

A solution of 6-(3-aminoazetidin-1-yl)-N-[1-(5-{2-[(dimethylamino)methyl]phenyl}thiophen-2-yl)ethyl]-2-methylpyrido[3,4-d]pyrimidin-4-amine (100 mg, 211 μmol, described in example 93), cyclopropylacetic acid (21.1 mg, 211 μmol), PyBOP (220 mg, 422 μmol) and N,N-diisopropylethylamine (180 μL, 1.1 mmol) in THF (2.0 mL) was stirred at room temperature overnight. H₂O was added, the mixture extracted with DCM and the solvent removed in vacuo. Purification by preparative HPLC (basic conditions) gave the title compound as a yellow solid (31.9 mg, 26%). ¹H-NMR (400 MHz, DMSO-d₆): δ [ppm]=8.66 (s, 1H), 8.47 (br d, 1H), 8.40 (d, 1H), 7.45-7.40 (m, 1H), 7.38-7.35 (m, 1H), 7.34-7.26 (m, 2H), 7.19 (d, 1H), 7.11-7.06 (m, 2H), 5.92 (br quin, 1H), 4.70-4.58 (m, 1H), 4.26 (t, 2H), 3.79 (dd, 2H), 3.35 (br s, 2H), 2.43 (s, 3H), 2.10 (s, 6H), 1.99 (d, 2H), 1.72 (d, 3H), 1.02-0.89 (m, 1H), 0.46-0.38 (m, 2H), 0.11 (q, 2H). LC-MS (Method 10): m/z: [M+H]⁺=556, Rₜ=1.35 min.

### EXAMPLE 92

### N-[1-(4-{[1-(5-{2-[(dimethylamino)methyl]phenyl}thiophen-2-yl)ethyl]amino}-2-methylpyrido[3,4-d]pyrimidin-6-yl)azetidin-3-yl]-2-(morpholin-4-yl)acetamide

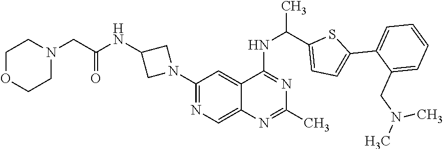 [SMILES: Cc1nc(NC(C)c2ccc(-c3ccccc3CN(C)C)s2)c2cc(N3CC(NC(=O)CN4CCOCC4)C3)ncc2n1]

A solution of 6-(3-aminoazetidin-1-yl)-N-[1-(5-{2-[(dimethylamino)methyl]phenyl}thiophen-2-yl)ethyl]-2-methylpyrido[3,4-d]pyrimidin-4-amine (100 mg, 211 μmol, described in example 93), morpholin-4-ylacetic acid (30.6 mg, 211 μmol), PyBOP (220 mg, 422 μmol) and N,N-diisopropylethylamine (180 μL, 1.1 mmol) in THF (2.0 mL) was stirred at room temperature overnight. H₂O was added, the mixture extracted with DCM and the solvent removed in vacuo. Purification by preparative HPLC (basic conditions) gave the title compound as a yellow solid (37.5 mg, 28%). ¹H-NMR (400 MHz, DMSO-d₆): δ [ppm]=8.66 (s, 1H), 8.48 (d, 1H), 8.42 (d, 1H), 7.44-7.40 (m, 1H), 7.38-7.35 (m, 1H), 7.34-7.26 (m, 2H), 7.19 (d, 1H), 7.10-7.07 (m, 2H), 5.92 (quin, 1H), 4.77-4.66 (m, 1H), 4.24 (td, 2H), 3.89 (dd, 2H), 3.61-3.57 (m, 4H), 3.35 (s, 2H), 2.93 (s, 2H), 2.43 (s, 3H), 2.42-2.39 (m, 4H), 2.10 (s, 6H), 1.72 (d, 3H). LC-MS (Method 10): m/z: [M+H]⁺=601, Rₜ=1.29 min.

### EXAMPLE 93

### 6-(3-aminoazetidin-1-yl)-N-[1-(5-{2-[(dimethylamino)methyl]phenyl}thiophen-2-yl)ethyl]-2-methylpyrido[3,4-d]pyrimidin-4-amine

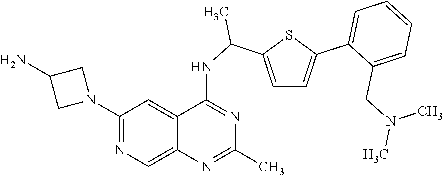 [SMILES: Cc1nc(NC(C)c2ccc(-c3ccccc3CN(C)C)s2)c2cc(N3CC(N)C3)ncc2n1]

### Step a

### 2-(5-{1-[(6-fluoro-2-methylpyrido[3,4-d]pyrimidin-4-y0amino]ethyl}thiophen-2-yl)benzaldehyde

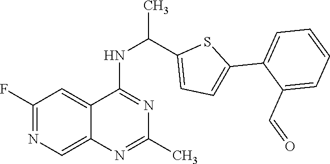 [SMILES: Cc1nc(NC(C)c2ccc(-c3ccccc3C=O)s2)c2cc(F)ncc2n1]

Under argon, N-[1-(5-bromothiophen-2-yl)ethyl]-6-fluoro-2-methylpyrido[3,4-d]pyrimidin-4-amine (5.07 g, 13.8 mmol, described in example 35), (2-formylphenyl)boronic acid (2.28 g, 15.2 mmol), K₂CO₃ (7.63 g, 55.2 mmol) and Pd(PPh3)4 (1.60 g, 1.38 mmol) in dioxane (69 mL) and H₂O (14 mL) were stirred at 110° C. for 8 hours. H₂O was added, the mixture extracted with DCM and the solvent removed in vacuo. Purification by column chromatography (silica gel, MeOH/EtOAc 0-10%) gave the title compound (5.90 g, quantitative). LC-MS (Method 10): m/z: [M+H]⁺=393, Rₜ=1.30 min.

### Step b

### N-[1-(5-{2-[(dimethylamino)methyl]phenyl}thiophen-2-yl)ethyl]-6-fluoro-2-methylpyrido[3,4-d]pyrimidin-4-amine

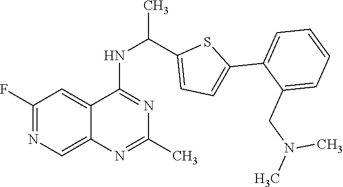 [SMILES: Cc1nc(NC(C)c2ccc(-c3ccccc3CN(C)C)s2)c2cc(F)ncc2n1]

To 2-(5-{1-[(6-fluoro-2-methylpyrido[3,4-d]pyrimidin-4-yl)amino]ethyl}thiophen-2-yl)benzaldehyde (5.42 g, 13.8 mmol), N-methylmethanamine (14 mL, 2.0 M, 28 mmol) and acetic acid (1.6 mL, 28 mmol) in 1,2-dichloroethane (140 mL) was added NaBH(OAc)₃ (5.85 g, 27.6 mmol) and the solution stirred at room temperature overnight. The reaction was quenched with aqueous NaOH (1M, 1.0 mL), the mixture extracted with DCM and the solvent removed in vacuo. Purification by column chromatography (silica gel, MeOH/EtOAc 0-20% then MeOH/EtOAc 20%) gave the title compound as a light brown solid (5.59 g, 96%). LC-MS (Method 10): m/z: [M+H]⁺=422, Rₜ=1.45 min.

### Step c

### tert-butyl [1-(4-{[1-(5-{2-[(dimethylamino)methyl]phenyl}thiophen-2-yl)ethyl]amino}-2-methylpyrido[3,4-d]pyrimidin-6-yl)azetidin-3-yl]carbamate

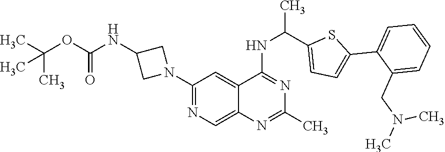 [SMILES: Cc1nc(NC(C)c2ccc(-c3ccccc3CN(C)C)s2)c2cc(N3CC(NC(=O)OC(C)(C)C)C3)ncc2n1]

A solution of N-[1-(5-{2-[(dimethylamino)methyl]phenyl}thiophen-2-yl)ethyl]-6-fluoro-2-methylpyrido[3,4-d]pyrimidin-4-amine (3.00 g, 7.12 mmol), tert-butyl azetidin-3-ylcarbamate (3.68 g, 21.4 mmol) and triethylamine (5.0 mL, 36 mmol) in DMSO (70 mL) was stirred at 120° C. over the weekend. The reaction mixture was poured into H₂O (250 mL) and stirred at room temperature during 10 minutes (formation of a brown gunk). The solvent was poured out and the viscous solid dried in vacuo to give the title compound. LC-MS (Method 10): m/z: [M+H]⁺=574, Rₜ=1.46 min.

### Step d

### 6-(3-aminoazetidin-1-yl)-N-[(1S)-1-(5-{2-[(dimethylamino)methyl]phenyl}thiophen-2-yl)-ethyl]-2-methylpyrido[3,4-d]pyrimidin-4-amine

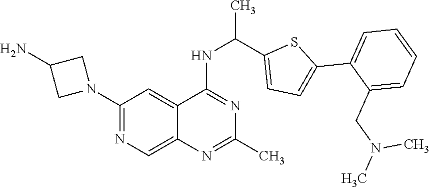 [SMILES: Cc1nc(NC(C)c2ccc(-c3ccccc3CN(C)C)s2)c2cc(N3CC(N)C3)ncc2n1]

To a solution of tert-butyl [1-(4-{[1-(5-{2-[(dimethylaminolmethyl]phenyl}thiophen-2-yl)ethyl]amino}-2-methylpyrido[3,4-d]pyrimidin-6-yl)azetidin-3-yl]carbamate (4.08 g, 7.12 mmol) in DCM (70 mL) was added TFA (5.5 mL, 71 mmol) and the reaction mixture stirred at room temperature overnight. The solvent was removed in vacuo and the residue then taken up in Et₂O and stirred during 2 hours. The mixture was left to settle, the solvent poured out, the residue taken up in Et₂O again and stirred during 30 minutes. The obtained precipitate was then filtered and dried to give the title compound as a yellow solid (2.23 g, 66%).¹H-NMR (400 MHz, DMSO-d₆): δ [ppm]=8.63 (s, 1H), 8.47 (d, 1H), 7.44-7.40 (m, 1H), 7.38-7.35 (m, 1H), 7.34-7.26 (m, 2H), 7.18 (d, 1H), 7.08 (dd, 1H), 7.01 (s, 1H), 5.92 (quin, 1H), 4.16 (t, 2H), 3.83 (quin, 1H), 3.61-3.54 (m, 2H), 3.35 (s, 2H), 2.42 (s, 3H), 2.10 (s, 6H), 1.72 (d, 3H). LC-MS (Method 10): m/z: [M+H]⁺=474, Rₜ=1.20 min.

### EXAMPLE 94

### N-[1-(4-{[1-(5-{2-[(dimethylamino)methyl]phenyl}thiophen-2-yl)ethyl]amino}-2-methylpyrido[3,4-d]pyrimidin-6-yl)azetidin-3-yl]benzenesulfonamide

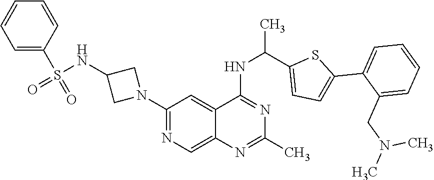 [SMILES: Cc1nc(NC(C)c2ccc(-c3ccccc3CN(C)C)s2)c2cc(N3CC(NS(=O)(=O)c4ccccc4)C3)ncc2n1]

To a solution of 6-(3-aminoazetidin-1-yl)-N-[1-(5-{2-[(dimethylamino)methyl]phenyl}-thiophen-2-yl)ethyl]-2-methylpyrido[3,4-d]pyrimidin-4-amine (100 mg, 211 μmol, described in example 93) and triethylamine (150 μL, 1.1 mmol) in THF (2.0 mL) was added benzenesulfonyl chloride (32 μL, 250 μmol) dropwise and the reaction mixture then stirred at room temperature overnight. H₂O was added, the mixture extracted with DCM and the solvent removed in vacuo. Purification by preparative HPLC (basic conditions, twice) gave the title compound as a yellow solid (6.90 mg, 5%). ¹H-NMR (400 MHz, DMSO-d₆): δ [ppm]=7.79-7.74 (m, 2H), 7.70-7.59 (m, 3H), 7.46-7.42 (m, 1H), 7.39-7.35 (m, 1H), 7.34-7.24 (m, 4H), 7.15 (d, 1H), 6.98 (dd, 1H), 5.70 (quin, 1H), 4.52 (br d, 1H), 4.33 (br d, 1H), 3.63 (br s, 2H), 3.36 (s, 2H), 2.37 (br s, 3H), 2.12 (s, 6H), 1.60 (d, 3H). LC-MS (Method 10): m/z: [M+H]⁺=614, Rₜ=0.76 min.

### EXAMPLE 95

### N-[1-(4-{[1-(5-{2-[(dimethylamino)methyl]phenyl}thiophen-2-yl)ethyl]amino}-2-methylpyrido[3,4-d]pyrimidin-6-yl)azetidin-3-yl]-1-phenylmethanesulfonamide

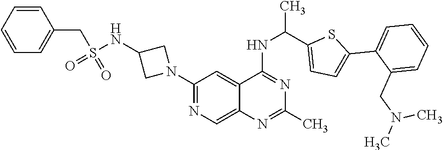 [SMILES: Cc1nc(NC(C)c2ccc(-c3ccccc3CN(C)C)s2)c2cc(N3CC(NS(=O)(=O)Cc4ccccc4)C3)ncc2n1]

To a solution of 6-(3-aminoazetidin-1-yl)-N-[1-(5-{2-[(dimethylamino)methyl]phenyl}-thiophen-2-yl)ethyl]-2-methylpyrido[3,4-d]pyrimidin-4-amine (100 mg, 211 μmol, described in example 93) and triethylamine (150 μL, 1.1 mmol) in THF (1.0 mL) was added phenylmethanesulfonyl chloride (48.3 mg, 253 μmol) in 1.0 mL THF dropwise and the reaction mixture then stirred at room temperature overnight. H₂O was added, the mixture extracted with DCM and the solvent removed in vacuo. Purification by preparative HPLC (basic conditions, twice) gave the title compound as a yellow solid (13.0 mg, 8%). ¹H-NMR (400 MHz, DMSO-d₆): δ [ppm]=7.43 (dd, 1H), 7.40-7.32 (m, 7H), 7.32-7.25 (m, 4H), 7.16 (d, 1H), 7.00 (dd, 1H), 5.75 (quin, 1H), 4.57 (d, 1H), 4.43-4.34 (m, 2H), 4.32 (d, 2H), 3.36 (s, 3H), 2.39 (s, 3H), 2.12 (s, 6H), 1.65 (d, 3H). LC-MS (Method 10): m/z: [M+H]⁺=628, Rₜ=0.74 min.

### EXAMPLE 96

### 1-[1-(4-{[1-(5-{2-[(dimethylamino)methyl]phenyl}thiophen-2-yl)ethyl]amino}-2-methylpyrido[3,4-d]pyrimidin-6-yl)azetidin-3-yl]-3-phenylurea

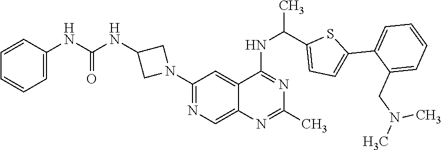 [SMILES: Cc1nc(NC(C)c2ccc(-c3ccccc3CN(C)C)s2)c2cc(N3CC(NC(=O)Nc4ccccc4)C3)ncc2n1]

A solution of 6-(3-aminoazetidin-1-yl)-N-[1-(5-{2-[(dimethylamino)methyl]phenyl}thiophen-2-yl)ethyl]-2-methylpyrido[3,4-d]pyrimidin-4-amine (100 mg, 211 μmol, described in example 93), isocyanatobenzene (23 μL, 210 μmol) and N,N-diisopropylethylamine (180 μL, 1.1 mmol) in THF (2.0 mL) was stirred at room temperature overnight. H₂O was added, the mixture extracted with DCM and the solvent removed in vacuo. Purification by preparative HPLC (basic conditions) gave the title compound as a yellow solid (13.0 mg, 8%). ¹H-NMR (400 MHz, DMSO-d₆): δ [ppm]=8.67 (d, 1H), 8.52 (s, 1H), 8.48 (d, 1H), 7.44-7.35 (m, 4H), 7.33-7.26 (m, 2H), 7.25-7.17 (m, 3H), 7.10-7.07 (m, 2H), 6.90 (tt, 1H), 6.87 (d, 1H), 5.93 (quin, 1H), 4.68-4.56 (m, 1H), 4.29 (t, 2H), 3.82 (dd, 2H), 3.35 (s, 2H), 2.44 (s, 3H), 2.10 (s, 6H), 1.75-1.68 (m, 3H). LC-MS (Method 10): m/z: [M+H]⁺=593, Rₜ=1.35 min.

### EXAMPLE 97

### tert-butyl (3R)-3-[acetyl(3-{[1-(4-{[1-(5-{2-[(dimethylamino)methyl]phenyl}-2-thienyl)-ethyl]amino}-2-methylpyrido[3,4-d]pyrimidin-6-yl)azetidin-3-yl]carbamoyl}-phenyl)amino]pyrrolidine-1-carboxylate

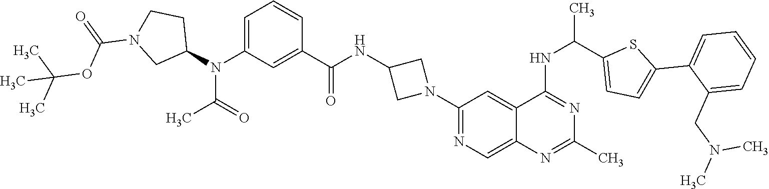 [SMILES: CC(=O)N(c1cccc(C(=O)NC2CN(c3cc4c(NC(C)c5ccc(-c6ccccc6CN(C)C)s5)nc(C)nc4cn3)C2)c1)[C@@H]1CCN(C(=O)OC(C)(C)C)C1]

A solution of 6-(3-aminoazetidin-1-yl)-N-[1-(5-{2-[(dimethylamino)methyl]phenyl}thiophen-2-yl)ethyl]-2-methylpyrido[3,4-d]pyrimidin-4-amine (100 mg, 211 μmol, described in example 93), 3-{acetyl[(3R)-1-(tert-butoxycarbonyl)pyrrolidin-3-yl]amino}benzoic acid (73.6 mg, 211 μmol), PyBOP (220 mg, 422 μmol) and N,N-diisopropylethylamine (180 μL, 1.1 mmol) in THF (2.0 mL) was stirred at room temperature overnight. H₂O was added, the mixture extracted with DCM and the solvent removed in vacuo. Purification by preparative HPLC (basic conditions) gave the title compound as a yellow solid (8.90 mg, 5%). ¹H-NMR (400 MHz, DMSO-d₆): δ [ppm]=9.06 (d, 1H), 8.68 (s, 1H), 8.50 (d, 1H), 8.18 (s, 1H), 7.94 (d, 1H), 7.79 (br s, 1H), 7.60-7.53 (m, 1H), 7.49 (br s, 1H), 7.42 (dd, 1H), 7.38-7.34 (m, 1H), 7.33-7.26 (m, 2H), 7.18 (d, 1H), 7.13 (s, 1H), 7.09 (dd, 1H), 5.93 (quin, 1H), 4.92 (dt, 2H), 4.36 (t, 2H), 4.05-3.97 (m, 2H), 3.61-3.48 (m, 1H), 3.11-3.00 (m, 2H), 2.90 (br s, 1H), 2.44 (s, 3H), 2.10 (s, 6H), 1.72 (d, 3H), 1.65 (s, 3H), 1.29 (s, 9H). LC-MS (Method 10): m/z: [M±H]⁺=804, Rₜ=1.42 min.

### EXAMPLE 98

### tert-butyl 4-[acetyl(3-{[1-(4-{[1-(5-{2-[(dimethylamino)methyl]phenyl}thiophen-2-yl)ethyl]amino}-2-methylpyrido[3,4-d]pyrimidin-6-yl)azetidin-3-yl]carbamoyl}-phenyl)amino]piperidine-1-carboxylate

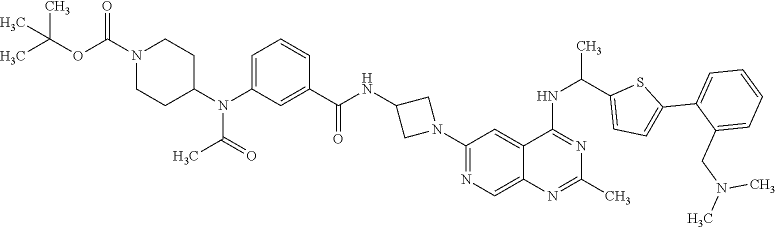 [SMILES: CC(=O)N(c1cccc(C(=O)NC2CN(c3cc4c(NC(C)c5ccc(-c6ccccc6CN(C)C)s5)nc(C)nc4cn3)C2)c1)C1CCN(C(=O)OC(C)(C)C)CC1]

A solution of 6-(3-aminoazetidin-1-yl)-N-[1-(5-{2-[(dimethylamino)methyl]phenyl }thiophen-2-yl)ethyl]-2-methylpyrido[3,4-d]pyrimidin-4-amine (100 mg, 211 μmol, described in example 93), 3-{acetyl[1-(tert-butoxycarbonyl)piperidin-4-yl]amino}benzoic acid (76.5 mg, 211 μmol), PyBOP (220 mg, 422 μmol) and N,N-diisopropylethylamine (180 μL, 1.1 mmol) in THF (2.0 mL) was stirred at room temperature overnight. H₂O was added, the mixture extracted with DCM and the solvent removed in vacuo. Purification by preparative HPLC (basic conditions) gave the title compound as a yellow solid (8.40 mg, 5%). ¹H-NMR (400 MHz, DMSO-d₆): δ [ppm]=9.07 (d, 1H), 8.68 (s, 1H), 8.50 (d, 1H), 7.94 (br d, 1H), 7.69 (s, 1H), 7.57 (t, 1H), 7.44-7.39 (m, 2H), 7.38-7.35 (m, 1H), 7.33-7.26 (m, 2H), 7.19 (d, 1H), 7.13 (s, 1H), 7.09 (dd, 1H), 5.93 (quin, 1H), 4.91 (sxt, 1H), 4.63-4.53 (m, 1H), 4.35 (t, 2H), 4.05-3.98 (m, 2H), 3.97-3.87 (m, 2H), 2.44 (s, 3H), 2.10 (s, 6H), 1.77-1.69 (m, 6H), 1.63 (s, 3H), 1.29 (s, 9H), 1.07-0.94 (m, 2H). LC-MS (Method 10): m/z: [M+H]⁺=818, Rₜ=1.45 min.

### EXAMPLE 99

### 6-[3-(benzylamino)azetidin-1-yl]-N-[1-(5-{2-[(dimethylamino)methyl]phenyl}thiophen-2-yl)ethyl]-2-methylpyrido[3,4-d]pyrimidin-4-amine

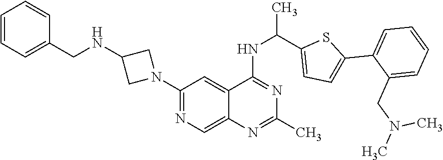 [SMILES: Cc1nc(NC(C)c2ccc(-c3ccccc3CN(C)C)s2)c2cc(N3CC(NCc4ccccc4)C3)ncc2n1]

A solution of 6-(3-aminoazetidin-1-yl)-N-[1-(5-{2-[(dimethylamino)methyl]phenyl}thiophen-2-yl)ethyl]-2-methylpyrido[3,4-d]pyrimidin-4-amine (100 mg, 211 μmol, described in example 93), (bromomethyl)benzene (33 μL, 270 μmol) and triethylamine (150 μL, 1.1 mmol) in THF (2.0 mL) was stirred at room temperature overnight. H₂O was added, the mixture extracted with DCM and the solvent removed in vacuo. Purification by preparative HPLC (basic conditions followed by acidic conditions) gave the title compound as a yellow solid (3.00 mg, 3%). ¹H-NMR (400 MHz, DMSO-d₆): δ [ppm]=8.62 (s, 1H), 8.47 (d, 1H), 8.27 (br s, 1H), 7.44-7.40 (m, 1H), 7.38-7.28 (m, 8H), 7.24 (br dt, 1H), 7.18 (d, 1H), 7.08 (dd, 1H), 7.01 (s, 1H), 5.92 (t, 1H), 4.10 (t, 2H), 3.74-3.64 (m, 5H), 2.42 (s, 3H), 2.10 (s, 6H), 1.71 (d, 3H). LC-MS (Method 10): m/z: [M+H]⁺=563, Rₜ=1.45 min.

### EXAMPLE 100

### N-[1-(4-{[1-(5-{2-[(dimethylamino)methyl]phenyl}thiophen-2-yl)ethyl]amino}-2-methylpyrido[3,4-d]pyrimidin-6-yl)azetidin-3-yl]thiomorpholine-4-carboxamide 1,1-dioxide

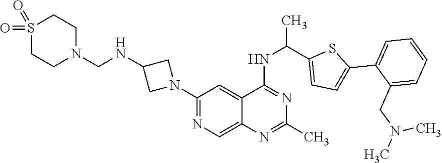 [SMILES: Cc1nc(NC(C)c2ccc(-c3ccccc3CN(C)C)s2)c2cc(N3CC(NCN4CCS(=O)(=O)CC4)C3)ncc2n1]

To a solution of thiomorpholine 1,1-dioxide (28.5 mg, 211 μmol) and triethylamine (150 μL, 1.1 mmol) in DMF (2.0 mL) was added 4-nitrophenyl carbonochloridate (42.6 mg, 211 μmol) and the reaction mixture stirred a room temperature during 30 minutes. 6-(3-aminoazetidin-1-yl)-N-[1-(5-{2-[(dimethylamino)methyl]phenyl}thiophen-2-yl)ethyl]-2-methylpyrido [3 ,4-d]pyrimidin-4-amine (100 mg, 211 μmol, described in example 93) was then added and the reaction mixture stirred at 120° C. overnight. Purification by preparative HPLC (basic conditions, thrice) gave the title compound as a yellow solid (3.20 mg, 2%). ¹H-NMR (400 MHz, DMSO-d₆): δ [ppm]=8.66 (s, 1H), 8.48 (d, 1H), 7.42 (dd, 1H), 7.38-7.28 (m, 5H), 7.19 (d, 1H), 7.10-7.06 (m, 2H), 5.98-5.87 (m, 1H), 4.62-4.53 (m, 1H), 4.24 (t, 2H), 3.90-3.84 (m, 2H), 3.76 (br s, 4H), 3.10-3.06 (m, 4H), 2.43 (s, 3H), 2.14-2.12 (m, 1H), 2.11 (s, 6H), 1.72 (d, 3H). LC-MS (Method 10): m/z: [M+H]⁺=635, Rₜ=1.21 min.

### EXPERIMENTAL SECTION—BIOLOGICAL ASSAYS

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

6,7-dimethoxy-N-[(1R)-1-(1-naphthyl)ethyl]quinazolin-4-amine, which was used to calibrate the assay, was prepared as follows:

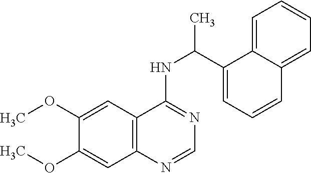 [SMILES: COc1cc2ncnc(NC(C)c3cccc4ccccc34)c2cc1OC]

To 4-chloro-6,7-dimethoxyquinazoline (100 mg, 0.445 mmol, commercially available) in 1.7 mL DMSO was added (1R)-1-(1-naphthyl)ethanamine (76 mg, 0.445 mmol, commercially available) and N-ethyl-N-isopropylpropan-2-amine (202 μl, 1.16 mmol). The reaction was stirred at 100° C. overnight, cooled to ambient temperature and filtered. After removal of the solvent under reduced pressure the crude product was purified via HPLC chromatography to yield the title compound (118 mg, 73%). ¹H-NMR (400 MHz ,DMSO-d₆), d [ppm]=1.72 (3H), 3.90 (6H), 6.32-6.41 (1H), 7.09 (1H), 7.46-7.58 (3H), 7.64-7.69 (1H), 7.78 (2H), 7.92-7.97 (1H), 8.18-8.24 (2H), 8.28 (1H).

The in vitro activity of the compounds of the present invention can be demonstrated in the following assays:

### Biochemical Assay 1: hK-RasG12C Interaction Assay with hSOS1

This assay quantifies the equilibrium interaction of human SOS1 (hSOS1) with human K-Ras^{G12C} (hK-RasG12C). Detection of the interaction is achieved by measuring homogenous time-resolved fluorescence resonance energy transfer (HTRF) from antiGST-Europium (FRET donor) bound to GST-K-RasG12C to anti-6His-XL665 bound to His-tagged hSOS1 (FRET-acceptor).

The assay buffer contained 5 mM HEPES pH 7.4 (Applichem), 150 mM NaCl (Sigma), 10 mM EDTA (Promega), 1 mM DTT (Thermofisher), 0.05% BSA Fraction V, pH 7.0, (ICN Biomedicals), 0.0025% (v/v) Igepal (Sigma) and 100 mM KF (FLUKA).

The expression and purification of N-terminal GST-tagged hK-RasG12C and N-terminal His-tagged hSOS1 is described below. Concentrations of protein batches used were optimized to be within the linear range of the HTRF signal. A Ras working solution was prepared in assay buffer containing typically 10 nM GST-hK-RasG12C and 2 nM antiGST-Eu(K) (Cisbio, France). A SOS1 working solution was prepared in assay buffer containing typically 20 nM His-hSOS1 and 10 nM anti-6His-XL665 (Cisbio, France). An inhibitor control solution was prepared in assay buffer containing 10 nM anti-6His-XL665 without hSOS1.

Fifty nl of a 100-fold concentrated solution of the test compound in DMSO were transferred into a black microtiter test plate (384 or 1536, Greiner Bio-One, Germany) For this, either a Hummingbird liquid handler (Digilab, Mass., USA) or an Echo acoustic system (Labcyte, Calif., USA) was used.

All steps of the assay were performed at 20° C. A volume of 2.5 μl of the Ras working solution was added to all wells of the test plate using a Multidrop dispenser (Thermo Labsystems). After 2 mM preincubation, 2.5 μl of the SOS1 working solution were added to all wells except for those wells at the side of the test plate that were subsequently filled with 2.5 μl of the inhibitor control solution. After 60 mM incubation the fluorescence was measured with a Pherastar (BMG, Germany) using the HTRF module (excitation 337 nm, emission 1:620 nm, emission 2:665 nm).

The ratiometric data (emission 2 divided by emission 1) were normalized using the controls (DMSO=0% inhibition, inhibition control wells with inhibitor control solution=100% inhibition). Compounds were tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0.073 nM). IC50 values were calculated by 4-Parameter fitting using a commercial software package (Genedata Screener, Switzerland).

### Biochemical Assay 2: hK-RasG12C Activation Assay by hSOS1 at High GTP Concentration

This assay quantifies human SOS1-mediated nucleotide exchange of human K-Ras^{G12C} (hK-RasG12C) preloaded with a fluorescent GTP-analog and in presence of an excess of free GTP. Loaded hK-RasG12C generates a high HTRF-signal by energy transfer from antiGST-Terbium (FRET donor) bound to hK-Ras to the loaded fluorescent GDP analog (FRET-acceptor). hSOS1 activity exchanges the fluorescent GDP for non-fluorescent GTP and therefore leads to a reduction of the HTRF signal.

The fluorescent GDP-analog EDA-GDP-Dy647P1 (2′/3′-O-(2-Aminoethyl-carbamoyl)-guanosine-5′-diphosphate labelled with Dy647P1 (Dyomics GmbH, Germany)) was synthesized by Jena Biosciences GmbH (Germany) and supplied as a 1 mM aqueous solution.

The expression and purification of N-terminal GST-tagged human K-RasG12C and N-terminal His-tagged human SOS1 is described below. Concentrations of protein batches used were optimized to be within the linear range of the HTRF signal.

Preparation of GST-tagged hK-RasG12C loaded with fluorescent nucleotide was performed as follows: incubation of 11.5 μM hK-Ras^{G12C} with 5-fold excess GDP-Dy647 nucleotide (54 μM) in 500 μl NLS-buffer (RAS activation Kit Jena Bioscience, Kat. # PR-950) for 10 mM at 37° C. Addition of 20 μl 1 M MgCl₂ (Sigma) to final 40 mM and store on ice. Purification into buffer (10 mM HEPES pH 7.4 (Applichem), 150 mM NaCl (Sigma), 5 mM MgCl₂ (Sigma)) by use of a PD-Minitrap desalting column (GE Healthcare). Concentration of 1 ml purified hK-Ras-GDP-Dy647 is approx. 4-5 μM.

The assay buffer contained 10 mM HEPES pH 7.4 (Applichem), 150 mM NaCl (Sigma), 5 mM MgCl₂ (Sigma), 1 mM DTT (Thermofisher), 0.05% BSA Fraction V, pH 7.0, (ICN Biomedicals), 0.0025% (v/v) Igepal (Sigma).

A Ras working solution was prepared in assay buffer containing typically 80 nM loaded GST-hK-RasG12C-EDA-GDP-Dy647P1 and 2 nM antiGST-Tb (Cisbio, France). A hSOS1 working solution was prepared in assay buffer containing typically 8 nM His-hSOS1 and 100 μM GTP (Jena Bioscience, Germany) An inhibitor control solution was prepared in assay buffer containing the same concentration of hSOS1 without GTP.

Alternatively, the inhibitor control solution was prepared by supplementing the hSOS1 working solution with 20 μM of 6,7-dimethoxy-N-[(1R)-1-(1-naphthyl)ethyl]quinazolin-4-amine which was used to calibrate the assay.

Fifty nl of a 100-fold concentrated solution of the test compound in DMSO were transferred into a black microtiter test plate (384 or 1536, Greiner Bio-One, Germany) For this, either a Hummingbird liquid handler (Digilab, Mass., USA) or an Echo acoustic system (Labcyte, Calif., USA) was used.

All steps of the assay were performed at 20° C. A volume of 2.5 μl of the Ras working solution was added to all wells of the test plate using a Multidrop dispenser (Thermo Labsystems). After 2 min. preincubation, 2.5 μl of the hSOS1 working solution were added to all wells except for those wells at the side of the test plate that were subsequently filled with 2.5 μl of the inhibitor control solution. After 20 min. incubation the fluorescence was measured with a Pherastar (BMG, Germany) using the HTRF module (excitation 337 nm, emission 1:620 nm, emission 2:665 nm).

The ratiometric data (emission 2 divided by emission 1) were normalized using the controls (DMSO=0% inhibition, inhibition control wells with inhibitor control solution =100% inhibition). Compounds were tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0.073 nM). IC50 values were calculated by 4-Parameter fitting using a commercial software package (Genedata Screener, Switzerland).

### Biochemical Assay 3: hK-RasG12C Activation Assay by hSOS1

K-Ras is a small GTPase that can bind GDP and GTP. The guanine nucleotide exchange factor SOS1 catalyzes the activation of K-Ras by promoting the exchange of GDP to GTP. SOS1 binds to K-Ras-GDP thereby opening the GDP-binding pocket to facilitate GDP release. Rebinding of excess nucleotide leads to dissociation of the K-Ras-SOS1 intermediate complex leaving K-Ras loaded with the nucleotide.

This assay quantifies human SOS1-(hSOS1-)mediated loading of human K-Ras^{G12C}-GDP (hK-RasG12C-GDP) with a fluorescent GTP-analog. Detection of successful loading is achieved by measuring homogenous time-resolved fluorescence resonance energy transfer (HTRF) from antiGST-Terbium (FRET donor) bound to GST-hK-RasG12C (see below) to the loaded fluorescent GTP analog (FRET-acceptor).

The fluorescent GTP-analog EDA-GTP-Dy647P1 (2′/3′-O-(2-Aminoethyl-carbamoyl)-guanosine-5′-triphosphate labelled with Dy647P1 (Dyomics GmbH, Germany)) was synthesized by Jena Biosciences GmbH (Germany) and supplied as a 1 mM aqueous solution.

The assay buffer contained 10 mM HEPES pH 7.4 (Applichem), 150 mM NaCl (Sigma), 5 mM MgCl₂ (Sigma), 1 mM DTT (Thermofisher), 0.05% BSA Fraction V, pH 7.0, (ICN Biomedicals), 0.0025% (v/v) Igepal (Sigma).

The expression and purification of N-terminal GST-tagged human K-RasG12C and N-terminal His-tagged hSOS1 is described below. Concentrations of protein batches used were optimized to be within the linear range of the HTRF signal. A hRas working solution was prepared in assay buffer containing typically 100 nM GST-hK-RasG12C and 2 nM antiGST-Tb (Cisbio, France). A hSOS1 working solution was prepared in assay buffer containing typically 20 nM hSOS1 and 200 nM EDA-GTP-Dy647P1. An inhibitor control solution was prepared in assay buffer containing 200 nM EDA-GTP-Dy647P1 without hSOS1.

Fifty nl of a 100-fold concentrated solution of the test compound in DMSO were transferred into a black microtiter test plate (384 or 1536, Greiner Bio-One, Germany) For this, either a Hummingbird liquid handler (Digilab, Mass., USA) or an Echo acoustic system (Labcyte, Calif., USA) was used.

All steps of the assay were performed at 20° C. A volume of 2.5 μl of the hRas working solution was added to all wells of the test plate using a Multidrop dispenser (Thermo Labsystems). After 10 min. preincubation, 2.5 μl of the hSOS1 working solution were added to all wells except for those wells at the side of the test plate that were subsequently filled with 2.5 μl of the inhibitor control solution. After 30 mM incubation the fluorescence was measured with a Pherastar (BMG, Germany) using the HTRF module (excitation 337 nm, emission 1: 620 nm, emission 2:665 nm).

The ratiometric data (emission 2 divided by emission 1) were normalized using the controls (DMSO=0% inhibition, inhibition control wells with inhibitor control solution =100% inhibition). Compounds were tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0.073 nM). IC50 values were calculated by 4-Parameter fitting using a commercial software package (Genedata Screener, Switzerland).

### Biochemical Assay 4: hK-RasG12C Activation Assay by hSOS2

This assay quantifies hSOS2-mediated loading of hK-Ras^{G12C}-GDP (hK-RasG12C-GDP) with a fluorescent GTP-analog. Detection of successful loading is achieved by measuring homogenous time-resolved fluorescence resonance energy transfer (HTRF) from antiGST-Terbium (FRET donor) bound to GST-hK-RasG12C to the loaded fluorescent GTP analog (FRET-acceptor).

The fluorescent GTP-analog EDA-GTP-Dy647P1 (2′/3′-O-(2-Aminoethyl-carbamoyl)-guanosine-5′-triphosphate labelled with Dy647P1 (Dyomics GmbH, Germany)) was synthesized by Jena Biosciences GmbH (Germany) and supplied as a 1 mM aqueous solution.

The assay buffer contained 10 mM HEPES pH 7.4 (Applichem), 150 mM NaCl (Sigma), 5 mM MgCl₂ (Sigma), 1 mM DTT (Thermofisher), 0.05% BSA Fraction V, pH 7.0, (ICN Biomedicals), 0.0025% (v/v) Igepal (Sigma).

The expression and purification of N-terminal GST-tagged hK-RasG12C and N-terminal His-tagged hSOS2 is described below. Concentrations of protein batches used were optimized to be within the linear range of the HTRF signal. A hRas working solution was prepared in assay buffer containing typically 100 nM GST-hK-RasG12C and 2 nM antiGST-Tb (Cisbio, France). A hSOS2 working solution was prepared in assay buffer containing typically 20 nM hSOS2 and 200 nM EDA-GTP-Dy647P1. An inhibitor control solution was prepared in assay buffer containing 200 nM EDA-GTP-Dy647P1 without hSOS2.

Fifty nl of a 100-fold concentrated solution of the test compound in DMSO were transferred into a black microtiter test plate (384 or 1536, Greiner Bio-One, Germany) For this, either a Hummingbird liquid handler (Digilab, Mass., USA) or an Echo acoustic system (Labcyte, Calif., USA) was used.

All steps of the assay were performed at 20° C. A volume of 2.5 μl of the hRas working solution was added to all wells of the test plate using a Multidrop dispenser (Thermo Labsystems). After 10 min. preincubation, 2.5 μl of the hSOS2 working solution were added to all wells except for those wells at the side of the test plate that were subsequently filled with 2.5 μl of the inhibitor control solution. After 30 mM incubation the fluorescence was measured with a Pherastar (BMG, Germany) using the HTRF module (excitation 337 nm, emission 1:620 nm, emission 2:665 nm).

The ratiometric data (emission 2 divided by emission 1) were normalized using the controls (DMSO =0% inhibition, inhibition control wells with inhibitor control solution =100% inhibition). Compounds were tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0.073 nM). IC50 values were calculated by 4-Parameter fitting using a commercial software package (Genedata Screener, Switzerland).

### EGFR Kinase Assay

EGFR inhibitory activity of compounds of the present invention was quantified employing the TR-FRET based EGFR assay as described in the following paragraphs.

Epidermal Growth Factor Receptor (EGFR) affinity purified from human carcinoma A431 cells (Sigma-Aldrich, # E3641) was used as kinase. As substrate for the kinase reaction the biotinylated peptide biotin-Ahx-AEEEEYFELVAKKK (SEQ ID NO: 8, C-terminus in amid form) was used which can be purchased e.g. form the company Biosyntan GmbH (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of EGFR in aqueous assay buffer [50 mM Hepes/HCl pH 7.0, 1 mM MgCl₂, 5 mM MnCl₂, 0.5 mM activated sodium ortho-vanadate, 0.005% (v/v) Tween-20]were added and the mixture was incubated for 15 mM at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (1.67 μM =>final conc. in the 5 μL assay volume is 1μM) in assay buffer and the resulting mixture was incubated for a reaction time of 20 mM at 22° C. The concentration of EGFR was adjusted depending of the activity of the enzyme lot and was chosen appropriate to have the assay in the linear range, typical concentration were about 3 U/ml. The reaction was stopped by the addition of 5 μl of a solution of HTRF detection reagents (0.1 μM streptavidine-XL665 [Cis Biointernational]and 1 nM PT66-Tb-Cryptate, an terbium-cryptate labelled anti-phospho-tyrosine antibody from Cis Biointernational [instead of the PT66-Tb-cryptate PT66-Eu-Chelate from Perkin Elmer can also be used]) in an aqueous EDTA-solution (80 mM EDTA, 0.2% (w/v) bovine serum albumin in 50 mM HEPES pH 7.5).

The resulting mixture was incubated 1 h at 22° C. to allow the binding of the biotinylated phosphorylated peptide to the streptavidine-XL665 and the PT66-Eu-Chelate. Subsequently the amount of phosphorylated substrate was evaluated by measurement of the resonance energy transfer from the PT66-Tb-Cryptate to the streptavidine-XL665. Therefore, the fluorescence emissions at 620 nm and 665 nm after excitation at 337 nm were measured in a HTRF reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor =0% inhibition, all other assay components but no enzyme =100% inhibition). Usually the test compounds were tested on the same microtiterplate in 11 different concentrations in the range of 20 μM to 0.072 nM (e.g. 20 μM, 5.7 μM, 1.6 μM, 0.47 μM, 0.13 μM, 38 nM, 11 nM, 3.1 nM, 0.89 nM, 0.25 nM and 0.072 nM, the dilution series prepared separately before the assay on the level of the 100fold concentrated solutions in DMSO by serial dilutions, the exact concentrations may vary depending on the pipettor used) in duplicate values for each concentration and IC50 values were calculated by a 4 parameter fit.

### KRAS Cellular Assays

### 3D-Softagar MiaPaca-2 (ATCC CRL-1420) and NCI-H1792 (ATCC CRL-5895)

Day 1: Softagar (Select Agar, Invitrogen, 3% in ddH₂O autoclaved) is boiled and tempered at 48° C. Medium (MiaPaca-2: DMEM/Ham's F12; [Biochrom; # FG 4815, with stable Glutamine]10% FCS and 2.5% Horse Serum, H1792: RPMI 1640; [Biochrom; # FG 1215, with stable Glutamine and 10%FCS]) is tempered to 37° C.; Agar (3%) is diluted 1:5 in medium (=0.6%) and 50 μl/well plated into 96 well plates (Corning, # 3904), wait at room temperature until the agar is solid. 3% agar is diluted to 0.25% in medium (1:12 dilution) and tempered at 42° C. Cells are trypsinized, counted and tempered at 37° C.; cells (MiaPaCa-2: 125-150, NCI-H1792: 1000) are resuspended in 100 μl 0.25% Agar and plated. Wait at room temperature until the agar is solid. Overlay wells with 50 μl medium. Plate sister wells in separate plate for time zero determination. All plates are incubated overnight 37° C. and 5% CO2.

Day 2: Measurement of time zero values: Add 40 μl Cell Titer 96 Aqueous Solution (Promega) per well, (light sensitive) and incubate in the dark at 37° Cand 5% CO2. Absorption is measured at 490 nm and reference wavelength 660 nm. DMSO-prediluted test compounds are added with HP Dispenser to a final DMSO concentration of 0.3%.

Day 10: Measurement of test compound and control treated wells with Cell Titer 96 AQueous according to time zero. The IC50 values were determined using the four parameter fit.

### Active RAS in Calu-1 cells (CLS 300141)

40.000 Calu-1 cells are seeded in 96 well plate (NUNC161093) for 48 h at 37° C./5%CO2 (10%FBS (S0615), DMEM/Ham's F-12 (Biochrom; # FG 4815), 2 mM L-Glutamine) After that, medium is changed to FBS-free medium and the cells were incubated for further 24 h at 37° C./5%CO2. Cells are treated with varying concentrations of DMSO-prediluted test compounds (final 0.1%) for 30 min. at 37° C./5%CO2. Supernatant with test compounds is discarded and, after that, treated cells are stimulated with 100 ng/m1 EGF (Sigma# E9644, diluted in serum free medium) for 3 minutes. Cells were treated with lysis buffer and all next steps were performed on ice according to the supplier's manual of G-LISA Kit (Cytoskeleton BK131, Ras Activation Assay). Finally, the content of active Ras is measured by detecting the absorbance at 490 nm (Tecan Sunrise). The value of EGF-stimulated cells is set as 100%, whereas the value of untreated cells is set as 0%. The IC50 values were determined using the four parameter fit.

### P-EGFR assay (In-Cell Western) in Hela cells (ATCC CCL-2)

After stimulation with EGF, the EGF receptor autophosphorylates at Y1173. In-cell Western assay simultaneously detect two targets at 700 and 800 nm using two spectrally distinct near-infrared dyes. With a specific antibody, phosphorylated EGFR can be quantified and the samples can be normalized with total EGFR antibody parallel.

25000 Hela cells are seeded in 96 well plate (NUNC161093) for 24 h at 37° C./5%CO2 (10%FBS (S0615), DMEM/Ham's F-12 (Biochrom; # FG 4815), 2 mM L-Glutamine) After that, medium is changed to FBS-free medium and the cells were incubated for further 24 h at 37° C./5%CO2. Cells are treated with varying concentrations of DMSO-prediluted test compounds (final 0.1%) for 30 minutes and finally with 100 ng/ml EGF (Sigma# E9644, diluted in serum free medium) for 2 minutes.

Cells are treated according the manual of EGFR Near Infrared In-Cell ELISA Kit (Pierce # 62210). If not specified, all buffers and antibodies are part of this kit.

Cells are fixed with 4% formaldehyde, washed twice with 100 μl per well with TRIS-buffered saline with Surfact-Amps 20, permeabilized with 100 μl TRIS-buffered saline with Surfact-Amps X-100, wash again with 100 μl TRIS-buffered saline, and finally 200 μl blocking buffer are added for 60 minutes at room temperature. Fixed and washed cells are incubated with primary antibody mix (P-EGFR; EGFR) overnight at 2-8° C. After washing with 100 μl TRIS-buffered saline with Surfact-Amps 20, secondary IRDye-labeled antibody mix (DyLight 800 Goat Anti-Rabbit IgG, Pierce SA5-35571; DyLight 680 Goat Anti-Mouse IgG, Pierce 35518) is added for lh at room temperature and washed again. Plates are scanned with LiCor Odyssey Infrared Imager at 800 nm for P-EGFR and at 700 nm for total EGFR. The quotient of 800 nm and 700 nm for EGF only treated cells is set as 100% and the quotient of 800 nm and 700 nm of untreated cells is set as 0%. The IC50 values were determined using the four parameter fit.

TABLE 1
IC50 values of the compounds of examples 1 to 458 in in vitro assays 1 to
3 and the EGFR-assay (“n.d.” means “not determined”)

| | | Assay 2 (hKRAS- | Assay 3 (hKRAS- | |
|---|---|---|---|---|
| | Assay 1 (hKRAS, | Activation by | Activation by | |
| | hSOS Interaction) | hSOS high GTP) | hSOS no GTP) | EGFR |
| | activity (expressed | activity (expressed | activity (expressed | kinase |
| Example | as IC50, or as % | as IC50, or as % | as IC50, or as % | inhibition |
| No | inhibition at 20 μM) | inhibition at 20 μM) | inhibition at 20 μM) | IC50 |
| 1 | 1.07E−6 | 9.30E−7 | 6.36E−6 | >2.00E−5 |
| 2 | 2.46E−6 | 2.51E−6 | >2.00E−5 | >2.00E−5 |
| 3 | 2.15E−7 | 2.62E−7 | 3.55E−7 | >2.00E−5 |
| 4 | 3.55E−7 | 4.23E−7 | 5.55E−7 | >2.00E−5 |
| 5 | 4.91E−7 | 9.94E−7 | 2.15E−6 | >2.00E−5 |
| 6 | 3.93E−7 | 1.12E−6 | 1.69E−6 | >2.00E−5 |
| 7 | 6.33E−7 | 1.30E−6 | 2.38E−6 | >2.00E−5 |
| 8 | 5.49E−6 | 4.89E−6 | >2.00E−5 | >2.00E−5 |
| 9 | 53.95% | 1.79E−5 | >2.00E−5 | >2.00E−5 |
| 10 | 1.14E−6 | 9.34E−7 | 8.35E−6 | >2.00E−5 |
| 11 | 1.79E−6 | 1.96E−6 | 2.49E−6 | >2.00E−5 |
| 12 | 5.84E−6 | 6.45E−6 | 8.17E−6 | >2.00E−5 |
| 13 | 1.22E−6 | 1.78E−6 | 7.98E−6 | >2.00E−5 |
| 14 | 1.65E−6 | 1.95E−6 | 1.26E−5 | >2.00E−5 |
| 15 | 1.48E−6 | 1.90E−6 | 8.79E−6 | >2.00E−5 |
| 16 | 9.63E−7 | 9.01E−7 | 4.34E−6 | >2.00E−5 |
| 17 | 9.19E−6 | 7.73E−6 | ! 4.00E−5 | >2.00E−5 |
| 18 | 1.11E−6 | 1.01E−6 | 4.54E−6 | >2.00E−5 |
| 19 | 2.03E−6 | | 1.25E−5 | >2.00E−5 |
| 20 | 30.04% | >2.00E−5 | ! 4.00E−5 | >2.00E−5 |
| 21 | 26.32% | >2.00E−5 | ! 4.00E−5 | >2.00E−5 |
| 22 | 1.62E−6 | 1.96E−6 | 1.25E−5 | >2.00E−5 |
| 23 | 4.35E−6 | 4.45E−6 | 3.28E−6 | >2.00E−5 |
| 24 | 9.41E−6 | 9.71E−6 | >2.00E−5 | >2.00E−5 |
| 25 | 23.71% | >2.00E−5 | >2.00E−5 | >2.00E−5 |
| 26 | 2.75E−6 | 3.04E−6 | >2.00E−5 | >2.00E−5 |
| 27 | 2.58E−6 | 1.84E−6 | 1.06E−5 | >2.00E−5 |
| 28 | 49.80% | >2.00E−5 | >2.00E−5 | >2.00E−5 |
| 29 | 1.74E−5 | >2.00E−5 | >2.00E−5 | >2.00E−5 |
| 30 | 5.43E−6 | 5.74E−6 | ! 2.78E−5 | >2.00E−5 |
| 31 | 1.77E−5 | ! 4.00E−5 | >2.00E−5 | >2.00E−5 |
| 32 | 22.37% | >2.00E−5 | >2.00E−5 | >2.00E−5 |
| 33 | 29.27% | >2.00E−5 | >2.00E−5 | >2.00E−5 |
| 34 | 36.69% | >2.00E−5 | >2.00E−5 | >2.00E−5 |
| 35 | 23.49% | >2.00E−5 | >2.00E−5 | >2.00E−5 |
| 36 | 9.03E−6 | 1.90E−5 | >2.00E−5 | >2.00E−5 |
| 37 | 9.05E−8 | 1.35E−7 | 1.09E−7 | >2.00E−5 |
| 38 | 2.18E−7 | 6.01E−7 | 2.69E−7 | >2.00E−5 |
| 39 | 8.76E−7 | 1.36E−6 | 1.64E−6 | >2.00E−5 |
| 40 | 1.32E−5 | 8.66E−6 | >2.00E−5 | >2.00E−5 |
| 41 | 1.60E−6 | 2.31E−6 | 3.88E−6 | >2.00E−5 |
| 42 | 1.87E−7 | 2.15E−7 | 3.66E−7 | >2.00E−5 |
| 43 | 2.96E−7 | 3.80E−7 | 4.46E−7 | >2.00E−5 |
| 44 | 9.85E−7 | 1.06E−6 | 1.64E−6 | >2.00E−5 |
| 45 | 9.18E−8 | 5.06E−7 | 4.59E−7 | >2.00E−5 |
| 46 | 1.29E−6 | 4.22E−6 | 1.28E−5 | >2.00E−5 |
| 47 | 2.23E−8 | 3.64E−8 | 3.67E−8 | >2.00E−5 |
| 48 | 3.88E−8 | 1.41E−7 | 1.14E−7 | >2.00E−5 |
| 49 | 1.21E−6 | 3.63E−6 | 6.99E−6 | >2.00E−5 |
| 50 | 2.82E−8 | 4.88E−8 | 5.48E−8 | >2.00E−5 |
| 51 | 3.29E−8 | 7.78E−8 | 8.95E−8 | >2.00E−5 |
| 52 | 2.02E−7 | 8.56E−7 | 5.32E−7 | >2.00E−5 |
| 53 | 5.47E−8 | 2.23E−7 | 2.39E−7 | >2.00E−5 |
| 54 | 4.96E−8 | 1.61E−7 | 1.89E−7 | >2.00E−5 |
| 55 | 4.41E−8 | 8.54E−8 | 7.28E−8 | >2.00E−5 |
| 56 | 3.94E−8 | 9.39E−8 | 8.95E−8 | >2.00E−5 |
| 57 | 1.97E−6 | 1.23E−6 | 1.78E−6 | >2.00E−5 |
| 58 | 1.17E−7 | 1.41E−7 | 1.55E−7 | >2.00E−5 |
| 59 | 6.62E−8 | 6.79E−8 | 6.77E−8 | >2.00E−5 |
| 60 | 2.88E−8 | 2.38E−8 | 3.98E−8 | >2.00E−5 |
| 61 | 3.60E−8 | 2.16E−8 | 4.04E−8 | >2.00E−5 |
| 62 | 2.91E−7 | 4.43E−7 | 7.35E−7 | >2.00E−5 |
| 63 | 4.36E−8 | 2.80E−8 | 6.11E−8 | >2.00E−5 |
| 64 | 5.95E−8 | 6.26E−8 | 8.03E−8 | >2.00E−5 |
| 65 | 4.22E−8 | 3.28E−8 | 5.51E−8 | >2.00E−5 |
| 66 | 3.04E−8 | 2.27E−8 | 4.33E−8 | >2.00E−5 |
| 67 | 4.20E−8 | 2.66E−8 | 6.33E−8 | >2.00E−5 |
| 68 | 43.80% | >2.00E−5 | >2.00E−5 | >2.00E−5 |
| 69 | 6.26E−8 | 9.83E−8 | 1.41E−7 | >2.00E−5 |
| 70 | 4.09E−7 | 9.51E−7 | 1.34E−6 | >2.00E−5 |
| 71 | 5.11E−8 | 5.55E−8 | 8.25E−8 | >2.00E−5 |
| 72 | 3.42E−8 | 4.21E−8 | 4.90E−8 | >2.00E−5 |
| 73 | 9.50E−8 | 1.29E−7 | 1.38E−7 | >2.00E−5 |
| 74 | 5.52E−8 | 9.28E−8 | 1.07E−7 | >2.00E−5 |
| 75 | 4.18E−6 | 3.19E−6 | 1.52E−5 | >2.00E−5 |
| 76 | 4.52E−8 | 4.99E−8 | 7.30E−8 | >2.00E−5 |
| 77 | 4.98E−8 | 5.88E−8 | 8.43E−8 | >2.00E−5 |
| 78 | 1.28E−6 | 1.04E−6 | 2.27E−6 | >2.00E−5 |
| 79 | 2.09E−8 | 1.55E−8 | 2.44E−8 | >2.00E−5 |
| 80 | 2.82E−6 | 1.25E−6 | 1.85E−6 | >2.00E−5 |
| 81 | 2.86E−8 | 3.45E−8 | 3.09E−8 | >2.00E−5 |
| 82 | 5.43E−7 | 6.15E−7 | 5.94E−7 | >2.00E−5 |
| 83 | 5.75E−7 | 1.34E−6 | 1.09E−6 | >2.00E−5 |
| 84 | 5.13E−7 | 1.03E−6 | 1.21E−6 | >2.00E−5 |
| 85 | 8.34E−8 | 2.14E−7 | 2.12E−7 | >2.00E−5 |
| 86 | 1.19E−7 | 1.27E−7 | 1.44E−7 | >2.00E−5 |
| 87 | 7.10E−8 | 2.40E−7 | 2.28E−7 | >2.00E−5 |
| 88 | 5.95E−8 | 1.61E−7 | 1.73E−7 | >2.00E−5 |
| 89 | 4.44E−8 | 1.11E−7 | 1.32E−7 | >2.00E−5 |
| 90 | 6.30E−8 | 1.54E−7 | 1.90E−7 | >2.00E−5 |
| 91 | 5.51E−8 | 1.37E−7 | 1.79E−7 | >2.00E−5 |
| 92 | 4.54E−8 | 1.16E−7 | 1.55E−7 | >2.00E−5 |
| 93 | 3.35E−8 | 6.92E−8 | 8.29E−8 | >2.00E−5 |
| 94 | 5.72E−8 | 1.63E−7 | 1.70E−7 | >2.00E−5 |
| 95 | 1.07E−7 | 2.40E−7 | 2.04E−7 | >2.00E−5 |
| 96 | 7.66E−8 | 2.26E−7 | 2.06E−7 | >2.00E−5 |
| 97 | 2.93E−7 | 2.98E−7 | 5.54E−7 | Not measured |
| 98 | 2.71E−7 | 4.28E−7 | 6.20E−7 | Not measured |
| 99 | 5.32E−8 | 5.88E−8 | 8.25E−8 | Not measured |
| 100 | 8.58E−8 | 1.03E−7 | 1.56E−7 | Not measured |

As exemplified in table 1, the compounds of the present invention inhibit the binding of hSOS1 to hKRAS, which was measured in the biochemical hK-RasG12C -hSOS1 interaction assay (assay 1). The ability to inhibit the hKRAS-hSOS1 interaction results in the inhibition of hKRAS activation by the compounds, as measured in biochemical assay 3, which quantifies the hSOS1-mediated nucleotide exchange from hK-RasG12C-GDP to hK-RasG12C loaded with a fluorescent GTP-analog. Furthermore, the compounds of the present invention show the ability to inhibit the nucleotide exchange reaction catalyzed by hSOS1 in the presence of a high concentration of 50 μM GTP, as measured in assay 2. This ability increases the chance that the compounds will be able to inhibit hSOS1 mediated hKRAS-activation inside cells, where high GTP concentrations are present. The chemical structure of the compounds of the present invention is similar to known inhibitors of EGFR-kinase. As shown in table 1, most compounds are inactive against EGFR-kinase up to the highest concentration measured in the assay (>20 μM).

The assay data of the large number of compounds in table 1 gives evidence that compounds which have a pharmacological profile as tested according to assays 1 to 3 and as described in the preceding paragraph will be generally useful to inhibit hSOS1 mediated hKRAS-activation inside cells, where high GTP concentrations are present and activity against EGFR-kinase up to highest concentrations (>20 μM) will not be measured in the assay.

Therefore an even further aspect of the present invention refers to the use of a compound which inhibits the binding of hSOS1 to human H— or N— or K-RAS including their clinically known mutations and which inhibits the nucleotide exchange reaction catalyzed by hSOS1 in the presence of a concentration of 20 μM or lower, but which is substantially inactive against EGFR-kinase at concentrations of 20 μM or lower for the preparation of a medicament for the treatment or prophylaxis of a hyperproliferative disorder.

Particularly this aspect refers to the use of a compound which inhibits the binding of hSOS1 specifically to hK-RasG12C protein and which inhibits the nucleotide exchange reaction catalyzed by hSOS1 in the presence of a concentration of 20 μM or lower, but which is substantially inactive against EGFR-kinase at concentrations of 20 μM or lower for the preparation of a medicament for the treatment or prophylaxis of a hyperproliferative disorder.

### Expression of hK-RasG12C, hSOS1, hSOS1_12 and hSOS2 in E. coli

The applied DNA expression constructs encoding the following protein sequences and its corresponding DNA sequences were optimized for expression in E. coli and synthesized by the GeneArt Technology at Life Technologies:

Human K-Ras (P01116-2):

hK-RasG12C (amino acid 1-169)

Human SOS1 (Q07889):

hSOS1 (amino acid 564-1049)

hSOS1_12(amino acid 564-1049 which is fused at its N-terminus with the amino acid sequence GAMA

Human SOS2 (Q07890):

hSOS2 (amino acid 564-1043)

These expressions construct additionally encoded att-site sequences at the 5′and 3′ ends for subcloning into various destination vectors using the Gateway Technology as well as a TEV (Tobacco Etch Virus) protease site for proteolytic cleavage of tag sequences. The applied destination vectors were: pD-ECO1 (an in-house derivate of the pET vector series from Novagen with ampicillin resistance gene) which provides an N-terminal fusion of a GST-tag to the integrated gene of interest. pD-ECO5 (also an in-house derivative of the pET vector series with ampicillin resistance gene) which provides a N-terminal fusion of a His10-tag to the integrated gene. To generate the final expression vectors the expression construct of hK-Ras_G12C was cloned into pD-ECO1. hSOS1, hSOS1_12 as well as hSOS2 were cloned into pD-ECOS. The resulting expression vectors were termed pD-ECO1_hK-RasG12C, pD-ECO5_hSOS1, pD-ECO5_hSOS1_12, pD-ECO5_hSOS2

Sequences:
GST-hK-RasG12C (G12C mutation according to
numbering in P01116-2)
(SEQ ID NO: 1)
MSPILGYWKI KGLVQPTRLL LEYLEEKYEE HLYERDEGDK
WRNKKFELGL EFPNLPYYID GDVKLTQSMA IIRYIADKHN
MLGGCPKERA EISMLEGAVL DIRYGVSRIA YSKDFETLKV
DFLSKLPEML KMFEDRLCHK TYLNGDHVTH PDFMLYDALD
VVLYMDPMCL DAFPKLVCFK KRIEAIPQID KYLKSSKYIA
WPLQGWQATF GGGDHPPKSD PITSLYKKAG SDYDIPTTEN
LYFQGMTEYK LVVVGACGVG KSALTIQLIQ NHFVDEYDPT
IEDSYRKQVV IDGETCLLDI LDTAGQEEYS AMRDQYMRTG
EGFLCVFAIN NTKSPEDIHH YREQIKRVKD SEDVPMVLVG
NKCDLPSRTV DTKQAQDLAR SYGIPFIETS AKTRQGVDDA
FYTLVREIRK HKEK
His10-hSOS1
(SEQ ID NO: 2)
MGHHHHHHHH HHSSGHIEGR HMLETSLYKK AGSDYDIPTT
ENLYFQGEEQ MRLPSADVYR FAEPDSEENI IFEENMQPKA
GIPIIKAGTV IKLIERLTYH MYADPNFVRT FLTTYRSFCK
PQELLSLIIE RFEIPEPEPT EADRIAIENG DQPLSAELKR
FRKEYIQPVQ LRVLNVCRHW VEHHFYDFER DAYLLQRMEE
FIGTVRGKAM KKWVESITKI IQRKKIARDN GPGHNITFQS
SPPTVEWHIS RPGHIETFDL LTLHPIEIAR QLTLLESDLY
RAVQPSELVG SVWTKEDKEI NSPNLLKMIR HTTNLTLWFE
KCIVETENLE ERVAVVSRII EILQVFQELN NFNGVLEVVS
AMNSSPVYRL DHTFEQIPSR QKKILEEAHE LSEDHYKKYL
AKLRSINPPC VPFFGIYLTN ILKTEEGNPE VLKRHGKELI
NFSKRRKVAE ITGEIQQYQN QPYCLRVESD IKRFFENLNP
MGNSMEKEFT DYLFNKSLEI EPRNPKPLPR FPKKYSYPLK
SPGVRPSNPR PGT
His10-hSOS1_12
(SEQ ID NO: 3)
MGHHHHHHHH HHSSGHIEGR HMLETSLYKK AGSDYDIPTT
ENLYFQGAMA EEQMRLPSAD VYRFAEPDSE ENIIFEENMQ
PKAGIPIIKA GTVIKLIERL TYHMYADPNF VRTFLTTYRS
FCKPQELLSL IIERFEIPEP EPTEADRIAI ENGDQPLSAE
LKRFRKEYIQ PVQLRVLNVC RHWVEHHFYD FERDAYLLQR
MEEFIGTVRG KAMKKWVESI TKIIQRKKIA RDNGPGHNIT
FQSSPPTVEW HISRPGHIET FDLLTLHPIE IARQLTLLES
DLYRAVQPSE LVGSVWTKED KEINSPNLLK MIRHTTNLTL
WFEKCIVETE NLEERVAVVS RIIEILQVFQ ELNNFNGVLE
VVSAMNSSPV YRLDHTFEQI PSRQKKILEE AHELSEDHYK
KYLAKLRSIN PPCVPFFGIY LTNILKTEEG NPEVLKRHGK
ELINFSKRRK VAEITGEIQQ YQNQPYCLRV ESDIKRFFEN
LNPMGNSMEK EFTDYLFNKS LEIEPRNPKP LPRFPKKYSY
PLKSPGVRPS NPRPGT
hSOS1_12 (tag-free)
(SEQ ID NO: 4)
GAMAEEQMRL PSADVYRFAE PDSEENIIFE ENMQPKAGIP
IIKAGTVIKL IERLTYHMYA DPNFVRTFLT TYRSFCKPQE
LLSLIIERFE IPEPEPTEAD RIAIENGDQP LSAELKRFRK
EYIQPVQLRV LNVCRHWVEH HFYDFERDAY LLQRMEEFIG
TVRGKAMKKW VESITKIIQR KKIARDNGPG HNITFQSSPP
TVEWHISRPG HIETFDLLTL HPIEIARQLT LLESDLYRAV
QPSELVGSVW TKEDKEINSP NLLKMIRHTT NLTLWFEKCI
VETENLEERV AVVSRIIEIL QVFQELNNFN GVLEVVSAMN
SSPVYRLDHT FEQIPSRQKK ILEEAHELSE DHYKKYLAKL
RSINPPCVPF FGIYLTNILK TEEGNPEVLK RHGKELINFS
KRRKVAEITG EIQQYQNQPY CLRVESDIKR FFENLNPMGN
SMEKEFTDYL FNKSLEIEPR NPKPLPRFPK KYSYPLKSPG
VRPSNPRPGT
His10-hSOS2
(SEQ ID NO: 5)
MGHHHHHHHH HHSSGHIEGR HMLETSLYKK AGSDYDIPTT
ENLYFQGPLR LPSPEVYRFV VKDSEENIVF EDNLQSRSGI
PIIKGGTVVK LIERLTYHMY ADPNFVRTFL TTYRSFCKPQ
ELLSLLIERF EIPEPEPTDA DKLAIEKGEQ PISADLKRFR
KEYVQPVQLR ILNVFRHWVE HHFYDFERDL ELLERLESFI
SSVRGKAMKK WVESIAKIIR RKKQAQANGV SHNITFESPP
PPIEWHISKP GQFETFDLMT LHPIEIARQL TLLESDLYRK
VQPSELVGSV WTKEDKEINS PNLLKMIRHT TNLTLWFEKC
IVEAENFEER VAVLSRIIEI LQVFQDLNNF NGVLEIVSAV
NSVSVYRLDH TFEALQERKR KILDEAVELS QDHFKKYLVK
LKSINPPCVP FFGIYLTNIL KTEEGNNDFL KKKGKDLINF
SKRRKVAEIT GEIQQYQNQP YCLRIEPDMR RPFENLNPMG
SASEKEFTDY LFNKSLEIEP RNCKQPPRFP RKSTFSLKSP GIRPNTG

E. coli Expression

The expression vectors were transformed into E. coli strain BL21 (DE3). Cultivation of the transformed strains for expression was done in 10 L and 1 L fermenter.

The cultures were grown in Terrific Broth media (MP Biomedicals, Kat. # 113045032) with 200 ug/mL ampicillin at a temperature of 37° C. to a density of 0.6 (OD600), shifted to a temperature of 27° C. (for hK-Ras expression vectors) or 17° C. (for hSOS expression vectors), induced for expression with 100 mM IPTG and further cultivated for 24 hours.

Purification

After cultivation the transformed E. coli were harvested by centrifugation and the resulting pellet was suspended in a lysis buffer (see below) and lysed by passing three-times through a high pressure device (Microfluidics). The lysate was centrifuged (49000 g, 45 min., 4° C.) and the supernatant used for further purification.

An Äkta chromatography system was used for all further chromatography steps.

### Purification of GST-hK-RasG12C for Biochemical Assays

E. coli culture (transformed with pD-ECO1_hK-RasG12C) from a 10L fermenter was lysed in lysis buffer (50 mM Tris HCl7.5, 500 mM NaCl, 1 mM DTT, 0.5% CHAPS, Complete Protease Inhibitor Cocktail-(Roche)). As a first chromatography step the centrifuged lysate was incubated with 50 mL Glutathione Agarose 4B (Macherey-Nagel; 745500.100) in a spinner flask (16 h, 10° C.). The Glutathione Agarose 4B loaded with protein was transferred to a chromatography column connected to an Äkta chromatography system. The column was washed with wash buffer (50 mM Tris HCl7.5, 500 mM NaCl, 1 mM DTT) and the bound protein eluted with elution buffer (50 mM Tris HCl 7.5, 500 mM NaCl, 1 mM DTT, 15 mM Glutathione). The main fractions of the elution peak (monitored by OD280) were pooled. For further purification by size-exclusion chromatography the above eluate volume was applied to a column Superdex 200 HR prep grade (GE Healthcare) and the resulting peak fractions of the eluted fusion protein were collected. The final yield of hK-RasG12C was about 50 mg purified fusion protein per L culture and the final product concentration was about 1 mg/mL. Native mass spectrometry analyses of the final purified K-RasG12C demonstrated its homogeneous load with GDP.

### Purification of His10-hSOS1 and His10-hSOS2 for Biochemical Assays

E. coli transformed with pD-EC05_hSOS1 or pD-ECO5_hSOS1_12 were cultured and induced in a fermenter, harvested and lysed in lysis buffer (25 mM Tris HCl 7.5, 500 mM NaCl, 20 mM Imidazol, Complete EDTA-free (Roche)). For immobilized metal ion affinity chromatography (IMAC) the centrifuged lysate (50 000 xg, 45 min., 4° C.) was incubated with 30 mL Ni-NTA (Macherey-Nagel; # 745400.100) in a spinner flask (16 h, 4° C.) and subsequently transferred to a chromatography column connected to an Äkta chromatography system. The column was rinsed with wash buffer (25 mM Tris HCl 7.5, 500 mM NaCl, 20 mM Imidazol) and the bound protein eluted with a linear gradient (0-100%) of elution buffer (25 mM Tris HCl 7.5, 500 mM NaCl, 300 mM Imidazol). The main fractions of the elution peak (monitored by OD280) containing homogenous His10-hSOS were pooled. The final yield of His10-hSOS1 was about 110 mg purified protein per L culture and the final product concentration was about 2 mg/mL. For His10-hSOS2 the final yield was 190 mg per L culture and the product concentration 6 mg/mL.

### Purification of hSOS1_12

To produce tag-free hSOS1_12 the same process consisting of 4 chromatography steps applying an Äkta system was used as decribed here below for hSOS1.

His10-hSOS1_12 was expressed in E. coli transformed with pD-EC05_hSOS1_12 as described above.

For IMAC the centrifuged lysate was directly applied to a 30 mL (or 50 mL) column with Ni-NTA (Macherey-Nagel) in an Äkta system, rinsed with wash buffer (25 mM Tris HCl 7.5, 500 mM NaCl, 20 mM Imidazol) and the bound protein was eluted with a linear gradient (0-100%) of elution buffer (25 mM Tris HCl 7.5, 500 mM NaCl, 300 mM Imidazol). The main fractions of the elution peak (monitored by OD280) were passed over a HiPrep Desalting column (GE; # 17-5087-01) to change to the cleavage buffer (25 mM Tris HCl 7.5, 150 mM NaCl, 1 mM DTT). The adjusted protein solution was treated with purified His-TEV protease (ratio hSOS1: TEV, w/w, 30:1) for 16 h at 4° C. and afterwards passed over a Ni-NTA column to remove non-cleaved hSOS1 protein, cleaved tag and His-TEV. The pooled flow through fractions with the processed hSOS1 were concentrated using a Amicon Ultra 15 Ultracel-10 device (Centrifugal Filter 10000 NMWL; Merck-Millipore # UFC901024) and applied to size-exclusion chromatography column with Superdex 200 HR prep grade (GE Healthcare) in SEC buffer (25 mM Tris HCl 7.5, 100 mM NaCl). The final yield of tag-free protein for SOS1_12 was about 245 mg per liter cell culture was. The final product (tag-free) concentration for hSOS1_12 was 30.7 mg/mL.

### Complex Formation and Crystallization of hSOS1_12 and Example 81

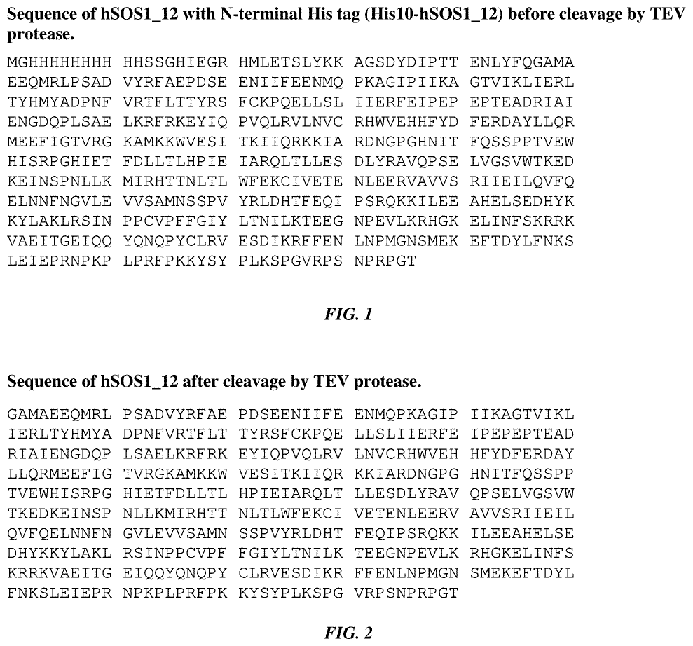

For crystallization of hSOS1 in complex with inhibitors, the hSOS1_12 construct was used. It is identical to the construct published by Freedman et al. (Ref. 1). It comprises of hSOS1 residues Glu564 to Thr1049 with an additional four amino acids (Gly-Ala-Met-Ala) at the N-terminus and is shown in FIGS. 1 and 2. For complex formation, frozen aliquots of the hSOS1_12 protein (concentration 30.7 mg/ml) in buffer (25 mM Tris HCl 7.5/50 mM NaCl/1 mM DTT) were thawed and Example 81 was added from a 200 mM DMSO stock solution to a final inhibitor concentration of 2 mM. The mix was incubated over night at 4° C. Crystals were grown at 20° C. using the hanging drop method. Drops were made from 1 μl hSOS1_12:inhibitor mix, 1 μl reservoir solution (100 mM HEPES pH 7.3, 24% (w/v) PEG 3350) and 0.2 μl seed stock. The seed stock was generated from hSOS1 crystals previously obtained in an initial screen using the same hSOS1_12 construct and a reservoir solution of 25% ethylene glycol. The crystals of the complex of hSOS1_12 and Example 81 grew within 2 days.

### Data Collection and Processing

A crystal of the complex of hSOS1_12 and Example 81 was briefly immerged in cryo buffer (reservoir solution supplemented with 15% ethylene glycol and 3 mM Example 81) and shock frozen in liquid nitrogen. A diffraction data set was collected at beamline 103 at the Diamond Light Source synchrotron (Oxfordshire, UK) at 100 K using a wavelength of 0.97625 A and a PILATUS3 6M detector. The diffraction images were processed using the programs XDS and XDSAPP. The crystal diffracted to a resolution of 2.0 A and belonged to space group P2₁2₁2₁ with unit cell dimensions of a=39.5 Å, b=84.3 ∈ and c=176.9 Å, with one hSOS1 molecule per asymmetric unit.

### Structure Determination and Refinement

The crystal form described here was first obtained and solved for a hSOS1_12 crystal grown in the presence of another inhibitor of the same chemical series, from a reservoir solution composed of 25% ethylene glycol. This initial structure was solved using the Molecular Replacement method with the program PHASER from the CCP4 program suite and the published structure of hSOS1 (PDB entry 2ii0, Ref. 1) as search model. The data set for hSOS1_12: Example 81 was then solved by Molecular Replacement using PHASER and an earlier in-house SOS1:inhibitor co-complex structure as starting model. A 3D model for Example 81 was generated using the program Discovery Studio (company Biovia) and parameter files for crystallographic refinement and model building were generated using software PRODRG. Example 81 was manually built into the electron density maps using the program COOT, followed by several cycles of refinement (using program REFMAC as part of the CCP4 program suite) and rebuilding in COOT. The final co-complex structure features an R(work) factor of 20.4% and an R(free) factor of 24.0%. The statistics of the data collection and refinement are summarized in Table 1.

TABLE 2
Data collection and refinement statistics
for the complex of hSOS1_12 and Example 81

hSOSl_12: Example 81

Data Collection:

| Source | Beamline I03, Diamond | |
|---|---|---|
| | Light Source, UK | |
| Wavelength [Å] | 0.97625 | |
| Space group (no.) | P2₁2₁2₁ | |
| Unit cell parameters, a, b, c [Å] | 39.5, 84.3, 176.9 | |
| Resolution limit [Å] | 1.97-48.3 | (1.97-2.09) |
| No. of reflections | 255731 | (28263) |
| No. of uniques | 42435 | (6358) |
| Multiplicity | 6.0 | (6.7) |
| I/sigI | 16.7 | (2.44) |
| R_meas [%] | 6.7 | (71.0) |
| Completeness [%] | 98.9 | (93.5) |

| | |
|---|---|
| B(Wilson) [Å²] | 43.5 |
| Mosaicity [deg] | 0.092 |

Refinement

| Resolution limit [Å] | 1.98-48.3 | (1.98-2.03) |
|---|---|---|
| Completeness [%] | 97.1 | (90.9) |
| No. of reflections overall/work/test | 41253/39228/2025 | |
| R (work)/R(free) [%] | 20.4/24.0 | (31.7/36.3) |

| | |
|---|---|
| Mean B value [Å²] | 32.7 |
| RMSD bond lengths [Å] | 0.007 |
| RMSD bond angles [deg] | 1.13 |

Values in brackets refer to the highest resolution shell.

### Absolute Configuration of Example 81 Bound to hSOS1_12

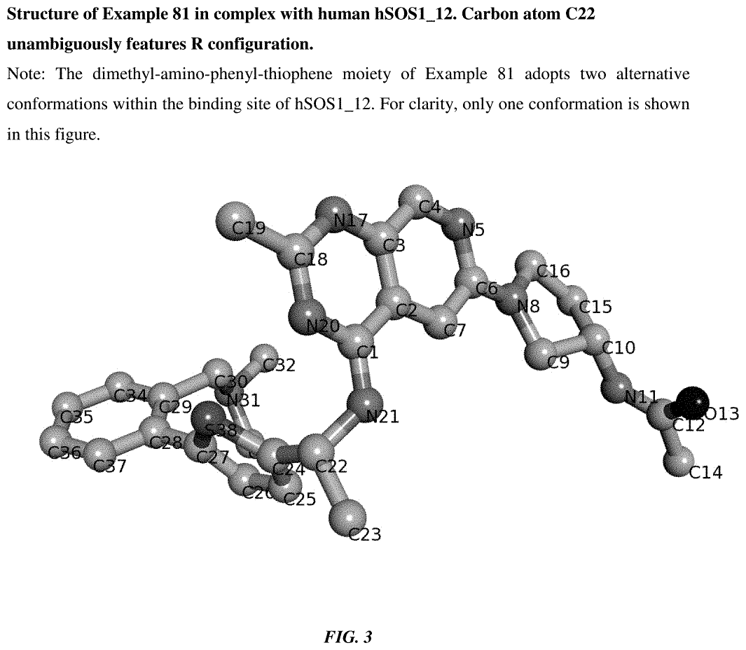

The complex of hSOS1_12 and Example 81 crystallizes with one hSOS1_12 molecule in the asymmetric unit. For co-crystallization, the active stereoisomer of Example 81 was employed (S configuration at C₁₂ and unknown conformation at C22). The electron density maps allowed the deduction of the configuration at C22 of the stereoisomer bound in the crystal. The stereo chemistry at the central carbon atom C22 of Example 81 (FIG. 3) is unambiguously defined by the knowledge of the stereo chemistry of the protein hSOS1_12. Example 81 unambiguously features the R configuration on carbon atom C22 (FIG. 3).

1. A compound of formula (I): 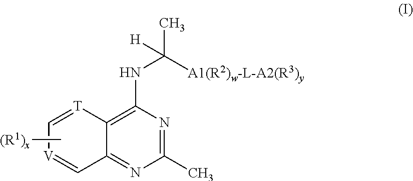
wherein:
R¹ is a substituent selected from the group consisting of:
a hydrogen atom,
a halogen atom,
a hydroxy group,
a cyano group,
a nitro group,
a C₁-C₆-alkylsulfanyl group,
—NR^{a}R^{b}, wherein R^{a} and R^{b} are independently selected from the group consisting of a hydrogen atom and C₁-C₆-alkyl,
C₁-C₆-alkyl,
C₁-C₆-alkoxy,
C₂-C₆-alkenyl,
C₂-C₆-alkynyl,
C₃-C₈-cycloalkyl,
C₄-C₈-cycloalkenyl,
4- to 7-membered heterocycloalkyl,
5- to 10 membered heterocycloalkenyl,
heterospirocycloalkyl,
fused heterocycloalkyl,
bridged heterocycloalkyl,
phenyl,
heteroaryl,
C₁-C₆-haloalkyl,
—C(═O)OH,
—C(═O)OR^{c}, wherein R^{c} is C₁-C₆-alkyl, C₃-C₆-alkenyl, C₃-C₆-alkynyl, C₃-C₈-cycloalkyl or C₄-C₈-cycloalkenyl,
—N═S(═O)(R^{d})R^{e}, wherein R^{d} and R^{e} are independently selected from the group consisting of C₁-C₆-alkyl, C₂-C₆-alkenyl, C₂-C₆-alkynyl, C₃-C₈-cycloalkyl and C₄-C₈-cycloalkenyl,
—NH—C(O)—C₁-C₆-alkyl,
—NH—C(O)—NR^{a}R^{b}, wherein R^{a} and R^{b} are selected independently from a hydrogen atom or a C₁-C₆-alkyl,
—NH—(CH₂)ₖ—NH—C(O)—C₁-C₆-alkyl, wherein k is 1 or 2,
—NH—(CH₂)ₗ—R^{f}, wherein 1 is 0, 1 or 2 and R^{f} stands for is a 4- to 7-membered heterocycloalkyl, heteroaryl or C₁-C₆-alkylsulfonyl,
whereby wherein in all foregoing definitions the C₁-C₆-alkyl-, C₁-C₆-alkoxy-, the 4- to 7-membered heterocycloalkyl and the heteroaryl can be optionally substituted, one or two or three times, identically or differently, with a halogen atom, hydroxy, oxo (═O), a cyano, nitro, C₁-C₆-alkyl, C₂-C₆-alkenyl, C₂-C₆-alkynyl, C₃-C₈-cycloalkyl, 4- to 7-membered heterocycloalkyl, C₁-C₆-alkoxy, C₁-C₆-haloalkyl, C₁-C₆-haloalkoxy, C₁-C₆-alkylsulfonyl, phenyl, benzyl, heteroaryl, —CH₂-heteroaryl, C₃-C₈-cycloalkoxy. phenyloxy, heteroaryloxy, —NH—C(O)—C₁-C₆-alkyl or NR^{a}R^{b}, wherein R^{a} and R^{b} are independently selected from the group consisting of a hydrogen atom and C₁-C₆-alkyl,
—O—(CH₂)_{z}-phenyl, —O(CH₂)_{z}—C₄-C₇-heterocycloalkyl, —O(CH₂)_{z}-heteroaryl, wherein z is 0, 1 or 2, and the phenyl, heterocycloalkyl and heteroaryl can optionally be substituted with a group selected from the group consisting of hydroxy, heterocycloalkyl, and heterocaclyoalkenyl, which both can be substituted with a methyl- and/or oxo-group, 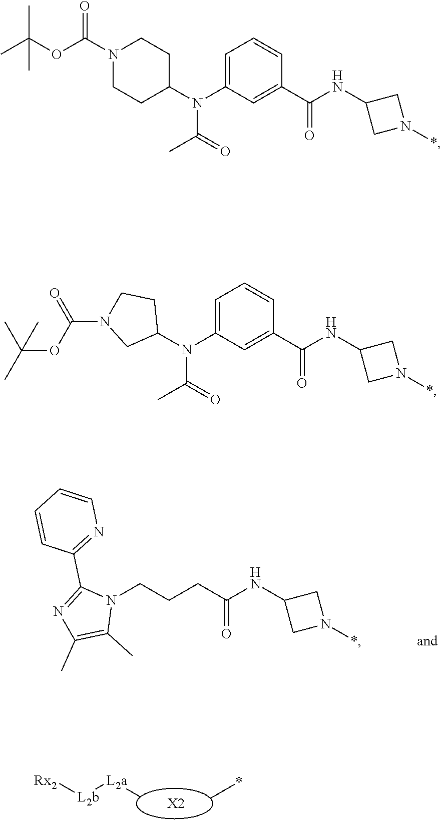
wherein L₂a is C(O),
L₂b is a bond or C₁-C₆-alkylene,
X2 is 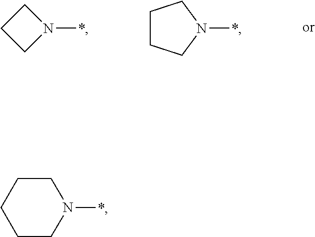
and Rx₂ is 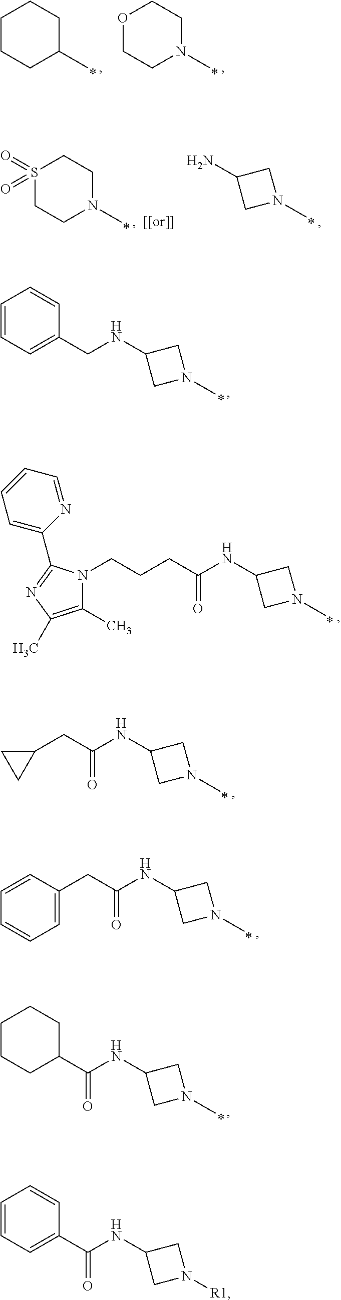 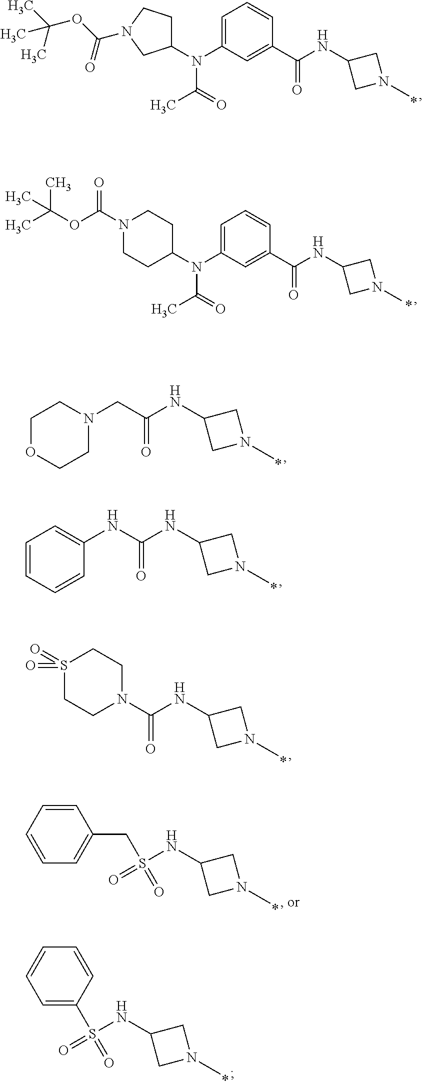
and wherein x is 1, 2 or 3,
A1 is a C₄-C₁₂-carbocyclic, heterocyclic, optionally bicyclic, optionally aromatic or optionally heteroaromatic ring system, wherein in a bicyclic aromatic or heteroaromatic ring system one or two double bonds can be hydrogenated,
R² is a substituent selected from the group consisting of:
a hydrogen atom,
a hydroxy group,
oxo (═O),
a halogen atom,
a cyano group,
C₁-C₆-alkyl,
C₁-C₆-alkoxy,
C₂-C₆-alkenyl,
C₃-C₈-cycloalkyl,
C₄-C₈-cycloalkenyl;
4- to 7-membered heterocycloalkyl,
—O—CH₂-4- to 7-membered heterocycloalkyl,
5- to 10-membered heterocycloalkenyl,
heterospirocycloalkyl,
fused heterocycloalkyl,
bridged heterocycloalkyl,
phenyl,
heteroaryl,
C₁-C₆-haloalkyl,
C₁-C₆-alkylsulfonyl,
—NR^{a}R^{b}, wherein R^{a} and R^{b} are independently selected from the group consisting of a hydrogen atom and C₁-C₆-alkyl,
—C(O)—NR^{a}R^{b}, wherein R^{a} and R^{b} are independently selected from the group consisting of hydrogen atom and C₁-C₆-alkyl,
—C(O)—O—R^{g}, wherein R^{g} is a hydrogen atom or C₁-C₆-alkyl,
—O—R^{h}, wherein R^{h} is C₁-C₆-alkyl, and
—CH₂—NR^{a}R^{b}, wherein R^{a} and R^{b} are independently selected from the group consisting of hydrogen atoms and C₁-C₆-alkyl,
and wherein w is 1 or 2,
and wherein A2(R³)_{y} is either a hydrogen atom or
A2 has the same meanings as the substituent A1 and
R³ is a substituent selected from the group consisting of:
a hydrogen atom,
a halogen atom,
a hydroxy group,
an oxo group,
a cyano group,
a nitro group,
C₁-C₆-alkyl,
C₂-C₆-alkenyl,
C₂-C₆-alkynyl,
C₃-C₈-cycloalkyl,
C₄-C₈-cycloalkenyl,
C₇-C₈-cycloalkynyl,
4- to 7-membered heterocycloalkyl,
5- to 10-membered heterocycloalkenyl,
phenyl,
heteroaryl,
C₁-C₆-haloalkyl,
which substituent is optionally substituted, one, two or three times, identically or differently, with a substituent selected from the group consisting of:
a halogen atom,
a hydroxy group,
an oxo (═O) group,
a cyano group,
C₁-C₆-alkyl,
C₁-C₆-alkoxy,
C₁-C₆-haloalkyl,
phenyl,
—C(O)NRⁱR^{j}. wherein Rⁱ and R^{j} are independently selected from the group consisting of a hydrogen atom a C₁-C₆-alkyl, and heteroaryl,
—NR^{k}R^{l}, wherein R^{k} and R^{l} are idependently selected from the group consisting of
a hydrogen atom,
C₁-C₆-alkyl,
C₂-C₆-alkenyl,
C₂-C₆-alkynyl,
C₃-C₈-cycloalkyl,
C₁-C₆-alkylsulfonyl,
phenyl,
heteroaryl,
4- to 7-membered heterocycloalkyl,
which are optionally substituted one, two or three times, identically or differently, with a substituent selected from the group consisting of
C₁-C₆-haloalkyl,
hydroxyl,
oxo (═O),
phenyl,
cyano,
C₁-C₆-alkoxy, and
heteroaryl, wherein the heteroaryl can optionally be substituted with a methyl group,
—CH₂—C(O)—R^{m}, wherein R^{m} is a bicyclic heteroaryl, which can be partially hydrogenated, a C₁-C₆-alkoxy or a group —NRⁿR^{o}, wherein Rⁿ and R^{o} are selected independently from the group consisting of hydrogen, C₁-C₆-alkyl, and phenyl, wherein the C₁-C₆-alkyl can optionally be substituted with a C₁-C₆-alkoxy or a phenyl, or —NRⁿR^{o} is a 4- to 7-membered azacycloalkyl, bound via the nitrogen atom to the rest of the molecule and which optionally contains one more heteroatom selected from the group consisting of nitrogen and oxygen, and
—C(═O)R^{p}, wherein R^{p} is selected from the group consisting of C₁-C₆-alkoxy, C₁-C₆-alkyl, which is optionally substituted, one, two or three times, identically or differently, with a substituent selected from the group consisting of hydroxyl and C₁-C₆-alkoxy, a mono- or bicyclic heteroaryl, and a 4- to 7-membered heterocycloalkyl or R^{p} a group —CH₂—NR^{q}R^{r}; wherein R^{q} and R^{r} are selected independently from the group consisting of hydrogen, phenyl and a C₁-C₆-alkyl, which may optionally be substituted up to threefold with fluorine, and
—NR^{s}R^{t}, wherein NR^{s}R^{t} is a 4- to 7-membered azacycloalkyl, bound via the nitrogen atom to the rest of the molecule, or a 6- to 10-membered azaspirocycloalkyl, which both may contain up to 2 further heteroatoms selected from the group consisting of nitrogen and oxygen and which both are optionally substituted one, two or three times, identically or differently, with a substituent selected from the group consisting of: hydroxy, oxo (═O), C₁-C₆-alkyl, C₁-C₆-hydroxyalkyl, —C(═O)OR^{u}, wherein Rⁿ is a C₁-C₆-alkyl, halogen, —N(C₁-C₆-alkyl)₂, —CH₂—N(C₁-C₆-alkyl)₂, and —C(O)NR^{a}R^{b}, wherein R^{a} and R^{b} are selected independently from the group consisting of a hydrogen atom and a C₁-C₆-alkyl,
—C(═O)R^{v}, —C(═O)NH₂, —C(═O)N(H)R^{v}, —C(═O)N(R^{v})R^{w}, —C(═O)OR^{v}, wherein R^{v} and R^{w} represent, independently from each other, a group selected from the group consisting of hydrogen, C₁-C₄-alkyl, C₁-C₄-haloalkyl, and phenyl, or a group (CH₂)₂—NR^{x}R^{y}, wherein R^{x} and R^{y} independently from each other are hydrogen, C₁-C₄-alkyl or (CH₂)₂N(CH₃)₂;
—NH₂,—NHR^{z}, —N(R^{z})R^{za}, —N(H)C(═O)R^{z}, —N(H)C(═O)OR^{z}, —N(H)S(═O)₂R^{z}, 4- to 7-membered heterocycloalkyl, heteroaryl, heterospirocycloalkyl, fused heterocycloalkyl, bridged heterocycloalkyl, wherein R^{z} and R^{za} represent, independently from each other, a group selected from the group consisting of C₁-C₄-alkyl, C₁-C₄-haloalkyh and phenyl,
C₁-C₆-alkoxy-, C₁-C₆-haloalkoxy-, —O—(CH₂)ₛ—C₃-C₈-cycloalkyl, —O—(CH₂)ₛ-phenyl, —O—(CH₂)ₛ-heterocycloalkyl, —O—(CH₂)ₛ-heteroaryl, wherein s is 0, 1, 2 or 3,
—S(═O)₂R^{z}, —S(═O)₂NH₂, —S(═O)₂NHR^{z}, and —S(═O)₂N(R^{z})R^{za}, wherein R^{z} and R^{za} represent, independently from each other, a group selected from the group consisting of C₁-C₄-alkyl, C₁-C₄-haloalkyl and phenyl,
wherein y is 1, 2 or 3, and
L is either a bond or O—(CH₂)^{k}, wherein k is 0, 1, 2 or 3, or a group CH═CH—(CH₂)ₙ, wherein n is 0, 1 or 2,
and either both T and V are nitrogen or T is carbon and V is nitrogen or T is nitrogen and V is carbon,
or a tautomer, an N-oxide, a hydrate, a solvate, or a salt thereof, or a mixture of the foregoing.

2. The compound according to claim 1 of formula (II) 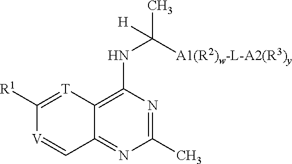
wherein:
R¹ is a substituent selected from the group consisting of:
a halogen atom,
a C₁-C₆-alkylsulfanyl group,
—NR^{a}R^{b}, wherein R^{a} and R^{b} are independently selected from the group consisting of a hydrogen atom and C₁-C₆-alkyl,
C₁-C₆-alkoxy,
4- to 7-membered heterocycloalkyl,
5- to 10 membered heterocycloalkenyl[M]_{z}
—NH—(CH₂)ₖ—NH—C(O)—C₁-C₆-alkyl, wherein k is 1 or 2,
—NH—(CH₂)ₗ—R^{f}, wherein 1 is 0, 1 or 2 and R^{f} is a 4- to 7-membered heterocycloalkyl, heteroaryl or C₁-C₆-alkylsulfonyl;
whereby in all foregoing definitions the C₁-C₆-alkyl-, C₁-C₆-alkoxy-, the 4- to 7-membered heterocycloalkyl and the heteroaryl can be optionally substituted, one or two or three times, identically or differently, with a hydroxy, oxo (═O), C₁-C₆-alkyl, C₃-C₈-cycloalkyl, 4- to 7-membered heterocycloalkyl, C₁-C₆-alkoxy, C₁-C₆-alkylsulfonyl, phenyl, benzyl, heteroaryl, -CH₂-heteroaryl, C₃-C₈-cycloalkoxy, phenyloxy, heteroaryloxy, —NH—C(O)-C i-C₆-alkyl or —R^{a}R^{b}, wherein R^{a} and R^{b} are independently selected from the group consisting of a hydrogen atom and C₁-C₆-alkyl,
—O—(CH₂)_{z}-phenyl, wherein z is 0, 1 or 2, and the phenyl, can optionally be substituted with a group selected from the group consisting of hydroxy, heterocycloalkyl, and heterocaclyoalkenyl, which both can be substituted with a methyl group, 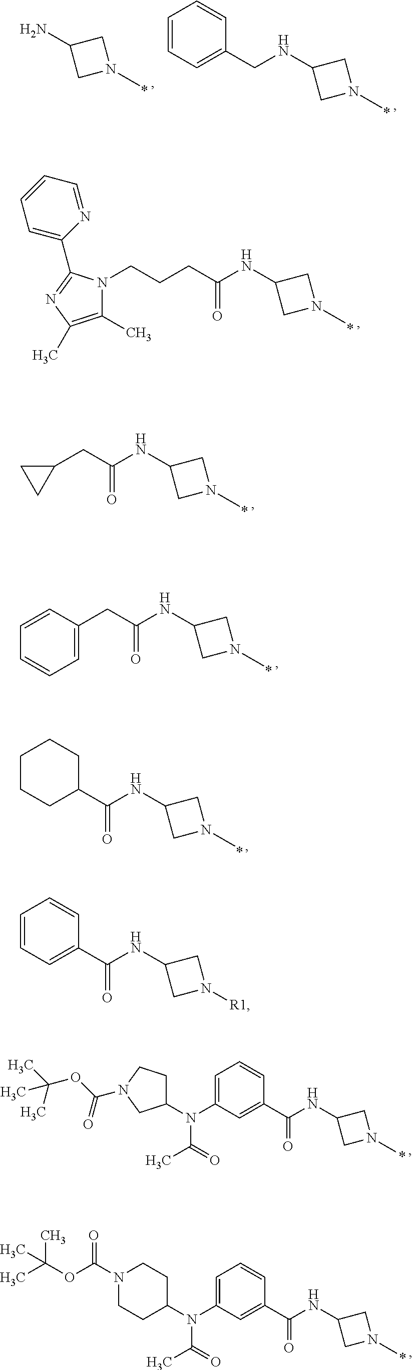 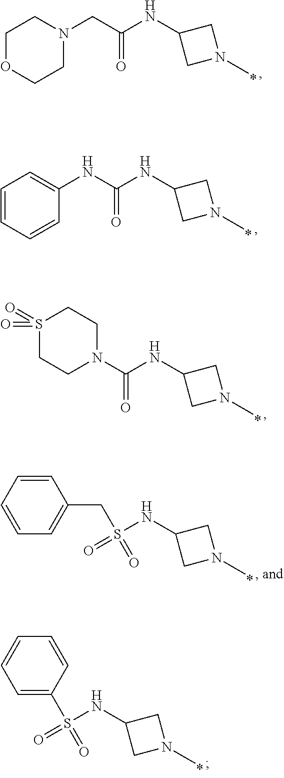 [SMILES: CN1CC(NC(=O)CN2CCOCC2)C1 ; CN1CC(NC(=O)Nc2ccccc2)C1 ; CN1CC(NC(=O)N2CCS(=O)(=O)CC2)C1 ; CN1CC(NS(=O)(=O)Cc2ccccc2)C1 ; CN1CC(NS(=O)(=O)c2ccccc2)C1]
A1 stands for is an optionally bicyclic C₅-C₉-aromatic or an optionally bicyclic C₅-C₉-heteroaromatic ring system;
R² is a substituent selected from the group consisting of:
a hydrogen atom,
a halogen atom,
C₁-C₆-alkyl,
C₃-C₈-cycloalkyl, and
C₁-C₆-alkylsulfonyl;
and wherein w is 0, 1 or 2;
and wherein A2(R³)_{y} stands either for a hydrogen atom or
A2 is phenyl and
R³ is
C₁-C₆-alkyl, which is substituted with a substituent selected from the group consisting of:
a hydroxy group, and
—NR^{k}R^{l}, wherein R^{k} and R^{l} are independently selected from the group consisting of
a hydrogen atom, and
C₁-C₆-alkyl,
wherein y is 1; and
L is a bond,
and either both T and V are nitrogen or T is carbon and V is nitrogen or T is nitrogen and V is carbon,
or a tautomer, an N-oxide, a hydrate, a solvate, or a salt thereof, or a mixture of the foregoing.

3. The compound according to claim 1, in which wherein:
R¹ is selected from the group consisting of
H, *-OCH₃, *-OC₂H₅, 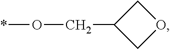 [SMILES: *OCC1COC1]
*-CH₂O H, *-C(O)OH, *-C(O)OCH₃, —Br, *-O—CH(CH₃)₂, *-O—(CH₂)₂CH(CH₃)₂, *-O—(CH₂)₃CH₃, *-O—(CH₂)₂O-CH₃, 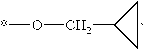 [SMILES: *OCC1CC1]
*-O—CH₂-Phenyl, *-N═S (O)(CH₃)₂, *-CH₃, 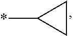 [SMILES: *C1CC1]
*-NH(CH₃),
*-N(CH₃)₂, *-NH₂, 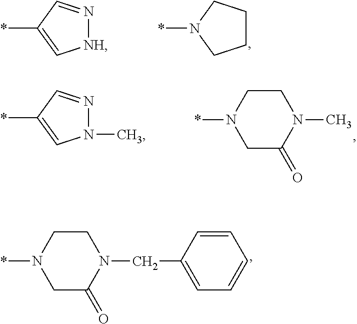
*-C(CH₃)₂—OH, 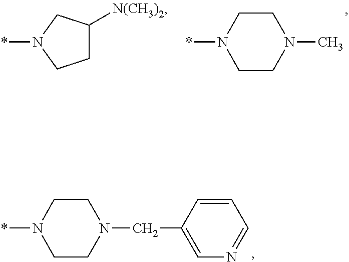
*-NH—(CH₂)₂—NH—C(O)—CH₃, *-NH—(CH₂)₂-morpholino, *-NH—C(O)—CH₃, *-NH—C(O)—NH—CH₃, *-NH—C(O)—N(CH₃)₂, *-NO₂, *-NH—S(O)₂—CH₃, *-N═S(O)(CH₃)₂, *-OH, *-O—(CH₂)₂—S(O)₂—CH₃, *-fluorine, 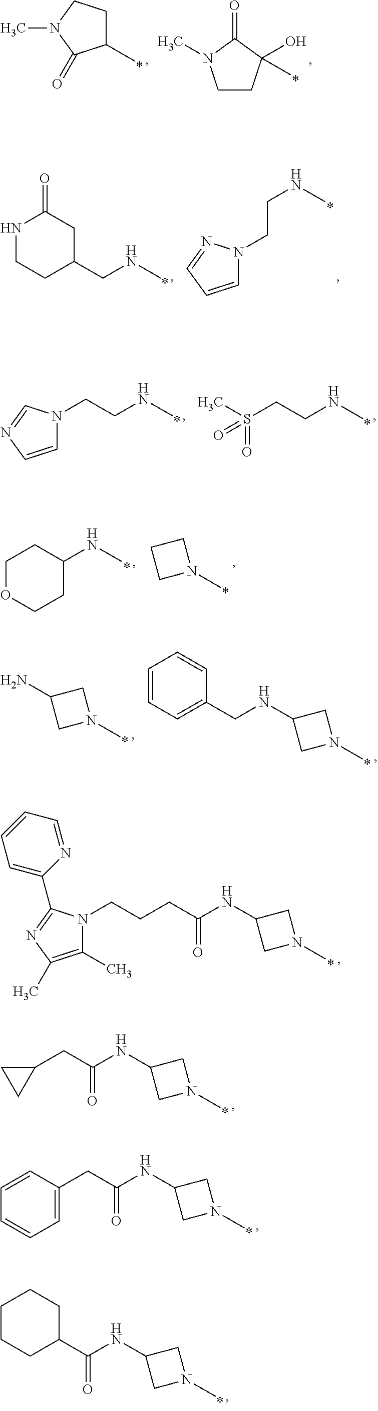 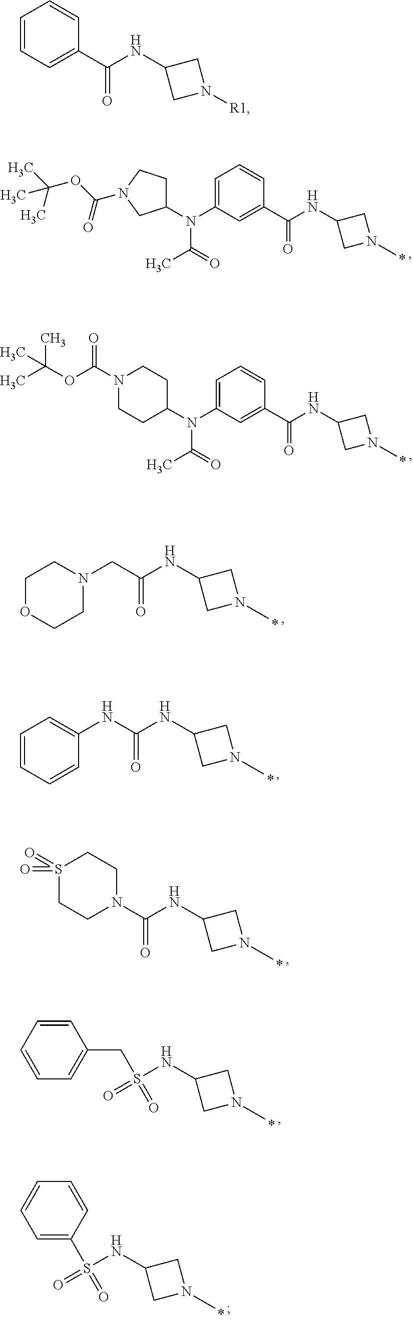 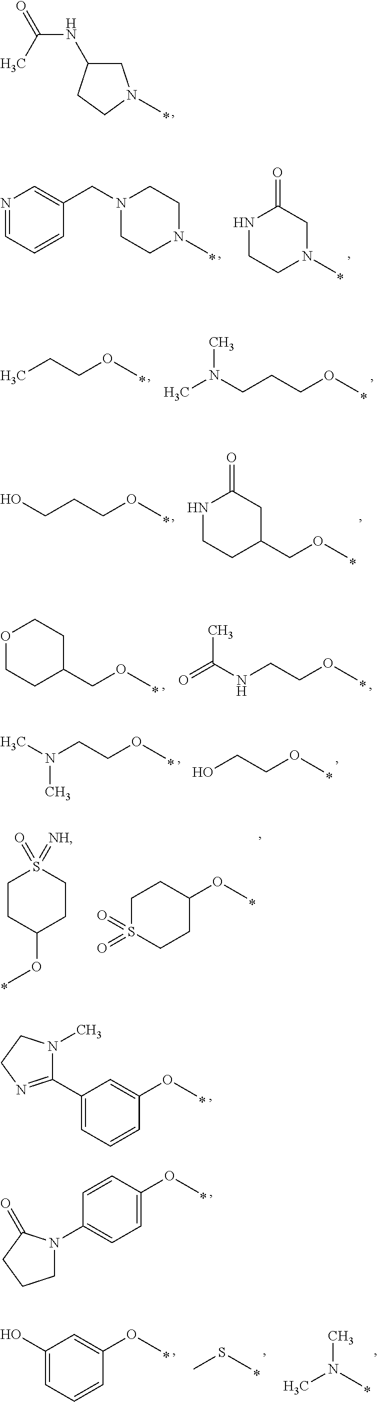 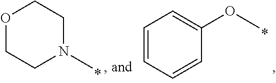
and
z is 1 or 2 and
x is 1 or 2 and wherein
A1 is selected from the group consisting of 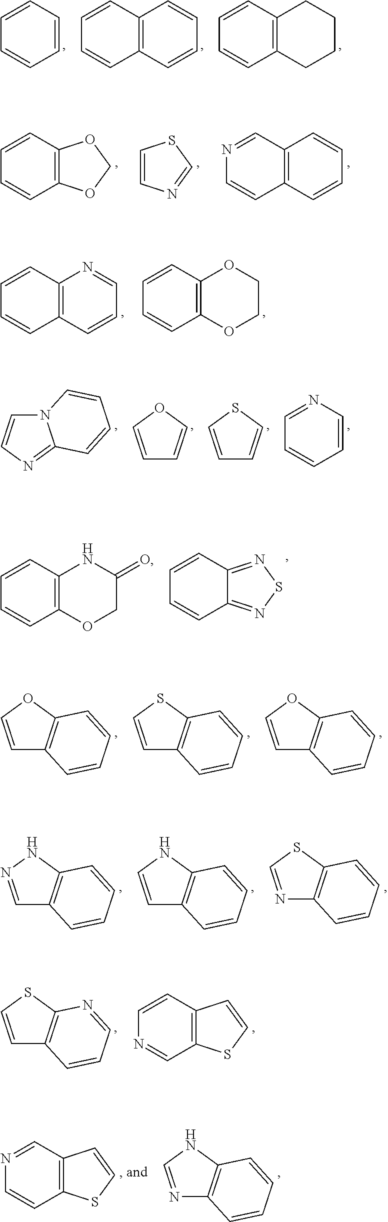
and
R² is selected from the group consisting of hydrogen, hydroxy, oxo (═O), cyano, cyclopropyl, 1,1-dimethylcyclopropyl, —C(═CH₂)CH₃, —C(CH₃)═CHCH₃, —CH═CH—(CH₂)₂CH₃, CH═CHCH₃, —CH═CH-cyclopropyl), —C(O)NH₂, C(O)OCH₃, —S(O)₂CH₃, —OCH₃, —CH₂NH₂, and a halogen atom, and
w is 1 or 2 and
A2 is selected from the group consisting of 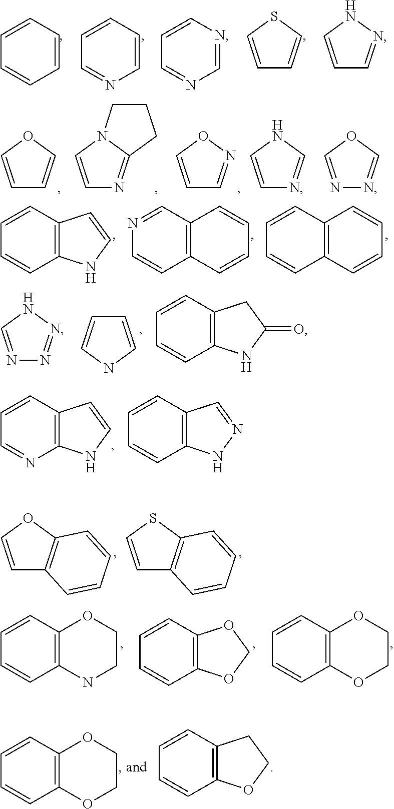
and
R³ is selected from the group consisting of
*-C(O)NH—(CH₂)₂CH₃,
*-C(O)—N(CH₃)₂,
*-C(O)—NH₂,
*-C(O)—NH—(CH₂)₂N(CH₃)₂,
*-CH₂—C(O)—NH₂,
hydrogen
*-F, *-Cl, *-Br,
*-C≡N; *-CH₃, *-C₂H₅, *-CH═CH₂,
*-CH₂—CN, *-CH(CH₃)—NH₂, *-CH═CH—CN,
*-C(O)—OH, *-C(O)—OCH₃, *-C(O)—CH₃, *-C(CH₃)₂—C(O)—OCH₃, *-C(CH₃)₂—CN, Oxo(═O), hydroxy, 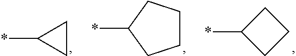
*-NH₂,
*-NH-C(O)CH₃,
*-NH—SO₂—CH₃,
*-NH—C(O)—O—C(CH₃)₃, 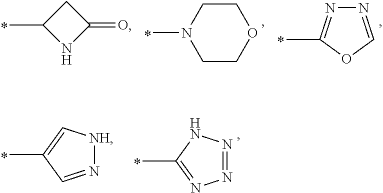
*-SO₂—CH₃,
*-SO₂—N(CH₃)₂,
*-SO₂—NH₂,
*-O—CH₂—CH₃, *-O—(CH₂)₂—CH₃, *-O—CF₃, 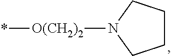
*-OCH₂-Cyclopropyl, *-OCH₃,
*-O(CH₂)₃—CH₃, *-OCH₂-Phenyl, *-O-Phenyl,
*-(CH₂)—OH, *-(CH₂)₂—OH,
*-(CH₂)—O—CH₃,
*-(CH₂)—O—CH₂—CH₃,
*-CH(OH)—CH₂-Phenyl,
*-CH(OH)—CH₂—CH₃,
*-CH(OH)—(CH₂)₂—CH₃,
*-CH(OH)—(CH₂)₃—CH₃,
*-CH(OH)—CH—(CH₃)₂,
*-CH(OH)-Phenyl,
*-CH(OH)-CN
*-CH(OH)—CH₂OH,
*-CH(OH)—CF₃,
*-CH(OH)—(CH₂)₂-Phenyl,
*-CH(OH)—C≡CH*-CH(NH₂)—CH₂—COOH,
*-CH₂—NH—SO₂—CH₃,
*-CH₂—NH—(CH₂)₃—CH₃,
*-CH₂—NH—CH₃,
*-CH₂—N(CH₃)₂,
*-CH₂—NH—C₂H₅, *-CH(CH₃)—NH₂,
*-CH₂—NH₂,
*-(CH₂)₂—NH₂, *-CH₂—NH—CH₂-Phenyl,
*-CH₂—N(C₂H₅)₂,
*-CH₂—NH—Cyclopropyl,
*-CH₂—NH—Cyclobutyl,
*-CH₂—NH—Cyclopentyl,
*-CH₂—NH-Pyridyl,
*-CH₂—NH-Phenyl,
*-CH₂—NH—(CH₂)₂—OH,
*-CH₂—N(CH₃)(CH₂)₂OH,
*-CH₂—NH—CH₂—CN,
*-CH₂—N(CH₃)—CH₂—CN,
*-CH₂—N(CH₃)—CH₂—CF₃,
*-CH₂—N(CH₃)—CH₂—CF₂H,
*-CH₂—NH—CH₂—CF₂H,
*-CH₂—NH—CH₂—CF₃,
*-CH₂—NH—(CH₂)₂—OCH₃, 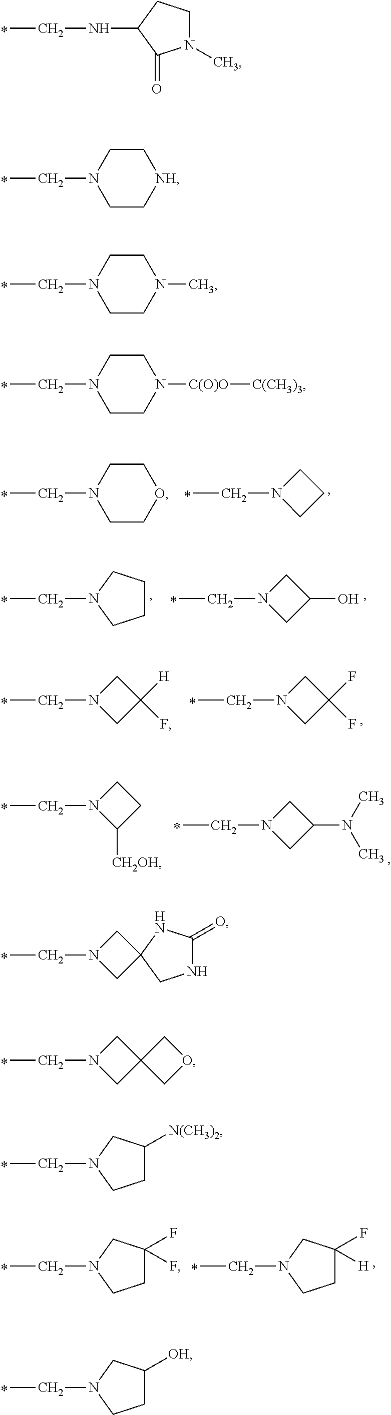 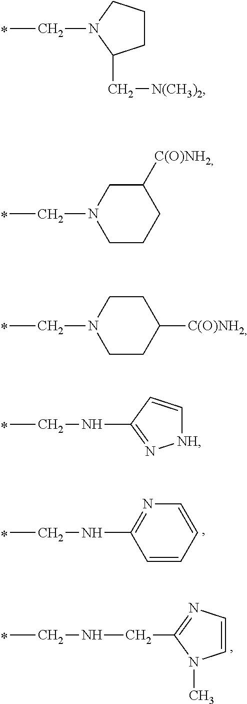
*-CH₂—NH—C(O)—O—C(CH₃)₃,
*-(CH₂)₂—NH—C(O)—O—C(CH₃)₃,
*-CH₂—NH—C(O)—CH₂—OH,
*-CH₂—NH—C(O)—CH₂—OCH₃,
*-CH—(CH₃)—NH—C(O)—O—C(CH₃)₃,
*-CH₂—NH—C(O)—CH₃, 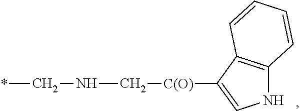
*-CH₂—NH—CH₂—C(O)—NH₂,
*-CH₂—NH—CH₂—C(O)—N(CH₃)₂,
*-CH₂—NH—CH₂—C(O)—OCH₃,
*-CH₂—NH—CH₂—C(O)—NHCH₃,
*-CH₂—NH—CH₂—C(O)—NH—(CH₂)₂—O—CH₃,
*-CH₂—NH—CH₂—C(O)—NH—CH₂-Phenyl 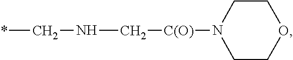
*-CH₂—NH—CH₂—C(O)—NH-Phenyl, 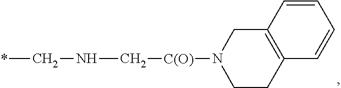
*-CH₂—NH—C(O)—CH₂—NH-Phenyl, 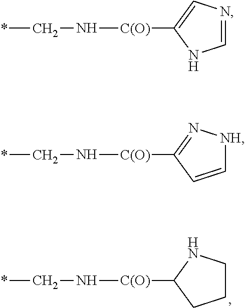
*-CH₂—NH—C(O)—CH₂—NH—CH₂—CF₃, and 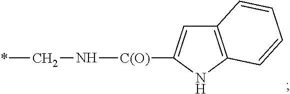
y is 1 or 2;
k is 1 or 2; and
n is0or 1;
or a tautomer, an N-oxide, a hydrate, a solvate, or a salt thereof, or a mixture of the foregoing

4. The compound according to claim 3,
wherein R¹ is selected from the group consisting of
H, *-OCH₃, *-OC₂H₅, 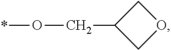 [SMILES: *OCC1COC1]
*-CH₂OH, *-C(O)OH, *-C(O)OCH₃, —Br, *-O—CH(CH₃)₂, *-O—(CH₂)₂CH(CH₃)₂, *-O—(CH₂)₃CH₃, *-O—(CH₂)₂O—CH₃, 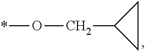 [SMILES: *OCC1CC1]
*-O—CH₂-Phenyl, *-N═S (O)(CH₃)₂, *-CH₃, 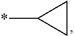 [SMILES: *C1CC1]
*-NH(CH₃), *-N(CH₃)₂, *-NH₂, 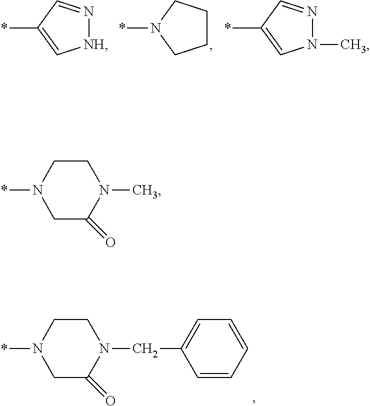
*-C(CH₃)₂—OH, 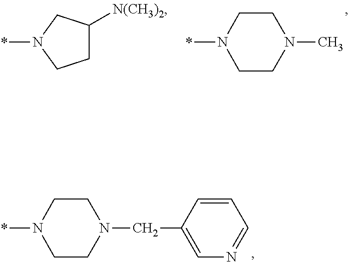
*-NH—(CH₂)_{2—}NH—C(O)—CH₃, *-NH—(CH₂)₂-morpholino, *-NH—C(O)—CH₃, *-NH—C(O)—NH—CH₃, *-NH—C(O)—N(CH₃)₂, *-NO₂, *-NH—S(O)₂—CH₃, *-N═S(O)(CH₃)₂, *-OH, and *-O—(CH₂)₂—S(O)₂—CH₃;
or a tautomer, an N-oxide, a hydrate, a solvate, or a salt thereof, or a mixture of the foregoing.

5. The compound according to claim 1,
wherein R¹ is selected from the group consisting of
H, *-OCH₃, —OC₂H₅, 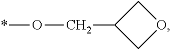 [SMILES: *OCC1COC1]
*-CH₂OH, *-C(O)OH, *-C(O)OCH₃, —Br, —O—CH(CH₃)₂, —O—(CH₂)₂CH(CH₃)₂, —O—(CH₂)₃CH₃, —O—(CH₂)₂O—CH₃, 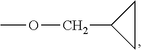
—O—CH₂-Phenyl, —N═S (O)(CH₃)₂, —CH₃, 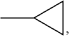
—NH(CH₃), —N(CH₃)₂, —NH₂, 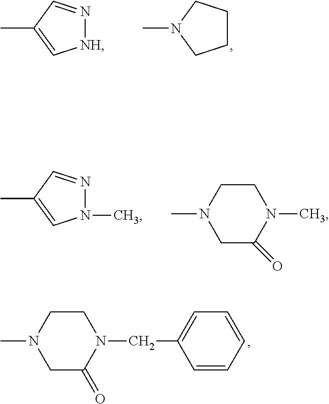
—C(CH₃)₂—OH, 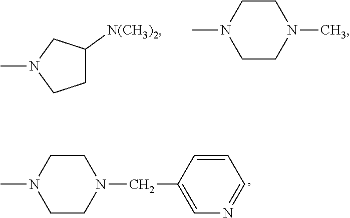
—NH—(CH₂)_{2—}NH—C(O)—CH₃, —NH—(CH₂)₂-morpholino, —NH—C(O)—CH₃, —NH—C(O)—NH—CH₃, —NH—C(O)—N(CH₃)₂, —NO₂, —NH—S(O)₂—CH₃, —N═S(O)(CH₃)₂, —OH, —O—(CH₂)₂—S(O)₂—CH₃, *-fluorine, 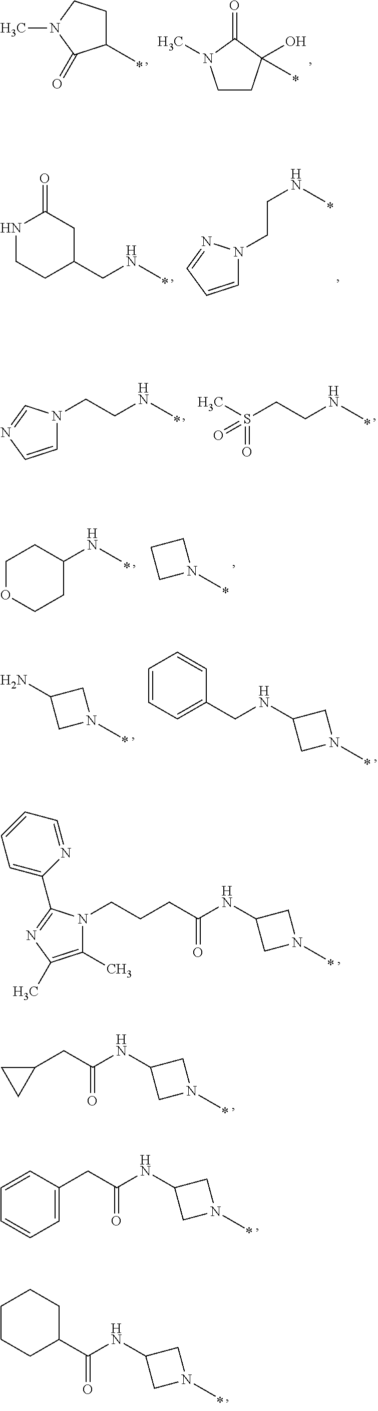 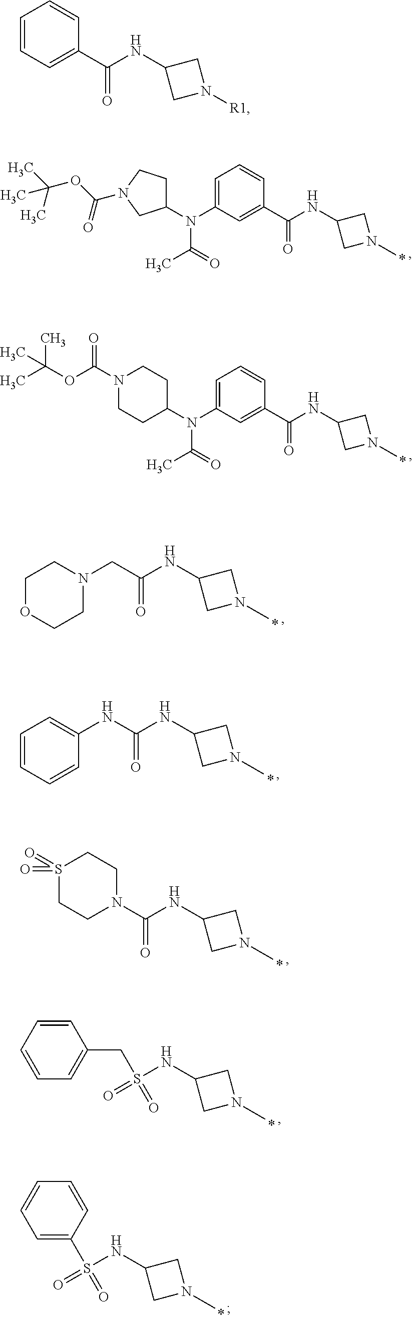 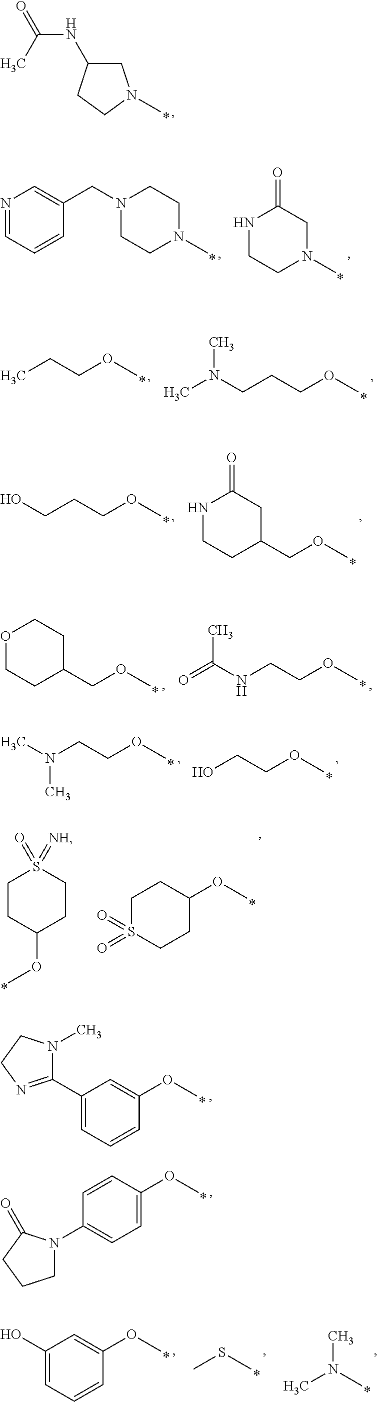 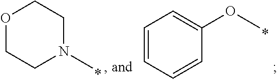
or a tautomer, an N-oxide, a hydrate, a solvate, or a salt thereof, or a mixture of the foregoing.

6. The compound according to claim 5,
wherein R¹ is selected from the group consisting of
H, OCH₃, —OC₂H₅, 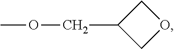
—CH₂OH, —C(O)OH, —C(O)OCH₃, —Br, —O—CH(CH₃)₂, —O—(CH₂)₂CH(CH₃)₂, —O—(CH₂)₃CH₃, —O—(CH₂)₂O—CH₃, 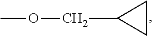
—O—CH₂-Phenyl, —N═S (O)(CH₃)₂, —CH₃, 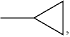
—NH(CH₃), —N(CH₃)₂, —NH₂, 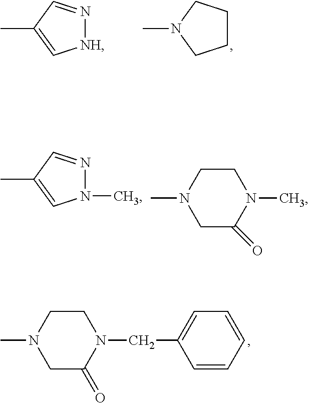
—C(CH₃)₂—OH, 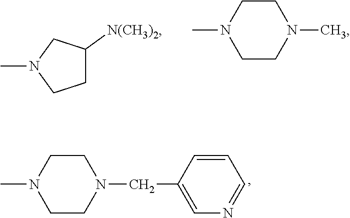
—NH—(CH₂)_{2—}NH—C(O)—CH₃, —NH—(CH₂)₂-morpholino, —NH—C(O)—CH₃, —NH—C(O)—NH—CH₃,
—NH—C(O)—N(CH₃)₂, —NO₂, —NH—S(O)₂—CH₃, —N═S(O)(CH₃)₂, —OH, and —O—(CH₂)₂—S(O)₂—CH₃;
or a tautomer, an N-oxide, a hydrate, a solvate, or a salt thereof, or a mixture of the foregoing.

7. The compound according to claim 1,
wherein A1 is selected from the group consisting of 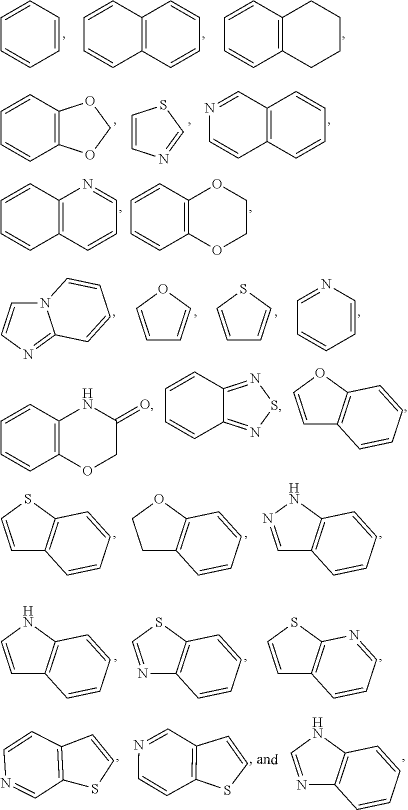
or a stereoisomer, a tautomer, an N-oxide, a hydrate, a solvate, or a salt thereof, or a mixture of the foregoing.

8. The compound according to claim 7, wherein A1 is a phenyl ring; or a tautomer, an N-oxide, a hydrate, a solvate, or a salt thereof, or a mixture of the foregoing.

9. The compound according to claim 7, wherein A1 is a thienyl ring; or a tautomer, an N-oxide, a hydrate, a solvate, or a salt thereof, or a mixture of the foregoing.

10. The compound according to claim 1, wherein A2 is selected from the group consisting of 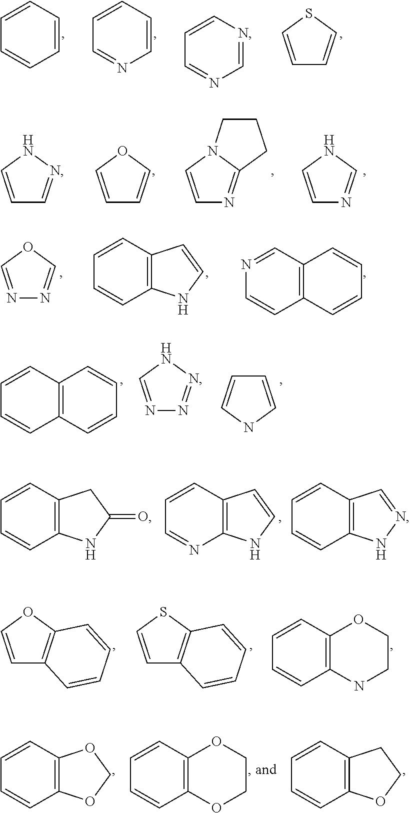
or a stereoisomer, a tautomer, an N-oxide, a hydrate, a solvate, or a salt thereof, or a mixture of the foregoing.

11. The compound according to claim 10, wherein A2 is a phenyl ring; or a tautomer, an N-oxide, a hydrate, a solvate, or a salt thereof, or a mixture of the foregoing.

12. The compound according to claim 1, wherein R³ is selected from the group consisting of
*-C(O)NH—(CH₂)₂CH₃,
*-C(O)—N(CH₃)₂,
*-C(O)—NH₂,
*-C(O)—NH—(CH₂)₂N(CH₃)₂,
*-CH₂—C(O)—NH₂,
hydrogen,
*-F, *-Cl, *-Br,*-C≡N, *-CF₃, *-CH₃, *-C₂H₅, *-CH═CH₂,
*-CH₂—CN, *-CH(CH₃)—NH₂, *-CH═CH—CN,
—C(O)—OH, *-C(O)—OCH₃, *-C(O)—CH₃, *-C(CH₃)₂—C(O)—OCH₃,
*-C(CH₃)₂—CN, Oxo(═O), hydroxy, 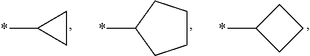
*-NH₂,
*-NH—C(O)CH₃,
*-NH—SO₂—CH₃,
*-NH—C(O)—O—C(CH₃)₃, 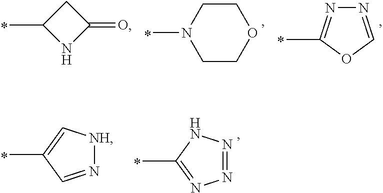
*-SO₂—CH₃,
*-SO₂—N(CH₃)₂,
*-SO₂—NH₂,
*-O—CH₂—CH₃, *-O—(CH₂)₂—CH₃, *-O—CF₃, 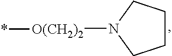
*-OCH₂-Cyclopropyl, *-OCH₃,
*-O(CH₂)₃—CH₃, *-OCH₂-Phenyl, *-O-Phenyl,
*-(CH₂)—OH₂—(CH₂)₂—OH,
*-(CH₂)—O—CH₃,
*-(CH₂)—O—CH₂-CH₃,
*-CH(OH)—CH₂-Phenyl,
*-CH(OH)—CH₂-CH₃,
*-CH(OH)—(CH₂)₂—CH₃,
*-CH(OH)—(CH₂)₃—CH₃,
*-CH(OH)—CH—(CH₃)₂,
*-CH(OH)-Phenyl,
*-CH(OH)—CN,
*-CH(OH)—CH₂OH,
*-CH(OH)—CF₃,
*-CH(OH)—(CH₂)₂-Phenyl,
*-CH(OH)-(OH)—C≡CH,
-CH(NH₂)—CH₂—COOH,
*-CH₂—NH—SO₂—CH₃,
*-CH₂—NH—(CH₂)₃—CH₃,
*-CH₂—NH—CH₃,
*-CH₂—N(CH₃)₂,
*-CH₂—NH—C₂H₅,
*-CH(CH₃)—NH₂,
*-CH₂—NH₂,
*-(CH₂)₂—NH₂,
*-CH₂—NH—CH₂-Phenyl,
*-CH₂—N(C₂H₅)₂,
*-CH₂—NH—Cyclopropyl,
*-CH₂—NH—Cyclobutyl,
* -CH₂—NH—Cyclopentyl,
*-CH₂—NH-Pyridyl,
*-CH₂—NH-Phenyl,
*-CH₂—NH—(CH₂)₂—OH,
*-CH₂—N(CH₃)(CH₂)₂OH,
*-CH₂—NH—CH₂—CN,
*-CH₂—N(CH₃)—CH₂—CN
*-CH₂—N(CH₃)—CH₂—CF₃,
*-CH₂—N(CH₃)—CH₂—CF₂H,
*-CH₂—NH—CH₂—CF₂H,
*-CH₂—NH—CH₂—CF₃,
*-CH₂—NH—(CH₂)₂—OCH₃, 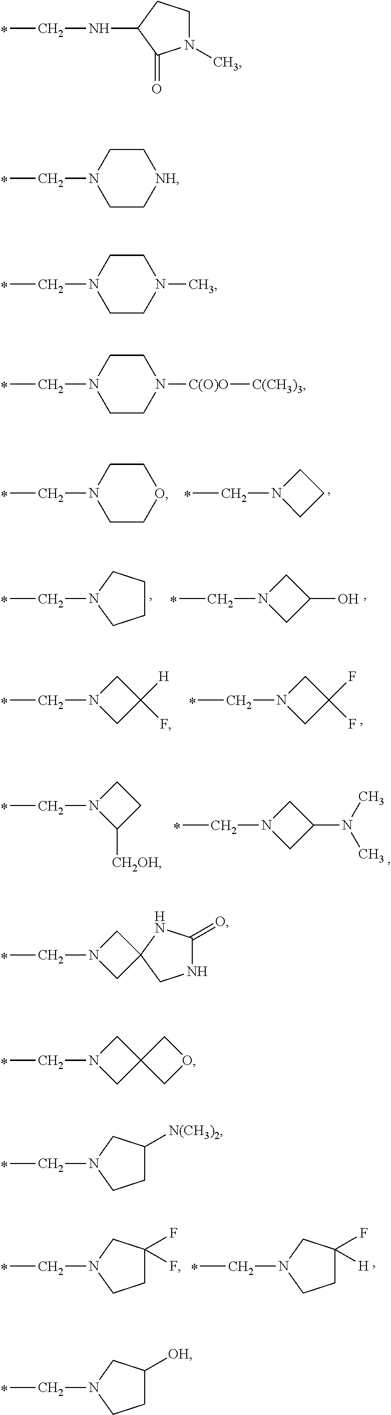 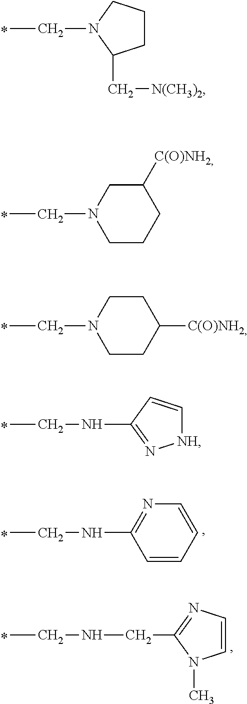
*-CH₂—NH—C(O)—O—C(CH₃)₃,
*-(CH₂)₂—NH—C(O)—O—C(CH₃)₃,
*-CH₂—NH—C(O)—CH₂—OH,
*-CH₂—NH—C(O)—CH₂—OCH₃,
*-CH—(CH₃)—NH—C(O)—O—C(CH₃)₃,
*-CH₂—NH—C(O)—CH₃, 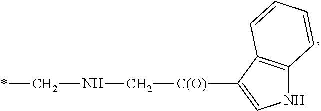
*-CH₂—NH—CH₂—C(O)—NH₂,
*-CH₂—NH—CH₂—C(O)—N(CH₃)₂,
*-CH₂—NH—CH₂—C(O)—OCH₃,
*-CH₂—NH—CH₂—C(O)—NHCH₃,
*-CH₂—NH—CH₂—C(O)—NH—(CH₂)₂—O—CH₃,
*-CH₂—NH—CH₂—C(O)—NH—CH₂-Phenyl, 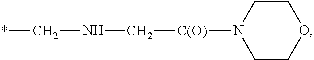
*-CH₂—NH—CH₂—C(O)—NH-Phenyl, 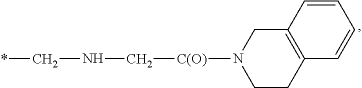
*-CH₂—NH—C(O)—CH₂—NH-Phenyl, 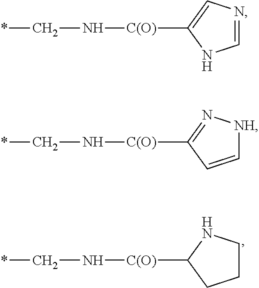
*-CH₂—NH—C(O)—CH₂—NH—CH₂-CF₃ and 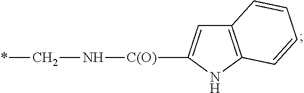
or a tautomer, an N-oxide, a hydrate, a solvate, or a salt thereof, or a mixture of the foregoing.

13. The compound according to claim 1, wherein R³ is a C₁- or C₂-alkyl substituted with a hydroxyl or a C₁-C₆-alkoxy; or a tautomer, an N-oxide, a hydrate, a solvate, or a salt thereof, or a mixture of the foregoing.

14. The compound according to claim 1, wherein x is 1 or 2; or a tautomer, an N-oxide, a hydrate, a solvate, or a salt thereof, or a mixture of the foregoing.

15. The compound according to claim 1, wherein y is 1 or 2; or a tautomer, an N-oxide, a hydrate, a solvate, or a salt thereof, or a mixture of the foregoing.

16. The compound according to claim 1, wherein z is 1 or 2; or a tautomer, an N-oxide, a hydrate, a solvate, or a salt thereof, or a mixture of the foregoing.

17. The compound according to claim 1, which is selected from the group consisting of:

| | |
|---|---|
| Example 1 | 6-ethoxy-N-[(1R)-1-(4-fluorophenyl)ethyl]-2-methylpyrido[3,4- |
| | d]pyrimidin-4-amine; |
| Example 2 | N-[(1R)-1-(4-fluorophenyl)ethyl]-6-methoxy-2-methylpyrido[3,4- |
| | d]pyrimidin-4-amine; |
| Example 3 | N-[1-(1-benzothiophen-4-yl)ethyl]-6-ethoxy-2-methylpyrido[3,4- |
| | d]pyrimidin-4-amine; |
| Example 4 | N-[1-(1-benzothiophen-4-yl)ethyl]-6-methoxy-2-methylpyrido[3,4- |
| | d]pyrimidin-4-amine; |
| Example 5 | N-[(1R)-1-(3-bromophenyl)ethyl]-6-ethoxy-2-methylpyrido[3,4- |
| | d]pyrimidin-4-amine; |
| Example 6 | N-[(1R)-1-(3-chlorophenyl)ethyl]-6-ethoxy-2-methylpyrido[3,4- |
| | d]pyrimidin-4-amine; |
| Example 7 | 6-ethoxy-2-methyl-N-{(1R)-1-[3- |
| | (methylsulfonyl)phenyl]ethyl}pyrido[3,4-d]pyrimidin-4-amine; |
| Example 8 | N-[(1R)-1-(3-chloro-4-fluorophenyl)ethyl]-6-methoxy-2- |
| | methylpyrido[3,4-d]pyrimidin-4-amine; |
| Example 9 | 6-fluoro-N-[(1R)-1-(4-fluorophenyl)ethyl]-2-methylpyrido[3,4- |
| | d]pyrimidin-4-amine; |
| Example 10 | N-[(1R)-1-(4-fluorophenyl)ethyl]-6-(2-methoxyethoxy)-2- |
| | methylpyrido[3,4-d]pyrimidin-4-amine; |
| Example 11 | N-[(1R)-1-(4-fluorophenyl)ethyl]-2-methyl-6-(tetrahydro-2H-pyran- |
| | 4-ylmethoxy)pyrido[3,4-d]pyrimidin-4-amine; |
| Example 12 | N-[(1R)-1-(3-cyclopropyl-4-fluorophenyl)ethyl]-6-methoxy-2- |
| | methylpyrido[3,4-d]pyrimidin-4-amine; |
| Example 13 | N-[(1R)-1-(4-bromophenyl)ethyl]-6-ethoxy-2-methylpyrido[3,4- |
| | d]pyrimidin-4-amine; |
| Example 14 | 6-methoxy-2-methyl-N-[1-(1-methyl-1H-indazol-4- |
| | yl)ethyl]pyrido[3,4-d]pyrimidin-4-amine; |
| Example 15 | N-[(1R)-1-(4-fluorophenyl)ethyl]-2-methyl-6-(propan-2- |
| | yloxy)pyrido[3,4-d]pyrimidin-4-amine; |
| Example 16 | N-[(1R)-1-(4-fluorophenyl)ethyl]-2-methyl-6- |
| | (methylsulfanyl)pyrido[3,4-d]pyrimidin-4-amine; |
| Example 17 | 6-methoxy-2-methyl-N-[1-(2-methyl-2H-indazol-4- |
| | yl)ethyl]pyrido[3,4-d]pyrimidin-4-amine; |
| Example 18 | 4-{(1R)-1-[(6-ethoxy-2-methylpyrido[3,4-d]pyrimidin-4- |
| | yl)amino]ethyl}benzonitrile; |
| Example 19 | 6-ethoxy-2-methyl-N-[(1R)-1-phenylethyl]pyrido[3,4-d]pyrimidin-4- |
| | amine; |
| Example 20 | 6-(benzyloxy)-N-[(1R)-1-(3-bromophenyl)ethyl]-2- |
| | methylpyrimido[5,4-d]pyrimidin-4-amine; |
| Example 21 | N8-[(1R)-1-(3-bromophenyl)ethyl]-N2,N2,6-trimethylpyrimido[5,4- |
| | d]pyrimidine-2,8-diamine; |
| Example 22 | N-[(1R)-1-(3-bromophenyl)ethyl]-2-methyl-6-(morpholin-4- |
| | yl)pyrimido[5,4-d]pyrimidin-4-amine; |
| Example 23 | N-[(1R)-1-(3-bromophenyl)ethyl]-6-ethoxy-2-methylpyrimido[5,4- |
| | d]pyrimidin-4-amine; |
| Example 24 | N-[(1R)-1-(3-bromophenyl)ethyl]-6-methoxy-2-methylpyrimido[5,4- |
| | d]pyrimidin-4-amine; |
| Example 25 | N-[(1R)-1-(3-bromophenyl)ethyl]-2-methyl-6-phenoxypyrimido[5,4- |
| | d]pyrimidin-4-amine; |
| Example 26 | N-[(1R)-1-(3-bromophenyl)ethyl]-6-(2-methoxyethoxy)-2- |
| | methylpyrimido[5,4-d]pyrimidin-4-amine; |
| Example 27 | N-[(1R)-1-(3-bromophenyl)ethyl]-2-methyl-6-(morpholin-4- |
| | yl)pyrido[3,2-d]pyrimidin-4-amine; |
| Example 28 | N-[(1R)-1-(3-bromophenyl)ethyl]-6-methoxy-2-methylpyrido[3,2- |
| | d]pyrimidin-4-amine; |
| Example 29 | N-[(1R)-1-(3-bromophenyl)ethyl]-6-ethoxy-2-methylpyrido[3,2- |
| | d]pyrimidin-4-amine; |
| Example 30 | N-[(1R)-1-(3-bromophenyl)ethyl]-6-(2-methoxyethoxy)-2- |
| | methylpyrido[3,2-d]pyrimidin-4-amine; |
| Example 31 | N-[(1R)-1-(3-bromophenyl)ethyl]-6-(2-methoxyethoxy)-2- |
| | methylpyrido[3,2-d]pyrimidin-4-amine; |
| Example 32 | N-[(1R)-1-(3-bromophenyl)ethyl]-2-methyl-6-phenoxypyrido[3,2- |
| | d]pyrimidin-4-amine; |
| Example 33 | 6-(benzyloxy)-N-[(1R)-1-(3-bromophenyl)ethyl]-2-methylpyrido[3,2- |
| | d]pyrimidin-4-amine; |
| Example 34 | N-[(1R)-1-(3-bromophenyl)ethyl]-6-fluoro-2-methylpyrido[3,4- |
| | d]pyrimidin-4-amine; |
| Example 35 | N-[1-(5-bromothiophen-2-yl)ethyl]-6-fluoro-2-methylpyrido[3,4- |
| | d]pyrimidin-4-amine; |
| Example 36 | 6-fluoro-2-methyl-N-[1-(5-{2- |
| | [(methylamino)methyl]phenyl}thiophen-2-yl)ethyl]pyrido[3,4- |
| | d]pyrimidin-4-amine; |
| Example 37 | 4-{[(2-methyl-4-{[1-(5-{2-[(methylamino)methyl]phenyl}thiophen-2- |
| | yl)ethyl]amino}pyrido[3,4-d]pyrimidin-6-yl)oxy]methyl}piperidin-2- |
| | one; |
| Example 38 | 1-{4-[(2-methyl-4-{[1-(5-{2-[(methylamino)methyl]phenyl}thiophen- |
| | 2-yl)ethyl]amino}pyrido[3,4-d]pyrimidin-6-yl)oxy]phenyl}pyrrolidin- |
| | 2-one; |
| Example 39 | N4-[(1R)-1-(3-bromophenyl)ethyl]-2-methyl-N6-[2-(morpholin-4- |
| | yl)ethyl]pyrido[3,4-d]pyrimidine-4,6-diamine; |
| Example 40 | 3-[(4-{[(1R)-1-(3-bromophenyl)ethyl]amino}-2-methylpyrido[3,4- |
| | d]pyrimidin-6-yl)oxy]phenol; |
| Example 41 | N-[(1R)-1-(3-bromophenyl)ethyl]-2-methyl-6-[3-(1-methyl-4,5- |
| | dihydro-1H-imidazol-2-yl)phenoxy]pyrido[3,4-d]pyrimidin-4-amine; |
| Example 42 | N-[1-(4-{[(1R)-1-(3-bromophenyl)ethyl]amino}-2-methylpyrido[3,4- |
| | d]pyrimidin-6-yl)pyrrolidin-3-yl]acetamide; |
| Example 43 | N-[(1R)-1-(3-bromophenyl)ethyl]-2-methyl-6-[4-(pyridin-3- |
| | ylmethyl)piperazin-1-yl]pyrido[3,4-d]pyrimidin-4-amine; |
| Example 44 | N-{2-[(4-{[(1R)-1-(3-bromophenyl)ethyl]amino}-2- |
| | methylpyrido[3,4-d]pyrimidin-6-yl)amino]ethyl}acetamide; |
| Example 45 | 4-(4-{[(1R)-1-(3-bromophenyl)ethyl]amino}-2-methylpyrido[3,4- |
| | d]pyrimidin-6-yl)piperazin-2-one; |
| Example 46 | N4-[(1R)-1-(3-bromophenyl)ethyl]-2-methyl-N6-(tetrahydro-2H- |
| | pyran-4-yl)pyrido[3,4-d]pyrimidine-4,6-diamine; |
| Example 47 | N-[1-(4-{[1-(5-{2-[(dimethylamino)methyl]phenyl}thiophen-2- |
| | yl)ethyl]amino}-2-methylpyrido[3,4-d]pyrimidin-6-yl)pyrrolidin-3- |
| | yl]acetamide; |
| Example 48 | N4-[1-(5-{2-[(dimethylamino)methyl]phenyl}-2-thienyl)ethyl]-2- |
| | methyl-N6-[2-(morpholin-4-yl)ethyl]pyrido[3,4-d]pyrimidine-4,6- |
| | diamine; |
| Example 49 | N4-[(1R)-1-(3-bromophenyl)ethyl]-2-methyl-N6-[2-(1H-pyrazol-1- |
| | yl)ethyl]pyrido[3,4-d]pyrimidine-4,6-diamine; |
| Example 50 | 4-(4-{[1-(5-{2-[(dimethylamino)methyl]phenyl}thiophen-2- |
| | yl)ethyl]amino}-2-methylpyrido[3,4-d]pyrimidin-6-yl)piperazin-2- |
| | one; |
| Example 51 | N-{2-[(4-{[1-(5-{2-[(dimethylamino)methyl]phenyl}thiophen-2- |
| | yl)ethyl]amino}-2-methylpyrido[3,4-d]pyrimidin-6- |
| | yl)amino]ethyl}acetamide; |
| Example 52 | 3-[(4-{[1-(5-{2-[(dimethylamino)methyl]phenyl}thiophen-2- |
| | yl)ethyl]amino}-2-methylpyrido[3,4-d]pyrimidin-6-yl)oxy]phenol; |
| Example 53 | N4-[1-(5-{2-[(dimethylamino)methyl]phenyl}-2-thienyl)ethyl]-2- |
| | methyl-N6-(tetrahydro-2H-pyran-4-yl)pyrido[3,4-d]pyrimidine-4,6- |
| | diamine; |
| Example 54 | N4-[1-(5-{2-[(dimethylamino)methyl]phenyl}-2-thienyl)ethyl]-2- |
| | methyl-N6-[2-(1H-pyrazol-1-yl)ethyl]pyrido[3,4-d]pyrimidine-4,6- |
| | diamine; |
| Example 55 | N-[1-(5-{2-[(dimethylamino)methyl]phenyl}thiophen-2-yl)ethyl]-2- |
| | methyl-6-[4-(pyridin-3-ylmethyl)piperazin-1-yl]pyrido[3,4- |
| | d]pyrimidin-4-amine; |
| Example 56 | N4-[1-(5-{2-[(dimethylamino)methyl]phenyl}-2-thienyl)ethyl]-N6-[2- |
| | (1H-imidazol-1-yl)ethyl]-2-methylpyrido[3,4-d]pyrimidine-4,6- |
| | diamine; |
| Example 57 | N-[(1R)-1-(3-bromophenyl)ethyl]-6-methoxy-2-methylpyrido[3,4- |
| | d]pyrimidin-4-amine; |
| Example 58 | N-[1-(5-{2-[(dimethylamino)methyl]phenyl}thiophen-2-yl)ethyl]-2- |
| | methyl-6-[3-(1-methyl-4,5-dihydro-1H-imidazol-2- |
| | yl)phenoxy]pyrido[3,4-d]pyrimidin-4-amine; |
| Example 59 | N-[1-(5-{2-[(dimethylamino)methyl]phenyl}thiophen-2-yl)ethyl]-2- |
| | methyl-6-(methylsulfanyl)pyrido[3,4-d]pyrimidin-4-amine; |
| Example 60 | N-[(3R)-1-(4-{[1-(5-{2-[(dimethylamino)methyl]phenyl}thiophen-2- |
| | yl)ethyl]amino}-2-methylpyrido[3,4-d]pyrimidin-6-yl)pyrrolidin-3- |
| | yl]acetamide; |
| Example 61 | N-[(3S)-1-(4-{[(1S)-1-(5-{2- |
| | [(dimethylamino)methyl]phenyl}thiophen-2-yl)ethyl]amino}-2- |
| | methylpyrido[3,4-d]pyrimidin-6-yl)pyrrolidin-3-yl]acetamide; |
| Example 62 | N-{2-[(4-{[(1R)-1-(3-bromophenyl)ethyl]amino}-2- |
| | methylpyrido[3,4-d]pyrimidin-6-yl)oxy]ethyl}acetamide; |
| Example 63 | N-[1-(5-{2-[(dimethylamino)methyl]phenyl}thiophen-2-yl)ethyl]-6- |
| | ethoxy-2-methylpyrido[3,4-d]pyrimidin-4-amine; |
| Example 64 | N-[1-(5-{2-[(dimethylamino)methyl]phenyl}thiophen-2-yl)ethyl]-6- |
| | methoxy-2-methylpyrido[3,4-d]pyrimidin-4-amine; |
| Example 65 | N-[1-(5-{2-[(dimethylamino)methyl]phenyl}thiophen-2-yl)ethyl]-2- |
| | methyl-6-propoxypyrido[3,4-d]pyrimidin-4-amine; |
| Example 66 | 6-[2-(dimethylamino)ethoxy]-N-[1-(5-{2-[(dimethylamino)methyl]- |
| | phenyl}thiophen-2-yl)ethyl]-2-methylpyrido[3,4-d]pyrimidin-4- |
| | amine; |
| Example 67 | N-[1-(5-{2-[(dimethylamino)methyl]phenyl}thiophen-2-yl)ethyl]-6- |
| | [(1,1-dioxidotetrahydro-2H-thiopyran-4-yl)oxy]-2-methylpyrido[3,4- |
| | d]pyrimidin-4-amine; |
| Example 68 | [2-(5-{1-[(6-fluoro-2-methylpyrido[3,4-d]pyrimidin-4- |
| | yl)amino]ethyl}thiophen-2-yl)phenyl]methanol; |
| Example 69 | 3-(4-{[1-(5-{2-[(dimethylamino)methyl]phenyl}thiophen-2- |
| | yl)ethyl]amino}-2-methylpyrido[3,4-d]pyrimidin-6-yl)-1- |
| | methylpyrrolidin-2-one; |
| Example 70 | N-[(1R)-1-(3-bromophenyl)ethyl]-6-[3-(dimethylamino)propoxy]-2- |
| | methylpyrido[3,4-d]pyrimidin-4-amine; |
| Example 71 | 6-(azetidin-1-yl)-N-[1-(5-{2- |
| | [(dimethylamino)methyl]phenyl}thiophen-2-yl)ethyl]-2- |
| | methylpyrido[3,4-d]pyrimidin-4-amine; |
| Example 72 | N-[1-(5-{2-[(dimethylamino)methyl]phenyl}thiophen-2-yl)ethyl]-6- |
| | [3-(dimethylamino)propoxy]-2-methylpyrido[3,4-d]pyrimidin-4- |
| | amine; |
| Example 73 | 6-(cyclopropylmethoxy)-N-[1-(5-{2- |
| | [(dimethylamino)methyl]phenyl}thiophen-2-yl)ethyl]-2- |
| | methylpyrido[3,4-d]pyrimidin-4-amine; |
| Example 74 | 2-[(4-{[1-(5-{2-[(dimethylamino)methyl]phenyl}thiophen-2- |
| | yl)ethyl]amino}-2-methylpyrido[3,4-d]pyrimidin-6-yl)oxy]ethanol; |
| Example 75 | N-[(1R)-1-(3-bromophenyl)ethyl]-6-(cyclopropylmethoxy)-2- |
| | methylpyrido[3,4-d]pyrimidin-4-amine; |
| Example 76 | 3-[(4-{[1-(5-{2-[(dimethylamino)methyl]phenyl}thiophen-2- |
| | yl)ethyl]amino}-2-methylpyrido[3,4-d]pyrimidin-6-yl)oxy]propan-1- |
| | ol; |
| Example 77 | N-[1-(5-{2-[(dimethylamino)methyl]phenyl}-2-thienyl)ethyl]-6-[(1- |
| | imino-1-oxidohexahydro-1lambda4-thiopyran-4-yl)oxy]-2- |
| | methylpyrido[3,4-d]pyrimidin-4-amine; |
| Example 78 | N-[(1R)-1-(3-bromophenyl)ethyl]-6-[2-(dimethylamino)ethoxy]-2- |
| | methylpyrido[3,4-d]pyrimidin-4-amine; |
| Example 79 | N-[(3R)-1-(4-{[1-(5-{2-[(dimethylamino)methyl]phenyl}thiophen-2- |
| | yl)ethyl]amino}-2-methylpyrido[3,4-d]pyrimidin-6-yl)pyrrolidin-3- |
| | yl]acetamide (diastereomer 1); |
| Example 80 | N-[(3R)-1-(4-{[1-(5-{2-[(dimethylamino)methyl]phenyl}thiophen-2- |
| | yl)ethyl]amino}-2-methylpyrido[3,4-d]pyrimidin-6-yl)pyrrolidin-3- |
| | yl]acetamide (diastereomer 2); |
| Example 81 | N-[(3S)-1-(4-{[1-(5-{2-[(dimethylamino)methyl]phenyl}thiophen-2- |
| | yl)ethyl]amino}-2-methylpyrido[3,4-d]pyrimidin-6-yl)pyrrolidin-3- |
| | yl]acetamide (diastereomer 1); |
| Example 82 | N-[(3S)-1-(4-{[1-(5-{2-[(dimethylamino)methyl]phenyl}thiophen-2- |
| | yl)ethyl]amino}-2-methylpyrido[3,4-d]pyrimidin-6-yl)pyrrolidin-3- |
| | yl]acetamide (diastereomer 2); |
| Example 83 | N4-[(1R)-1-(3-bromophenyl)ethyl]-2-methyl-N6-[2- |
| | (methylsulfonyl)ethyl]pyrido[3,4-d]pyrimidine-4,6-diamine; |
| Example 84 | N4-[(1R)-1-(3-bromophenyl)ethyl]-N6-[2-(1H-imidazol-1-yl)ethyl]- |
| | 2-methylpyrido[3,4-d]pyrimidine-4,6-diamine; |
| Example 85 | 3-(4-{[1-(5-{2-[(dimethylamino)methyl]phenyl}thiophen-2-yl)ethyl]- |
| | amino}-2-methylpyrido[3,4-d]pyrimidin-6-yl)-3-hydroxy-1- |
| | methylpyrrolidin-2-one; |
| Example 86 | 4-{[(4-{[1-(5-{2-[(dimethylamino)methyl]phenyl}thiophen-2- |
| | yl)ethyl]amino}-2-methylpyrido[3,4-d]pyrimidin-6- |
| | yl)amino]methyl}piperidin-2-one; |
| Example 87 | N-[1-(4-{[1-(5-{2-[(dimethylamino)methyl]phenyl}thiophen-2- |
| | yl)ethyl]amino}-2-methylpyrido[3,4-d]pyrimidin-6-yl)azetidin-3-yl]- |
| | 4-[4,5-dimethyl-2-(pyridin-2-yl)-1H-imidazol-1-yl]butanamide; |
| Example 88 | N-[1-(4-{[1-(5-{2-[(dimethylamino)methyl]phenyl}thiophen-2- |
| | yl)ethyl]amino}-2-methylpyrido[3,4-d]pyrimidin-6-yl)azetidin-3- |
| | yl]benzamide; |
| Example 89 | N-[1-(4-{[1-(5-{2-[(dimethylamino)methyl]phenyl}thiophen-2- |
| | yl)ethyl]amino}-2-methylpyrido[3,4-d]pyrimidin-6-yl)azetidin-3-yl]- |
| | 2-phenylacetamide; |
| Example 90 | N-[1-(4-{[1-(5-{2-[(dimethylamino)methyl]phenyl}thiophen-2- |
| | yl)ethyl]amino}-2-methylpyrido[3,4-d]pyrimidin-6-yl)azetidin-3- |
| | yl]cyclohexanecarboxamide; |
| Example 91 | 2-cyclopropyl-N-[1-(4-{[1-(5-{2- |
| | [(dimethylamino)methyl]phenyl}thiophen-2-yl)ethyl]amino}-2- |
| | methylpyrido[3,4-d]pyrimidin-6-yl)azetidin-3-yl]acetamide; |
| Example 92 | N-[1-(4-{[1-(5-{2-[(dimethylamino)methyl]phenyl}thiophen-2- |
| | yl)ethyl]amino}-2-methylpyrido[3,4-d]pyrimidin-6-yl)azetidin-3-yl]- |
| | 2-(morpholin-4-yl)acetamide; |
| Example 93 | 6-(3-aminoazetidin-1-yl)-N-[1-(5-{2- |
| | [(dimethylamino)methyl]phenyl}thiophen-2-yl)ethyl]-2- |
| | methylpyrido[3,4-d]pyrimidin-4-amine; |
| Example 94 | N-[1-(4-{[1-(5-{2-[(dimethylamino)methyl]phenyl}thiophen-2- |
| | yl)ethyl]amino}-2-methylpyrido[3,4-d]pyrimidin-6-yl)azetidin-3- |
| | yl]benzenesulfonamide; |
| Example 95 | N-[1-(4-{[1-(5-{2-[(dimethylamino)methyl]phenyl}thiophen-2- |
| | yl)ethyl]amino}-2-methylpyrido[3,4-d]pyrimidin-6-yl)azetidin-3-yl]- |
| | 1-phenylmethanesulfonamide; |
| Example 96 | 1-[1-(4-{[1-(5-{2-[(dimethylamino)methyl]phenyl}thiophen-2- |
| | yl)ethyl]amino}-2-methylpyrido[3,4-d]pyrimidin-6-yl)azetidin-3-yl]- |
| | 3-phenylurea; |
| Example 97 | tert-butyl (3R)-3-[acetyl(3-{[1-(4-{[1-(5-{2- |
| | [(dimethylamino)methyl]phenyl}-2-thienyl)ethyl]amino}-2- |
| | methylpyrido[3,4-d]pyrimidin-6-yl)azetidin-3-yl]carbamoyl}- |
| | phenyl)amino]pyrrolidine-1-carboxylate; |
| Example 98 | tert-butyl 4-[acetyl(3-{[1-(4-{[1-(5-{2- |
| | [(dimethylamino)methyl]phenyl}thiophen-2-yl)ethyl]amino}-2- |
| | methylpyrido[3,4-d]pyrimidin-6-yl)azetidin-3-yl]carbamoyl}- |
| | phenyl)amino]piperidine-1-carboxylate; |
| Example 99 | 6-[3-(benzylamino)azetidin-1-yl]-N-[1-(5-{2-[(dimethylamino)- |
| | methyl]phenyl}thiophen-2-yl)ethyl]-2-methylpyrido[3,4-d]pyrimidin- |
| | 4-amine; and |
| Example 100 | N-[1-(4-{[1-(5-{2-[(dimethylamino)methyl]phenyl}thiophen-2- |
| | yl)ethyl]amino}-2-methylpyrido[3,4-d]pyrimidin-6-yl)azetidin-3- |
| | yl]thiomorpholine-4-carboxamide 1,1-dioxide; |

or a stereoisomer, a tautomer, an N-oxide, a hydrate, a solvate, or a salt thereof, or a mixture of the foregoing.

18. The compound according to claim 1 wherein the carbon atom between the nitrogen atom and the substituent A1 is in R-configuration.

19. The compound according to claim 1, wherein R² is selected from the group consisting of hydrogen, hydroxyl, oxo (═O), cyanocyclopropyl, 1,1-dimethylcyclopropyl, —C(═CH₂)CH₃, —C(CH₃)═CHCH₃, —CH═CH—(CH₂)₂CH₃, CH═CHCH₃, —CH═CH-cyclopropyl, —C(O)NH₂, C(O)OCH₃, —S(O)₂CH₃, —OCH₃, —CH₂NH₂, and a halogen atom.

20. The compound according to claim 1, wherein R³ is a C₁- or C₂-alkyl substituted with an amino group —NR^{k}R^{l}, wherein R^{k} and R^{l} have the meanings as defined in claim 1.
